(19) United States
(12) Patent Application Publication
(10) Pub. No.: US 2025/0270627 A1
(43) Pub. Date: Aug. 28, 2025

(54) Genotyping Methods and Systems

(71) Applicant: Illumina, Inc., San Diego, CA (US)

(72) Inventors: Andrew OSTROW, San Diego, CA (US); Bret BARNES, San Diego, CA (US); Thu NGUYEN, San Diego, CA (US); Karen SHANNON, San Diego, CA (US); Carey DAVIS, San Diego, CA (US); Brian CHIN, San Diego, CA (US); Sarah HANSON, San Diego, CA (US)

(21) Appl. No.: 18/702,060

(22) PCT Filed: Oct. 22, 2021
(86) PCT No.: PCT/US2021/056305

§ 371 Date: Apr. 17, 2024

(51) Int. Cl. C12Q 1/6827; C12Q 1/6806; C12Q 1/686; C12Q 1/6876

(52) U.S. Cl. CPC C12Q 1/6827; C12Q 1/6806; C12Q 1/686; C12Q 1/6876; C12Q 2600/106; C12Q 2600/156

(57) ABSTRACT

Provided herein are compositions and methods related to probe-based genotyping, such as pharmacogenomic genotyping.

### CROSS REFERENCE TO RELATED APPLICATION

This application is a US 371 National Stage Application from PCT/2021/056305 filed Oct. 22, 2021, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 29, 2021, is named 35629-0037WO1_SL.txt and is 652,900 bytes in size.

### TECHNICAL FIELD

Provided herein are compositions and methods related to probe based genotyping methods and systems of, for example, high homology regions such as PGx genes.

### BACKGROUND

The study of genetically influenced variations in drug response and adverse drug reactions (ADRs) is known as pharmacogenomics (PGx). See, e.g., Arbitrio et al., “Pharmacogenomic Profiling of ADME Gene Variants: Current Challenges and Validation Perspectives,” High Throughput 7 (4): 40 (2018). Polymorphic variants in genes related to drug absorption, distribution, metabolism, and excretion (ADME) contribute to variability amongst individuals in drug efficacy and adverse effects. Id. Identification of PGx markers, therefore, enables optimization of treatment based on an individual's genotype. However, pharmacogenomic marker genotyping is complicated by high homozygosity.

### SUMMARY

The present application is based, at least in part, on the discovery that probe based genotyping of high homology regions, such as PGx genes, is improved, for example, by combining targeted enrichment with whole-genome amplification.

Thus, described herein are methods of genotyping one or more pharmacogenomic markers, including: obtaining a nucleic acid sample; amplifying a first portion of the genomic DNA sample by whole genome amplification (WGA), thereby producing a WGA sample portion; amplifying a second portion of the genomic DNA sample by a targeted gene amplification (TGA) method that selectively amplifies one or more pharmacogenomic genes or fragments thereof, thereby producing a TGA sample portion; optionally combining the whole genome amplified sample portion and the target amplified sample portion to produce a combined WGA/TGA sample portion; fragmenting the WGA or WGA/TGA sample; hybridizing the WGA and TGA samples or the WGA/TGA combined sample to a plurality of probes complementary to one or more of the pharmacogenomic genes or fragments thereof; and detecting hybridization, thereby genotyping a pharmacogenomic marker.

In some embodiments, the nucleic acid sample is a genomic DNA sample is from a human subject.

In some embodiments, the one or more pharmacogenomic genes are selected from the group consisting of ABCB1, ABCC4, ABCG2, ACE, ALDH1A1, ALK, BCKDK, BCR, BRAF, C8orf34, CACNA1S, CES1, CFTR, CHRNA3, COL22A1, COQ2, CRHR1, CYP19A1, CYP21A2, CYP2A6, CYP2A7P1, CYP2B6, CYP2C19, CYP2C8, CYP2C9, CYP2D6, CYP2E1, CYP3A, CYP3A5, CYP4F2, DPYD, DYNC2H1, EGFR, EPHX1, ERBB2, F5, FCGR3A, FKBP5, G6PD, GGCX, GSTM1, GSTT1, HCP5, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PRSS53, PSORS1C1, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT1A5, UGT2B17, UGT2B28, VDR, VKORC1, YEATS4, ZNRD1-AS1, and combinations thereof.

In some embodiments, the one or more pharmacogenomic genes are selected from the group consisting of BCKDK, CACNA1S, CFTR, CYP2A7P1, CYP2B6, CYP2B6, CYP2C19, CYP2C9, CYP2D6, CYP3A, CYP3A5, CYP4F2, DPYD, F5, G6PD, HCP5, HLA-A, IFNL3, NUDT15, PRSS53, PSORS1C1, RYR1, SLCO1B1, TPMT, UGT1A1, VKORC1, ZNRD1-AS1, and combinations thereof.

In some embodiments, the one or more pharmacogenomic genes are selected from the group consisting of ABCB1, ABCC4, ABCG2, ACE, ALDH1A1, ALK, BCR, BRAF, C8orf34, CACNA1S, CES1, CFTR, CHRNA3, COL22A1, COQ2, CRHR1, CYP19A1, CYP21A2, CYP2A6, CYP2B6, CYP2C19, CYP2C8, CYP2C9, CYP2D6, CYP2E1, CYP3A5, CYP4F2, DPYD, DYNC2H1, EGFR, EPHX1, ERBB2, F5, FCGR3A, FKBP5, G6PD, GGCX, GSTM1, GSTT1, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT2B17, UGT2B28, VDR, VKORC1, YEATS4, and combinations thereof.

In some embodiments, the one or more pharmacogenomic markers are selected from those in Table 9.

In some embodiments, the one or more pharmacogenomic markers are copy number variants in ABCB1, ABCC4, ABCG2, ACE, ALDH1A1, ALK, BCR, BRAF, C8orf34, CACNA1S, CES1, CFTR, CHRNA3, COL22A1, COQ2, CRHR1, CYP19A1, CYP21A2, CYP2A6, CYP2B6, CYP2C19, CYP2C8, CYP2C9, CYP2D6, CYP2E1, CYP3A5, CYP4F2, DPYD, DYNC2H1, EGFR, EPHX1, ERBB2, F5, FCGR3A, FKBP5, G6PD, GGCX, GSTM1, GSTT1, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT2B17, UGT2B28, VDR, VKORC1, YEATS4, and combinations thereof.

In some embodiments, the one or more pharmacogenomic markers are selected from the group consisting of those in Table 9, copy number variants in ABCB1, ABCC4, ABCG2, ACE, ALDH1A1, ALK, BCR, BRAF, C8orf34, CACNA1S, CES1, CFTR, CHRNA3, COL22A1, COQ2, CRHR1, CYP19A1, CYP21A2, CYP2A6, CYP2B6, CYP2C19, CYP2C8, CYP2C9, CYP2D6, CYP2E1, CYP3A5, CYP4F2, DPYD, DYNC2H1, EGFR, EPHX1, ERBB2, F5, FCGR3A, FKBP5, G6PD, GGCX, GSTM1, GSTT1, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT2B17, UGT2B28, VDR, VKORC1, YEATS4, and combinations thereof.

In some embodiments, amplifying a second portion of the genomic DNA sample comprises amplifying one or more regions of one or more pharmacogenomic genes or fragments thereof that differ in their nucleotide sequence from one or more pseudogenes of the one or more pharmacogenomic genes.

In some embodiments, the one or more regions of the one or more pharmacogenomic genes or fragments thereof share at least 99%, 98%, 97%, 96%, 95%, 94%, 93%, 92%, 91%, or 90% identity in its nucleic acid sequence with the corresponding one or more pseudogenes or fragments thereof.

In some embodiments, detecting hybridization comprises single-base extension (SBE), allele-specific primer extension (ASPE), or both SBE and ASPE.

In some embodiments, amplifying comprises a PCR reaction with a dNTP mixture comprising dATP, dTTP, dGTP, dCTP, and dUTP and a dUTP incorporating polymerase.

In some embodiments, fragmenting comprises incubating with uracil DNA glycosylase (UDG).

Also described herein are methods of selecting a drug treatment for a patient in need thereof, including: identifying a patient in need of a drug treatment; determining, or having determined, the genotype of pharmacogenomic marker(s) according to any of the methods described herein; and based on said genotyping, selecting a drug treatment for the patient.

In some embodiments, selecting a drug treatment for the patient comprises determining that a drug is suitable for administration to the patient by identifying one or more drug interactions with the genotype of one or more the pharmacogenomic markers, and, optionally, administering a drug with a positive treatment outcome associated with one or more of the genotypes of the one or more pharmacogenomic markers and/or not administering a drug with a negative treatment outcome associated with one or more of the genotypes of the one or more pharmacogenomic markers.

In some embodiments, selecting a drug treatment includes determining that a drug is not suitable for administration to the patient and, optionally, not administering the drug.

Also described herein are array compositions comprising: a solid surface; and one or more nucleic acids selected from the group consisting of nucleic acids comprising or consisting of the sequences of SEQ ID NOS: 22-2217, wherein the one or more nucleic acids are bound to the solid surface.

In some embodiments, the array composition comprises: (i) at least 100 nucleic acids selected from the group consisting of nucleic acids comprising or consisting of the sequences of SEQ ID NOS: 22-2217; or (ii) at least 500 nucleic acids selected from the group consisting of nucleic acids comprising or consisting of the sequences of SEQ ID NOS: 22-2217; (iii) at least 1000 nucleic acids selected from the group consisting of nucleic acids comprising or consisting of the sequences of SEQ ID NOS: 22-2217; or (iv) at least 1500 nucleic acids selected from the group consisting of nucleic acids comprising or consisting of the sequences of SEQ ID NOS: 22-2217.

Also described herein are methods of amplifying a target nucleic acid by polymerase chain reaction comprising contacting the target nucleic acid with a composition comprising one or more nucleic acids selected from the group of nucleic acids comprising or consisting of SEQ ID NOS: 4-19 and a polymerase.

In some embodiments, the composition comprises nucleic acids comprising or consisting of: (i) SEQ ID NO:4 and SEQ ID NO:5; (ii) SEQ ID NO:6 and SEQ ID NO:7; (iii) SEQ ID NO: 8 and SEQ ID NO:9; (iv) SEQ ID NO:10 and SEQ ID NO:11; (v) SEQ ID NO:12 and SEQ ID NO: 13; (vi) SEQ ID NO:14 and SEQ ID NO:15; (vii) SEQ ID NO:16 and SEQ ID NO:17; and/or (viii) SEQ ID NO:18 and SEQ ID NO:19.

In some embodiments, the composition comprises oligonucleotides comprising or consisting of the sequences of SEQ ID NOS: 4, 5, 6, 7, 8, 9, 10, 11, 12, 13, 14, 15, 16, 17, 18, and 19.

Also described herein are compositions comprising one or more nucleic acids selected from the group of nucleic acids comprising or consisting of SEQ ID NOs: 4-19, a polymerase, and a target nucleic acid.

Also described herein are kits comprising a composition comprising nucleic acids comprising or consisting of: (i) SEQ ID NO:4 and SEQ ID NO:5; (ii) SEQ ID NO:6 and SEQ ID NO: 7; (iii) SEQ ID NO:8 and SEQ ID NO:9; (iv) SEQ ID NO:10 and SEQ ID NO:11; (v) SEQ ID NO: 12 and SEQ ID NO:13; (vi) SEQ ID NO: 14 and SEQ ID NO:15; (vii) SEQ ID NO:16 and SEQ ID NO:17; and/or (viii) SEQ ID NO: 18 and SEQ ID NO:19.

In some embodiments, the kit further comprises a dNTP mixture and a polymerase.

In some embodiments, the dNTP mixture comprises dUTP and the polymerase is a dUTP incorporating polymerase.

In some embodiments, the kit further comprises uracil DNA nucleotide glycosylase.

In some embodiments, the kit further comprises an array composition described herein.

In some embodiments, the kit further comprises random oligonucleotides for WGA.

As used herein, the term “pharmacogenomic marker” refers to a genetic variant, such as a single nucleotide variant (SNV) or copy number variant (CNV) in a gene associated with drug metabolism.

As used herein, the term “pharmacogenomic gene” refers to a gene associated with drug metabolism.

As used herein, the term “fragmenting” in the context of fragmenting nucleic acid(s) refers to the process of breaking down a nucleic acid into units of shorter lengths.

As used herein, the term “whole genome amplification” (WGA) refers to a process by which a starting amount of nucleic acid (e.g., genomic DNA) is amplified, with all regions of the genome targeted for amplification. An example of such a process is multi displacement amplification (MDA).

As used herein, the term “targeted gene amplification” (TGA) refers to a process in which particular nucleic acids (e.g., target genomic regions) are isolated and amplified (for example, by PCR).

As used herein, the term “genotyping” refers to determining a genetic constitution of an individual at a genomic locus. For example, genotyping includes determining the genetic constitution of the alleles present a genomic locus (e.g., alleles at a SNV locus) and/or the number of copies of a gene or allele at a genomic locus.

By leveraging pharmacogenomics technologies, healthcare providers will ultimately be able to maximize the intended use of a medication or treatment, reduce adverse drug reactions, speed time to achieving the therapeutic benefit of a drug, decrease the chance of side effects or dependency, and decrease the cost of healthcare expenditures. For example, costs can be reduced by using the methods described herein to identify the most appropriate and affordable drug the first time, by reducing adverse drug reactions early in treatment, and thus reducing hospital length of stays, reducing hospital readmissions, and reducing ER visits.

Throughout this application, various embodiments may be presented in a range format. It should be understood that the description in a range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a flowchart that shows one example of an Infinium® assay workflow that incorporates targeted gene amplification (TGA).

FIG. 2A is a schematic diagram that shows an exemplary Infinium® assay workflow.

FIG. 2B is a flowchart that shows one example of an Infinium® assay workflow.

FIGS. 3A-3C are a series of flow charts that show an overview of a three-day workflow for PGx genotyping using the Infinium® Global Diversity Array with Enhanced PGx. Day 1: FIG. 3A; Day 2: FIG. 3B; Day 3: FIG. 3C.

FIGS. 4A and 4B are graphic representations of results for design verification testing runs of Infinium® with PGx content enabled. FIG. 4A: Call Rate passes acceptance criteria of ≥99.5% of probes receiving a call indicating high quality and reproducible data generated from the PGx with TGA workflow; FIG. 4B: Log R Dev, a metric comparing test signal to reference signal, passes acceptance criteria of >0.200, indicating low noise in the data.

FIG. 5 is a bar graph showing concordance of PGx variants covered by the PCR module in the Infinium® workflow with PGx content enabled.

FIG. 6 is a series of plots showing concordance of regions of CYP2D6 with and without amplicon coverage. In the top row, signal from WGA alone is shown for regions of CYP2D6 containing key PGx variants, and colored by the genotype taken from 1000 Genomes reference data. Overlap of genotype clusters results from interference from pseudogenes generating noise in these regions, leading to inaccurate genotyping calls. Noise shifts cluster over one another, yielding a suppressed Call Rate of 80.1%. In the bottom row, TGA is included in the workflow as described herein, and amplicons from the regions are present in large molar excess over background WGA DNA. This approach leads to genotyping of the on-target amplicons, and minimization of signal from WGA, yielding distinct clusters and a high Call Rate of 99.3%.

FIGS. 7A and 7B are graphs that show that suboptimal NaOH levels decrease CNV (high % GC) probe intensity. The two graphs show normalized signal intensity (FIG. 7A) and Log R Mean v. NaOH molarity (FIG. 7B) for low [OH] v. High [OH] for 1399 CYP2D6 probes.

FIGS. 8A and 8B are a pair of graphs that show higher [OH] increases signal stability, leading to guard-banded CNV calling capability for the CYP2D6 SNP caller. Nominal signal intensity (FIG. 8A; dashed lines, from top to bottom: 95%, 90%, 85%; bars, from left to right, CYP2D6.intron.2_acc, CYP2D6.p5_acc, CYP2D6.exon.9_acc) and Log R Mean (FIG. 8B).

FIG. 9 is a plot of results from using a probe that shows high levels of noise in the control (WGA only, Control) evidenced by a theta value far from any canonical cluster (0, 0.5, or 1), is inhibited in the presence of multiplex PCR-amplified material that does not contain uracil (WGA+mPCR, “mPCR”), and yields an AB call at the canonical cluster position in the presence of uracil-containing multiplex PCR (mUPCR+WGA, “mUPCR”), demonstrating rescue of an inhibited probe via uracil incorporation of target amplicons during PCR and subsequent fragmentation.

### DETAILED DESCRIPTION

Provided herein are methods and compositions for genotyping. The methods and compositions described herein are useful, for example, for determining the genotype of a pharmacogene (PGx gene) (see, e.g., Katara and Yadav, “Pharmacogenes (PGx-genes): Current Understanding and Future Directions,” Gene 718:144050 (2019)) and, for example, selecting a treatment based on the genotype.

The methods described herein include, for example, genotyping one or more pharmacogenomic markers, which are described in more detail below. In some cases, the genotyping includes a target enrichment step, which is also described in more detail below. In some cases, the target enrichment includes PCR amplification that incorporates dUTP into the amplification product, and, in, thus, in some cases, fragmentation includes Uracil-DNA Glycosylase (UDG) digestion.

PGx marker genotypes are useful, for example, for selecting treatments suitable for administration (e.g., which are expected to be well-tolerated based on genotype). Thus, also described herein are methods for selecting drug treatment(s) for patient(s).

The compositions described herein include, for example, compositions comprising PCR primers for targeted enrichment and/or whole genome amplification, as well as probes for capture of, for example, PGx genes, and arrays to which the probes are bound. The compositions, in some cases, are provided as part of a kit.

### Pharmacogenomic Markers

The study of genetically influenced variations in drug response and adverse drug reactions (ADRs) is known as pharmacogenomics (PGx). See, e.g., Arbitrio et al., “Pharmacogenomic Profiling of ADME Gene Variants: Current Challenges and Validation Perspectives,” High Throughput 7 (4): 40 (2018). Polymorphic variants in genes related to drug absorption, distribution, metabolism, and excretion (ADME) contribute to variability amongst individuals in drug efficacy and adverse effects. Id. Identification of PGx markers, therefore, enables optimization of treatment based on an individual's genotype.

Genetic variants of PGx genes can be referred to at the allele level, for example by star allele nomenclature (see, e.g., Kalman et al., “Pharmacogenetic Allele Nomenclature: International Workgroup Recommendations for Test Result Reporting, Clin. Pharmacol. Ther. 99 (2): 172-85 (2016)) and/or Human Genome Variation Society (HGVS) nomenclature (see den Dunnen et al., “HGVS Recommendations for the Description of Sequence Variants: 2016 Update,” Human Mutation, 37 (6): 654-9 (2016)). Genetic variants of PGx genes can also be referred to at the nucleotide level, for example by HGVS nomenclature. A variant allele can comprise a series of variants at the nucleotide level.

Thus, in some instances, the pharmacogenomics marker is an allele of a PGx gene. In some instances, the pharmacogenomics marker is a mutation in a PGx gene at the nucleic and/or amino acid level, relative to a reference sequence. In some instances, the reference sequence is a reference genome. In some instances, the reference sequence is a wild-type allele.

In some instances, the mutation is selected from the group consisting of a substitution, a deletion, an insertion, a duplication, an inversion, a deletion-insertion, a repeat, and combinations thereof. In some instances, the mutation is a single nucleotide variant (SNV) (e.g., a single base substitution). In some instances, the mutation is a copy number variant (CNV).

In some instances, the pharmacogenomics gene is selected from the group consisting of ABCB1, ABCC4, ABCG2, ACE, ALDH1A1, ALK, BCKDK, BCR, BRAF, C8orf34, CACNA1S, CES1, CFTR, CHRNA3, COL22A1, COQ2, CRHR1, CYP19A1, CYP21A2, CYP2A6, CYP2A7P1, CYP2B6, CYP2C19, CYP2C8, CYP2C9, CYP2D6, CYP2E1, CYP3A, CYP3A5, CYP4F2, DPYD, DYNC2H1, EGFR, EPHX1, ERBB2, F5, FCGR3A, FKBP5, G6PD, GGCX, GSTM1, GSTT1, HCP5, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PRSS53, PSORS1C1, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT1A5, UGT2B17, UGT2B28, VDR, VKORC1, YEATS4, ZNRD1-AS1, and combinations thereof.

In some instances, the pharmacogenomics gene is selected from the group consisting of BCKDK, CACNA1S, CFTR, CYP2A7P1, CYP2B6, CYP2B6, CYP2C19, CYP2C9, CYP2D6, CYP3A, CYP3A5, CYP4F2, DPYD, F5, G6PD, HCP5, HLA-A, IFNL3, NUDT15, PRSS53, PSORS1C1, RYR1, SLCO1B1, TPMT, UGT1A1, VKORC1, ZNRD1-AS1, and combinations thereof, and the pharmacogenomics marker is a SNV. Representative SNV pharmacogenomics markers are set forth in Table 9, below.

In some instances, the pharmacogenomics is selected from the group consisting of ABCB1, ABCC4, ABCG2, ACE, ALDH1A1, ALK, BCR, BRAF, C8orf34, CACNA1S, CES1, CFTR, CHRNA3, COL22A1, COQ2, CRHR1, CYP19A1, CYP21A2, CYP2A6, CYP2B6, CYP2C19, CYP2C8, CYP2C9, CYP2D6, CYP2E1, CYP3A5, CYP4F2, DPYD, DYNC2H1, EGFR, EPHX1, ERBB2, F5, FCGR3A, FKBP5, G6PD, GGCX, GSTM1, GSTT1, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT2B17, UGT2B28, VDR, VKORC1, YEATS4, and combinations thereof, and the pharmacogenomic marker is a copy number variation (CNV).

### Genotyping Pharmacogenomic Markers

### Target Enrichment

The methods described herein can include enrichment, e.g., of a pharmacogenomic marker. In some instances, enrichment comprises targeted amplification of a high homology region, for example, a PGx gene or fragment thereof.

In some instances, the PGx gene is selected from the group consisting of CYP2D6 (NCBI Gene ID: 1565; SEQ ID NO: 1), CYP2B6 (NCBI Gene ID: 1555; SEQ ID NO: 2), and TPMT (NCBI Gene ID: 7172; SEQ ID NO: 3), and combinations thereof.

In some instances, the enrichment comprises targeted amplification of one or more particular exon(s) of a PGx gene or fragment thereof.

In some instances, the particular exon(s) of the PGx gene or fragment thereof is selected from the group consisting of CYP2D6 Exon 1 (E1), CYP2D6 Exon 2 (E2), CYP2D6 Exon 3 (E3), CYP2D6 Exon 4 (E4), CYP2D6 Exon 5 (E5), CYP2D6 Exon 6 (E6), CYP2D6 Exon 7 (E7), CYP2D6 Exon 8 (E8), CYP2D6 Exon 8 (E9), CYP2B6 Exon 1 (E1), CYP2B6 Exon 2 (E2), CYP2B6 Exon 3 (E3), CYP2B6 Exon 4 (E4), CYP2B6 Exon 5 (E5), CYP2B6 Exon 6 (E6), CYP2B6 Exon 7 (E7), CYP2B6 Exon 8 (E8), CYP2B6 Exon 8 (E9), and combinations thereof.

The locations of the exons for CYP2D6 and CYP2B6, with respect to SEQ ID NO: 1 and SEQ ID NO: 2, respectively are shown below in the sequences section, are indicated in Table 1.

TABLE 1
PGx Gene Exons

| Gene | Exon | Location |
|---|---|---|
| CYP2D6 | 1 | 1 . . . 199 |
| CYP2D6 | 2 | 902 . . . 1073 |
| CYP2D6 | 3 | 1626 . . . 1778 |
| CYP2D6 | 4 | 1867 . . . 2027 |
| CYP2D6 | 5 | 2461 . . . 2637 |
| CYP2D6 | 6 | 2828 . . . 2969 |
| CYP2D6 | 7 | 3177 . . . 3364 |
| CYP2D6 | 8 | 3819 . . . 3960 |
| CYP2D6 | 9 | 4059 . . . 4312 |
| CYP2B6 | 1 | 1 . . . 195 |
| CYP2B6 | 2 | 12720 . . . 12882 |
| CYP2B6 | 3 | 13016 . . . 13165 |
| CYP2B6 | 4 | 15624 . . . 15784 |
| CYP2B6 | 5 | 17938 . . . 18114 |
| CYP2B6 | 6 | 18713 . . . 18854 |
| CYP2B6 | 7 | 21017 . . . 21204 |
| CYP2B6 | 8 | 21393 . . . 21534 |
| CYP2B6 | 9 | 25365 . . . 27117 |

In some instances, the amplified portion of the PGx gene or fragment thereof shares 99% or more, e.g., at least 98%, 97%, 96%, 95%, 94%, 93%, 92%, 91%, or 90% identity with a related pseudogene.

In some instances the PGx gene is CYP2D6 and the related pseudogene is cytochrome P450, subfamily IID, polypeptide 7b pseudogene (CYP2D7BP; NCBI Gene ID 1566), cytochrome P450 family 2 subfamily D member 6 pseudogene (LOC101929829; NCBI Gene ID 10929829), or cytochrome P450 family 2 subfamily D member 8 pseudogene (CYP2D8P; NCBI Gene ID 1568).

In some instances the PGx gene is CYP2B6 and the related pseudogene is cytochrome P450 family 2 subfamily member 7 pseudogene (CYP2B7; NCBI Gene ID 1556)

In some instances the PGx gene is TPMT and the related pseudogene is thiopurine S-methyltransferase pseudogene 1 (TPMTP1; NCBI Gene ID 400650), thiopurine S-methyltransferase pseudogene 2 (TMPTP2; NCBI Gene ID 100420393), thiopurine S-methyltransferase pseudogene 3 (TMPTP3; NCBI Gene ID 100129277), or thiopurine S-methyltransferase pseudogene 4 (TMPTP4; NCBI Gene ID 100129298).

Representative PCR primers for amplification of PGx genes or fragments thereof are shown in Table 22. Thus, provided herein are nucleic acid sequences comprising or consisting of any one or more of SEQ ID NOs: 4-19.

TABLE 2
PGx Gene PCR Primers

| | Primer | | Primer Sequence | SEQ ID |
|---|---|---|---|---|
| Target | Pair | Primer ID | (5′→3′) | NO: |
| CYP2D6 E1 | 1 | CYP2D6 E1 F | CAGAGGAGCCCATTTGGTAGT | 4 |
| CYP2D6 E1 | 1 | CYP2D6 E1 R | CCAGCCTGTGGTTTCACC | 5 |
| CYP2D6 E2 | 2 | CYP2D6 E2 F | GGACTACGGTCATCACCCA | 6 |
| CYP2D6 E2 | 2 | CYP2D6 E2 R | GCTTGACAAGAGGCCCTG | 7 |
| CYP2D6 E3-4 | 3 | CYP2D6 E3-4 F | GGCTAATGCCTTCATGGCC | 8 |
| CYP2D6 E3-4 | 3 | CYP2D6 E3-4 R | GCAAATCCTGCTCTTCCG | 9 |
| CYP2D6 E5-6 | 4 | CYP2D6 E5-6 F | GTACCTCCTATCCACGTCAGAGA | 10 |
| CYP2D6 E5-6 | 4 | CYP2D6 E5-6 R | CATCCCGGCAGAGAACAG | 11 |
| CYP2D6 E7 | 5 | CYP2D6 E7 F | CCTGACCTCCTCCAACATAGG | 12 |
| CYP2D6 E7 | 5 | CYP2D6 E7 R | CACCCAAGTGGGGACAGT | 13 |
| CYP2B6 E1 | 6 | CYP2B6 E1 F | CTTCCCCAAGTACCAAGGCA | 14 |
| CYP2B6 E1 | 6 | CYP2B6 E1 R | GACATACATATACCCACAAACCCACAC | 15 |
| CYP2B6 E10 | 7 | CYP2B6 E10 F | ACTCCTGCACTCACTTGCAA | 16 |
| CYP2B6 E10 | 7 | CYP2B6 E10 R | AATCTGTTGCAGTGGACATTTG | 17 |
| TPMT F | 8 | TPMT F | TGAAGTACCAGCATGCACC | 18 |
| TPMT R | 8 | TPMT R | GAGCCACAAGCCTTATAGCC | 19 |

In some instances, enrichment of multiple pharmacogenomics markers is carried out simultaneously, e.g., by multiplex PCR. In some instances, the multiplex PCR is carried out using a mixture of primers. In some instances, the multiplex PCR is carried out using a mixture of primers, e.g., a mixture of one or more primers pairs shown in Table 2, e.g., at least 2, e.g., at least 3, 4, 5, 6, 7, or 8 primer pairs shown in Table 2.

Thus, provided herein are compositions comprising one or more nucleic acid sequences comprising or consisting of one or more of SEQ ID NOs: 4-19, e.g., at least 2, e.g., at least 3, 4, 5, 6, 7, 8, 9, 10, 11, 12, 13, 14, 15, or 16 of SEQ ID NOS: 4-19, e.g., one or more of the primer pairs shown in Table 2, e.g., at least 2, e.g., at least 3, 4, 5 6, 7, or 8 of the primer pairs show in Table 2. In some instances, the composition comprises nucleic acid sequences comprising or consisting of each of SEQ ID NOs: 4-19. In some instances, the composition is a non-naturally occurring composition.

In some instances, the concentration of each of the one or more nucleic acid sequences in the composition is a value from 0.1 μM to 1.0 μM. In some instances, the concentration of each of the one or more nucleic acid sequences in the composition is, independently, about 0.25 μM, about 0.5 μM, or about 0.75 μM.

Useful concentrations for each primer both in the PCR reaction mixture and the primer mixture are also shown in Table 3. In some instances, the concentration of the one or more nucleic acid sequences is as shown in Table 3, or about the concentration shown in Table 3.

TABLE 3
Multiplex PCR Primer Mixture

| | | Exemplary | | Exemplary | |
|---|---|---|---|---|---|
| Primer | Primer | Concentration | | Concentration in | |
| Pair | ID | in Rxn | | Primer Mixture | |
| 1 | CYP2D6 E1 F | 0.100 | μM | 0.25 | μM |
| 1 | CYP2D6 E1 R | 0.100 | μM | 0.25 | μM |
| 2 | CYP2D6 E2 F | 0.100 | μM | 0.25 | μM |
| 2 | CYP2D6 E2 R | 0.100 | μM | 0.25 | μM |
| 3 | CYP2D6 E3-4 F | 0.300 | μM | 0.75 | μM |
| 3 | CYP2D6 E3-4 R | 0.300 | μM | 0.75 | μM |
| 4 | CYP2D6 E5-6 F | 0.200 | μM | 0.5 | μM |
| 4 | CYP2D6 E5-6 R | 0.200 | μM | 0.5 | μM |
| 5 | CYP2D6 E7 F | 0.200 | μM | 0.5 | μM |
| 5 | CYP2D6 E7 R | 0.200 | μM | 0.5 | μM |
| 6 | CYP2B6 E1 F | 0.100 | μM | 0.25 | μM |
| 6 | CYP2B6 E1 R | 0.100 | μM | 0.25 | μM |
| 7 | CYP2B6 E10 F | 0.100 | μM | 0.25 | μM |
| 7 | CYP2B6 E10 R | 0.100 | μM | 0.25 | μM |
| 8 | TPMT F | 0.100 | μM | 0.25 | μM |
| 8 | TPMT R | 0.100 | μM | 0.25 | μM |

In some instances, the mixture of primers further comprises a primer pair that amplifies a known sequence, as a positive control. For example, the primer pair Lambda F (GCTGACGTTACTGACGTGGT; SEQ ID NO:20) and Lambda R (CAGGCGGCCTTTAGTGATGA; SEQ ID NO:21) can be added to the primer mixture to be used as positive controls, e.g., at 0.025 μM in the reaction and 0.0625 μM in the primer mixture. In this case, Lambda Template DNA is added to the PCR reaction and detection of amplified Lambda DNA serves as a positive control. Thus, in some instances, Lambda Template dsDNA (10 ng/μL) is added, e.g., at or at about 16 ng/μL to the reaction, or 40 pg/μL to the primer mixture.

In some instances, the PCR is carried out using dNTP(s). In some cases, the dNTP(s) are selected from the group consisting of dATP, dTTP, dGTP, dCTP, and combinations thereof. In some cases, the dNTP(s) are selected from the group consisting of dATP, dTTP, dGTP, dCTP, dUTP, and combinations thereof.

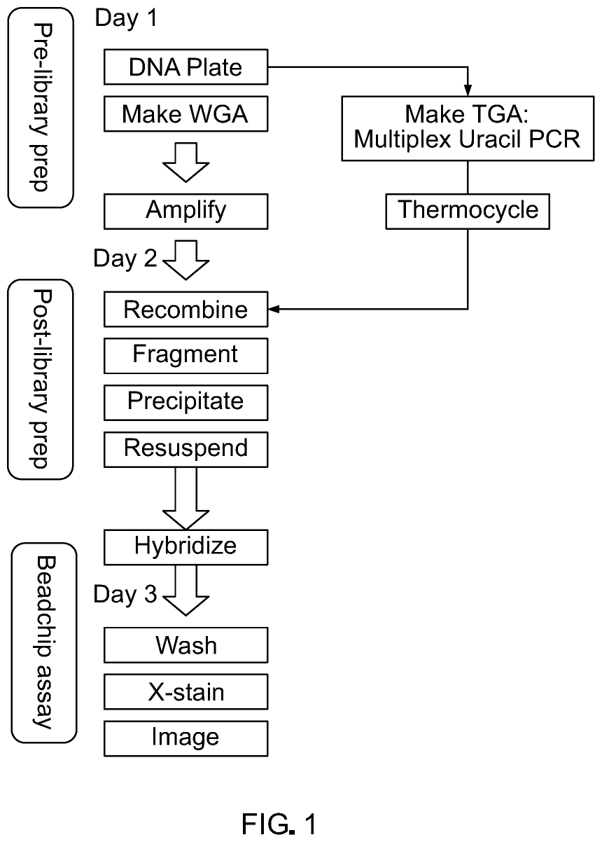

An example of a genotyping method that incorporates enrichment of a pharmacogenomics marker (in this case, targeted gene amplification, “TGA”) is shown in FIG. 1. FIG. 1 is a flowchart that shows one example of an Infinium® assay workflow that incorporates targeted gene amplification (TGA). In this example, pre-library prep is carried out by providing a nucleic acid sample (e.g., a genomic DNA sample), and then carrying out TGA and whole genome amplification (WGA) on the sample in parallel to create a TGA sample and WGA sample.

In this example, for WGA, sodium hydroxide (NaOH) is placed under a layer of mineral oil to prevent acidification and evaporation. The DNA sample is added to NaOH and allowed to incubate for 10 minutes to denature it, followed by addition of random 9-mer oligos, polymerase, dNTPs including dUTP, and other buffer components. During incubation, random oligos bind the single-stranded DNA, which are then extended by the polymerase. A strand-displacing polymerase is used, and as DNA is displaced and made single-stranded, random oligos bind those, and the process continues until limiting components are exhausted. During this time, dUTP is incorporated into the WGA product as uracil.

During TGA, in this example, PCR occurs with primers specific to the regions of interest amplify target regions. A uracil-incorporating polymerase is selected, and dUTP is incorporated into amplicons as uracil. The TGA reaction is optimized to produce a consistent yield across DNA inputs and sample types, ensuring that a predictable and optimal amount of product DNA will be delivered onto the BeadChip® for genotyping, that any non-target product is minimized and/or does not produce inaccurate results or impede accurate copy number variation calling.

Then post-library prep in this example is carried out by recombining the TGA and WGA samples to create a combined TGA/WGA sample and then proceeding with a standard Infinium® workflow for the remainder of the post-library prep as follows: completing post-library prep by fragmenting the TGA/WGA sample, precipitating the nucleic acids of the TGA/WGA sample to produce a precipitate, re-suspending the precipitate.

The recombine step in this example, in which DNA products from the WGA and TGA processes, is optimized to ensure that the TGA amplicons consistently provide accurately genotyping signal for their regions, while WGA has enough material to provide accurate genotyping and CNV signal for other regions. After recombine, samples are incubated with uracil DNA glycosylase (UDG) to remove uracil, leaving abasic sites in products of both WGA and TGA, and are then isopropyl alcohol precipitated. Samples are then resuspended in hybridization buffer, and incubated at 95° C. to finish sample resuspension, fragment DNA at abasic sites, and denature DNA in preparation for hybridization.

Then the BeadChip® assay in this example is carried out, also using a standard Infinium® workflow, as follows: the re-suspended sample is hybridized to the BeadChip®, the BeadChip® is washed, X-stained, and imaged.

In this example, during hybridization, TGA product amplicons outcompete WGA from target regions and regions of high homology, hybridizing to target probes in much greater numbers. This leads to the majority of signal produced by these target probes in subsequent signal generation steps, and the ability to call accurate genotypes. The TGA targets are designed to leave regions open for CNV calling, where WGA signal is used to accurately call regions of interest. Hybridized BeadChips® are then washed of excess DNA, and X-Stained. During XStain, a single base extension occurs on the probe to copy variant information over from hybridized regions, WGA and TGA targets are removed, and then extended probes are differentially stained with a two dye scheme. Signal amplification occurs through layering primary and secondary antibodies. BeadChips® are washed and given a protective coating, and then imaged (scanned), yielding data to process for genotyping and CNV calling.

In this example, CNVs are called according to the methods described in US Patent Application Publication No. US2020/0381079, which is hereby incorporated by reference in its entirety

### Samples and Sample Preparation

Provided herein are methods for genotyping markers, e.g., PGx markers, in a nucleic acid sample, e.g., a DNA and/or RNA sample.

In some instances, the nucleic acid sample is a DNA sample. In some instances, the DNA sample is a genomic DNA sample. In some instances, the DNA sample is a genomic DNA sample from an individual.

In some instances, the nucleic acid sample is a RNA sample. In some instances, the RNA sample is a mRNA sample. In some instances the RNA sample is an mRNA sample from an individual.

In some instances, the individual is a subject in need of treatment. In some instances, the individual is a mammal. In some instances, the individual is a human. In some instances, the individual is a human subject in need of treatment with a drug.

In some instances, the methods include quantifying the nucleic acid sample. In some instances, the methods include adjusting the concentration of the nucleic acid sample. When, for example, WGA and TGA are carried out separately, in some cases the concentration of the portion of the nucleic acid sample used for WGA is adjusted to a first concentration prior to WGA and the portion of the nucleic acid sample used for TGA is adjusted to a second concentration prior to TGA.

In some instances, the methods include amplifying the nucleic acid sample. In some instances, amplification comprises whole genome amplification (WGA), e.g., as described herein. In some instances, amplification includes targeted gene amplification (TGA) (e.g., PCR amplification), e.g., as described herein. In some instances, amplification includes both WGA and TGA. In some instances, a first portion of the sample is amplified by WGA and a second portion of the sample is amplified by TGA. In some instances, the whole genome amplified portion of the sample and targeted amplified portion of the sample are combined after the respective amplifications.

In some instances, the nucleic acid sample is fragmented. In some instances, the nucleic acid sample is fragmented after amplification, e.g., after WGA and/or TGA.

### Whole Genome Amplification

In some instances, the genotyping methods includes an amplification step, e.g., a whole genome amplification (WGA) step. Whole genome amplification methods are known and described in the art. See, e.g., Borgstrom, “Comparison of Whole Genome Amplification Techniques for Human Single Cell Exome Sequencing,” PLOS ONE 12 (2): e0171566 (2017); see also Gonzales-Pena et al., “Accurate Genomic Variant Detection in Single Cells with Primary Template-Directed Amplification,” PNAS 118 (24): e2024176118 (2021).

In some instances, whole genome amplification comprises single cell comparative genomic hybridization (SCOMP) (e.g., AMPLI1™, Silicon Biosystems), multiple displacement amplification (MDA) (e.g., REPLI-g™, Qiagen), single-cell MDA (SCMDA), amplification with degenerate oligonucleotide primed polymerase chain reaction (DOP-PCR), displacement pre-amplification combined with PCR amplification (e.g., MALBAC®, Yikongenomics, or PicoPlex™, Rubicon Genomics), linear amplification via transposon insertion (LIANTI), primary template-directed amplification (PTA), or variations or combination thereof. See Borgstrom 2017; Gonzales-Pena et al.

In some instances, the starting amount of nucleic acid before WGA is from 1 ng to 1 μg, e.g., from 20 ng to 200 ng. In some instances, the starting amount of nucleic acid before WGA is 100 ng or about 100 ng.

Whole genome amplification can involve denaturation of double stranded nucleic acid. In some instances, denaturation is achieved using sodium hydroxide (NaOH). In some instances, the concentration of NaOH in the reaction mixture is from 0.03 M to 0.2 M, e.g., from 0.04 M to 0.16 M, e.g., from 0.044 M to 1.33 M, e.g., from 0.044 M to 0.088 M. In some cases, the concentration of NaOH in the reaction mixture is 0.044 M or about 0.044 M.

In some instances, denaturation is carried out from 5 to 60 min, e.g., from 10 to 30 min, e.g., about 30 min, e.g., about 10 min. In some instances, denaturation is carried out for less than 30 minutes.

In some instances, WGA is carried out with a random primer. In some instances, WGA is carried out with a polymerase that lacks 5′→3′ and/or 3′→5′ exonuclease activity. In some instances, the polymerase is Exo-Minus Klenow DNA Polymerase (an N-terminal truncation of DNA Polymerase with mutations D355A and E357A).

### Targeted Enrichment

The methods described herein can include enrichment of a particular target, e.g., by targeted gene amplification (TGA), e.g., as described above, instead of or in addition to whole genome amplification.

In some instances, the starting amount of nucleic acid before TGA is from 1 ng to 1 μg, e.g., from 20 ng to 200 ng. In some instances, the starting amount of nucleic acid before TGA is 100 ng or about 100 ng.

In some instances, TGA is carried out.

In some instances, WGA and TGA are carried out in parallel on different portions of the same starting nucleic acid sample, e.g., different portions of the same nucleic acid sample. In some instances, the whole genome amplified portion and the targeted gene amplification portion are recombined before hybridization to an array, e.g., before fragmentation. An example of an Infinium® assay workflow that incorporates TGA is shown in FIG. 1.

### DNA Fragmentation

In some instances, the genotyping methods include nucleic acid fragmentation. Nucleic acid fragmentation can be achieved by a number of methods known and described in the art. See, e.g., Sapojnikova et al., “A Comparison of DNA Fragmentation Methods-Applications for the Biochip Technology,” J. Biotechnol. 256:1-5 (2017).

In some instances, nucleic acid fragmentation comprises sonication, enzymatic digestion, or a combination thereof. In some instances, nucleic acid fragmentation comprises Uracil-DNA Glycosylase (UDG) digestion. See, e.g., von Wintzingerode et al., “Base-Specific Fragmentation of Amplified 16S rRNA Genes Analyzed by Mass Spectrometry: A Tool for Rapid Bacterial Identification,” PNAS 99 (10): 7039-44 (2002).

In some instances, the average size of the nucleic acid fragments after fragmentation is from about 50 to about 150 nucleotides. In some cases, the average size of the nucleic acid fragments after fragmentation is about 80 nucleotides. In some instances, the size of the nucleic acid fragments after fragmentation is from 50 to 150 nucleotides.

### Probes and Arrays

In some instances, the pharmacogenomics marker is identified by hybridization of the sample, e.g., to a probe, e.g., a probe on a microarray. In some instances, the array is a bead array.

### Probes

Thus, provided herein are PGx capture probes. In some cases, the PGx capture probe is an oligonucleotide comprising a capture domain (e.g., a nucleotide sequence) that can specifically bind (e.g., hybridize) to a target PGx analyte (e.g., PGx gene or fragment thereof) within a sample.

In some instances, the PGx capture probe binds to a PGx gene or fragment thereof selected from the group consisting of ABCB1, ABCC4, ABCG2, ACE, ALDH1A1, ALK, BCKDK, BCR, BRAF, C8orf34, CACNA1S, CES1, CFTR, CHRNA3, COL22A1, COQ2, CRHR1, CYP19A1, CYP21A2, CYP2A6, CYP2A7P1, CYP2B6, CYP2C19, CYP2C8, CYP2C9, CYP2D6, CYP2E1, CYP3A, CYP3A5, CYP4F2, DPYD, DYNC2H1, EGFR, EPHX1, ERBB2, F5, FCGR3A, FKBP5, G6PD, GGCX, GSTM1, GSTT1, HCP5, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PRSS53, PSORS1C1, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT1A5, UGT2B17, UGT2B28, VDR, VKORC1, YEATS4, ZNRD1-AS1, and combinations thereof.

In some instances, the PGx capture probe is a PGx capture probes from Table 9, below. Also provided herein are pluralities of PGx capture probes.

In some instances, one or more, e.g., at least 2, e.g., at least 3, 4, 5, 6, 7, 8, 9 10, 20, 30, 40, 50, 60, 70, 80, 90, 100, 110, 120, 130, 140 150, 160, 170, 180, 190, 200, 210 or more PGx genes selected from the group consisting of ABCB1, ABCC4, ABCG2, ACE, ALDH1A1, ALK, BCKDK, BCR, BRAF, C8orf34, CACNA1S, CES1, CFTR, CHRNA3, COL22A1, COQ2, CRHR1, CYP19A1, CYP21A2, CYP2A6, CYP2A7P1, CYP2B6, CYP2C19, CYP2C8, CYP2C9, CYP2D6, CYP2E1, CYP3A, CYP3A5, CYP4F2, DPYD, DYNC2H1, EGFR, EPHX1, ERBB2, F5, FCGR3A, FKBP5, G6PD, GGCX, GSTM1, GSTT1, HCP5, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PRSS53, PSORS1C1, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT1A5, UGT2B17, UGT2B28, VDR, VKORC1, YEATS4, ZNRD1-AS1, and combinations thereof, are captured by the plurality of PGx capture probes.

In some instances, the plurality of PGx capture probes comprises one or more, e.g., at least 50 60, 70 80, 90, 100, 200, 300, 400, 500, 600, 700, 800, 900, 1000, 1100, 1200, 1300, 1400, 1500, or 1600 of the PGx probes in Table 9, below.

### Arrays

Array based genotypic methods, in general, are known and described in the art. See, e.g., Ragoussis, “Genotyping Technologies for Genetic Research,” Annu. Rev. Genomics Hum. Genet. 10:117-33 (2009).

Suitable microarrays include, but are not limited to, BeadArray™ products, including, for example, GoldenGate® and Infinium® Genotyping Assays, DASL® and DirectHyb Assays. In some instances, the microarrays is an Infinium® microarray. The Infinium® BeadArray™ platform is described, for example, in Steemers and Gunderson, “Whole Genome Genotyping Technologies on the BeadArray™ Platform,” Biotechnol. J. 2:41-9 (2007). In some instances, the array utilizes allele-specific primer extension (ASPE) biochemical scoring. In some instances, the array utilizes single-base extension (SBE) biochemical scoring. See id. In some instances, the array utilizes SBE biochemical scoring in combination with ASPE dual probe design, e.g., to detect AT and GC polymorphisms.

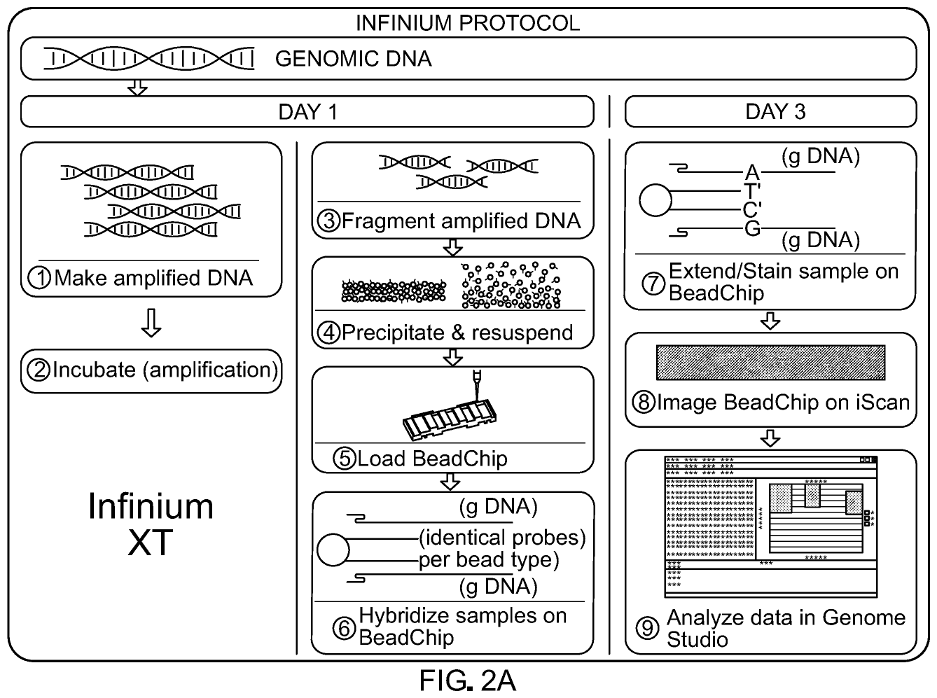

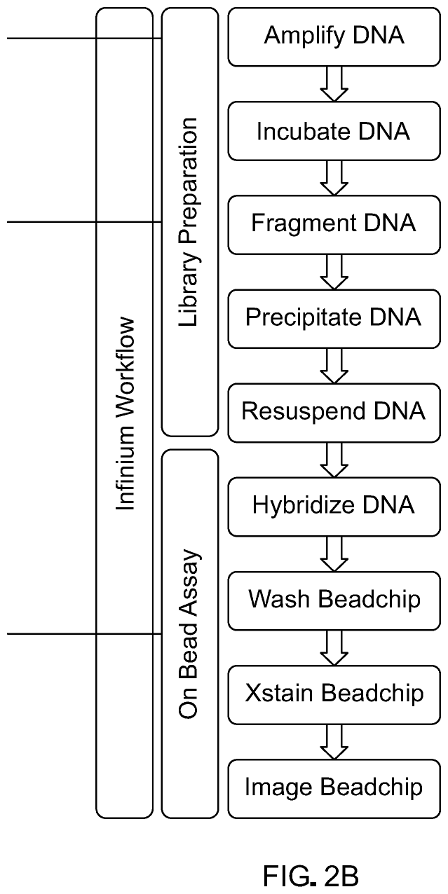

A useful Infinium® assay workflow is shown in FIG. 2A and FIG. 2B. First, nucleic acid, e.g., genomic DNA, is amplified; then, the amplified DNA is fragmented, precipitated and resuspended, loaded on the BeadChip®, extended, stained, imaged, and analyzed. This example is carried out as described above with respect to FIG. 1, except the TGA portion of the workflow is not included.

Thus, provided herein are arrays comprising PGx capture probes, e.g., the pluralities of PGx capture probes described herein. In some instances, the array comprises a surface, e.g., beads, to which the PGx capture probes are bound. In some instances, the capture probes are bound directly to the surface. In some instances, the capture probes are bound indirectly to the surface.

In some instances, at least 10, e.g., at least 20, 30, 40, 50, 60 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 510, 520, 530, 540, 550, 560, 570, 580, 590, or 600 pharmacogenomics markers, e.g., the PGx markers described herein, are genotyped using the same microarray.

In some instances, 10 to 600, e.g., 10 to 550, 10 to 500, 10 to 450, 10 to 400, 10 to 350, 10 to 300, 10 to 250, 10 to 200, 10 to 150, 10 to 100, 10 to 50, 50 to 600, 50 to 550, 50 to 500, 50 to 450, 50 to 400, 50 to 350, 50 to 300, 50 to 250, 50 to 200, 50 to 150, 50 to 100, 100 to 600, 100 to 550, 100 to 500, 100 to 450, 100 to 400, 100 to 350, 100 to 300, 100 to 250, 100 to 200, 100 to 150, 150 to 600, 150 to 550, 150 to 500, 150 to 450, 150 to 400, 150 to 350, 150 to 300, 150 to 250, 150 to 200, 200 to 600, 200 to 550, 200 to 500, 200 to 450, 200 to 400, 200 to 350, 200 to 300, 200 to 250, 250 to 600, 250 to 550, 250 to 500, 250 to 450, 250 to 400, 250 to 350, 250 to 300, 300 to 600, 300 to 550, 300 to 500, 300 to 450, 300 to 400, 300 to 350, 350 to 600, 350 to 550, 350 to 500, 350 to 450, 350 to 400, 400 to 600, 400 to 550, 400 to 500, 400 to 450, 450 to 600, 450 to 550, 450 to 500, 500 to 600, 500 to 550, or 550 to 600 PGx markers, e.g., the PGx markers described herein, are genotyped using the same microarray.

In some instances, the genotyping methods include single-base extension (SBE) (depicted, e.g., as step 7 of FIG. 2B and the Xstain BeadChip® step in FIG. 2B).

### Kits

Provided herein are kits comprising reagent(s) suitable for carrying out the methods described herein.

In some instances, the kit comprises PCR primer(s) for targeted gene amplification of PGx genes or fragments thereof, e.g., as described above. In some cases, the kit further comprises control primer(s) and/or oligonucleotides. In some cases, the kit comprises a composition or compositions comprising the PCR primer(s) and/or oligonucleotide(s). In some cases, the composition(s) comprising the PCR primer(s) and/or oligonucleotide(s) further comprise a buffer.

In some instances, the kit comprises one or more polymerase enzyme(s), e.g., DNA polymerase enzyme(s). In some cases, the polymerase enzyme is a dU-incorporating polymerase. In some cases the kit comprises a composition comprising the polymerase enzyme. In some cases, the composition further comprises a buffer.

In some instances, the kit comprises dNTP(s). In some cases, the dNTP(s) are selected from the group consisting of dATP, dGTP, dCTP, dTTP, and combinations thereof. In some cases, the dNTP(s) are selected from the group consisting of dATP, dGTP, dCTP, dTTP, dUTP, and combinations thereof. In some cases, the kit comprises a composition comprising the dNTP(s). In some cases, the composition comprising the dNTP(s) further comprises a buffer.

In some instances, the kit comprises a composition comprising one or more polymerase enzyme(s), e.g., as described above, and dNTP(s), e.g., as described above.

In some instances, the kit comprises a uracil DNA nucleotide glycosylase. In some cases, the kit comprises a composition comprising a uracil DNA nucleotide glycosylase. In some cases, the composition further comprises a buffer.

In some instances, the kit comprises random oligonucleotide(s), e.g., for WGA. In some cases, the kit comprises a composition comprising random oligonucleotide(s), e.g., for WGA. In some cases, the composition further comprises a buffer.

In some instances, the kit comprises salt(s) and/or buffer(s) for DNA precipitation.

In some instances, the kit comprises control oligonucleotide(s) and/or buffer(s) for DNA resuspension.

In some instances, the kit comprises protein(s) and/or buffer(s) for washing the DNA BeadChip® surface.

In some instances, the kit comprises protein(s) and/or buffer(s) for buffer exchange.

In some instances, the kit comprises labeled ddNTP(s).

In some instances, the kit comprises binding molecule(s), e.g., antibodie(s) that bind to ddNTP(s).

In some instances, the kit comprises binding molecule(s), e.g., antibodie(s) that bind to the binding molecule(s) that bind to ddNTPs. In some cases, the antibodie(s) are labeled.

### Treatment Selection

Provided herein are methods for selecting a drug treatment for a subject in need thereof.

In some cases, the methods include obtaining a genotype of a PGx gene for the subject, e.g., a PGx gene described herein, e.g., by any of the methods described herein, and, based on the genotype, selecting a drug treatment for a subject in need thereof.

In some instances, selecting a drug treatment includes determining that a drug is suitable for administration to the patient. In some instances, selecting a drug treatment includes determining that a drug is not suitable for administration to the patient and, in some instances, determining that an alternate drug is more suitable for administration to the patient. In some instances, selecting a drug treatment includes identifying a genotype-drug response interaction, and, based on the identified interaction, selecting a drug treatment for the patient.

In some cases, identifying a genotype-drug response interaction includes querying a database, such as PharmGKB, to identify the genotype-drug response interaction. In some cases, a genotype-drug response interaction includes consulting guidelines on genotype and drug selection from organizations such as: the Clinical Pharmacogenetic Implementation Consortium (CPIC) (a collaborator of PharmGKB), the Dutch Pharmacogenetics Working Group (DPWG), the Canadian Pharmacogenomics Network for Drug Safety (CPNDS), or other groups. In some instances, a genotype-drug response interaction is identified by pharmacogenomic prescribing recommendations found in publications. In some instances, a genotype-drug response interaction is identified by an algorithm.

In some cases, the genotype-drug response interaction is a positive interaction. That is, the genotype, e.g., the genotype of a PGx marker, e.g., a PGx marker described herein, is associated with a positive treatment outcome using a drug. Thus, in some cases, selecting a drug treatment includes identifying a genotype associated with a positive treatment outcome to a drug, and administering that drug to the patient.

In some cases, the genotype-drug responses interaction is a negative interaction. That is, the genotype is associated with a negative treatment outcome using a drug. Thus, in some cases, selecting a drug treatment includes identifying a genotype associated with a negative treatment outcome to a drug, and not administering that drug to the patient and/or administering a different drug to the patient.

In some instances, the drug is selected from the group consisting of abacavir, allopurinol, amikacin, amitriptyline, atazanavir, atomoxetine, azathioprine, capecitabine, carbamazepine, celecoxib, citalopram, clopidogrel, codeine, desflurane, efavirenz, enflurane, escitalopram, fluorouracil, flurbiprofen, fosphenytoin, gentamicin, halothane, ibuprofen, irinotecan, isoflurane, ivacaftor, kanamycin, lansoprazole, lornoxicam, meloxicam, mercaptopurine, methoxyflurane, nortriptyline, omeprazole, ondansetron, oxcarbazepine, pantoprazole, paromomycin, paroxetine, peginterferon alfa-2a, peginterferon alfa-2b, phenytoin, piroxicam, pitolisant, plazomicin, rasburicase, sevoflurane, simvastatin, siponimod, streptomycin, succinylcholine, tacrolimus, tafenoquine, tamoxifen, tenoxicam, thioguanine, tobramycin, tramadol, tropisetron, voriconazole, warfarin, aspirin, divalproex sodium, eliglustat, hydralazine, oliceridine, pimozide, tetrabenazine, valproic acid, velaglucerase alfa, venlafaxine, vortioxetine, acenocoumarol, aripiprazole, belinostat, brivaracetam, carglumic acid, chloramphenicol, chlorpropamide, ciprofloxacin, clomipramine, dapsone, desipramine, dexlansoprazole, dimercaprol, doxepin, fluvoxamine, glibenclamide, glimepiride, glipizide, hydrocodone, imipramine, mafenide, mesalazine, methadone, methylene blue, moxifloxacin, mycophenolic acid, nalidixic acid, nitrofurantoin, norfloxacin, pegloticase, phenazopyridine, phenprocoumon, primaquine, probenecid, quinine, risperidone, rosuvastatin, sertraline, sodium nitrite, sulfacetamide, sulfadiazine, sulfamethoxazole/trimethoprim, sulfasalazine, sulfisoxazole, trimipramine, amifampridine, amifampridine phosphate, amoxapine, amphetamine, aripiprazole lauroxil, atenolol, avatrombopag, brexpiprazole, bucindolol, bupropion, carisoprodol, carvedilol, cevimeline, clobazam, clozapine, dabrafenib, daunorubicin, deutetrabenazine, dextromethorphan, diazepam, dolutegravir, donepezil, doxorubicin, dronabinol, elagolix, erdafitinib, flecainide, gefitinib, haloperidol, hydroxychloroquine, iloperidone, labetalol, lapatinib, lesinurad, lidocaine, 1-methylfolate, lofexidine, meclizine, metoclopramide, metoprolol, mirabegron, mirtazapine, mivacurium, nebivolol, nevirapine, nilotinib, pazopanib, perphenazine, procainamide, propafenone, propranolol, protriptyline, raltegravir, tamsulosin, thioridazine, timolol, valbenazine, zuclopenthixol, aceclofenac, adalimumab, alfentanil, amisulpride, ataluren, buprenorphine, carbimazole, carboplatin, chloroquine, cyclosporine, darifenacin, diclofenac, digoxin, dolasetron, duloxetine, eltrombopag, esomeprazole, etanercept, fentanyl, fesoterodine, flibanserin, fluoxetine, galantamine, hormonal contraceptives for systemic use, hydromorphone, indomethacin, infliximab, isoniazid, levomethadone, lumiracoxib, methazolamide, methimazole, methotrexate, methylphenidate, modafinil, morphine, nabumetone, naloxone, naltrexone, naproxen, olanzapine, oxycodone, paliperidone, palonosetron, pravastatin, propylthiouracil, quetiapine, quinidine, rabeprazole, remifentanil, rosiglitazone, sirolimus, sufentanil, tegafur, terbinafine, tolterodine, vitamin c, ziprasidone, interferon alfa-2b, recombinant, midazolam, nicotine, oxazepam, alendronate, atorvastatin, budesonide, caffeine, captopril, cerivastatin, cetuximab, cisplatin, cyclophosphamide, epirubicin, etoposide, fluticasone propionate, fluticasone/salmeterol, fluvastatin, furosemide, gemcitabine, hydrochlorothiazide, idarubicin, lamotrigine, latanoprost, metformin, oxaliplatin, raloxifene, ribavirin, risedronate, rituximab, salbutamol, salmeterol, sildenafil, spironolactone, tenofovir, triamcinolone, and combinations thereof.

In some instances, the PGx gene is ABCG2 and the drug is rosuvastatin.

In some instances, the PGx gene is ABL2 and the drug is valproic acid.

In some instances, the PGx gene is ASL and the drug is valproic acid.

In some instances, the PGx gene is CACNA1S and the drug is selected from the group consisting of desflurane, enflurane, halothane, isoflurane, methoxyflurane, sevoflurane, succinylcholine, and combinations thereof.

In some instances, the PGx gene is CFTR and the drug is ivacaftor.

In some instances, the PGx gene is CPS1 and the drug is valproic acid.

In some instances, the PGx gene is CYP2B6 and the drug is selected from the group consisting of efavirenz, methadone, and combinations thereof.

In some instances, the PGx gene is CYP2C19 and the drug is selected from the group consisting of amitriptyline, brivaracetam, citalopram, clomipramine, clopidogrel, dexlansoprazole, doxepin, escitalopram, imipramine, lansoprazole, omeprazole, pantoprazole, sertraline, trimipramine, voriconazole, and combinations thereof.

In some instances, the PGx gene is CYP2C19 and the drug is selected from the group consisting of acenocoumarol, celecoxib, flurbiprofen, fosphenytoin, ibuprofen, lornoxicam, meloxicam, phenytoin, piroxicam, siponimod, tenoxicam, warfarin, amitriptyline, aripiprazole, atomoxetine, clomipramine, codeine, desipramine, doxepin, eliglustat, fluvoxamine, hydrocodone, imipramine, nortriptyline, oliceridine, ondansetron, paroxetine, pimozide, pitolisant, risperidone, tamoxifen, tetrabenazine, tramadol, trimipramine, tropisetron, venlafaxine, vortioxetine, and combinations thereof.

In some instances, the PGx gene is CYP3A5 and the drug is tacrolimus.

In some instances, the PGx gene is CYP4F2 and the drug is selected from the group consisting of acenocoumarol, phenprocoumon, warfarin, and combinations thereof.

In some instances, the PGx gene is DPYD and the drug is selected from the group consisting of capecitabine, fluorouracil, and combinations thereof.

In some instances, the PGx gene is G6PD and the drug is selected from the group consisting of aspirin, chloramphenicol, chlorpropamide, ciprofloxacin, dapsone, dimercaprol, glibenclamide, glimepiride, glipizide, mafenide, mesalazine, methylene blue, moxifloxacin, nalidixic acid, nitrofurantoin, norfloxacin, pegloticase, phenazopyridine, primaquine, probenecid, quinine, rasburicase, sodium nitrite, sulfacetamide, sulfadiazine, sulfamethoxazole/trimethoprim, sulfasalazine, sulfisoxazole, tafenoquine, and combinations thereof.

In some instances, the PGx gene is GBA and the drug is velaglucerase alfa

In some instances, the PGx gene is HLA-A and the drug is carbamezapine.

In some instances, the PGx gene is HLA-B and the drug is selected from the group consisting of abacavir, allopurinol, carbamazepine, fosphenytoin, oxcarbazepine, phenytoin, and combinations thereof.

In some instances, the PGx gene is HPRT1 and the drug is mycophenolic acid.

In some instances, the PGx gene is IFNL3 and the drug is selected from the group consisting of peginterferon alfa-2a, peginterferon alfa-2b, and combinations thereof.

In some instances, the PGx gene is IFNL4 and the drug is selected from the group consisting of peginterferon alfa-2a, peginterferon-2b, and combinations thereof.

In some instances, the PGx gene is MT-RNRI and the drug is selected from the group consisting of amikacin, gentamicin, kanamycin, paromomycin, plazomicin, streptomycin, tobramycin, and combinations thereof.

In some instances, the PGx gene is NAGS and the drug is selected from the group consisting of arglumic acid, valproic acid, and combinations thereof.

In some instances, the PGx gene is NAT2 and the drug is hydralazine.

In some instances, the PGx gene is NUDT15 and the drug is selected from the group consisting of azathioprine, mercaptopurine, thioguanine, and combinations thereof.

In some instances, the PGx gene is OTC and the drug is valproic acid.

In some instances, the PGx gene is POLG and the drug is selected from the group consisting of divalproex sodium, valproic acid, and combinations thereof.

In some instances, the PGx gene is RYR1 and the drug is selected from the group consisting of desflurane, enflurane, halothane, isoflurane, methoxyflurane, sevoflurane, succinylcholine, and combinations thereof.

In some instances, the PGx gene is SCN1A and the drug is selected from the group consisting of carbamazepine, phenytoin, and combinations thereof.

In some instances, the PGx gene is SLCO1B1 and the drug is simvastatin.

In some instances, the PGx gene is TPMT and the drug is selected from the group consisting of mercaptopurine, thioguanine, and combinations thereof.

In some instances, the PGx gene is UGT1A1 and the drug is selected from the group consisting of atazanavir, belinostat, irinotecan, and combinations thereof.

In some instances, the PGx gene is VKORC1 and the drug is warfarin.

### EXAMPLES

The invention is further described in the following examples, which do not limit the scope of the invention described in the claims.

### Example 1: PGx Array Genotyping Protocol

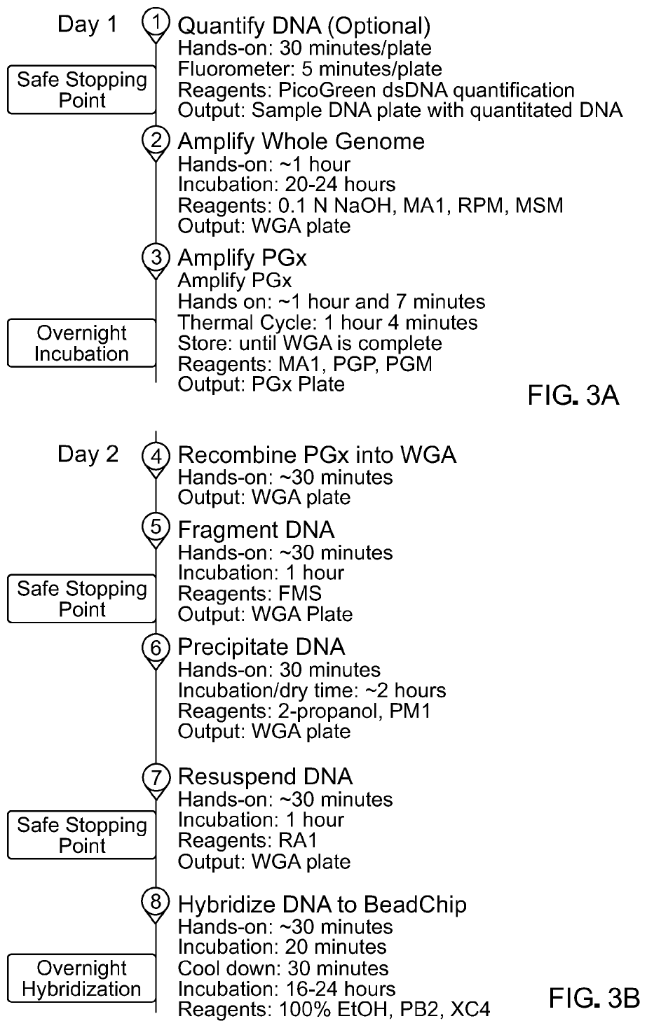

FIGS. 3A-3C show an overview of a workflow for PGx genotyping using the Infinium® Global Diversity Array with Enhanced PGx. In this example, on day 1, DNA is optionally quantified, whole genome amplified, and PGx amplified (e.g., by TGA). On day 2, the PGx amplified sample is recombined with the WGA, the recombined sample is fragmented, the nucleic acid precipitated and then re-suspended, and the re-suspended sample is hybridized to the BeadChip®. On day 3, the BeadChip® is washed, extended, stained, and imaged. Genotyping was carried out using Infinium® LCG assay chemistry. Steps 1, 2, and 5-11 were carried out according to the manufacturer instructions for the Infinium® Global Diversity Array. See Illumina® Infinium® LCG Assay Reference Guide, Document #15023139 v04 (2019), available at support.illumina.com, except DNA input was 5 μl of 20 ng/μl as opposed to 4 μl of 50 ng/μl. Steps 3 and 4, described below, are specific to the Enhanced PGx array.

### 3) Amplify PGx

1. Add 10 μl MAI to each well of the PGx plate.
2. Add 10 μl PGP to each well of the PGx plate. PGP is a primer mix containing the primers shown in Table 4, whose sequences are set forth in Table 2.

TABLE 4
PGP

| | | Amount in Rxn | | Amount/Concentration | |
|---|---|---|---|---|---|
| | | (10 μL per | | in PGP (1,100 μL | |
| ID | Component | reaction) | | volume) | |
| Diluent | RM, | to volume | | to volume | |
| | Resuspension | | | | |
| | Buffer, IDTE, | | | | |
| | pH 8.0 | | | | |
| CYP2D6 E1 F | Primer | 0.100 | μM | 0.25 | μM |
| CYP2D6 E1 R | Primer | 0.100 | μM | 0.25 | μM |
| CYP2D6 E2 F | Primer | 0.100 | μM | 0.25 | μM |
| CYP2D6 E2 R | Primer | 0.100 | μM | 0.25 | μM |
| CYP2D6 | Primer | 0.300 | μM | 0.75 | μM |
| E3-4 F | | | | | |
| CYP2D6 | Primer | 0.300 | μM | 0.75 | μM |
| E3-4 R | | | | | |
| CYP2D6 | Primer | 0.200 | μM | 0.5 | μM |
| E5-6 F | | | | | |
| CYP2D6 | Primer | 0.200 | μM | 0.5 | μM |
| E5-6 R | | | | | |
| CYP2D6 E7 F | Primer | 0.200 | μM | 0.5 | μM |
| CYP2D6 E7 R | Primer | 0.200 | μM | 0.5 | μM |
| CYP2B6 E1 F | Primer | 0.100 | μM | 0.25 | μM |
| CYP2B6 E1 R | Primer | 0.100 | μM | 0.25 | μM |
| CYP2B6 | Primer | 0.100 | μM | 0.25 | μM |
| E10 F | | | | | |
| CYP2B6 | Primer | 0.100 | μM | 0.25 | μM |
| E10 R | | | | | |
| TPMT F | Primer | 0.100 | μM | 0.25 | μM |
| TPMT R | Primer | 0.100 | μM | 0.25 | μM |
| Lambda F | Primer | 0.025 | μM | 0.0625 | μM |
| Lambda R | Primer | 0.025 | μM | 0.0625 | μM |
| Lambda | Lambda, | 16 | pg/μL | 40 | pg/μL |
| Template, | 10 ng/μL | | | | |
| dsDNA | | | | | |

3. Add 10 μl PGM to each well of the PGx plate. PGM contains the components shown in Table 5:

TABLE 5
PGM

| | | | Amount/Concentration | |
|---|---|---|---|---|
| | Amount in Rxn (10 | | in PGM (1,100 μL | |
| Component | μL per reaction) | | volume) | |
| Water, Nuclease-Free | to volume | | to volume | |
| dUTP, 100 mM Solution | 0.1 | mM | 0.25 | mM |
| dTTP, 100 mM Solution | 0.2 | mM | 0.5 | mM |
| dGTP, 100 mM Solution | 0.2 | mM | 0.5 | mM |
| dCTP, 100 mM Solution | 0.2 | mM | 0.5 | mM |
| dATP, 100 mM Solution | 0.2 | mM | 0.5 | mM |
| 5x Buffer Q5U | 0.8 | x | 2 | x |
| Q5U Polymerase | 0.05 | Units/μL | 0.125 | Units/μL |

4. Transfer 5 μl DNA sample from the DNA plate to the corresponding wells of the PGx plate.
5. Apply Microseal ‘B’ to the PGx plate and make sure each well is tightly sealed with no bubbles or gaps in the film.
6. Vortex at 1600 rpm for 15 seconds, and then pulse centrifuge at 280×g for 1 minute.
7. PCR at 98° C. for 30 s, followed by 11 cycles of 98° C. for 30 s, 76° C. for 30 s, 72° C. for 30 s, followed by 21 cycles of 98° C. for 30 s, 66° C. for 30 s, 72° C. for 30 s, followed by 72° C. for 5 minutes. Hold at 4° C.

### 4) Recombine WGA and Amplified PGx

1. Transfer 6.5 μL of the PGx product from the PGx plate directly into the corresponding wells of the WGA plate.
2. Proceed to fragmentation.

### Example 2: PGx Array Genotyping is Accurate, Precise, and Reproducible

Genotyping was carried out on the Infinium® Global Diversity Array with Enhanced PGx array as described in Example 1 on 192 samples representing a broad array of PGx SNV and CNV genotypes (replicates 1-5) and 384 samples including the 192 from replicates 1-5 and an additional 192 CNV samples (replicate 6). SNVs were called using the GenomeStudio® Genotyping Module described, for example, in Gunderson et al., “Whole-Genome Genotyping,” Methods Enzymol. 410:359-76 (2006). CNVs were called according to the methods described in US20200381079, which is hereby incorporated by reference in its entirety.

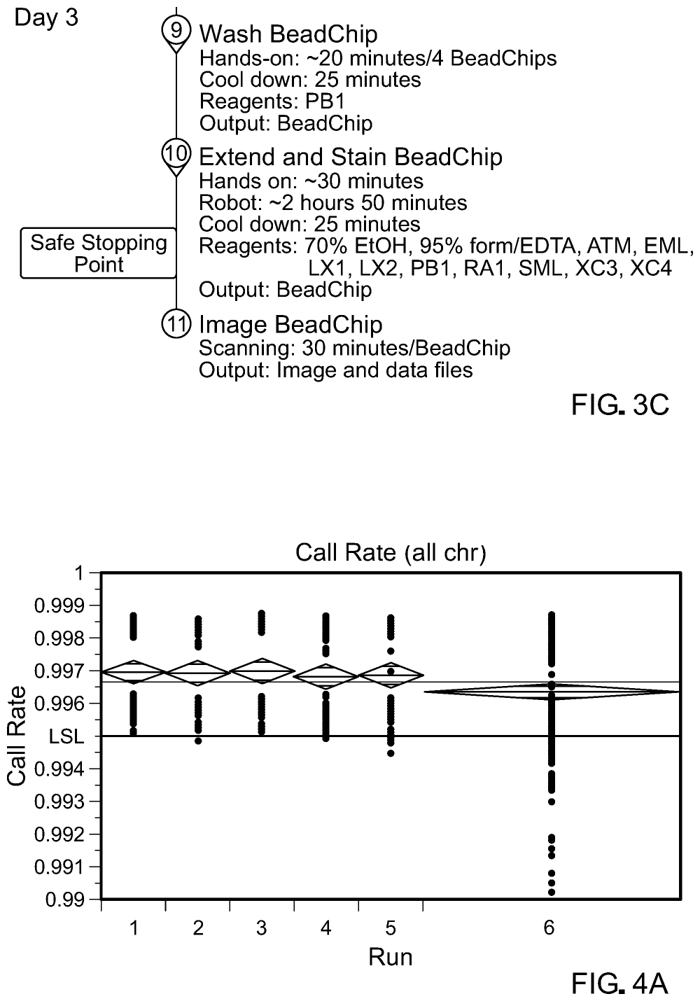

As shown in FIG. 4A, call rate was greater than 99% across the entire array, while, as shown in FIG. 4B, LogRDev was less than 0.15. As shown in Table 6, 5 samples (0.6%, indicated with *) failed the first pass and were re-queued. They were successful on the second pass. For the PGx content, as shown in FIG. 4C, call rate was greater than 99% and LogRDev was less than 0.17.

TABLE 6
Array Genotyping Statistics

| | No. | Call | | Failures | PGx | PGx |
|---|---|---|---|---|---|---|
| Replicate | Samples | Rate | LogRDev | (%) | CR | LogRDev |
| 1 | 96 | 0.997 | 0.147 | 0 | 0.9995 | 0.1634 |
| 2 | 95* | 0.997 | 0.146 | 1 | 0.9995 | 0.1702 |
| 3 | 96 | 0.997 | 0.145 | 0 | 0.9996 | 0.1724 |
| 4 | 96 | 0.997 | 0.147 | 0 | 0.9996 | 0.1731 |
| 5 | 96 | 0.997 | 0.144 | 0 | 0.9995 | 0.1753 |
| 6 | 380* | 0.997 | 0.152 | 1 | 0.9992 | 0.1648 |
| All | 859* | 0.997 | 0.148 | 0.6 | 0.9995 | 0.1682 |

### Example 3: PGx Content Underpinning Star Alleles is Accurate

Genotyping was carried out on the Infinium® Global Diversity Array with Enhanced PGx array as described in Example 1. Small variants in star alleles were called using the GenomeStudio® Genotyping Modconule described, for example, in Gunderson et al., “Whole-Genome Genotyping,” Methods Enzymol. 410:359-76 (2006). Genotyping statistics are shown in Table 7.

TABLE 7
PGx Genotyping Statistics (Small Variants)

| | | | | Minor | |
|---|---|---|---|---|---|
| | PGx | Possible | Possible | Alleles | |
| | Variants | Major | Minor | Called | Concordance |
| PGx Content | Evaluated | Alleles | Alleles | Correctly | to NGS |
| PGx (all) | 80/542 | 15741 | 902 | 894 | 99.1% |
| PGx (TGA | 28/89 | 4544 | 212 | 208 | 98.1% |
| targets) | | | | | |

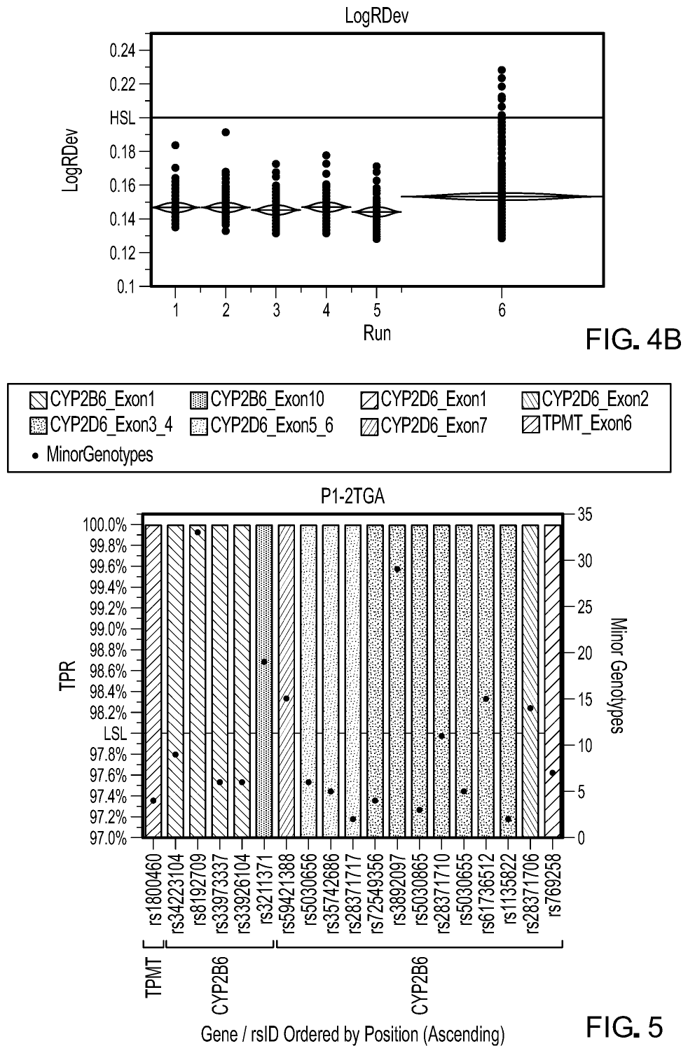

True positive rates for 19 TGA targets is shown in FIG. 5. Concordance to orthogonal data was found to be similar for regions with and without TGA coverage (98.1% and 99.1%, respectively) showing that TGA rescues the ability to accurately assess the noisy region of high homology that it was selected to improve. Samples were selected to maximize coverage of minor variants to assess accuracy.

### Example 4: PGx Content is Accurate, Precise, and Reproducible

Genotyping was carried out on the Infinium® Global Diversity Array with Enhanced PGx array as described in Example 1. Small variants in star alleles were called using the GenomeStudio® Genotyping Module described, for example, in Gunderson et al., “Whole-Genome Genotyping,” Methods Enzymol. 410:359-76 (2006).

As shown in Table 8, the system is accurate, precise, and reproducible for genotyping PGx small variants.

TABLE 8
PGx Genotyping Statistics (Small Variants) -
Accuracy, Precision, and Reproducibility

| | | Avg |
|---|---|---|
| Test | Conditions | Reproducibility |
| Concordance | Run 1-5 versus Gentrain Genotype calls | 0.9999 |
| Repeatability, | SNV plate: 3 samples (9X) | PGx: 0.9999 |
| (intra-run) | DNA input, ng: 200 (7X), 100 (1X), | All: 0.9999 |
| | 400 (1X) | |
| | Multiple plate/BeadChip ® loading | |
| | positions | |
| Repeatability | Three runs (Run 1-3) | PGx: 1.000 |
| (inter-run) | 3 samples (9X); 21 samples (1X0 | All: 0.999 |
| | Same operator for manual library prep, | |
| | reagents, and instruments, on the same | |
| | day | |
| Reproducibility | Five runs (Run 1-5) | PGx: 0.9999 |
| (inter-run) | 3 samples (9X); 21 samples (1X) | All: 0.9999 |
| | 3 operators, 2 Tecans for X-stain, 3 | |
| | TGA kits | |

### Example 5: Improved Stability for Improved CNV Calling

To understand the impact of the first step of whole genome amplification (denaturation of DNA by addition of sodium hydroxide (NaOH)) on CNV calling capability, a titration of NaOH molarity/normality into denaturation was performed, with a functional assay readout. To adjust the hydroxide concentration of the denaturation reaction, molarity of stock NaOH was adjusted while holding the volume added constant. The assay metric Log R Mean, a measure of how similarity of test data to reference data, was used as a proxy for CNV calling capability, with results trending closer to 0 indicating higher capability. Additionally, normalized signal (R) was measured across the hydroxide titration to understand stability of signal. Signal that trends higher or lower than reference is considered a gain or loss of copy number (respectively) due to the mechanics of the CNV Caller.

A second titration was run varying the input volumes and normalities of NaOH, with data being analyzed by 1) an Illumina-developed CYP2D6 CNV Caller, and 2) comparison of normalized signal over probes sorted by GC content. For testing of the CNV caller, 96 samples with a spread of CYP2D6 copy numbers were selected.

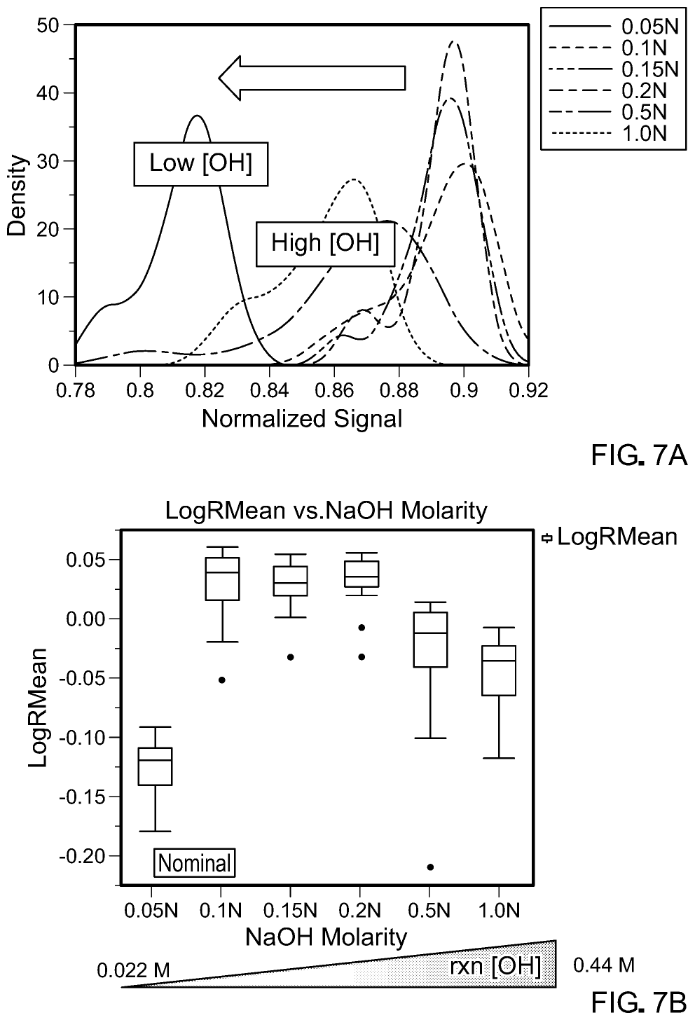

Signal stabilized between addition of 0.1 N and 0.2 N NaOH (0.044 M and 0.088 M hydroxide concentration in DNA denaturation, respectively), with normalized signal and Log R Mean shifting lower at 0.05 M and 0.5 M NaOH (0.022 N and 0.22 N hydroxide concentration in DNA denaturation, respectively) (FIGS. 7A and 7B). FIG. 7A shows distributions of normalized signal (R) resulting from denaturation conditions with varying normality of NaOH. Distributions around 0.9 are optimal, resulting from a range of NaOH normalities from 0.1 to 0.2. Increasing or decreasing NaOH de-stabilizes signal, resulting in decreases in normalized signal that lead to errors in CNV calling. FIG. 7B shows signal displayed as Log R Mean, a metric comparing test data to reference data, where stable Log R Mean indicates stable signal (Log R Mean is the average Log R Ratio in a set of probes; see online at illumina.com/Documents/products/technotes/technote_cytoanalysis.pdf). Log R Mean is stable for denaturation conditions using 0.1 to 0.2 N NaOH, indicating that CNV calling is stable and accurate in this range.

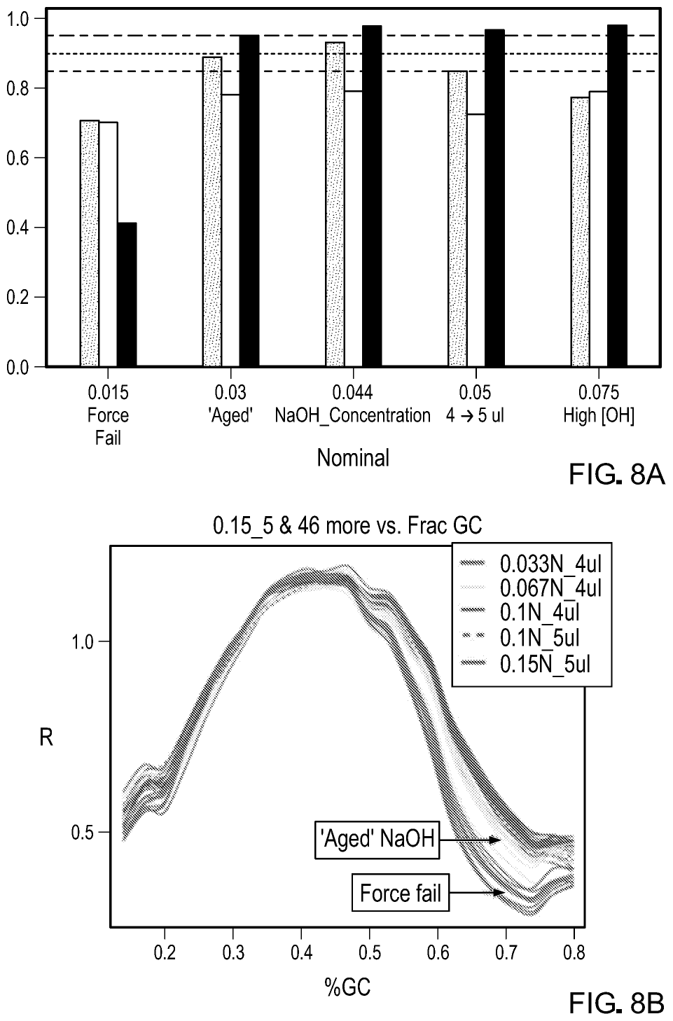

Additionally, it was found that lowering the addition of NaOH to 4 μl of 0.033 N NaOH suppressed the ability of the CNV Caller to accurately call CNV content. Signal trended by GC content, with less than 0.044 N (4 μl of 0.1 N NaOH added) yielding suppressed signal at high and low % GC probes (FIGS. 8A and 8B). FIG. 8A shows CNV Caller F measure (a metric incorporating both accuracy and precision) for three regions of CYP2D6. Force Fail shows low F measures indicating poor CNV caller performance with a hydroxide concentration of 0.015 M in denaturation. Denaturation conditions with hydroxide concentration of 0.03 M through 0.075 M yield similar F measures, indicating high quality and stable CNV calling capability. Together, this result shows that the hydroxide concentration in denaturation is a critical parameter to enable accurate CNV assessment, and that optimizing this parameter increases CNV calling capability.

Denaturation reaction conditions lead to shifts in signal that impact the capability of CNV Calling in genotyping. This can be a source of bias that leads to inaccurate CNV calling, with accuracy of genotyping methods improved through optimization of denaturation. Because the mechanism of CNV calling methods typically rely on comparison of a test sample to a reference sample, or test region to a reference region, and because denaturation leads to shifting in a GC-content dependent manner, optimization of denaturation and other workflow conditions that impact signal bias is expected to improve CNV calling across genomics technologies.

CNV calling is accomplished through measuring direct and relative amounts of DNA present for regions of interest. This can be done by comparing the amount of DNA, or a proxy like signal generated from DNA, in a target region relative to a control region, or a specific sample against a reference sample, as two examples. In taking such measurements, biases present in the assay have the potential to impact the results. One such bias relates to the DNA base content, often considered as the aggregate of G+C (GC content), of the genome. For example, a polymerase may more efficiently amplify some regions than others, and this can relate to the GC content of these regions. There can be additional layers of variability in a process, for example the bias can be increased or decreased in strength by increasing or decreasing the concentration of enzymes or other reaction components. In general, reducing such biases will increase the ability to calibrate the assay, thereby increasing the accuracy of results.

In determining CNV states in PGx regions, a key source of bias was found to be the sodium hydroxide (NaOH) step. The concentration of hydroxide (OH) compared to the concentration of the DNA sample was determined to be a critical parameter, with incomplete DNA denaturation occurring in a biased manner, leading to a signal bias that related to the GC content of the regions in question. Because PGx content (e.g. CYP2D6) is significantly higher GC content compared with the average across the genome, these regions were more heavily affected by this effect. It was determined that the denaturation step had to be modified to include a minimum hydroxide concentration compared with DNA concentration, which was achieved by adjusting the amount of NaOH added at a specific normality. Additionally, because NaOH acidifies when exposed to the atmosphere, lowering the hydroxide concentration of the solution, limits were placed on how long the reagent can be left out before being added to a reaction. Addition of mineral oil to the WGA reaction helps reduce acidification, and adding NaOH and DNA under a layer of mineral oil is another necessary step to ensure high quality CNV calling.

### Example 6: Improved Pseudogene Disambiguation Using TGA

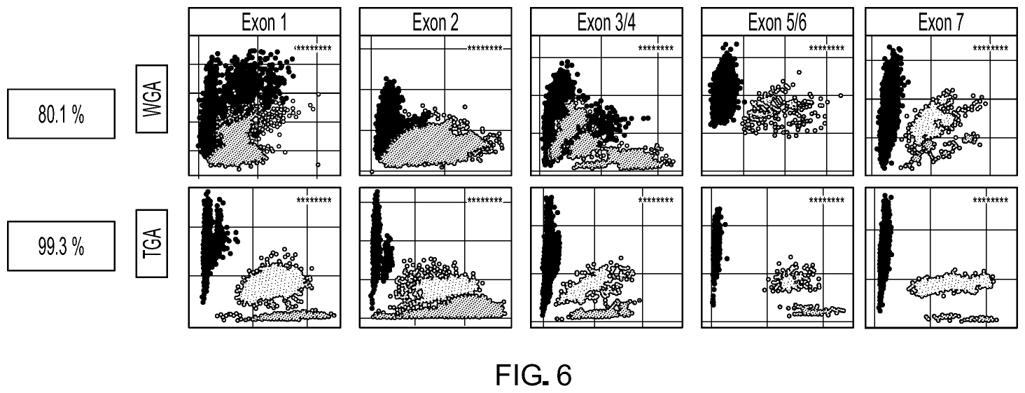

288 samples tested on a prototype GDA-PGx chip showed that incorporating the TGA step in the workflow improved signal disambiguation for regions covered by TGA amplicons. Signal for probes under five regions of CYP2D6 are shown with (top) or without (bottom) the TGA step added (FIG. 6). Without signal disambiguation, covered variants were called with 80.1% accuracy, while adding TGA improved accuracy to 99.3%.

The source of the noise in CYP2D6 regions shown in FIG. 6 is two highly homologous pseudogenes, CYP2D7 and CYP2D8. When genotyping WGA material alone, roughly equivalent amounts of DNA from each of the three regions is present in the library, and all of them can potentially hybridize on capture probes intended only for CYP2D6. The result is that signal from CYP2D6 is highly polluted with noise from CYP2D7 and CYP2D8 (80.1%, FIG. 6, top). In other genes, the same dynamic can play out with pseudogenes, other genes of high homology to target, or under other conditions of high homology.

To circumvent this challenge, material from the TGA process, in this case amplified via multiplex PCR, is added to the WGA before hybridization to the array. This process is optimized to ensure that signal is generated from amplified, on-target material, and that background WGA is reduced to noise. This results in genotyping of the desired amplified material, making it possible to obtain accurate genotyping results (99.3%, FIG. 6. bottom).

This process is designed to leave some areas uncovered by amplicons, to enable CNV calling to be performed on background WGA signal.

### Example 7: Improving Workflow Incorporation and Probe Performance with Uracil PCR

In some cases, increasing the amount of DNA in the hybridization solution can paradoxically lead to a decrease in signal at target probes, inhibiting either/both binding of target DNA to probes or signal generation of captured DNA. Incorporating amplified DNA into genomic DNA samples before WGA occurs leads to a rescue of this phenotype, but is not compatible with lab workflows. This creates challenges to the incorporation of amplified DNA into the genotyping workflow, and operational constraints around handling of samples, e.g. physical separation of pre- and post-amplification rooms, limit the options to address the issue.

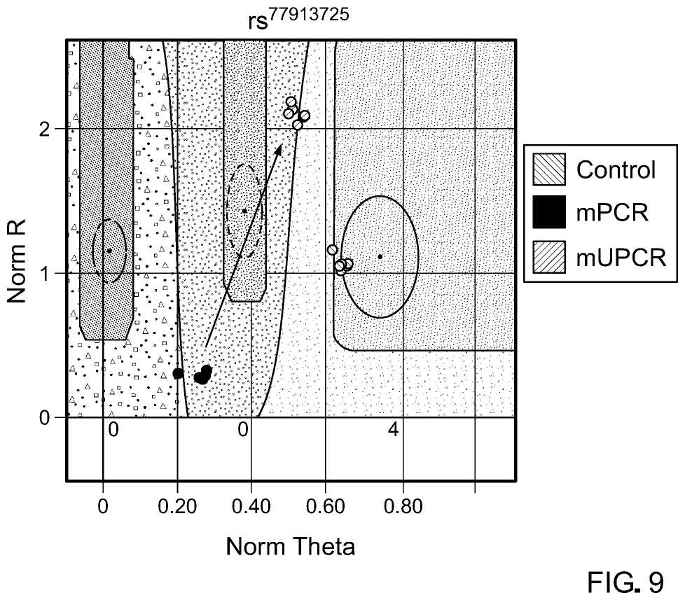

To circumvent the issue of both PCR material handling and signal decrease with increasing DNA concentration, the TGA step was modified to include uracil in the PCR step. This enabled target regions to be amplified and then integrated into the workflow before the U-dependent fragmentation step, yielding fragmented amplicons that are able to bind probe sequences and generate signal. FIG. 9 shows results of using a probe that shows high levels of noise in the control (WGA only, “Control”—points in the middle of the right half of the graph) evidenced by a theta value far from any canonical cluster (0, 0.5, or 1), is inhibited in the presence of multiplex PCR-amplified material that does not contain uracil (WGA+mPCR, “mPCR”—points in lower left of the graph), and yields an AB call at the canonical cluster position in the presence of uracil-containing multiplex PCR (mUPCR+WGA, “mUPCR”—points clustered in the middle of the upper half of the graph), demonstrating rescue of an inhibited probe by fragmentation of the target amplicon.

### Sequences

SEQ ID NO: 1 (CYP2D6)
>NG_008376.4:5001-9312 Homo sapiens cytochrome P450 family 2 subfamily D member 6
(CYP2D6), RefSeqGene (LRG_303) on chromosome 22
ATTTGGTAGTGAGGCAGGTATGGGGCTAGAAGCACTGGTGCCCCTGGCCGTGATAGTGGCCATCTTCCTGCTCCTGG
TGGACCTGATGCACCGGCGCCAACGCTGGGCTGCACGCTACCCACCAGGCCCCCTGCCACTGCCCGGGCTGGGCAAC
CTGCTGCATGTGGACTTCCAGAACACACCATACTGCTTCGACCAGGTGAGGGAGGAGGTCCTGGAGGGCGGCAGAGG
TGCTGAGGCTCCCCTACCAGAAGCAAACATGGATGGTGGGTGAAACCACAGGCTGGACCAGAAGCCAGGCTGAGAAG
GGGAAGCAGGTTTGGGGGACGTCCTGGAGAAGGGCATTTATACATGGCATGAAGGACTGGATTTTCCAAAGGCCAAG
GAAGAGTAGGGCAAGGGCCTGGAGGTGGAGCTGGACTTGGCAGTGGGCATGCAAGCCCATTGGGCAACATATGTTAT
GGAGTACAAAGTCCCTTCTGCTGACACCAGAAGGAAAGGCCTTGGGAATGGAAGATGAGTTAGTCCTGAGTGCCGTT
TAAATCACGAAATCGAGGATGAAGGGGGTGCAGTGACCCGGTTCAAACCTTTTGCACTGTGGGTCCTCGGGCCTCAC
TGCTCACCGGCATGGACCATCATCTGGGAATGGGATGCTAACTGGGGCCTCTCGGCAATTTTGGTGACTCTTGCAAG
GTCATACCTGGGTGACGCATCCAAACTGAGTTCCTCCATCACAGAAGGTGTGACCCCCACCCCCGCCCCACGATCAG
GAGGCTGGGTCTCCTCCTTCCACCTGCTCACTCCTGGTAGCCCCGGGGGTCGTCCAAGGTTCAAATAGGACTAGGAC
CTGTAGTCTGGGGTGATCCTGGCTTGACAAGAGGCCCTGACCCTCCCTCTGCAGTTGCGGCGCCGCTTCGGGGACGT
GTTCAGCCTGCAGCTGGCCTGGACGCCGGTGGTCGTGCTCAATGGGCTGGCGGCCGTGCGCGAGGCGCTGGTGACCC
ACGGCGAGGACACCGCCGACCGCCCGCCTGTGCCCATCACCCAGATCCTGGGTTTCGGGCCGCGTTCCCAAGGCAAG
CAGCGGTGGGGACAGAGACAGATTTCCGTGGGACCCGGGTGGGTGATGACCGTAGTCCGAGCTGGGCAGAGAGGGCG
CGGGGTCGTGGACATGAAACAGGCCAGCGAGTGGGGACAGCGGGCCAAGAAACCACCTGCACTAGGGAGGTGTGAGC
ATGGGGACGAGGGCGGGGCTTGTGACGAGTGGGCGGGGCCACTGCCGAGACCTGGCAGGAGCCCAATGGGTGAGGCT
GGCGCATTTCCCAGCTGGAATCCGGTGTCGAAGTGGGGGGCGGGGACCGCACCTGTGCTGTAAGCTCAGTGTGGGTG
GCGCGGGGCCCGCGGGGTCTTCCCTGAGTGCAAAGGCGGTCAGGGTGGGCAGAGACGAGGTGGGGCAAAGCCCTGCC
CCAGCCAAGGGAGCAAGGTGGATGCACAAAGAGTGGGCCCTGTGACCAGCTGGACAGAGCCAGGGACTGCGGGAGAC
CAGGGGGAGCATAGGGTTGGAGTGGGTGGTGGATGGTGGGGCTAATGCCTTCATGGCCACGCGCACGTGCCCGTCCC
ACCCCCAGGGGTGTTCCTGGCGCGCTATGGGCCCGCGTGGCGCGAGCAGAGGCGCTTCTCCGTGTCCACCTTGCGCA
ACTTGGGCCTGGGCAAGAAGTCGCTGGAGCAGTGGGTGACCGAGGAGGCCGCCTGCCTTTGTGCCGCCTTCGCCAAC
CACTCCGGTGGGTGATGGGCAGAAGGGCACAAAGCGGGAACTGGGAAGGCGGGGGACGGGGAAGGCGACCCCTTACC
CGCATCTCCCACCCCCAGGACGCCCCTTTCGCCCCAACGGTCTCTTGGACAAAGCCGTGAGCAACGTGATCGCCTCC
CTCACCTGCGGGCGCCGCTTCGAGTACGACGACCCTCGCTTCCTCAGGCTGCTGGACCTAGCTCAGGAGGGACTGAA
GGAGGAGTCGGGCTTTCTGCGCGAGGTGCGGAGCGAGAGACCGAGGAGTCTCTGCAGGGCGAGCTCCCGAGAGGTGC
CGGGGCTGGACTGGGGCCTCGGAAGAGCAGGATTTGCATAGATGGGTTTGGGAAAGGACATTCCAGGAGACCCCACT
GTAAGAAGGGCCTGGAGGAGGAGGGGACATCTCAGACATGGTCGTGGGAGAGGTGTGCCCGGGTCAGGGGGCACCAG
GAGAGGCCAAGGACTCTGTACCTCCTATCCACGTCAGAGATTTCGATTTTAGGTTTCTCCTCTGGGCAAGGAGAGAG
GGTGGAGGCTGGCACTTGGGGAGGGACTTGGTGAGGTCAGTGGTAAGGACAGGCAGGCCCTGGGTCTACCTGGAGAT
GGCTGGGGCCTGAGACTTGTCCAGGTGAACGCAGAGCACAGGAGGGATTGAGACCCCGTTCTGTCTGGTGTAGGTGC
TGAATGCTGTCCCCGTCCTCCTGCATATCCCAGCGCTGGCTGGCAAGGTCCTACGCTTCCAAAAGGCTTTCCTGACC
CAGCTGGATGAGCTGCTAACTGAGCACAGGATGACCTGGGACCCAGCCCAGCCCCCCCGAGACCTGACTGAGGCCTT
CCTGGCAGAGATGGAGAAGGTGAGAGTGGCTGCCACGGTGGGGGGCAAGGGTGGTGGGTTGAGCGTCCCAGGAGGAA
TGAGGGGAGGCTGGGCAAAAGGTTGGACCAGTGCATCACCCGGCGAGCCGCATCTGGGCTGACAGGTGCAGAATTGG
AGGTCATTTGGGGGCTACCCCGTTCTGTCCCGAGTATGCTCTCGGCCCTGCTCAGGCCAAGGGGAACCCTGAGAGCA
GCTTCAATGATGAGAACCTGCGCATAGTGGTGGCTGACCTGTTCTCTGCCGGGATGGTGACCACCTCGACCACGCTG
GCCTGGGGCCTCCTGCTCATGATCCTACATCCGGATGTGCAGCGTGAGCCCATCTGGGAAACAGTGCAGGGGCCGAG
GGAGGAAGGGTACAGGCGGGGGCCCATGAACTTTGCTGGGACACCCGGGGCTCCAAGCACAGGCTTGACCAGGATCC
TGTAAGCCTGACCTCCTCCAACATAGGAGGCAAGAAGGAGTGTCAGGGCCGGACCCCCTGGGTGCTGACCCATTGTG
GGGACGCATGTCTGTCCAGGCCGTGTCCAACAGGAGATCGACGACGTGATAGGGCAGGTGCGGCGACCAGAGATGGG
TGACCAGGCTCACATGCCCTACACCACTGCCGTGATTCATGAGGTGCAGCGCTTTGGGGACATCGTCCCCCTGGGTG
TGACCCATATGACATCCCGTGACATCGAAGTACAGGGCTTCCGCATCCCTAAGGTAGGCCTGGCGCCCTCCTCACCC
CAGCTCAGCACCAGCACCTGGTGATAGCCCCAGCATGGCTACTGCCAGGTGGGCCCACTCTAGGAACCCTGGCCACC
TAGTCCTCAATGCCACCACACTGACTGTCCCCACTTGGGTGGGGGGTCCAGAGTATAGGCAGGGCTGGCCTGTCCAT
CCAGAGCCCCCGTCTAGTGGGGAGACAAACCAGGACCTGCCAGAATGTTGGAGGACCCAACGCCTGCAGGGAGAGGG
GGCAGTGTGGGTGCCTCTGAGAGGTGTGACTGCGCCCTGCTGTGGGGTCGGAGAGGGTACTGTGGAGCTTCTCGGGC
GCAGGACTAGTTGACAGAGTCCAGCTGTGTGCCAGGCAGTGTGTGTCCCCCGTGTGTTTGGTGGCAGGGGTCCCAGC
ATCCTAGAGTCCAGTCCCCACTCTCACCCTGCATCTCCTGCCCAGGGAACGACACTCATCACCAACCTGTCATCGGT
GCTGAAGGATGAGGCCGTCTGGGAGAAGCCCTTCCGCTTCCACCCCGAACACTTCCTGGATGCCCAGGGCCACTTTG
TGAAGCCGGAGGCCTTCCTGCCTTTCTCAGCAGGTGCCTGTGGGGAGCCCGGCTCCCTGTCCCCTTCCGTGGAGTCT
TGCAGGGGTATCACCCAGGAGCCAGGCTCACTGACGCCCCTCCCCTCCCCACAGGCCGCCGTGCATGCCTCGGGGAG
CCCCTGGCCCGCATGGAGCTCTTCCTCTTCTTCACCTCCCTGCTGCAGCACTTCAGCTTCTCGGTGCCCACTGGACA
GCCCCGGCCCAGCCACCATGGTGTCTTTGCTTTCCTGGTGAGCCCATCCCCCTATGAGCTTTGTGCTGTGCCCCGCT
AGAATGGGGTACCTAGTCCCCAGCCTGCTCCCTAGCCAGAGGCTCTAATGTACAATAAAGCAATGTGGTAGTTCCAA
SEQ ID NO: 2 (CYP2B6)
Homo sapiens cytochrome P450 family 2 subfamily B member 6 (CYP2B6), RefSeqGene
(LRG_1267) on chromosome 19
NCBI Reference Sequence: NG_007929.1
>NG_007929.1:4984-32100 Homo sapiens cytochrome P450 family 2 subfamily B member 6
(CYP2B6), RefSeqGene (LRG_1267) on chromosome 19
GTGCAGGGCAGTCAGACCAGGACCATGGAACTCAGCGTCCTCCTCTTCCTTGCACTCCTCACAGGACTCTTGCTACT
CCTGGTTCAGCGCCACCCTAACACCCATGACCGCCTCCCACCAGGGCCCCGCCCTCTGCCCCTTTTGGGAAACCTTC
TGCAGATGGATAGAAGAGGCCTACTCAAATCCTTTCTGAGGGTAAGACACAGACGAATGGGGTCTGAGGGTGAGCTG
CTTCTTGCCTTGGTACTTGGGGAAGCTTCACCAAACAGAATGAGGCAGACTTCCAGAGTCAGGGGTGGCACGGGCAT
GGTTGGTGAGTACGGAGCATGGTGAAGCATGATGGGTGGTATTATTAGGAGAAAAGCATCAAATTAAATTTAGCAGA
GTTTATTTGAGCAAAGAAGTGACTCATGAGTTGGACAGCTCCCTAAACCAGGAAAGACACCACACGGCAGTATGGTC
AAGTGGTATTTACAGGCAGAAAAAGGAGGTGACATACAGAAACAGCCTGATTGGCCACAGATCACAGCTTGCCTTAC
TTGGTCACAATCTGAGCAGTTTGCAGCCTGTGTGGACTGAAAGCCCAGCTGCTCTGATTAGCCAACACTTGGCTACT
TGTCACAAGAATATATTCATTTGGGCCAGGTGCAGTGGCTCATGCCTGTAATCCCAGTGCTTTTGGAGGCCGAGGTG
GTGGATCACCTGAGGTCAGGAGTTCGAGACCAGCCTGGCCAACATGGTGAAACCTTGTCTCTACTAAAAATACAAAT
ATTAGCTGGGCATAGTGATGCGTGCCTGTAATCTCAGCTACCCAGGAGGCTGAGGAAGGAGAATCACTTGAATCCAG
GAGGCAGAGGTTGCAGTGAGCCAAAATCTTACCACTGCACTCCATCCTGGGTGACAGAGTGAGACTCCTTCTCAAAA
AAAAAAAAAAAAAAAAGAATATACTCCCAAGTTAGGTTGCAGTTCACTCTACAGAGAGAGCTTTAGGTCAAATTTAA
TTTAATTAAACAATTCTCCCCTTTTGGTCAGCCTCAAAATTTTGAGATTGACCAAAACCTTGGGCATCAACATTACT
TCTGTCACCATCATAATGGACTTGTCTGCTCTCAGTATGGAATTCACAATGGACAATGTCAACGTAGTTGAGTGATT
CTTTACCTTTTCTTCATGTTTTTGTTGTTCCCACTGTAATGAGCCCACTGGATGTACAAAGAATGGCTGCATATGAG
CATTTAAGACTCTTTTTTTTTCTGAGACAGGGCCTCACTCTGTCAGCCAGGCTGAAGTGCTGTGGCATGATCACGTC
TCACTGCAGCCTTGACCTCCCAAGGCTCAAGTGATCCTCCTGCCTCAGCCCCCCAAGTAGCTGGAACTACAGGTGCA
TGCCACCACGCCCAGCTAATTTTTGTATTTTTTGTAGAGACAGGGTTTTGCCATGTTGCCCAGACTGGTCTTAAACT
CCTGGGCTCAAGCAATCCACCTGCCTCGGCCTCCCAAAGTGCTAGGATTACATGTGTGAGCCACCGCACCCGGCCAA
GACTCTTGAGAAAATACAACACATCAGGGAGACTGTTATGATGGCTCTCAGGAGGGTAATACGAAGAAAATGAAGTC
ACTGGGCCTGTAATAAACTTTGAGGAATGTGGACTTGGGGGTATAGATAAGGTCCACTGTCCACAGAGAGAAGAAAG
GCTGTTAATAGTCTCTTTTAACTTGAGTGTGTCCATGAACCAAACTGATCAAAATCGAATAATTCGAAGTTCAGACA
ATAAAGATAGTTCAATAGTATTAGAGTCCAATTGGTCATAGATTTTGTTCAGGGCATGATGGTAATTAAGGACCAGA
GCTTGCTATAAAATAACTTGATTTATAGAGACATTCATTTGTAGTTGGCCTGGTAACATATAGTATCCTGGAGACCC
ACTAGAAGAAACATTAAGAGTAGAAAAGTTTGGGATAGCCAGGCTTGCTGTGTTAGTCCATTCTCACACTGTTATAA
AGACATACCTGAGACTGGGTAATTTATAAACAAAAGGGATGTAACTGACTCACAGTTCCACATGGCTGGGGAGGCCC
CAGGAAAATACAATTCATGGCAAAAGGTGAATGAGAAGCAGGAAACTTACAATCATGATGGAAGGTGAAGGAGAAGC
AAGTACCTTCTTCACAAGGTGGCAGGAAAAAGAGAGAGAGCCAAGGGGGAAGAGCCTCTTATAAAACCATCAGATCT
TGTGAGAACTCACTCACTATCACAAGAACAGCATGGGGGAAACCGCCCCCAGGATCCAGTTACCTCCTACTAGGTCC
TTCCCTCCACACCTGGGGATTACAATTCAAGATGAGATTTGGGTGAGGACACAGAGCCAAACCATATCACTTACCAT
CACCATTCAGGATGCTTGCAAACCAACTGCTAGCTGCACCTGTAAACACATATCTGTTTCTTTCCCCTGAGAAATGT
CCTTAGTGTATTTGTGGCAGTGTCTAGAGAAACAGCAGTGTCAGCCGCATTTTAAATTAAGTTATCTGCACTAGTGA
ATTCACTGGAAAGATAAGAGCAATATTTGGTTTTCTTCAGCACCATACACAAGCCTCCAAGATGGGCATAGAGGAGA
TCTAAAATTGCGTGATGTTCCATTAAGCGTTTTTGTTGCCACAAATGTTCTCATCTCAGTTTGGAGAGTGGCTTCTA
CCCATCTGAACTCCTTGGAGGTTCAATTAGCTGCAAAATTCAAGATGTCCCTTAATGTATAACTTAGCCTCAGATTC
CATACAACTGTCACCCAAATACCACCAAGAATGAGCACCCAGGAACCCAACTGGAACCTTTTCTGAACAGAAACCAA
CTTATCTTCGTCGATTTTGAGGTTGATAGTAATTTCAGTTATTGACTGTTTTGGCTTTTAACTATGGGAGGTATTAG
GAAACTCTCAGGGAAACAATTTGGAAAGCAGCAGTGAGCTAGGCCAAATAGCAAGTTCTGGACCTGTGAGGAGAAAG
AACAGAGTAAGCAAACCTCAAGATACTCAAGGTAGGCACTCGTGGTGTTGGAAAAGAGGGTCACCTACTGGCATTAG
AGCAGAGATCAGTTAGATTTGTTTACCCATAAGTCTGCATAGCTCCTGAACAAGGTGGGAAACTTACTTTTTTGTGG
TCTTTTTCTAGCATGCTGCGAAGGTGCATAACCACATTTAGTTGGAAAGAGACTTTACTGTATTTACTTATTTATTT
GTTTTTAATAGAGTTGGGGTCGTGCTATGTTGAACTCTTGGCCTCAAGCAATCCTCCCATCTCAATCTCCCAAAGTG
CTGGGATTACAAGCATGAGCCACCATGCCTGGCCACTTTACATATTTAATCCAGTAACATTACACACGCAATTGCCC
ACACCCCCATAGGTAGTCCCCAGGTCTTGCATACGGGATGCCTGGAAGCAAAATATGCCTTTTGCAGCCATTATTCA
GATACATTTCCTATATTTAGTAGTGATTATGTTATTAGCTAGTTAATAGTATGTTATTACGTACTGTTATTATATTA
ACTAACTAATAACATAATCACTACTAAATATTTCCAGTGAGTGCAAAAAAGCAAGTGGCAATGATGTCTAGAATATC
AAGATATAGCTTTCCACTCCTCCTTTGGGGTTTCTGGGTGATTCTCATTGGGAACATGAAGAGGCATTGGCACCAGT
GAAATTATTTCCTGATTTTGGGGCGTTGGTTCAGAAACTCAACAACTCCTTGTTTTTTTTGTTTGTTTGCTTGTTTT
GCTAGAGTATAAGCCTTTGCTAAAGCCATTCACAGATTATAGTCCTATGGATTTTCTTGTAAGGAAAGGGAAAGGGT
TAGGACAGCAAGAAATGGGGAAGAAAGGATAAAAGATAATGCTTTCATGATGGAAGAGAAATCTTGATCCACAATCT
TGGAAAAGCTGTCCGCATATAAGATGCCAACTGCTTCTGGGGAAAAACTTCCCTGGTCAGCTTTGCCTTAAGGTCTC
CAACAGACATACAGTTCTAGGAGTCTAGAAGGGTCCTTTCCAATGGAGAGATGTGGATCCAAGATCCGAGACCCTGA
CATTTTGCTACAGAGAAGAACTTGGCATTGTCCTTCCCAATGGAGTACAAGGAACAGTCTTAGAAGAACTTGGTACG
GTCTCTTCCAATGGAGTTCAAGGACAGTTTGTCTGGTGTCATTTCCAAAGGGCCCAACCTCTAAATTCTAGATCATG
AAAGGTCTGGTTGTCATCAACCGATGTGTCATCAATGACTCATTTTACCTGGTGAAAACATGCTTTGGCATAAAGTA
TTATAGCCTTGCATTATTGAGTCATATCAGAGTTTATAAGAGTGGGAGATACATGAGATTCTATTATTAGGGGCATA
GGCCCTCTATTACTATTTTACAAGAGATCTATCTATGTCTTTCCAGTAGGAGTGGATCTGATTGCCATCAATCAATA
ATACCTGAGACCAAGGGACTCCAATCAATTCAGCATGCTTTGCCTAATGATATTTGTTTGTAATACTGTTGCGGGAC
AATCAAAGACTGGAGAGACCAAAAAAGGTTCAGGAGAGTTTATTAAATTAAGGTGATCACCGGTTCAGCCAGACATA
CATCCAGAAAGTCTGAGCCCCGAACAAAGGCTTTTCCTACTTTTAAACATATTAAGGTGGGAACTACATGAGGCAGG
AAGCCAGTTTCAGAAGTGAGAAACAAAGCAGTTAAATAACATTTCTTACATCTTGAGAAAGACATGTCTTGCAACCT
AACCTTATCGGTCCGGTGACCCTGCAGCTGTGCAGGAACTCACTGGGCCTGTAATAAACTTTGAGGGATGTGGAGTT
GGGGAGTATAGGTAAGGTCCACTGTCCACAGAGAGAAGACAGGCTGTTAATATTCTCTTTTAACTTGAATGTAAGGT
GTGGTCATACTTTGCAGCAACCTTAAGAGGATTTTAAAATTTATATTACTACTACTATTAGGTTATAGTTGATTTCA
TTAATTCCTTCTTCAATACCTTATTTAACTGTTTTACCACTTGTCTAGTGAAACAAGTACCTCTGTCACTGGAGAGT
TCTCCAGGAATGCCCAGTAAGGAAATACATTTTCTAATAACCTTTTATCTACGGTTATGGCATTGATTGATCTTTGT
GCATAGAAATGCTTTAATACAACCAGAAAACATGCAATGAAGCTGGCAGTTGAATTAACTCCAGCTTCAAGTGTTCA
AATGATCTACCAAGTGCCAGAAATATATCACCTGAGGTTTTTGTTGTCTTACTAGAATTATGGATTTGATAAACCAA
ATATTAGTTATAAACCATTTAGTAATCTTAGAATAGTCACCTCATCAATATTTTTTCACTGTTTGGATCATTTTCTC
TCTTCTATGATGAGTCATGGAATACGGAGCTTTTAGTAATGGAAATTTTAAGAACTCAGGAAGGACCAGGCGGCCAT
CTAGGGTCTCCATGAGTGCATGCTTCACATTGGAATTACAGACTCTAAAGTACAAATTTTAATACAATGAGTTGCAA
TTTATGCTTCTCTAATTCAGGTACATAACACTGGTTTATTAAATAGGTTATCATAGGTAATTTGATGGTGCCATTGC
ACTCCAGCCTGGGTAACAGATTGAGACCCTGTCTCTGAATTGGAACTGCACGGGGGCACTGTCCTTGGAGGGGTGAA
TGGGCATGAAGAGGTGTCTGGGTATGAGCCACAGGTATAGAATTTCACTCTTCTCTGCCATCCTCTGTTACATCCTG
GGTACCTGCCTGCCACTGAAAGAATGAGGTAAAAGAGGTGGTGGCACGAATCAAATAGATCTTGCTGTGCCAATGAG
AGAGAGCAGACTAGCCCATGTCAGTGCCAGGAGAGTGGAGGAGAGAGGGAGAGCAGGAGAGGAGTGTGGGTAGGGAG
TGCTCATCAACAGTACACATAGTGCCCTATACCGGTAACTGCCACTGGCTCAGTATTTACCTGGGTTATCACTGCTT
ACCATGCCTGATTTTATGATTAATTATCTACTTATCATTACTAATCCATCAACCCACTTTCCAATGGGAGAATTAGA
ACACTGACAATACCTTCCAGCTCCTCTTCCCCTTCCCCCTCCCATGTTGACCATACTCCTGAATCTTAGGCTCGTTA
TCCTTTTACTATTAATAGAGTTATTTTTTTAATTTCTGATCAATGGTCTTTTTAATGATACCAAGTACAGAGTATAT
ATGCCAATACTACTATGAATTTTTAAATTATTTGCTCAGACAGAATACATGGACATACAAATGATGAATGTGATAAT
TGTCATACATATATATTTATCTTAGACACTTAGTCAAAACATGTGGTCTTTGGTTATCAGTTAGACACTGTCACTTT
ACCTGGAAGATACAAGGTAATTGGTCACAGACTCTCAAATTATGAAACATTTAGATTTTTCAGGGGAATGACATGGA
GGGAGCCAAGGAGTCTTATGATTAGATAAGATGTTGTTTGGGTGGCTCACGACACCACTGGGCAACACTCAAAGAGG
TGGTGGCTTATACCTGTAATCCCAACACTTTGGGAGGCTGAGGTGGGAGGATCGCTTGAAGCCAGGAGTTAGAAACC
AGCCTGGGCAACCAAACAAGACCTGGCTCTACAAAAAAGTTTTAAAAATTTAGCCAGGCATGGTGGCATGTGCCTGT
AGTCCCAGCTACTTGGGAGACTGAGGCAGGAGGATGACTTGAGCCTTGTAGTTTGAGGCTGCAGTGAGCTATGATCA
CGTCACTGCCCTCCAGCCTGGGCACAGAGCAAGACCCTGTCTCTTAAAAAAAAATCATCTGCAATGTGAGGAGTGAT
AACATTTAGGAACGTGTGTATAGGTTTAAATGCTGGTCAAAGACATCCTACACAATTCGCTGAACCTTCTCTCTAAG
GGTTTTTTCCCAAGCTCTGCAGACGCTATCTGGGCACAAATCATGCCTCTGTTAACAGAATTTGCTGTTCCTTCTAG
CTCTTGGTATCCCACTGCCCGCTTTCTTTATGAAAGCTGGTATGGTCATTGAATATCCCAATCCTTTACAAATTTGG
AAACAAGCAAAACTGTCAATGAAATTTGTATTTGCCTAAAATGAGTTTTCTTTCTTTCTTTTTTATTATTATTATAC
TTTAAGTTTTAGGGTACATGTGCACATTGTGCAGGTTAGTTACATACGTATACATGTGCCATGCTGGTGCGCTGCAC
CCACTAACTCGTCATCTAGCATTAGGTATATCTCCCAATGCTATCCCTCCCCCCTCCCCCCACCCCACAACAGTCCC
CAGAGTGTGATGTTTCCCTTCCTGTGTCCATGTGATCTCATTGTTCAATTCCCACCTATGAGTGAGAATATGTGGTG
TTTGGTTTTTTTGTTCTTGCGATAGTTTACTGAGAATGATGATTTCCAATTTCATCCATGTCCCTACAAAGGACATG
AACTCATCATTTTTTATGGCTGCATAGTATTCCATGGTGTATATGTGCCACATTTTCTTAATCCAGTCTATCATTGT
TGGACATTTGGGTTGGTTCCAAGTCTTTGCTATTGTGAATAATGCCGCAATAAACATACATGTGCATGTGTCTTTAT
AGCAGCATGATTTATAGTCCTTTGGGTATATACCCAGTAATGGGATGGCTGGGTCAAATGGTATTTCTAGTTCTAGA
TCCCTGAGGAATCGCCACACTGACTTCCACAATGGTTGAACTAGTTTACAGTCCCACCAACAGTGTAAAAGTGTTCC
TATTTCTCCACATCTTCTCCAGCACCTGTTGTTTCCTGACTTTTTAATGATTGCCATTCTAACTGGTGTGAGATGGT
ATCTCATTGTGGTTTTGATTTGCATTTCTCTGATGGCCATTGATGTTGAGCATTTTTTCATGTGTTTTTTGGCTGCA
TAAATGTCTTCTTTTGAAAAGTGTCTGTTCATGTCTTTCGCCCACTTTTTGATGGGGTTGTTTTTTTCTTGTAAATT
TGTTTGAGTTCATTGTAGATTCTGGATATTAGCCCTTTGTCAGATGAGTAGGTTGCGAAAATTTTCTCCCATTTTGT
AGGTTGCCTGTTCACTCTGATGGTAGTTTCTTTTGCTGTGCAGAAGCTCTTTAGTTTAATTAGATCCCATTTGTCAA
TTTTGTCTTTTGTTGCCATTGGTTTTGGTTTTTTAGACATGAAGTCCTTGCCCATGCCTATGTCCTGAATGGTAATG
CCTAGGTTTTCTTCTAGGGTTTTTATGGTTTTAGGTCTAACGTTTAAGTCTTTAATCCATCTTGAATTGATTTTGTA
TACTAAAATGAGTTTTCAAAAGGATCTTTGTGGCTACCTTATTAGTTCATAGAAAGTGGAGGCTTGTCTGGAATGAT
ATTAAAGAATTTTTTTCATTTTAATTGTTTTAGAGACAGGGTCTCAGTCTGTTACCCAGGCTGGAGTACAATGGCAC
AGTCATAGCTCACTGCAGCCTTGAACTCCTGTGCTCAGGAGATTCTCCCACCTTAGCCTCCAGAGTAGCTGGGACTA
AAAGTGTGAGCCACCATGCCCCACTATTTATTTTTGTAGAGATGTGTTTGGGGGACGGTCTCACTATGTTGCCTAGG
CTGGTCTCGAACTCCTGGACTCAAGCAATCCTCCTGCCTCAACCTCCCAAAGCATTGGGATAAGTTTTGCATAGACA
TGTTTGACCCTCTCCCTTCCTTTATTGCAGCAAAGATTTCCATTTTCTCACAGGGAGGACTTGGGGCAAATTGTTTT
TGTCGTTGTTATTGTTATTTGAGGATGTGGGTGGGTCATCTTGGTTAATGTCAGTGCAAGAACGGTGTCTGCTCCTC
ATTATTGGGGTTCCTTAGCTATTCAGGCAGGCAGCAGCTCCCTTGAGAAGACTTCCTGACCCCCAGGTCCCCATCAT
ATGCTCTCATACACCAGCTGCCCCTCCTTACTGAGCCTATGTCCTTGATAGTGCTGCATTTACCTGTGATCCTGTGG
CTGAGCTCTCCCTCACACTTCCAGACAAGGAAGGCCTGTGAGGTATCAGCACAGTGCTGAACATAGTACCTGGTACA
CAATAGGCATTTAGTAAATATGTGAACAGGAACAAATGAAGGAGTCAGTGAGTGAAAGCTCCAAGCCTGACTCAGCG
GAACTGGCAGTCGGCCAGGGCCTACAAAGTGCTGCCTGGCCCTCAGTAGGGGGTGGCAGATCTGGGGATCCCTCTTC
ACCAAATAGAGTTGCATCATACAGGTAAAGGTCCCAAGTGCCTATTGTTTTCTTTTCATCATTTTCCATGTGTGACA
AGAGTATCACGCAATCATGTGAATCAATGGACTTAGTTTTCTCACTAGACTAATGTGTCTAGGAATTATCTGTGTTG
TCATGGGGATTTTCCAGGTGTCATTTACAACTACCTGTGGACAAGATGAGGTGCTACCCTCATCTTAGAATTAGGGA
TGGTGGCTGGGCATGGCAGCCCATGCCTATAATCTCAGCACTTTGGGAGGCTGAAGCAGGTGGATCAACTGAGGTCG
GGAGTTCGAGACCAACCTGACCAACATGGAGAAACCCTGTCTGTACTAAAAAACACAAAACTAGCTGGGCATGGTGG
CACATGTCTGTAATCCCAGCTACTCGGGAGGCTGAGGCAGGAGAATTGCTTGAACCCAGGAGGCGGAGTTTGCCCTG
AGCTGAGATTGCATCATTGCACTCCATCATGGGCAACAAAAGTGAAACTCCATCTCAAAAAAAACCCAAACAGAAAC
AAACAAACAAACAAAATTAGTGATAGTGATGCTCAGCCTGGGGAAAGCATTTGTCCAGTGGCACACAACTGGGAAAA
GGGGAAGCTGAGATCCAGCAGGAGGTCTGTCTCCAAAGCCCTCCTAGACTAAAGCTGCTTAACAATTTGTGGATTAT
GAAATTCTCATAGAGTTTGATTAAAGCTGTGGCCCCCTCTTTTCCAAATAGGCACATACATGTTTGCATAAAGTTGT
GAGGCTTCACACACTCCCTAAAACTCTTTCATAAACCTTCCAAGGATCCTCTAGGTATTCACGACCATCTATTATGA
TTGCATCTCTTGGGGGTGGGGTAAAGAGGGAGGGCATGAGCAAGTGTGCATCAGGGCTGAGGAAGGTGGCGCTGTTG
CTTCTCCATTTCCCAATAAGCTTCCAGATTCTTTTTGATGTCAGAGGGATGTGGGCTCGTGTCTCAGATTCAACCCG
TATGCATTGAGTCACAGTTTTCTCTTCCGTCAGTTAAGATCATGACAATGAGAATGTGTGCCCCTAAGGTGGTTGTG
AGGATTAAATGGGATATTGCATACAGCTAGTATATAATAAGTGCTCATTAAATGGCAACTACCTTGATCCACTCATT
CATTTATTCACGAATCCAATAACAATTCACTGGGCACTTTCTATGTACCAGGAAATAGTCTAGGAATTGATGATGTG
GCATCTTGGACAAGACATACGAGGTCACTGCGCTTATGGACATTCCATTGGCAGAGACAGATAAGCAAAAAATAAAC
AGATAAGGAAATGTTAGGTGGAGAAGAGCTACAATTAAACTAAAGCAGGGGGATATTTCAGACAGTGATGAGAACTA
CGTTAGATTGAGTGGTTAGGGGAAGACTGTCTTGTTTTTTTTTCTGAGCAGAGACAATGAAAGATTCAGGTGGAAGG
CACTAGAAGGAGGCAGGGGTGGCTACAAGAATCCTGAAACAGGAAAAATGTCAGAGAACAACATGGAGGAATGAGTG
GGAGGAGAAGTCAGAGCGGTAATAGGGCCACGTCATGTAGGACCATGTGAACCCTCAGTGGAGACTTCAGATCTTAT
TCTCAGCAAAGTGGGAATATTGTAGGCCTTGGTGAGCACGTGGATATTAATTATTTTACATTTTAGTGGGGTTACCC
TGGCTTGTGGGGCAGTGAAACAATTGTTAAGTGGAGTAAGCATGTCATCCAGAAGTCCAGTGAAGGGGCTTTGCAGA
GATCAATGCAGGGCATTTGTAAGTAAACTTAACAAATTTTATGACTTTTATGTCAAGTTTTTATTGAACTTGAACAT
CACTGTAAAAAGCGCACAGCTCAATGAATTTTCACAAACTGAACATACTCATGTAATTGACATGCAGGCCAAGTAGC
AAGAATGCCCCGAAACCCTGCACCCTGTTCTTTTCTATTCACTATCCACCTACCATCTTGATCATGGGTTGATTTTA
TCTGTGTATGAACTTCTTTTTTGTTTTTGTTTGTTTGTTCCTGAGATTGAATTTTGCTCTGTCGCCCAGGCTGGAGT
GCAGTGGCACTATCTCGCCTCACTGCAACCTCCGCCTCCCCGGGCTCAAGCAATTCTCTCGTCTCAGCCTCCCGAGT
AGCTGGGACTACAGGTGAGAGCCACCATGCCTGGCTAATTTTTGTATTTTTAGTAGAGATGGGTTTTCACCATGTTG
GTCAGGCTGGACTCAAACTCCTGACCTCGTGATCAGCCTGCCTCAGCCTCCCAAATTGCTGGGATTACAGATGTGAG
TCACCACGCCTGGCCCTGTGTTTGAACTTCATACACATAAAATTATGATGCATTGACTCTTTTGTGCCTAGTTGCTT
CCACTCAACATTATGCCTGTGAGTTTCAGCCACGTTGTCGCCTGTAGCTGTGGTTTGTTCTTTTTTGTTGCTGTATA
CTAGTCTATTGTGAGAACCCTCACTGATTGTCTATATCTATATTGATGAGCATTTTATTAAGAAAGCTTCTGTGAAT
ATTTTGTTCATTTTTTGGTGAACACACATACACATTTTTGCTGGGCATGTACCTGGGAGTGAAGTGGCTGGTTTCCT
GGGTATCCATTTGTTCAGCTTCAGGAAATACTGCCCAGCAGTTCCCCAAGTGGCTGCACAGTAATCCCACCTTATCC
ACTGGAGATATCTTCTGAGACCCCCAGTGGATGCCTGAAACCCCACATAATACTGAGCACTATGCATACTCTTTTTT
TTTCCTATACAATCACATTATATAGGGAGGGTGGTATACACAGTGCAGACATGGTGGACAAAGGATGATTCGTGTCC
GGGGTGGGACAGAGTGGATGGTGAGAGATATCATCATCCTACTCAGAATGATGCACAACTTAAAACTTACGAATTCT
TTATTTCTGGAATTTTCCATTTAATATTTTCAGACTGCGATTAGCTGCAGGTAACTGAAACTGCAAAAAGCAAAACC
ACAGATCATAAGAAGTATGCGGTGGGGGTGATATTCATCGTATTTTATCAACCATCTTTACTGTTTAGGGCACAAGC
CAATCAGAGCAGACCCTGGCTGGGCCACCCATTAACCTAAGCTTGTCCAACCTGCCTTATTTTGTTGTTGTTCTGTT
TTGTTTTGTTTTAGCTTTTTAGCAGCCTGAAGCCATGGTTTTCAGTTTCTGTCTCCAGTGATACACAGAAAGGAAGG
ATGAGGAAGGGGCTTTACTGGCCCAACCAGAAACGGAAACTAAGAACCCATGACTGTATTCTCTCCCTTGGACAGCG
TTAACCATTAACCCTTAATTGCTGGGTCCCAGCAGGGGAAAGGGCAGCCTGGGGAGGCGGATGTTGGGGAGGGGCTA
ATTACCAATCTGGTATGTAAGTATTTTGATAGTTTTACAAATGAGGTGTATGCTGACTAACAGCCACCCCTGGTGTG
GATGTGATTGGCAGTTCCGAGAGAAATATGGGGACGTCTTCACGGTACACCTGGGACCGAGGCCCGTGGTCATGCTG
TGTGGAGTAGAGGCCATACGGGAGGCCCTTGTGGACAAGGCTGAGGCCTTCTCTGGCCGGGGAAAAATCGCCATGGT
CGACCCATTCTTCCGGGGATATGGTGAGAGCCTCAGAGGCACTGGGAGGGGGCGGGTGGGGGGTGCATCAGGGAAGG
GAGTATATGGGAGGAAGAAGGACTCAGAGCCTTCTTCCAACTTCTTCTACAACCAACCCACACCTCCCCTGCACCCC
AGGTGTGATCTTTGCCAATGGAAACCGCTGGAAGGTGCTTCGGCGATTCTCTGTGACCACTATGAGGGACTTCGGGA
TGGGAAAGCGGAGTGTGGAGGAGCGGATTCAGGAGGAGGCTCAGTGTCTGATAGAGGAGCTTCGGAAATCCAAGGGT
GAGTCCTGGGGGATGAATAGGAAAGAAAGACAATGAAACACTGAGAGATGCAGGTGCACGGGAATAGAAAGACAGAG
AGGTATATAAGGGCACAGACAGAGACAGACGAAACTGGAGACACCATCAGACAGAGGGATAGAGACAGAGAGGGAGA
GAGACAGGGGAATAGAGAGGGATGGGGATGGGCAGGAGAGAAACACAGAGAGCCAGGGAAAGAGAGAGATGCCAGGT
GTATAATGTCCAAGAGTTACTCAAAGAGGCTGGATGTGATGACTCTCACCTGTAATCCCAGTACTTTGGGAAGCTCA
GGCAGGAGGATTGCTTGAGGCCAAGAGTTGGAGAACAGCCTGGGCAACATAATGAGATCCTGTCTCTACACAATATA
GAAAAGAAGTGAGCCACGCATGGTGGTGTGTGCCTGTAGTCCCAGCTACTCAGGAGGCTAAGGTGGGACTACAGGAT
CACTTGAGCCCAGGAGGTTGAGGTTGCAGTGAGCTGTGATTGTACCACTGCACTCCAGCCTGGACAACAGAGCAAGA
TCCTGTCTCAAACAAACAAACAAACCCTCAAAGACATATAATTTCATGGATCAATTGTGTCTGTCAAAGTCAAAAAC
GGAAGTTAAGTAAAAGAAAAAAACTACAGACATTTAACAAATAATGAACTGTGTTTTCCTTGCCCTGGGTGAAGTGC
TGATGAGCTGGCAGTGAGCAGACAGGCCAGGTGGGGTGTTCTGCCCGGGTGCAGCTGGAGGGGTCATCAAAGAATCA
CTAGGTTATTTTTGAGTTCTCCATAACTTGGTGTCTGTGAGACATGTAGGTGAAGGGTCTCTGGCTAGCACCTCCAT
CTCATTCATGCCAGGTGTTTACCATCTCTCTTATCAAAATTTCTCAAGAGACTCTGGGATGTAAATGCAGAGGCTGC
ATGGGGAGGTAGAGACCCAGGAGCTATAGGGAAACGGGGACAAGAAGACTGAAGAGAAGGACAGGAAGAAACAGTGA
CACAGGCAGGAGGAAAGAGACAGATGGAGGGACCAAAACAAAAAAATATAGGTTGGGTATGGGGCTCATGCCTGTAA
TCCCAGCACTTTGGGAGGCTGAGGCTGGCGGATCATTTGAGGCCAGCAGTTCAAAACCAGCCTGGCCAACATGGTGA
AACCCCATCTGTGCTAAAAATACAAAAATTAGCCAGGCTTGGTAGCACGTGCCTTTAATCCCAGCTACTCAGGAGGC
TGAGACAGGAGAATTGATTGAGCCTGGGAAATGGAGGTTGCCGTGAGCTAAGATCACACTACTGCACTCCAGTCTGC
ATGATAGAGTGAGACTCTGTCTCCAAAAATAATAATAATAATAATAAAATAAAGAACGGCAGGGGGGAGACAAATAT
ACACACAGAGAGACAGAAAGAAACAAAGGCAAAGAGAAATTGAGGCAGAGAAAATTAGAGAGACAGACAGACAAAGC
TTAGGAAAAGGTCTGCAGAGGAATGAGAGAAGACAGGCAAGTGAGAACCAGAGAGAGGCTGCACTAACCTGATGTTC
TTGGGTCCTTACAGACCACTCTCCCTCCAGCTGGGGCCAGTGCTGAGCCTGGTGTATACAGGTATCACTTAACAAGT
ACAGAATAATTCCCAGAAGACTGGAGAGCCCTAGATGTGGAAAGAAGAGATTAAGGGGGAGTAATAGGTAGGGGTGG
AAAGATGGTTTTTTATTTGTTTTAAATTAGAGACGGGGTCTCACTCTGTACCCAGGCTGGAGTGCAGTGGCACGATC
ATAGCTCACTGCAGCCTCAAACTCCTGGGCCCATGTGATCCTCCCACTTCAGCCCCTGGACTATTTCAACTGGGACT
ACAGGCATGTGCCACCATGTCTAGCTACATTTTTTTTTTTTTTTTTTTGTAGAGACAGGGTCTCCTTATGTTGCCCA
GGCTGGTCTTGAATTCCTGATCCTTTTGAATCAGGCTCCCGAAGTGCTGGGTTTAGAGGTATGGGCCCCTGTGCCCA
CCCAGGGCTTTTTAATTTATATAAGCAATTGATTGAACACCTACTCTGCCCAGCCCCTATCCCTGGGATTTAACTGT
ACTCACTCCCAGAGTCAGAGGTGGGGCCTGAGAGGAGGTGCAGAGTGAGAACCGGCTGCATGGACTCTATAGCTGTG
TTGCCTGGGTCTAAATCCTGGCCTCAGTAATGAGTAGCTGTGCAACTTTGGTCAAATTACTCAGCCTCTCGGTCTGC
CCATCTATAAACTGGAGCTAATAATCAAATTGCATCTGCCTCACATTGTTGTAGTGAGAGTTCAATGGAATTACGCG
TGACGTGCTGGTACATAATTAGCTGTTACGGTTATTCTCATGTTTACCATTACTGAGTGATGGCAGACAATCACACA
GAGATAGGTGACAGCCTGATGTTCCCCAGGCACTTCAGTCTGTGTCCTTGACCTGCTGCTTCTTCCTAGGGGCCCTC
ATGGACCCCACCTTCCTCTTCCAGTCCATTACCGCCAACATCATCTGCTCCATCGTCTTTGGAAAACGATTCCACTA
CCAAGATCAAGAGTTCCTGAAGATGCTGAACTTGTTCTACCAGACTTTTTCACTCATCAGCTCTGTATTCGGCCAGG
TCAGGGAGACGGAGAGGGACAGGGGGTGTGGGGGTGAGGTGAACACCCAGAACACACGAGAAAAGGATGACCTGTCT
TGGGGGCTCAGAAATGCAGCTTATCCTTGGAAGAAACGCAGACATGTGAAGAATCAGGGACATGGAGACCTGGAGGG
AGGAGAGACGGTGAGACAGGGATAGAGACACTGAGAGAGAGAATGAGGCGTGATGGGGAGGCAGAAATAGAGTCAGA
GAGAGACTGAGAGAAGGAAGATGAGCAAAAACAAGACAAAGAAGAGCAGAAATCAAGAGATTCTGAGAGACAGAGTT
GATGAGAATGAGTGTGAAAGAGAGGGAGAGAGAGAGAACGAATAAGGCTTTGGGCTTCATGTCTATTCTGCTCCTGG
ATGTCATTTCTGTTTTATTTTTTTTAGACGGAGTCTCGCTGTTTCATTCCAGCTGGGGTGTAGTGTTGCCATCTTGG
CTTACTGCAACCTCCACCTCCCGGGTTCAAGTGATTCTCCTGCCTCAGCCTCCCAAGTAGCTGGGACTGCAGGCATG
TGCCACCACACCTGGCTAATTTTTTTTTTTTTCTTTTCGAGACAGAGCCTCGCTCTGTTGCCTAGGCTGGAGTGCAG
TGGCACAATCTCAGCTCACTGCAACTTCCACCTCCCTGGTTCAAGCAATTCCCCTGCCTCAGCCTCCTGTAGCTGGG
ATTACAGGCGCCTGCCGCTATGCCAGGCTAATGTTTTTGTATTTTTAGTAGAGACGGGGTTTCGCCATGTTGGCCAG
GCTGGTCTCTAACTCCTGACCTCAAGTGATCTGCCCGCCTCAGACTCCTAAAGTGCTGGAATTACAGGTGTGAGCCA
CCATGCCCAGACTGCTTCTGGTTCTTCTGTATCCTTGCTTCTCAGTCTTTGGTAAAGCTCTCCACCTAAAGAAAATG
AAGGATAAATGACAATAAGGAACAGCATTTCTTCATTTTCTCCCATTTCTCCTTCTCCCTCTGTGTTTTTTTTTTTA
ACTTTCCCCAGATTGTAAAGGCAGTCTTCTGCTCTTTTAAAACAAAATACTAAAATGTCTCCTTATTTATTAACCTG
GAAATATGCCTATTACATATTAAATTTAAGAATATCAAGCTGCAGAACAGTATGCATAGCTGTAGTTTGTTGTTGTT
GTTGTTTTCAGACAGTATCTTGCTCTGTTACTCAGGCTGGAGTGCAGTAGTGTGATCTCAACTCCCTGCCACCTCCA
CCTCCCAGGTTCAAGCAATTCTCATGGCTCAGCCTCCCGAGCAGCCGGGACTATAGGCGAGCGCCACCACACCCAGC
TAATTTTTTTGTATTTTTAGTAGAGATGGTGTTTCACCATGTCGGCCAGGCTGGTCAACATAGCTACAGCTATTAAG
CAGGGATGTATGTTGGATTCACATGTGGGGTTGTCACAGTTATGGATTTTCAGGACCCTACTTTCTGGGCGGTCTGA
TCTGGAAAGTCTGGGATGGGGCCCAAGGTGAGTACTTGTAACAAGCCCGACCAGTAATTCTAATGTTCTCCTCCCAC
CGAGAACCACAGAGAAAAGTCTGGAAGGAGACCCACCAGACAGTTAACAATGGTTATCTCTAGAAGGAGAGATTAAG
AAGGAAATTTACATCTGACTATATATGTTTGCATTTTTGCAATTATTTGCAATAAATTAGGCATTCCATTCTTCATC
AAAGTAATAGAAATAACCTCCAAAATACGTAGTCCTAACATGTCAGCAGGCTTATCTTGTGTAAGAATCATTTTATT
AATATCTGACACAGCAAGGGAGATGAGGAGAGGTGGGAAGAGGGAGAGAAAAGTATGAGAAAGACAAATAAACAGGC
TGAGGTAGACAATGGGTGACACAGAAAGGAAGTGAGACAGAGACTAAGAGAGATAGAAAGGAGAGAGGCAGGGAGAT
GGGGCAGAGGCCAAGAAAAAGACAGAAGGATGAGGGAGGAAGATGCAGAAAGAGGTAAATGTGAGATAGATCAAAGG
AGATATAGAGTCAGTGAGTGAGGGGTTCAGAGGCAGAGGGGAGTGGGGAAGTGGGGTTCCCATGGAGGGATTGGGGC
CCAGGAGGCGCTCTCTCCCTGTGACCTGCTAGCTCAGCCCTAGGCAAACCTCACCACCCCTTCTTTCTTGCAGCTGT
TTGAGCTCTTCTCTGGCTTCTTGAAATACTTTCCTGGGGCACACAGGCAAGTTTACAAAAACCTGCAGGAAATCAAT
GCTTACATTGGCCACAGTGTGGAGAAGCACCGTGAAACCCTGGACCCCAGCGCCCCCAAGGACCTCATCGACACCTA
CCTGCTCCACATGGAAAAAGTGGGGTCTGGGAGAGGAAAAAGGGAAGGGAGGGGAGGGAGGGCAAGATGGAGAGGTG
AGAAGAGGGAGGGAAAAGGGGTAGGGAAGGGGAAGATGGGGAGGGAAGAAGAAAGACTAGGGAGGGGAGAATAGGGA
AAGGGAGGAGAGAACATGAGGAAGGAAAGAAAGATGAGGTGAAAGGAGGGAGAAAATAGGGAGGAGGAACTGAGACA
GGGAGAGAGGGGAGGTGGGAAGACAGAATGAAAGACAGAGGGAGAGAGAGAGAAGACTGGCTGAGGAAGGAATTCGG
GGCAAGGGACAAAAATACAGCAACAAGAGAAAAAACTCACAGAGGCAGAAAGAGACGGGGACAAAAAGAGAGAAACA
CATCAAAGAGATGTGGAGAGAGATAGAAACAGAGTTAGGAAGACTAAAGAGAGGCTGAGAGAGATGAGTTAGAGATA
CGCGGTTGGATGTGTAGAGGACAGAGAAAAGCAAACTGGGCCAGATAGTGTCAAAGACCTTTAGGCCAACGGAGGGC
AGCCAGGGAGATGGGCGTATACACAGCAAGGCTACAGCCTCCCCTGACCCTCCCCTTCCTTCCCTACTGTGGACGCA
GGAGAAATCCAACGCACACAGTGAATTCAGCCACCAGAACCTCAACCTCAACACGCTCTCGCTCTTCTTTGCTGGCA
CTGAGACCACCAGCACCACTCTCCGCTACGGCTTCCTGCTCATGCTCAAATACCCTCATGTTGCAGGTGGGCCAGGG
ACAGCCAGTCAAGGGGGTCTTCTGACCTCCTTCTGAGCTGCAGAAATGGGGCTATGGGTACCACCTGGATGAGAGAG
GGGATGCTGGCTTCCTATTCTGGGAGCACTGTAGGCTCTGGGCTAGATTCCAACCAAGCCAATTCTGTTGGTGGATG
CATGGATGCATGAAGAATCTGTCCATGCGTTCTCCCACTGTTTTCTTCCATCACTTAAGGATTTTTTGTTCTAAGGT
TTTTGTTTGTTTGTTTGTTTTTTGTTTTTTGGTTTTTTTTTTTTTGTCTTTTTTGAGACAGAGTCTCGCTCTGTCAC
CCAGGCTGGAGTGCAGTGGCATGCTCTTGGCTTACTGCAAGCTTCACTTCCAGGGTTCACGCCATTCTCCTGCCTCA
GCTTCCCGAGTAGCTGGAACTACAGGCGCCTGCCACCACACCCGGCTAATTTTTTGTGTTTTTAGTAGAGATGGGGT
TTAACCATGTTAGCCAGGATGGTCTCGATCTCCTGACCTCATGATGTACAAATTTAGGGGGTACATATGCAGTTTTG
TTACATGCGTAGGTTTTGTAATGGTCAAGTTTGGGCTGTTAGGGTATTTATCACCCAAACAGTGTACATTGTACCCA
TTAAGTAATTTCTCATCATTCACCCCCCTTCTGCTCCCTCACTCTTCTGAGTCTTCACTGTGTATCATTCCTCTCTC
TGTGTCCATGTGTACACATTTTGTAGCACTCACTCATGAGTGAGAACATGCAATATTTGACTTTCTGTGCCATCACT
TAAAAATCGATCCATCCACTTATCATTTCATCTATTCATTCTTTAATTCATTAATTAAAGAATGTATAATTACTGAT
TCTTTCATTTATGATTCATCTAAGGACATATACTGTCATTCATTTATTTGGATTACTTGATCCACAAGTTGATCCTT
TGAAACAGTGGTATGTTGATGGACTATTTGTCATTGATTCATTGGCTCATTCATTCATTCATTCATGCATTCATCCA
TCCTTCTACGAACTAGGTTTCACTCTTATCCTTCCATGAGTCAACCTTTCAATTCACCCTTAATCCATCCGTGATTT
CCTTCATTTATCACAATAATTCACTCATTTATTCACCTCTGATCTACTTATCATTCAATCCAGCCTTTCATTTACTC
CTTTATTTACTCATACTTCACTAATTTAATTATTCACTTGCTCTTCCATAAATCTAGCCATTCATGTGATTATTCAT
TAATTGGGTTCATTGATTTCTTTGTCTATGGATCATTCATTAGTGATTAATTAATCAATCCATCTATTAATTGATAA
GTAAATACAGACATCCATTTATTGGTTTGTTCATTTATTCATCAATCTTTCCATCCATGAATTGATCTATTGATTGA
TTGATTGATGTTTTTATCCAGTTGTTCATGAATTCATCTATTGTTCTATTACACTGTTATATACCTGAGGACCAAGA
ATAGTGTCTCAACTATATTATAAACACAATAAATATTAGTTCATTTTCTACTCATCTTAGAGAGGGTGTTTTGAGAG
GTTTGTAGCCTGGAGTTCTTAATCTGAAATTCCATGCAAAATCGTGGTGTGTGTGTGCATGTGTGTGTGTGTACCTG
GGCATGTGGGAAGAGGATCTGTAATATTCATTAGATTTCAAGCAGTGAGAATTCTTGTTGCTTCCTCCCTCCTCCCC
TCACCCCATGCTGATTACTTTGAGGGGTATCAAGGATCTAACCCTTTACTATAGGTTTTTCATTGGTCAATAGAAAG
TAGTGTCCTTGCTGAAAGGTCTCTTTTTAAAAAAATTTTTTTTTCTTTTGAGATGGAGTCTTGCTCTGTCACCCAGT
CTAGAGTGCAGTGGCATGATCTCGGCTCACTGCAACCTCCACCTCCTGGGTTCAAGTGATTCTCCTGCCTCAGCCTC
CTGAGTAGCTAGGAATACAGGTGTGCACCAGCACACCCAGCTAATTTTTTGTATTTTTAGGAGAGACGGGATTTTGC
CATGTTGGCCAGGCTCATCTTGAACTCCTGACCTCAAGGAATCCACCCACCTCAACCTCCAAAATTGCTGGGATTAC
AGGCATGAGCCACCATGCCTGGCCTGAAATGCCTCTTTAAAATGAGATTCATTGGTCTTCTTTTCTGTACAGAGAGA
GTCTACAGGGAGATTGAACAGGTGATTGGCCCACATCGCCCTCCAGAGCTTCATGACCGAGCCAAAATGCCATACAC
AGAGGCAGTCATCTATGAGATTCAGAGATTTTCCGACCTTCTCCCCATGGGTGTGCCCCACATTGTCACCCAACACA
CCAGCTTCCGAGGGTACATCATCCCCAAGGTAAGACCGGCTGGAACCCCATAGCCCTCCTGTTTGGGCATCCTGGAT
TCTCTTAATCCCCGAACTCAACCTTTTGTTAGCTCCTTAATTGAGTCCCGTTGTTTTTGTTTTTTGTATTTCTTTTT
TGTGGAGTGTGTGGAGGGTTGGAGGGAATGGCAATATCTTTTGATCTTGTGATCCTCCCTCAGGACACAGAAGTATT
TCTCATCCTGAGCACTGCTCTCCATGACCCACACTACTTTGAAAAACCAGACGCCTTCAATCCTGACCACTTTCTGG
ATGCCAATGGGGCACTGAAAAAGACTGAAGCTTTTATCCCCTTCTCCTTAGGTAAGCTGGACCCACAATTTCTTTCC
CAGACACCAGAGGGCAGGTACTATCCCCAACTTGAGAAAAACAACGAGAGATACTGATTATTTGAGCACTTAATATA
TTCTGATTGCTTCACCTGCCTTATCCCATTCCATCTTCACTACAACCCTATAAGGAGGCTTGAGAAAGAAGATTACA
TTCCCAAAGGCACATCTTGGCAAGCAGGACCTTGGGCAAGTATTTTAACATCTCTAAACCTCAGTGAGTTCATTTTC
TTAAAAAGAAAAAATCTGTTGGGCACCACTGTAAGCCCAGTGCTGTACTGGGGGCTGAAGATAATGCATCAAACAAG
TCACACAGAGACAGGGTTCCTGCCCCAGGAAATTTAAAGTCCAGCAGGGAAGATGGGCATTCATCAAATAATAATAA
AATAATCATCTCATGAAATGAATGAATGCCTGCAACATGCTTAGAACTGCCTGGCACAAAGGACATGCTCACAAGGG
CAATTATTATTATAATTAGACATAATTGTGATAAGTGCTCTAAAGGGAGCTTTGGGAGCACAAAATAGGAAATAGTA
GCTAATCTTGTGGTGGGTCCGTAAGGAAAAGCTTACCAGAGGAACTGGCTTCCAAGCTAACATGTTCATGGGTGAGC
ATCAATCAACCATTGCAAAGTGTGTTCCAGGCAAAGGAAACAGCAAGGACAAAGGCCAAAGGTAGAAACGTGATATG
GCACCATTGAGAACCTATAGGAAGTCCAGTGAGCCTTGGGTGTAGATTGCAGAAGGAAATAAGACAAAGGGCTCATC
TGGGCAGGACCTTGAAGGTTGCAGGAGGAGTTTGGATTTATGGTCTAGCACTGGGAAGGTGGAAAAGGTCTTGACGT
GCTCTGACTTGTCCCAGTTCTCATCCTCTACTCTTTGGCTGGTTAAAAGAAAAACTTTAGACAAATTAAACTTAGCA
GAGTCTATCTGAACAAAGAAACAATTCATGAATTGGGCAGCACAAGGAACCAGTAAAGGTTCAGAGAGCTCCATCCA
GCAATGTGGTCAGGCACTATTTATCCACAGGGAAAGGAACTGAGGTACTGAAACAGCCTGATTGGTTACAGCTCTGT
GTTTGCCTTATCTGAGCATGTCTGGGCAGCTTGTAGCCTGTGACTGGCTGAAGCTTGGCTGCCCTGATTGTCCAAGG
TTACTTGTTACAAGAATATACTCTCAAGTTTGTTTACATGTTGAGTTACATTACAATTTGTTATGTAGGGAGGCTGC
TTTAGGCCAAATTTAATTGAATTTAACATGCACCATCCATGAAAGGCCCCAGAAACAAACCCCCAGTCTTGAGTTTC
ATCAGTGACTTCAGTGATCCAGGGTTTGGCCCAGCCCCCGGTTTTGCGCAGTACAGTGACCTCCTTCCACATTTTAT
CTTCAATGAAAATTGGGAACATGTGTGGTGATGGCTTGAATGTCAATGTCTTCTCTCAATTTTGCTATCTGAAGTTT
CTTTTTTTTTTTTTGTCTTCAGACGGAGTCTTCCTGTGTTGCCCAGGCTGGAGTGCAGTGTCACGATCACAGCTCAC
TGCAACCTCTGCCTCCCAGGTTCAAGTGATTCTCCTGCCTCAGCCTCCCGAGTACCTGAAACTACGGGCGCATGCCA
CCATGCCTGGCTATTTTTTTTTATTTTTAGTAGAGACTGGGTTTCACCGTGTTAGCCAGGATGGTGCTGATCTTCTG
ACCTTGTGATATGCCCACCTCAGTTTCTGAAAGTCCTGGGATTACAGGCATAAGCCACTGCGACTGGCCTGCTAATT
TGTTTTAAGAGAGGCAGAGATGTAGGGAAATAAGGACAGAGACAGATAAGTAACAAAGATAATGAGACTGAGAAACA
GAGTTAGGGACAAAGAGGGAACACATAGAGATAGAGATGGGGAGCCAGTGGCAGAAAGACAGAGGGCAAACCTCAGA
CAGTATACAGAGAGGAAGAGAGAGAGACACAGAGAGAGAGAGAGTCACGTAAGGAGAAGGAGGAGGAGGAGGAATAA
GAGGAGAGAGAGTAGGAAAAGGAAGGAGGAGGGGAGAAAGGAGGGAAAGGAGAGAAACAGAATAAGAGATAGGAAAA
AGAGAGAGTGACATAAAAAGAGAGGAAGAAAGAATGAACAAGACAAATAAGGTATCTTTAGAGGGAGATGAAAAGAA
AGAATGAGAGAGAAAGAGATTGGGGAGAAATCAGATTCAAATAGATGGAGATAGGAAGTTAAGCAAGATGGAAGAAA
GCCAAAAAAAGGAGAGGAAAAGAAGAAAAGCTGTGTCTGACAGGTATAGACAGAGAAAAAGACAGGAGTAGGGCTGC
CAGGGGCAGAAAGAAAGGTATAACCCAAGCCAAAAGGGTACTGCAGCCAAAGAAATTCGAAAGGTGTCCAAACAGAC
GGCCCCAGAGATGGGGAGGGCGTGATGAGAGGGAGATAATAAGCCTCAGGCTGTTGTGAAAAATGTTAGGTCACACA
AAGAGTTACAGAAGAACAGGCCCAGAGACCTGCCTGTTTCTAAGCTCATGTCACCCACCTTCTGGGTATGCCAAAGG
GATGTGGACACTTTTCCAAACACCTCCACATAGACACACTTGTCCAACAACTTGACAGGCATAGGGAATGCCTGAAA
ACTCACACTTGACATGGCCTTTCCAAGGTTTGCAGATTATGAAACACTGAAGTGAAGGGAAAGGCTCCCTTTGTCTC
TGCCTGAGCTTTTTCCAGCACCCTTTGTTCTTATTTTTCTCCCAATCTGTGGTTTAGAATCTACTGGGGGTTCCTTG
CACCTCTGAGAATCAGTGGAAGCCATAGACCCTCCCTGTCTCATACGCATCAGTCCCATTCACAATTTATATACAAT
TGGGCTTTTCCCCCCTTGAGCTCTTGAAGCCTGTGGATCTCAGGCTGAGACTAGAAGACAGCCTAGAGACACAGACA
CAGACACATGGCCAGAAACACATACCCCACACATACCCCAGAAACAAACACAAGGCAGTAACCAAACCTTAAGCCTC
CTTAGCACAAGCCAAGTGCTATTCTAAGAATTTTTACAATTGTCTCCTTTAACCTCACAAGAGCCCTTTGAGGGAGG
TGCCATTCTCCCCATATGAGAAGTGAAGAACACTAAGAATGTTGTCATAAATGTGCTCACACTCACACATGTACAGG
TACACACACACACACACACACACAGAACCAGAAATGGGTCATTATACTCTGTGGGTTTCACCAACCCTTTGGAACTG
TAACAGAAGCTAGAAACTCTCTTATATAAAAATGAACATGGCGCCGGGTGCAGTGGCTCATGCCTGTAATCCCACTT
TGGGAGGCCAAGGCAGGTGGATTATCTGAGGTCAGGAGCTCGAGGCTAGCCTGGCCAATATGATGATACCCCATGTC
TACTAAAAATAGAAGAAATTAGCCAGGCGTGGTGGCAGGTGCCTGTAATCCCAGCTACTTGGTAGGCTGAGGCACAA
GAATCATTTGAACCACCTGGGAGGTGGAGGTTGCAGTGAGCCAAGATGGTGCCACTGTGCTCCAGTCTGGGTGACAA
AGAGAGACTCCATCTCAAAAAAAAAAAAAAAAAAAAAAAAAAAAAAAAAAAGAGAGAGAGAGAAATGAACGTGGCAC
ATCCACTCAAAGATTTGCATCTGGTTTCAGAGCAGCTTCCTAAAAGTCCACCCTGAATTGTAGGTTAAAGGCCAGTC
TTATGCAAATCTGTTGCAGTGGACATTTGTGTCTGGGCTTAGGGACATGGCAGAGCGAAGTGTATGCACCTGCCCTG
TGCCCACACTGGTGACCTTCTGTGTCCACAGGGAAGCGGATTTGTCTTGGTGAAGGCATCGCCCGTGCGGAATTGTT
CCTCTTCTTCACCACCATCCTCCAGAACTTCTCCATGGCCAGCCCCGTGGCCCCAGAAGACATCGATCTGACACCCC
AGGAGTGTGGTGTGGGCAAAATACCCCCAACATACCAGATCCGCTTCCTGCCCCGCTGAAGGGGCTGAGGGAAGGGG
GTCAAAGGATTCCAGGGTCATTCAGTGTCCCCGCCTCTGTAGACAATGGCTCTGACTCCCCGCAACTTCCTGCCTCT
GAGAGACCTGCTACAAGCCAGCTTCCTTCCCCTCCATGGCACCAGTTGTCTGAGGTCACATTGCAAGTGAGTGCAGG
AGTGAGATTATCGAAAATTATAATATACAAAATCATATATATATATATGTTCTTGTTTTTTGAGACAGAGTCTCACA
CTGTTGCCCAGGCTGGAGTGCAGTGGCGTGATCTCGGCTCACTGCAACCTCCACCCCCGGGGATCAAGCAACTCTCC
TGCCTCAGCCTCCCTAGTAGCTGGGATTACAGGCATGCACTACCACGCTTGGCTAATTTTTGTATTTTTAGTAGAGA
TGGGGTTTCACTGTGTAGGCCAGGCTGGTCTCGAACTCCTGAACTCAAGTGATTCACCCACCTTAGCCTCCCAAAGT
GCTGGGATTACAGGCGTGAGTCACCGTGCCCAGCCATGTATATATATAATTTTAAAAATTAAGCTGAAATTCACATA
ACATAAAATTAGCTGTTTTAAAGTGTAAAATTTAGTGGCGTGTGGTTCATTCACAAAGCTGTACAACCACCACCATC
TAGTTCCAAACATTTTCTTTTTTTCTGAGATGGAGTCTCACTCTGTCACCCAGGTTCGAGTTCAGTGGTGCCATCTC
TGTCCACTGCAACCTCCACATCCTGGGTTCAAGTGATTCTCCTGCCTCAGCCTCTGGAGGAGCTGGTATCACAGGCG
TCCCCCACCACGCCTGGCTAAATTTTGTATTTTTAGGTGGTCTTGAACTCCTGATGTCAGGTGATTCTCCTAGCTCC
AAATGTTTTCATTATCTCTCCCCCAACAAAACCCATACCTATCAAGCTGTCACTCCCCATACCCCATTCTCTTTTTC
ATCTCGGCCCCTGTCAATCTGGTTTTTGTCACTATGGACTTACCAATTCTGAATATTTCCCATAAACAGAATCATAC
AATATTTGATTTTTTTTTTTTTTTTGAAACTAAGCCTTGCTCTGTCTCCCAGGCTGGAGTGCTATGGTGCAATTTTT
GTTCACTGCAACCTCTGCCTTCCAAGATCAAGAGATTCTCCAGTCTCAGCTCCCAAGTAGCTGGGATTACAGGCATG
TACTACCATGCCTGGCTAATTTTCTTGTAGTTTTAGTAGGGACATGTTGGCCAGGCTGGTGGTGAGCTCCTGGCCTC
AGGTGATCCACCCACCTCAGTGTTCCAAAGTGCTGATATTACAGGCATAATATGTGATCTTTTGTGTCTGGTTGCTT
TCATGTTGAATGCTATTTTTGAGGTTCATGCCTGTTGTAGACCACAGTCACACACTGCTGTAGTCTTCCCCAGTCCT
CATTCCCAGCTGCCTCTTCCTACTGCTTCCGTCTATCAAAAAGCCCCCTTGGCCCAGGTTCCCTGAGCTGTGGGATT
CTGCACTGGTGCTTTGGATTCCCTGATATGTTCCTTCAAATCTGCTGAGAATTAAATAAACATCTCTAAAGCCTGAC
CTCCCCACGTCAA
SEQ ID NO: 3 (TPMT)
Homo sapiens thiopurine S-methyltransferase (TPMT), RefSeqGene (LRG_874) on
chromosome 6
NCBI Reference Sequence: NG_012137.3
>NG_012137.3:5067-31833 Homo sapiens thiopurine S-methyltransferase (TPMT),
RefSeqGene (LRG_874) on chromosome 6
GTCATTGGTGGCGGAGGCAATGGCCGGCAACCAGCTGTAAGCGAGGTAGGCTCACTCGGGCGCGGAGGGTGCGGGTG
AGAAAGGGAACGATTTGCTAGGAGTGTATGCGCCCGTGCTAGGGCTCCCAGGAATTCTTACAAGCGTAGACAGCCTA
GCAATCAGCCCTGTGAAGTGGGGTTCAGGGCAGAAGCGGAAACTGGGAATAACGAGGTCGGACAGTAAATGACCTGA
ATCCAATGTCCAGGATTTAAAGTGCTTCCAAAGCACTGGTGTGTTTTAATTATTTTTTTTTCTTTTTTCTTTTAGAG
ACAGGGTCTCCCTCTGTCGCCCATGCTAGCGTGCAGTGGCACAATCATAGCTCACTGTAACCTGGAACTCCTGGGCT
CAAGTGATCTTCCCGCCCCAGCCTCCTCGGTAACTGGGACTACAGGCTCGCTGCACCACGCTTGGCTAATTTTTCAA
ATTTTTTTTTATAGAAACGGACTCTCGGTTTTTTCCCCAGCCTGGTGTATTAGTGATTTTTGGGAAAAAATAAGTAC
TGCCAAATAGTGAGTGATCCCTGGTGATGTGGGAAACTGGGGTGCTGTAAATATTCTGTCTTGCTCTGCGGTGGTTA
CACAGGTGTATGTATATGTAAAAAATTCACCGAGTCATACTCTTTTAAGATGTGTGCACCTTAGGAAAGTTTCTTCT
CAATTTTATGAAATTTAAAAACAAAACTATAGCAGAAGCTTTTGAAAAGGTGAGAATGGAGACTAGAACAACTTGAG
GACTTTGTTTGTGGGCAGAAATTTTTGTGAAATTTCCCTTCCTTAATTCTTGTAATGTGCACTTTTTTTGTATTATA
GAAATAAACTTTATTTCTCTAGAAATGATGTATACAAGTGGAGTCCATGCTGAGAAACTGTTAGAACAGCTAAGACG
TTGAGCCCAGGAGTTTGAGACCAGCCTGGGTAACATAGCAAGACCCTGTCTCTACAAAATATTTAAAAATTAGCCAG
GCATGGTGGTGCACCCTTATAGTCCCAGGTACTCAGGAGGTTGAAGTGAGAGGATCGCTTGAGCCCCAGTGTTGGAG
ACGGAAGTGTCATTACACCACTGCACTCCAGCCTGGGGGATAGAGAGAGACCCTGTCTCAAAAACAAACAACTAAGA
CATGATTAAGTTATGTGCATGATTTTGTTAGACAGTTCAAGTTTGTGTGAAAGGAATAGAGTTGTGCTACTCCAGGC
TGCTGTGGAACTCTGATAAAGATTATGCTTCTCTTTTAAGTAGAGAACTGCTTTATGAAGCCTTGAATTTAGGTTCC
AGAGTTTCTGAATGAAAATGAGAAAACTGCAACATTCTTTTGCTTTTCTGTACCCAATATAAATCAGATACTTTCTA
CTGACCTCTCAGAAAACTCAGAGTCACCTCCTGATACCATCAGGGTCTTGCAGAAGCCATTTGTCAACAAATATTTG
CTAAGAAGTGAATTCATGTTCCCAGGTAATGAGAGAAATGCCTCCTCCCTATATCCTAAGCATACTGAGGCCAAATT
TGGCAACAAAAGGGGAAATTTGCCTGAAATACATGCTTTTATGCAATGTTTGCTATTTCACAGATTAATCATTAAAT
TAGAACCACCAATAATAGCATGCTAGTATAATCCAAACTAGTTTCACTAATGATTTAAAGGGAATGTTAGAATAATG
GTGTTATATTGTCCTTAATATTGCAGAGTTGACATTGAAAGGAAAGGCATTTTCCTCATACATTCAGATGGATCTGC
AGTTATGGTCTGTCAGCTGAAGAATGAGAAGTTCATAATTTAGAAAGGAGAACTCTTTCTCATGAATGGTTTCAGAC
TGTAGAGCAGCCATTCTGACTGGCTGAGAAGTGTAGCCGCAGGCCAGAAGCCAGAAACTGACACTTCCAGGGAGGGG
CAAAGGGAACAGGAATTTATGCTGAGGAAGGTGGCCAAATATACATATTCAGTAAGCTGTAGGATGACTCATGAATA
TTTATGAAAGGAGGAAAGTACCCATGAGTAATTGAGCTTCATGGGTACCATGCACAAAAAATGGCGGTGTTACCACA
ATCACAGGATGGAGTTTTTGGCCCTCTTATATCAAAAGGTGAAGCAGAGGACAGAAAAACCCTTACTGTGCATTCTC
AGTAGACTGGCCAGAACCATTCTGTGGTCTGTGATCTCTTATCAGGCACAAAAGGAGGGGCAGCATCAGGGGGTTGG
TTGATATCAGTGGTAGAATCTTTTGAAAGGGCTGGTTTCCCTTAAGTCCTTATAGAACAAAGCCTAATTGTGGTTAG
CAAGAGAGGAGGTATAGAGTTGTATCTGACGCTCTATCTACTCTTGGCCCAGAACTCAGTTTTCAAGGTTACTCTGG
GGTCCCGTTAGCCAAGAAAGTGTGTAGTGAGCCAAGATCGCACCACTGCACTCCAGCCTGGGCAACAAGAGCGAAAC
TCCATCTCAAAAAAAAAAAAAAGAAAGAAAGAAAAGGAAAGAAAGTGTGCCTTCAGTCAGTTGAGAGGCTTTGAATT
TTATTGTTAGTTTACATTGTACATGTCTTTTTTCAGTTTCCTTAGTGTGCATTTCCCATGCAAAAAAAGAAGCTGTA
GAAAATTATTTGAAAATACAAATGTGTGGAGTAGAGATAGGATATTGAACTATGGTTCAGTATCTACTTTAATCTGT
TTTTGCTGTCTCCTGAAAATAACAGAATTTTTTTTTAAGGCATAAAGTACTAGGATAGCCAGGACAGGAGAGACAAA
ATTAGTAAAATTTTGGAAAGGCAAATGGACAAGTGGGAAAGGATTTTGCAACTTCAAGGAAGCTGAATTCTAAACCA
GCAATGAGAAAAGCCATTGAGCAGCCCAGTTTGCAATACAGAATCCCCAAATGCTCAGGATTAGCAGCACCAGGTAC
CTCCATAAAGTGTGAGAGTATGGTTGAAACAAGGAGGATTGCTTGAAATGCTGGTTCAGAACAGGCCAAATCCCCAG
ATCCTTTTCTCAACTTCTTGCAACCAAGTGTGTACATCTCACCACACCAGCAGGCTTTGGGGATTTATTCTCTGGAA
AGGGTAAAACAAAGAGTGTCTGTCTAGACTGAGAAGATATGATACACAGTTGAGCATGGGAGTATACAGGAAACGTG
AGGATTAATTGAATACAGCAAACTGGATGCTGGGACCCTCGCCCACTTCTCTTCTTTTCTTATATGAGTATATGACA
GTGGCTCCAACGACAGAATCCTTAAGACAGGAATTGGGAGATCCATCTATGGAAAGTCTGACTACCACAAGGAAATT
AAAGACATTAGCCAGGCATGAGGGTGTGCAGCTGCAGTCCCAGATACTTTAAAGGCTGAGGCGGGAGGATCACTTGA
GCCCAGAAGTTTGAGGCTGTATTGTGCCACTGCACTACAGCCTGGGTGACAGAGCGAGACCCTGCCTCAAATAAATA
AATAAATAAATAAATAAATAAATAAATAAATAAATAAATCTAGGTTTCCAAATGAAATCCACTCACATCACCCTACA
TTATAAGCCCCACCCACATTCTTTGCCTTCATTCATTTTATACCTATTCTTTTTTTTGTTTTTTTCTTAAGACAAGG
TCTTGATCTGTAGCCCAGGCTGGAGTGCAGTGGCACCATCATAGCTCACTGCAGCCTCGAACTCCTAGGCTCAAGCC
ATCCTCCTGCCTCAGCCTCCTGAGTAGCTAAGATTACAGGCATGCACCACCATGCCTGGCCAATTTTAAAACTGCTT
TTGTAGAGATGGAGTCTCACTATGTTGCCCAAGCTAGTCTCAAACTCCAGGCGTCAAGCCATCCTCCCACTTTGGTC
GCCCAAAGCACTGGGTGATTGATAGCAAAAAAAAAAAAAAAAAGAGAGAAGAAGTTAAAAAAATGAGAATTAAGGTA
AATCTTTCAGAAAATAGTACAAAAACATGAAGATATAGAAAAATAGAAAAAAATGAGAAATTTACCATACCGGGTCA
GAAGATTCTAGAGACAAATAATAGGGGTCCCAGAAGGAGAAATAAAAAAAGCAGAGGGATGGCCAGGCTCAGTGGCC
CACGCCTGGAATCCCAGCACTTTGGGAAGCCGAGGCTGGTGAATCACATGAGGCCAGGAGTTCAAGACCAGCCTGGC
CAACATGGTCAAATCTCATCTCTATTAAAAACACAAAAATTAGCTGGACCTGGTAGTGCATGCCTGTAATCCCAGCT
ACTCAGGAGGCTGAGGTGGGCGTGATTCTTCACTAAACCTTTAGGGTGCCCAACCCCCTATGGCCCCCACACTTCTA
ACCAAGAATTTGATACCCAGCAGAGCTATGCATCAAGGATGAGGAATAGTCTAACCGTGTTTTATACATGCAAAATG
GAAAAACATTCTTTTTCTTCCTTGTGCTCTTTCAGGAAGCTCTGGAGGTTAGGCTTCACCAATATAAGCGTGGTCAC
TAAAAAATAACAACAAAAAAAAGAGGAAGACATGAAATAAAGCTGGAGATTCAGACAAGAAGGAAATCTCCAGAATG
ATGATGAAGAGCGATCCCAGAAAGCTAGCTGTCTACCAAGATTAGAGAATAATCAGGGCAGATTCCAGGAGAGAATT
CTCCAAGATGATATTGATAGTATAGCTGATGACTGGAGAGACTGACTGTGTACTGATAGGAAATTGGGACAATTGGC
AGAATTTAGAGTTGAATTAGGAGTAAGTACAATAATGCCTACGTGATGAAGCCTACATAAAAACCCAAGAGGATGGG
GTTTGGAGAGCCTCCTAATAGCTCAACAGGTGAAGGTCCTGTAGCTAACACATCTCTTCAAGTATATTCTTCGCAGT
ATCCTTTATAATAAATGTGCAAAGATAACTAAGTATTTCTCTGAGGTTTGTGAGTTGCTCTAGCAAATTAAGCAAAC
CCAAAGTGGAGGGTGTGGGAACCCCAACTTGAAGCCAGTGGAGGCCTGGACTTGCAACTGGTGTCTAAGTGAGGAGG
GAAATCTTAGGGACTGAGCCCCCAACATATAGAATCTGACACTATCTCCAGGTAGGTAATGTCAGAATTAAATCAGA
AGATACCCAACTGGTGTCCAATATTTGGTGTGCTGGGGGAAGAACCCCCACATTTGGTCACAGAAATCTTGTGTGTT
GATGATTGGGGTGTGAGAGCAGAGGAAACACTAATATTAAGATAGAATTCAGTTTATTTAATGATCTCTTGTAATGT
GTATTAAGGGGTATTATTCTACCACCAGACGCACTGAAAGTAATTTTTTGTTATCACTTCTTATTGGATGTTGAAAT
GGCAAGGTGCAGTGACTCACACTTGTAATCCCAGTGCTTTGCGAGGCCAAGACAGAAGGATCCCTTGAGCCCAGGCG
TTCTTTGTTGTCTGGACAACAAAATGAGAACCATCTCTATTAAAAAATATATATATACAAAAATTAACCAGGTGTGG
TGGTGTGTACCTGAAGTCCCAGCTACTTGGGAGGCTGAAATGGGAGGATCGCTTGAGCCCAGGAGGTTGAAGCTTGC
AGTAAGCCAGGGTCTCCAGCCTGGGTGACAGAGCAAGACCTTGTCTCTGAAAAAAAAAAAAACAAGGAAAGAAAGAT
TAGCTGGGCATGATGGTGCACACCTGCAGTCCCAGCTACTTGGGAGGCTGAGGCAGCAGAATTGCTTGAACCTGGGA
GGCAGAGGTTGCAGTGAGCCAAGATCACACTACTGTACCCCAGGCTGGGAGACAGAGTGAGACTCCATCTCAAAAAT
AAAACCAGAAATAAAAAAAGAAAGAAATATTTTTAAAAAGTCATACTGCTAAGAATAATAGGTTTTCATTTAGTTCA
TCAATAGAATTCCAATGACCTTAAATGACAACATTGCAAATATTTTCCACGTAGGCACGGAAGACATATGCTTGTGA
GACAAAGGTGTCTCTGAAACTATGGATGGTACAAGAACTTCACTTGACATTGAAGAGTACTCGGATACTGAGGTACA
GAAAAACCAAGTACTAACTCTGGAAGAATGGCAAGACAAGTGGGTGAACGGCAAGACTGCTTTTCATCAGGAACAAG
GACATCAGTAAGAAATATTTGGTATACAATAGAGAAATGTTCTATCAAAAAGTGACTTTGAGATAGAAATAATAAAA
GTACACATTATTTGACTAACTTTTAGGATGCACATTTAATTCTTCACATTTTTGTGTCTCCCGAGTCACAGTGCATC
TGTGGCATGATGTAGTTTAATTGGCAGAGTCTTTTCTTCCCACAGTGTGTAAATAATGGTGTGGCTTACAAATGATG
ACATCTTAGATTTATAAAATATGTTCTTTGATCCCTACGGCACCTATGTATTGGCCCTGAATATTTATACCTACAAC
AGGTGCTCAAGCAATGTTTATGCTTACAATTCTTCATACTGAAAATCAGTGGTGTCTGACTTTGCTTTCCAATAGGA
TGTGTTCAGATGTTAAAGAATTATCTCTATGGTGGGAGAAAAGAAGTTGCGAGAAATTATAAAGTGAGTTAGGATGT
TGTGCAATGTAGTAGTAGTCTTCATAGCAGCAATAAATCATGGGACCAGTGCTGAGTAAAGTGGTTGTTAGAGACAT
TCCTTGTTCTGGGCAGCCACGCCCATCTCCCCAGGAGATGACATCATCATCATCATCATCATCATACCAGAAACACT
GGGTTATTTCTGTCATGTGGATGGCCTTAGAAGAGCCACATCTCCACAGAGGGAATTACAAGAAACTACAGGAGTTA
CACTTCTCAGGTTTACTTCCGAGGCTTGAGTACACGTTTAGAGAACGTACCATGAAATTAATGTTGAAAAATCCATG
GCTCCAGAGGCTCCATCACCCCCAAAATAGTACATATTTAGAGAATTAATAAAGAATTTTCAAAACATCCCCAAGAA
AAAAAAATTATTTTTAATTACTAATCAGGAAAGCCCTGGGTGTAAGTCAGATTTACCACTGACTGGGTGTGTGTCTG
AGCAAGTTACCTGTTTCTTCATATGTAAGCTGGAGATTATAATAATTAATAGTAACTGCTTATTAAGGTTGTTGGGA
ATATTAAGTGAGATAATGAATGAAAAGTGTTCACCTACCATACAATTGTCCTAAAACCTTTTTTTTTTTCCTCTTCA
GGCTATTAAAGAAGCATTTAGATACTTTCCTTAAAGGCAAGAGTGGACTGAGGGTATTTTTTCCTCTTTGCGGAAAA
GCGGTTGAGATGAAATGGTATGAGCAGATAAACATTAACAGAATTATCTTGCCTTAATGATGAATTCTTTGGGTGAT
TTAACAGGATGTGAGTTTTAAAACTCCATTTTCAGGAAATACACAGAAATTCCTTATTTTTCTATTAGTGATGGGTA
TATAATTGGAAATAATATTCTCAAACAGTAAGAAACAACAGAAACAGGTATGAAGTCTTTCTGGATTGAGTTTTGAA
ATTAAAAAATCAGTTTGGAGCCAATGGGGGGAGAAAAAACAGCCTTCCCTTTTACATTTCCTGGATCCTTGTATGTG
GTTCAGTTTTTGTAGGTGGTTTAGGAATTTAGCTAAGTTAATTTTTAGATATTTGAAATGGTGGTGGACAGAGAAAT
TCAAAATGAATTACAGCTTTTTGGCAGGACAATGTTCATAGTCCACAGTTCAGCTCAACCTTCCCCTCCCACCAGGG
AACACAGGAGTTTCTGTTATCGTGAATCATTGTTATAAAACCAGTGATTAAGAAAGTATTTCTTGTGAGTAATTCTC
TACAAAGAGAATTCACTTTAACAAGCCACTTTTACTATGAGTTACCCTAAAGTCACCTCGACATCATTCTCTATACT
GAACTCTCTTGTAATGATAATATACAGATACAATCATACCTGCCAAGATATGCATTTATTTATAAGCTTGAATGTTG
AGAACTTTGACCACTGAGCAGGAAAGAAAAAAGAATGTATGAAAAGGTTAGTAAATGCGCCAGGTGTGGTGGCCTCC
GCCTGTAATCCCAGCACTTTGGGAGGCCAAGACAGAAGGATCTCTTGAGCCCAGGAATTTGAGACCAGTCTGGACAA
CATAGACCCTGCCTCAAAAAATAAATTTAAAATAAAATATTAATAAATGTAAAATATAATAACCTAAAAAATGGTCT
ATTCTGAATATAAATATCGGTGACTAACGCAGAGTATGCATGCATTTTTTGGGAGTTGATTTTCAAGTAGAGATGAT
AGAATTTTTGCAGCCAGCAACCAATCAACTGTAGCTGAGAGCCTTTGTTAGAGGGCTGCTATGACTTTAAGATTCAA
GTGTGGTCATGAAATTGAGAATATTTAAACTATATTATGACATATATTTTAACCAATTCATTGAGCCAGAAGAGATA
GTTACTTTCAAAATTACCCTCAAAAAAAATTAACTCTATAAACAGATTGTCATGTGATGTCACCATAAATGGTAACA
ATGTAGTACAAACAGGTTCTGAGGCAACATTAGGAAAATGGTATGAAATAGCCAATTAAATGTGATTGTTGAATATT
TGGGGTCTAAAGCCATTTGTAATAAAGACATTACAAAAAATATCAGAACTGGCCAGGTGCGGTGGCTCACACCTTTA
ATTAGGTGAGCGGATCACTTGAGGTCAGGAGTTCGAGACCAGCCTGGCCAACATGGTGAAAACCCATCTCTACTAAA
AATACAAAAATTAGCCAGGCATGGCAGCGTACGCTTGTAATCCCAACTACCTGAGAAGCTGAAGCAGGAGAATTGCT
TGAATCCAGGAGGCGGAGGTTGCAATGAGCCCATGCACTCCAGCCTAGGTGACAGAGTGAGACTCTGTCTCCAAAAA
ATATATATATATGTATATATACGTAAATACAGTTATTTTTATGTATATATATATATGAACTGAGCTACTGTAATTAC
ATGTAAGAAGTTAAGAAGTTATGATAGTAGATGATTTTTCATTTATGTAACTGATCTATGTATTGAGGTCTATACAG
ACATGAGATGTGAATAGAAAAACTGACCATGAGCTAAGGCCAAAAAGCATTAAAACAAAATGTTTAAGATATTTAGG
TTTAGGTTTTTGGTTTTTTGAGACAGTCCCACTCTGTCACCCAGGGTGGAGTGCAGTGGCATGATCATAGCTCACTG
CAACCTTGAACTCCTGGGCTCAAGAGATCCTCCCTCCCAACATGCTGGGATAACAGGCATAAGCCACCACACTTGGT
CTTATTTTGTTTTTATTACAGGTTGTAGTAGGCTATGCTTCATTTTTCCATCTGTTGAATGGGAAATAAAAACACAT
ACAGCCAATTTGAATAATGTTTTTGTTTACATTTTATAACTTAAAAAATGTAGGGAGAATTTTTAAACTCCCTGTAT
GTTTACAGATATCCTAAGAAATACGAAAGATGAAAGACAAGTTATATGTTTCATTTTACAACTAAACAGTTCATGCT
CTGCCCACATTGAAAGTACGTACTCTTTTGTAGATAAAAGGTACAGTCAGTCCTGCTATACTTGTTTCAAAACTGAA
TTCTAACTCAATTGATATATTAGGGAAAAATTTGAGCATGAAATAAATTTCACATTTGCTTGTGTGTGATTTCATCT
ATAAGAAGCCTTAGGTAAAAGCAGAAAACTGCACCCAGTTGAACTAAACCAGGTATGAATAGATGGAATGCACCAAC
ATCCCCCAGCTACCTTAGTGAGCATTAGGTCTTTTTCACCGTAAAGTGCCATATTGTAGTATTTATCTATTTCTCAA
CCATTTAACATGTATAAAACTGTTATTATTAGGTTCCTGTTTTTGTTTTTTAATGTGGCACTGACCAAGTTTTTAAA
TGCTGTGCCCTCACTTTCCCCACCAGCCTGGGGATTTTTAGTGCGTGATTTAGCATAGTGCAGTGATTTTAGGAAGG
CACTTGTTACATTATAGCAGAACTATGTTTTTATTTCACTTGACATAAAATCATAACTGTGCCTAGATATTTGTACC
ACTGGTACTGTTTTCTCTTGAGTCCAAGATCCTGAGACTGAATTCTGTTCATTATAAAACTTACCAGGACTGTAATC
CCAGCACTTTGGGAGGCTGAGGCAGGCAGATCATTTGAGGTCAGGAGTTCGAGACCAGCCTGGCCAACACGGTGAAA
CCCCGTCTCTACTAAAAATACAAAAATTAGCCGGGTATGGTGGGGCACACCTGTAATCCCAGCTACTCTGGAGGCTG
AGGCAGGAGAATTGGTTGAACTCGGGAGGTGGAGGTTGCAGTGAGCTGAGATTGTGCCACTGTACTCCAGCCTGGCT
GACAGAGTGAGATTCTGTCTCAAAAAAAAAATTTTTTTTGAGGAAAAAATTTTCCTCTTTTCCTCAAAATTACCAGG
AAAAGCCCGGGTGTGGTGGCTCACGCCTGTAATCCCAGCACTTTGGGAGGCTGAGGCAGGTGGATCACCTGAGTTCG
GGAGCTCGAGACCAGCCTGGCCAACATGGTGAAACCCCGTCTCTACTAAAAATACAAAAATTAGCCAGGCATGGTGA
CAGGCACCTGTAATCCCAGCTACTCAGGAAGCTGAGACAGGAGAATCGCTTGAACCCAGGAGGTGGAGATTGCAGTG
AGCCGAGATTGTGCCATTGCACTCCATCCAGCCTGGGCAACAAAGTGAGACTCCGTCTCAAAAATAAATAAATAAAT
AAATAAAATTACCAGGACAATGCTATTTGTATATGATAAAATATACAAATTTTTGGATAGTGTTTATTTTAAGCAAA
TGGAAACTACAAGATTAGTATACAAATGAGTGCTAATGTTCTAGACCTGTTCACTTTAGTACAGTAGCTACTAGCCA
CATGTGGCTATTTAAATGTAAACTAAATAAAATGTAAAATTCAGTTCCTCAATCACGAGGAACCAATGTGGCTAATG
CCTATTTTATTGGACAGCGACAGATATAGACATTTTCATCAATGCAGAAAATTTTAATGGGCAGTGCTGATTTAGAG
AAATTGAAAAGCATTTCCTCTAGTCAAATCAATTTGTATTAAATCAGTATTTTGTTATATATCTATAATTACATTCC
AACTGTTTCATACATAAAAAAAGATATATATAATTTTCCAAATTTTTATTGTTTCCTGAATTCATATAAGTCAGTTT
TTCAGAATTTTTATAAGGTTTGAAAATAATATAGATCTGCTTTCCTGCATGTTCTTTGAAACCCTATGAACCTGAAT
TCATATAAATTCCTCTAAATTAAAGAAAATATATGCTTACTCTAATATAACCCTCTATTTAGTCATTTGAAAACATA
ATTTAAGTGTAAATGTATGATTTTATGCAGGTTTGCAGACCGGGGACACAGTGTAGTTGGTGTGGAAATCAGTGAAC
TTGGGATACAAGAATTTTTTACAGAGCAGAATCTTTCTTACTCAGAAGAACCAATCACCGAAATTCCTGGAACCAAA
GTATTTAAGGTTTGTTTTGATTTGGGTAAATAATTGTATCCATATCCCCACAAAAGTTTTTCTCAGTGTGAGTATTA
TGAGGATACCATTCATGTGTCCGATGGTTCCTATTTAGCACGCAGATTCACTGTAGATACTATATAGTATAAGAAGC
AAGGGCTTAAAAATATAGGTGATAGCTACCTAAATAGGTATAGACATATGTATATAAAAGCTGAGGTCAAAGCCTCT
CTGTACTCAAGCTTTTAGGCTTGTTTTATTTTTATTAACACGCATTTTCTGAGAACCCATCATGTGCCAGACCCTGC
CTAAGACATTGAAGAGATAAAGATAACACAGCACACCCCCCTCACTCCCACCCCTAAAGAATCTCTTAGTTTAGGAG
AGAAGAGAAACAGGGACAAAGCTATTTGTAATGCATGGAGCTAAGTGTAAAGACACAGGGTATTAAGGGAAGAGATG
GGAGTTACCTGCCCAACCTGGCGGTGCTTCCTGAAGCATGGTGACAGATAGAGCTGGAATCAGGCAGGGCCTGGGTC
CATGGTCTTGGACACCTCGGGAAGATACTACTTAAATCACCCATGAGGCCTCATAGGAAGTGATTTGAAGGGAAGTA
GCCTCCTGGCAGAGAGACAGGAGGCTATGGCAGTGGTCCAGGAAAGACATCATGAATCCCTGAGCAAGGAGAGTGAT
GGGGCATAGAGAGTGGTTAGGATGGATTAAAGCAACATTTACGATAGACCCGCAGGATTCTGTGACCGAATGAGGAC
CAAAGGAGCGATGAGTTCACATTTCTGGCTCAGATGACTGGCTGGATGGTCAAGAATAAATGAGAAGGGCAGAAAGA
ATGAATCTTTTTCTAGAGATGATGAGTTTAAGGGACCTGTGGAGTGGTGAAGGTTTTATGTCTGAAGCATGGAGTGA
GGTTTGGGCTCAAAATGATAGGTTTGGGAGTGGTCTTTATGCTGTGAGCAGAATGAGCCAGTGTAAATAAACAAACA
CTACCCAAGTGGGAACAAGCAGAGAGAATCTGGCAGAGACTCAAAGGCAGGCAGAGGAGTGGGGAAGCTTAAAAGTG
GACAAAAGTAAAAGCTCCCGGCATGTGCTGTTGCGAGGCTGTTGGCACAGGGAAGCTGGAGGTGGGCAACTAGGAAC
GGGGCATCCTATGTGATTGGTTTGGGGAGCATGTTTGCCTTTCTCTGGCTGATCCTAAATTGGAAGAAGTGTGGTAG
TAAAAATCAGGGAAGCTGGCAGTCATTGACCAAGTCACTGCAGAGGTTGTGAAGTGGCTACCTTGTCTGGGGTAAAT
TCCCCAGGTTCGTCGTCTTGTGCCAGGAAAATTTAGGACAGAGACACACATGAGGAGTTCAGGAGTGGAGGTTTAAT
AGGCAAAAAAAAAAGAGAAAGAGAAAATAAAACAGCTCTCTCTTTAGTGAGAGAGAGGGGACTTCCGAGAGGAAAAA
ACCTGCCAGGTGTAGAAGCGCCGGATTTTATAGTGCAGCCTGAGGAGTCAGTGTCTGATTTACATAGGGCTCACAGA
TTGGTTTGATCAAGTGTGATGTTTACATAACGTGCTGGGAAGGCTGGTCGCCCCACCCTAATCTTATTATGCAAATG
AACTCTTCCCTTCGCTGGCACCATGTTGTCTGCTTCTTACTCTACAGGTGGCTGACAGAGAAGGGAAGATAGAGCCA
CCATTTTGAACATGATTGGCACAAACTGCCGGCATCTATGTCTGCAGCTCGATTTTATAGGCTGCTCTTTGTTAGAA
AGGAAAAATGATTTGAGGCTGCTTTTCATTAAGAGGAAAACCTTCCGTATCCTCCCTATCTGCCTTAGTAATTTCTT
CTTAACTCCTGAATCAGTTGTGGGGTCAGAGTTGTATTGTCATATAGGATCTATTGTCCATCTGTATATTCAGTGTC
TCACAAAGTACTCTTTTCTAAATATTGAAATTAATTGCTCAGCTGGGCATGGTGGCTCATGCCTGTAATCCCAGCAC
TTTGGGTGGCCAAGGTGGGTGGATCACTTGAGGTCAGGAGTTCAGGACCAGCATGGCCAACATGGCGAAACCCCATC
TCTACTAAAAATGCAAAAATTAGCTGGCTGTGGTGGTGCATACCTATAATCCTAGCTACTCTGGAGGCTGAGGCAGG
AGAATCGCTTGAACCTGGGAGGTGGAGGTTGCAGCGAGCCAAGATCGCACCACTGCACTCCAGCCTGGGCAAGAGAG
TAAGACTCTGTCTCAAAAAAAAAAAAAAAAAATTGTTGCTCAGATCAGTAGGAAGCTTGATGGTATTTTTTATTTCA
CATCTTCCTCCCACCTCATTCTCAAAGCATTAAAGAATCCAAGGAGTTTACTCAGTCCAAAAAGGGCTAGTCCTGCT
TCCAGGCCCCTCTTGGAGCAGCAGTTCCCCGCCCAGGCTGCACATTTGAAATCATCTCTGGATCTTATAAAAATTCC
CAATCCAAAGGTCTTACCCCAGGCCAATTATATCAGAATCTATAGGGGTAGGGCCCTGGCATCAGTACTGTTTAAAG
CTCCCCTTGATTCCAATCTGCAGGCAAAGTTGAGATCCAGCTGTTCTCCTGCCTCCAGGCCCACCCATCTCTGGGCT
GACCCAGTGATCCCAGGCTGGTTGTCAAAGACCCTTCCTCCTCTACTTGTAAACCTGTGTGGGGACCTCATTTCAGA
ATAAAATCTAGCAGTCTTGTGTGCTGACACACCTTTCCTACACTGCCCTTCACCCCAGCTCCCAGGATCCTAAAGTC
TATGTTATATTGCCCCCTCTTGGAGTGTGCCCCCAGTCCACACCCTAGAGTGTCACCCTTTCAAAAGTCACCTCTTT
TTTTGTTTTATTCTCTTGATTTTTGTTGTTGTTGTTTATTTTTTAGGATTATTTTGCTCCAAGTGTTTGTTTTTGAG
ACAGGGTCTTACTCTCACACAGGCTGGAGGGCAGTGGCACAATCACAGCTCACTGCAGCCTTGACCTCCCAGGCTCA
AGTGACCCTCGCATGTCAGCCTCCCAGGTAGCTGGAACTACAGGTGTGTGCCACCACACCCATCTAATTTTTTCTGT
AGAGTTGGAGTTTTGCCATGTTGCCCAGGCTGGTCTCATACTCCTGGGCTCAAGCGATCTGCCTGCTTCAGCCTCCC
AAAGTGCTGGAATTACAGGTGTAAGCTGCCACACCTGGCCAAAAGTCACCTTTTGTTTAGTCTCCCTGATGCTCCCA
GGCAGGCCCCTGCTCTGCCTTCTGTGTTCACATAGTTCGCAGGCACAGAACCACAACAGAGCAGGCCATTCTGGATT
GTAATGTTCTCTGTGCCCACGCTTCTCTGCTCTAGTCTGTCCTCACTGTGGTCAGAGACTGTGTCGAATTCAAATCT
ACTGTCAAGAACCCAGGATAGATGTCTTGTCTATTAGGTACTCAGCAAATAAATGCTCATTGGCTGAATGAATGAGC
AAAAATGGAAGAATCAGCACAATTGCACATTTCTGGAATAACTTAAAATACCTGACTTTTCAGTACCACCCTGCCTG
ATAATTGGTTGACCTGCAGATTTATCTTTAGCAGAGTAAAAATATCACTCTGCTCGAGGAAACATTGTTAGAATTTT
ATAGCAAGCTCTGAAGTGAGTAACAGCCAACTGATCTTCAAAGTTGTCCTCTGTGATATTCCTCTGAGTTGGCCCAA
AAGAACCTCGCACAGGTTTTAAAAGAAAAAAATAAGGTGCCAATAAAGTGCAGTAAAATTATTTACTTTAATTATGT
TTAATTAACACATTCTTTCCCTAGAACTTTTGCTTTGCTTGGCCCTCTTTCCTTGACTATTCAAGTACTTACTAAGT
AAAAAAAAAAAATTTTTTTTTCATTATTTCATTACAGAGTTCTTCGGGGAACATTTCATTGTACTGTTGCAGTATTT
TTGATCTTCCCAGGTAGGTTGAATACTACATCTGCACTTTAAAAAATTTGAATGCTTGCCAGGCAGTGCAGGCATGG
GAGTGGAGGTGTCTTCCTCACTCTCTTCCTCCTGTGTAACATCCACAAAGCATTTTTTTGAATGTCTGTTCTGCAGA
TATTTTTATTACACACTCGTCTGCACACTTTAATGTGTTTTGTCTTTGGTTAGCTCCCAAACTATGGGAAACTGAGG
CAGCTAGGGAAAAAGAAAGGTGAGTAAGACAGTGTCTTCTACCTTGCACCTGGGCCTGTAATAGAAATGAATTTCAA
GTAGCCAAGGGAGATAAGAGCTCATCTCCTGAAAGTCCCTGATACCTGAGCCAGAGGCTGGGGGCAGAGTTGTTGCA
CACTGTCCTTTGTTCCTTCTTCATGTCCCCAAATCATAACAGAGTGGGGAGGCTGCTGCCACAGGCTCCTAAAACCA
TGAGGGGATGGACAGCTCTCCACACCCAGGTCCACACATTCCTCTAGGAGGAAACGCAGACGTGAGATCCTAATACC
TTGACGATTGTTGAAGTACCAGCATGCACCATGGGGGACGCTGCTCATCTTCTTAAAGATTTGATTTTTCTCCCATA
AAATGTTTTTTCTCTTTCTGGTAGGACAAATATTGGCAAATTTGACATGATTTGGGATAGAGGAGCATTAGTTGCCA
TCAATCCAGGTGATCGCAAATGGTAAGTAATTTTTCTTTTTTTGTTTAGCTGTCTTAATTTTTTAGTATACTATACT
TTTTCTGGGTTCTAGAAAATCAGCTTAGACTTCTATGAGTTTGAAATAGGTTATTATGTTTGGAATTTATAAAAACC
TAAATCCAATACTAGCTTTGTCTAAAAAGTATAGGATTTGACTACAGAGACCTGGGTGTAAGGCTATAAGGCTTGTG
GCTCAATCACTTTCAAGCTGTGTGGCCTTGGTCCAAGTATTCAACTTCTCTAAGCTTTGGTTTACTCCTGCAAAATG
GGAATATTTCTGTAATACTAACACAATCATCACCACCTCCACTAATATTACCAGGTAATACTTACTGGTCACTTGCC
TATTCCAGGTATTGTTCAAAATACTTTATGTGGGCTGGGTATGGTGGATCATACATGTAATCCCAGCACTGTGGGAG
GCCAAAGCAGGAGGATTACTTGAGCCCAGGAGTTTGAGACCAGTCTGGGCAACAAAGCAAGACGCCACCTCTACAAA
ATATTTTTAAAATTATCCGACCGTGGTGGTATGTGCTTGTAGTCCCAGCTACTTGGGAGGCTGAGGCAGGAGGGTGA
GCCCAGAAGTTCAAAGCTGCAGTGAGCTATGATCGTACCACTGCACTTCAACCTGAATGACAGAGCAAGACCTTGTC
TGTAAAATATTTAAAAAAAAAAAAAAACAAAAAAATCAAAATATTTCATGTGGTTTAACATTTAATTCTGTAAGGTA
TAAGCTACTATTCCATTATACAGATGAGGAAACTGAGGTCAAAGAGATTAAGTTACTTGTCCAAAGTTAAGCAGCTG
GTATGTGACAGAGTTGAGATTCAAAACCAGAGTGTTTGGCTTTGAAGTCCATACTTGTGGCCGGGTGTGGTGGCTCA
CGCCTGTAATCCTAGCACTTTGGGAGGCCAAGGTGGGCGGATCGCCTGAGGTCAGGAGTTCGAGACCAGCCTGGCCA
ACATGGTGAAACCCCATCTCTACTAAAAATACAAAAATCAGCCCAATGTGGTGGCCTGTAATCCCAGCTACTCAGGA
GGCTGTGGCAGGAGAATCACTTGAACCCAGGAGGCAGAGGTTGCAGTGAGCCAAGATGGCGCCATTGCACTCCAGCC
TGGACGACAAGAGCGACCCTCCATTTCAAAAAAATGAAATAAAATAAAGTCCACACTCATGATCCCTTTGTTAGACT
ACCTCAAAGGATTTTTGTGGGAATTGAAAGAAATGAAATATGTAGGAAATACCTTGTCATCTCTAAAGAACTGTATA
AATGAGAGCTGTTATTATTGAAGCATTGTTATTGATGTTTTTGATGACCATTCTTATTATACCGAGTCTTGTTAGAA
AATAAAATTAAAATATGAGTGAGGAAGCATACTGAGTAAAATGTGACCACATCTGTATACTCTTTCAATTAAAATAA
TGTCATTTATGTGCTTACTTGGATATAGCATGGAAATATTGAATTACTTAGTTGTATGGAGCAGACTCATCACTTGG
CCAAAAAGGAGGCTGGGCGCAGTGGCTCACGCCTGTAATCCCAGCACTTTAGGAGGCTGAAGTGGGTGGATCATGAG
GTCAGGAGTTCGAGACCAGCCTGGCCAACACAGTGAAACCCTATCTCTATAAAAATACAAAAATTAGCTGGGCTTGG
TGGCGCACACCTGTAGCCCCAGCTACTTGGGAGGCTGAGGCAGGAGAATTGCTTGAACCCGGCAGGTGGAGGTTGCA
GTGAGCCGAGATTGTGCCACGGCACTCCAGCCTGGGCAACGGAGTGAGATTTCATCTAAAAAAACAAACAAACAACC
AAAAAAAAAACAGGTTAGGCTCCACATCAGTGAAATAAGATTTTTATTTTTAAAAATTGTTTCATCAGTAAGGCTTA
CTGCCAAACCTAATCTTTTTAAATTAGACCATGCCGCATCTTAATTTTCATGCTCCTGTTTCCCTCTTTTTCTCACT
GCTCCTCCTTTATCTCTTTTCCAGAACTCCCATCCTCCTTGCATGCCTAAAAGTCGTGATTCCCCAAGGGACCACCA
TCCTCAGCCTTATGCTCTCACCCAAAAATTGCCAATATTTGTCCTACCAGAAAGAAAAAAAAATTTTATGAGAGAAA
AATCAAATCTTTTTTTTCGAGACAGAGTTTCGCTCTTGTTGCCCAGGCTGGAGTGCAATGGCACTATCTCGGCTCAC
CGCAACCTCTGCCTGCCAGGTTCAAGCGAATCTCCTGCCTCAGCCTCCCAGGTAGCTGGGATTATAGGCATGCTCCA
CCACACCCGGCTAATTTTTTTGTATTTTTAGTAGAGACGGGGTTTCTCCATGTTGGTCAGGCTGGTCTCGAACTCCG
GACCCCAGCTGATCCACCCGCCTCGGCCACCCGGTGCTGGGATTAGAGGTGTAAGCCACCACGCCTGGCTGAAAAAT
CAAATCTTTAAAGATCAATTCTGTTACTTTCATTCAGCTATTCAACAAATATTTACTAAGTTCCTGTGATGTTCCAA
ATAGATAGTATGCCAGAGGCGGGGCAAGTAACTGAACAGATTATACATGGTATTCGCTCTGACAGTCTTTACATTTC
CAGCTCTGTTCTTTCTCCCAAACATCCACATCACATGTCCAGTGTCTGCTTTGCACTTCCCATGTGACATCAGTGTC
CTTTAACAACTCAAAATATCACCGAGACCTCTGTCTCCGTGAGACACATTTGTATATGAAACCCCATCTGCTAATGT
CTAAATTTGTAGGTGTTTCCCACTCCCTGAAGATGATCATCCATTCCTTAGAGGTTAGTCCTTCTACAGGCTCTGGT
TGGCATGGTAAAATCCACCAAGCAGTGGGAAGCAAACTAAAAGTCACCACGTGCAGACCAAGAGCGGACCCAGCACT
TTGGGAGGCTGAGGTGGGCAGATCACCTGAGGTCAGTTATTTCCCCAGTGTCTGATATAATATATATTTAGCACCTA
GTAAGAGCTTAATAAATGTTGTTTAAATAAATGAATGGAAGAAGTGAACTGCACTCTGAGCAAGGGCGATATTAGTT
TACTATGTACTGGTAGTATCTTAGGGTTTTTTTTTGAGATGGAGTTCCACTCTTGTTGCCCAGGCTGGAGTGCAATG
GCATGATCTCGGCTCACTGCAACCCCTGCCTTCTGGGTTCAAGTGATTCTCCTGCCTCAGCCTCCAGAGTAGCTGGG
ATTACAGTAGCGCCACCACACCCAGCTAATTTTGTATTTTTACTAGAGACGGGGTTTCTCCATGTTGGTCAGGCTGG
TCTCGAACTCCCGACCTCAGGTGATCTGCCCACCTCGGCCTCCCAAAGTGCTGGGATTACAGATGTAAGTCACCGTG
CCCAGCCTTATGTTAGTATTTTATATATGTGCTGGTACTTAGTATCTTAGTTCTTTATCTACATGCTGGTACTCAGT
ACTTACCATGTTAGCACATTATATTGGTGCTTTCGTTAGGACTGGGTGTCTTCTCCTTAGATTGTACATTGTCAAGT
ACTGATGTATTTTCTGCTGTGACTCTCGAGTTCTTGCCTAATGTAAACCAGTAGGGCAGACCTCATTTTTCTGGACT
TTTTGAGACTGGTTGAGAGAGAAAAACTTCACATTCTAAGAGGAATGAAGTCTTTCAGTCCTAGGAAACCCTACCAA
ACCAGTCTGACATCCCCCTCATGGGGACAGCAAGTTTCTTCTGAAAAATGAGAATCAGAAAGTAACAGTTTCTGTTC
AAAAGTTTGCTTTGATGTGGTTCTTTCACCTACAAGGAAGAGTGATCCACTGGTGAATTCTCACAGTTTGTGAGGTG
GTTATTAGTCTTATCCTCATGTTACAGGTGAGGAAACTGAGGTACAGAAAGGTTAAGAATTTGTCAAGCTTACATAG
CCAGTCAGTAGAGGAGCTGGGGTTTGAGCCTGGGCATCTCATAGAGTCTGCACTTGCCTCCCCAACTTCTGGCAGAC
CTCTGGACATAGATCACAGTCTAGCCAGGCTCCATAGAAACTGGAGTGCCAAGAGCCCAGGCTCCTTCCTCAGTGTG
GGGAAGGGTGTCCATCTTTGTCCTTGGCCTGTCTCTCATGGCCTCTCTACCACCCTGCATGCGTCTCCTGTCCTCTC
CTTATCCCTCAGCTTCCTCCATTTTTATTCCCACATAGCTGACTTGCCTTCAACGGTCAGCCTCCTTGCTCCAACCA
CCATACAATACAAAAGCTCCAACTTTCAGTAGGACCACAGCTAATTAGCAATTCCTCTTTGGTCTCTTAATTCAGTT
TCCAAGTCAGTCATCTGATTGGTCAACTCATCGTCTGTACCTGGTCCAGCCCTGGCTTCTGCTCAGTCTATTGATGG
GTCCTGTTAGCATCAGCTTCCCACTCTGAGCCAATCAGACCTGGCTCCCTGAGGCACCTACTCAAAGGACAGTTCCA
TTGCACTCATGGTTTATGCCACACTTGCAGAGCTTTAATTTCATGCCACCTTTTTTTGGCCTGCTTGTTACTCTCAT
TTCTAACTAAATAGAGGAAAAAAATTGTGTTTTCTCTTACATAAGGCCCCTGGAGGGGTCCCAGTCACCCAGGATAT
TTGTTAAATTGCAGCATCCCAGGCCCCATGGCAAACTAACTGCATCGGTCTGTGGGGATGGAGCTCAGGCATCTTTT
CTTTCAACAAGCACCCCAGTGATTTTGACACCCAGCCTGGTCTAGAAACCACTGTCCTCAAGCACACAGGCATCCAA
GGTCAGCAGGCCCTCGAGTACCTCATCAGGCCAGAAGGAGTCTCTACCCATCCTTATGTAATGTCTTCTGCTCTGAT
CAGCTCTGGAAGCAGGCCTCGATTTGATTCCCATCTGGACCAATTCCCAGCTTAGGCAGGGGCCATAATTTGCTAAA
CCCATTTCTCACTTTTAAAATGAGAATCAACCTCTTGTGATTATTAGGAGAATTTAAACTCAGTAGTATCAGCGAAA
GTAACTTCTGGCTTCCTTCCCTGCCTTTTGTCCCTCCAGCCTTTGCCTGTGTAGAGAAATGTAACAAATACCTTCTT
GTGTTCTTTGTAGCTATGCAGATACAATGTTTTCCCTCCTGGGAAAGAAGTTTCAGTATCTCCTGTGTGTTCTTTCT
TATGATCCAACTAAACATCCAGGTAAAGTTGTTGGGTTTTTTTTTTTTCTTTTACCAGTTGCCTAGTTTCTTGATAT
AAATTCTATGACATGGATAAGAATTATTTTCTCCTAATTTATCTCATAGGAAGCATACTGCATATATACATGGAGAT
AATTCCAGTTTTGACTAATGGGAACTATAAGCATGTTTTTCTCTTCTATATTGTACATGATTTTCTTGTATTCCTTT
CCCATTTTGTTTACCATTCACAATTCAGACTAGAGAAGAGCGGGGAAATGGATGATCCTGATACAGAAGAGAAATCT
CATGAGTCCTATTGGGAAGAAGGAGGAATGTGGTTGGATATTGCATATCCGTATCACCCAAGTGGTAGTGATGTGTT
GGACAGATGCCTTCACATACACAAGCACGGGGTGAGAAGTGAATATGGGGATGCAGAAGTTGTGCAGGAAAAACTCT
AAATTATTGTCCTTTTTTAATTTCTGAAGTTTTATAATCTATCTATTGCATTCTTTTATTTGTTTTGAATCTGTTGG
GGGTATGTTTTCTCCTGGACTCATTTTAATGCATTCAAACATTGAAACTTAACCTTTGTCATGGCTGGAGAAATACA
TAGACTTTTAATTTGTAAGAGTCACCTTTTTGATTTTCAACTGAGCTTTTTAACATGCTTGTTTTGTAAATATTTAC
ATATGTTAGTATTTTGGTTTTTTTGTTTTGTTTTGTTTTTGAGACACAGTCTCACTCTGTGGCCCAGGCTGGAGTGC
AGTGGTTTCATCTCGGCTCACTGCAACGTCCGCCTTCCGGGTTCAAGTGATTCTCCTGCCTCAGCCTCCCAAGTAGC
TGGGATTACAGTGTATGCCATCACACCCAGCTAATTTCTGTATTTTTAGTAGAGACAGGATTTCACCATGTTGGCCA
AGCTGGTCTGGAACTCCTGACCTCAGGTGATCCGCCCTCCTCAGCCTCCCAAAGTGCTGGGATTACAGGCGTGAGCC
ACCGTTCCCAGCTAGTATTTTGTTTCTGCTTGAATAAATTGGGGTCTTAAAAACTCATTAAAAGATGAGACCTAGTG
TCCCTAATTAAATCAGAGACAATCAAATGGAAATAGAAATTAATAGAGGCAGAAGTTAGCTCTGCTAATCTGATTTA
AATTCCATTAATGAACAGAAGCCCTAACAAAGAACCTTTGGGAAGAAAATACAGCAGGCCCTGTCTGGAGGTGGAGT
CTGAGGATACTGCTCTTAGAAGGGGGCGGAAGCTCAGCTTCCTTATACTCTATATCCTGCAGCAGAGCGGGAGGGGT
GTATAAAATGTGGGTGAGTGCAGGAGTTGGTAGAAAATCAGTATGCTAGAAGATGGTTGAAACCCCCAAGGAATCTG
CTGATGGAATCCACTCTGATTTCAATGAGCCTAAGTCCCTTGACCGGTTTAGCTGTCTCATCTAAGCTAAAGATGGA
TATGATGGGGATGTAACATTGCCATCCTCTCAGTAAGTCAATTTATTTCTTTAGTATGAGATTTGCAAAAGAAGAAT
GAGAAGAACATGCCACATCATCACCTATTTGGATAATGAAGTTGTATGTAGAACAAGTACACCTACGTCATTGGAAA
TAAACTCAGAATTTATTTCTCTTTTACCTAGGTCCACCATTTTATGTTCCACATGCTGAAATTGAAAGGTTGTTTGG
TAAGTTTGTGGACTTATGCTGTAACTTTTTAAACAAAGTCAAGTTAACTCTTTTGGGCCTGGCGTGCTGGCTCATGC
CTGTATTTCCAGCACTTTGGGAGGCCAAGGTGGGCAGATCACCTGAGGCCAGGAGTTCGAGACCAGCCTGGCCAACA
TGGCGAAACCCTGTCTCTACAAAAATACAAAAATTAGGCCAGTGTGGTGGCATGTGCCTGTAGTCCCAACTATTTGA
GAGGCTGAGGCATGGGAATTGCTTGAACCTGGGAGGCAGAGAGTGTAGTGAGCCAAGATTGTGCCACTGCACTCCAG
CGTGGGTAACAGAGTGAGACTCCATCTAAAAAAAAAAAACCTATTGTGAACTATTAGTATATTAGTATCTAGAGTAT
GTCTATAAAATTTAAAAGATAAAGACCATTTTATTGAAAGTTGTTTTATCTTAGAAAAGGAACTAATCTCTGTAAAT
ATGCTCTGTATATATGCTATATGCTCTATGTTAAAGGTATTTGAACTTTTCTAGAGAGATGGTATATTTTTTTTTAT
TTGTTTATTTTTGAGATAGGATCTTGATCTGTCACCCAGGCTGGAGTACAGAAGTGCAATCACAGCTCACTGCAGCC
TCGACCTCCCTGGGCACAAGTGATCCTCCCACCTCAGCCTCCCAAGTTTCTGGGACCAGAGGCATGCACCACAATGC
TTAGCTAATTTTTCTATTTTTTTGTAGAGATGGGGTTTAACCACATTGCCCAGGCTGGTCTCGAACTCCTGGGCTCA
AGTGATCCTCTTGTCTTGGCCTCCCAAAGTACTGGGATTAGAGGCATGAGCCGCCATGCCCAGTGGTGTATTTATTT
TTACTCTTGGGAGTTAAAAGTGATAATAACAATTCAGAGTTCAGGAAATTTGCCCTTAGCATTCCTCTGTTTTGATT
AAAAAGAAGAGAAAAATGTTTAGTGTACACTGTTTTTAAAATAATTTCAAGGTTTTGTTTGTTTGTTTTGAGATGGA
GTTTCGCTCTTGTTGCCTAGGCTAGAGCGCGATGGTGCAATCTCAGCTCACTGCAACCTCCATCTCCCGGGTTCAAG
CGATTCTCCTGCCTCAACCTCCCAAGTAGCTGGGATTACAGGCATGTGCCACCATACCCAGCTCATTTTGTATTTTT
AGTAGAGACAGAGTTTCACCATCTTGGCCAGGCTGGTCATGAACTCCTGACCTCAAGTGATCCACCTGCCTCGGCCT
CCCAAAGTGTTGGGATTACAGGTGTGAGCCACCGCACCCAGCCAATTTTGAGTATTTTTAAAAGAATCCCTGATGTC
ATTCTTCATAGTATTTTAACATGTTACTCTTTCTTGTTTCAGGTAAAATATGCAATATACGTTGTCTTGAGAAGGTT
GATGCTTTTGAAGAACGACATAAAAGTTGGGGAATTGACTGTCTTTTTGAAAAGTTATATCTACTTACAGAAAAGTA
AATGAGACATAGATAAAATAAAATCACACTGACATGTTTTTGAGGAATTGAAAATTATGCTAAAGCCTGAAAATGTA
ATGGATGAATTTTTAAAATTGTTTATAAATCATATGATAGATCTTTACTAAAAATGGCTTTTTAGTAAAGCCATTTA
CTTTTTCTAAAAAAGTTTTAGAAGAAAAAGATGTAACTAAACTTTTAAAGTAGCTCCTTTGGAGAGGAGATTATGAT
GTGAAAGATTATGCCTATGTGTCTTGCAGATTGCAAGATATTTTACCAATCAGCATGTGTTACCTGTACAATTAAAA
AAATATTTCAAAATGCAATGCATATTAAATATAATACACACAGAAAAACTGGCATTTATTTTGTTTTATTTTTTTGA
GATGGAGTTTCGTTCTTGTTGCCCAACCTGGAGTGCAATGGTGCAATCTCAGCTCACTGCAACCTCTGCCTCCCAGG
TTCAGGTGATTCTCCTGCCTCAGCCTCCTGAGTAGCTGGGATTACAGGTGTGCGCCACCACGCCCAGCTAATTTTTT
GTATTTTTAGTAGAGACAGGGTTTCACCATGTTGGTCAGGCTGATCTCGAGCTCCTGACCTCAGGTGATCTACCCAC
CTCGGCCTCCCAAAGTGCTGGGATTACAGGCGTGAGCCACTGCACCTGGCCTGACATTCTTTATGAAATTTAGAATT
GTTGAAGAACTATAACATTTCAGTAGGGTTCAAGGTGGTCCCAAAAGTTATATAAAAGATTAGTTTTTACTATAAAC
CCTTGTCTTTTACTCAGATCCTAGCATCCCTTTTCACATGGTTTCTCCATGTATATAACAGAATCAAGAAACAAATT
TTAATTAAACAATCTGTAACAGAATCAAGAAACAAATACATTTTAATTAAACAATCTATATGGAACAAACATTCCCA
AATTCTAAGAATAAATTTTTCTTTAAGTTTTCTCTGAGTTTGGCAATTGTTGTTTTTTATAATTTAATCTGTTTAAA
TCATCAGGTCTTATAAAATATAATGTACTTAGAGCTGGATTCATGGCTGTTTATTATGAAAGGTTAGATTTCTCAGT
TCTTCTTTAACCACATTTTGTTATATCAGACAGTCCTCTATAACTCTGTACTACCCAACAACTAAATGGTTTAGATT
GTTTAGCTCATGTTAATAGGATGGTTGTGTATTATAAAAAACGAGTTACGTGTGTGTGTGCACGCATGCACGCACAT
GTGCTGGCTTAAAGGTTGTTAATGCAAGGTTTGGGGTCCCCTTTAACACTGGTGAAAGCTACGGTACTCTCCCCAGA
GATATGTCTTGTCAGCCTCTCTAGTTCCCCTTGGCCTGCATGTACAAACTTCTACCCTAGAAGCTCTCTGCCATCGA
TGTATTCTAATAGATTTGTAAGGCTATTAATTTGAAGCAACTCCTTGCTCACAGTGATTCTTGCTTCTCTGAGACCT
GCTCCCAGTCGATACTGTGGGCTTCAGAAGCCATGACTCCCCAACTCTGCCTGTATCACCGGTTGAATGGACAACTA
ACCCGAGCTGGACCAACACAATTCTCTCCAGAGACTTTTGATTTTACTTTTATGTAGAGACAGGGTCTCACTTTGTT
GCCCACGCTGATGTTGAACTTGACGTGAGGCCTCAAGCAGTCCTCCTGTCTTGGCCACCCAAAGTGCTAGGATTACA
GGTATGAGCCATTGCGCTGGCCCTCTTCATAGGCTTTTGGACTTGGGAATAGAAAAGCAACCCCGTCTCTACTAAAA
ATACAAAAAAATTAGCCAGGCGTGGTGGCACGTGCCTGTAATCCCAGCTACTTGGGAGGCTGAGGCAGGAGAATCAC
TTGAACCTAGGAGGCGGAGGTTGCAGTGAGCTGAGATCATGCCACTGCACGCAAGCCTGGGCAACAGAGCAAGACTC
TGTCTCAAAAGAAAGAAAAAGAAAAGAAAAAAAAGAAAGGCAAGTTGACTGCTGAAAGGGGAATCTGTGTACGCCTG
GGAGCTGTGGGGCAGCCACATTCCAGCACATGGATCTGAGAAACAGAACGCTGATCTGCAGAAAGAGATGAGAACCA
AAGAGAGGCCACCTGCGTCCTGGGTCCATTTTCATCCTCCCTGAAGCCCAGCTGCCCAGGGTGGGGAGAAACACCCT
GTGTCCATGGGATAGAGTCCTTTCCGCTTGCAGTTGTGCCCAAAGAATCTTAAATACAAATGAGATATCCTTAGGTA
GTTGATCATTTATGTAATATGTGTCTTCACTGGGGAATACTGACTTCCTAAAATCTCAAGATGGAAGATATACCACA
TGTAAATTATTTTAGAGCAATTAAATTGTTTTCAGGATTTTCCAAAAA

### Tables

TABLE 9
Pharmacogenomic Marker Mutations

| SNP ID | CHR | POS | REF | ALT | Gene | Haplotype |
|---|---|---|---|---|---|---|
| rs9923231 | 16 | 31096368 | C | A | BCKDK, PRSS53, | VKORC*2 |
| | | | | | VKORC1 | |
| rs9923231 | 16 | 31096368 | C | G | BCKDK, PRSS53, | VKORC*2 |
| | | | | | VKORC1 | |
| rs9923231 | 16 | 31096368 | C | T | BCKDK, PRSS53, | VKORC*2 |
| | | | | | VKORC1 | |
| rs1800559 | 1 | 201060815 | C | T | CACNA1S | |
| rs1800559 | 1 | 201060815 | C | A | CACNA1S | |
| rs772226819 | 1 | 201091993 | G | A | CACNA1S | |
| rs3745274 | 19 | 41006936 | G | A | CYP2A7P1, | |
| | | | | | CYP2B6 | |
| rs3745274 | 19 | 41006936 | G | T | CYP2A7P1, | |
| | | | | | CYP2B6 | |
| rs2279343 | 19 | 41009358 | A | G | CYP2B6 | |
| rs28399499 | 19 | 41012316 | T | C | CYP2B6 | |
| rs3211371 | 19 | 41016810 | C | A | CYP2B6 | |
| rs3211371 | 19 | 41016810 | C | T | CYP2B6 | |
| rs3211371 | 19 | 41016810 | C | G | CYP2B6 | |
| rs34223104 | 19 | 40991224 | T | C | CYP2B6 | |
| rs34826503 | 19 | 41012339 | C | T | CYP2B6 | |
| rs36056539 | 19 | 41006923 | C | T | CYP2B6 | |
| rs12248560 | 10 | 94761900 | C | T | CYP2C19 | CYP2C19*4 |
| rs12769205 | 10 | 94775367 | A | G | CYP2C19 | CYP2C19*2 |
| rs3758581 | 10 | 94842866 | A | G | CYP2C19 | CYP2C19*1 |
| rs4244285 | 10 | 94781859 | G | A | CYP2C19 | CYP2C19*2 |
| rs4986893 | 10 | 94780653 | G | A | CYP2C19 | CYP2C19*3 |
| rs1057910 | 10 | 94981296 | A | C | CYP2C9 | CYP2C9*3 |
| rs1799853 | 10 | 94942290 | C | T | CYP2C9 | CYP2C9*2 |
| rs28371685 | 10 | 94981224 | C | T | CYP2C9 | CYP2C9*11 |
| rs28371686 | 10 | 94981301 | C | G | CYP2C9 | CYP2C9*5 |
| rs7900194 | 10 | 94942309 | G | A | CYP2C9 | CYP2C9*8 |
| rs7900194 | 10 | 94942309 | G | A | CYP2C9 | CYP2C9*27 |
| rs7900194 | 10 | 94942309 | G | T | CYP2C9 | CYP2C9*8 |
| rs7900194 | 10 | 94942309 | G | T | CYP2C9 | CYP2C9*27 |
| rs9332094 | 10 | 94936917 | T | C | CYP2C9 | CYP2C9*8 |
| rs9332131 | 10 | 94949281 | GA | G | CYP2C9 | CYP2C9*6 |
| rs1065852 | 22 | 42130692 | G | A | CYP2D6 | CYP2D6*4 |
| rs1135822 | 22 | 42129180 | A | T | CYP2D6 | CYP2D6*49 |
| rs1135833 | 22 | 42126663 | G | C | CYP2D6 | CYP2D6*4 |
| rs1135835 | 22 | 42126660 | T | C | CYP2D6 | CYP2D6*4 |
| rs1135837 | 22 | 42126633 | C | A | CYP2D6 | CYP2D6*4 |
| rs1135838 | 22 | 42126627 | A | C | CYP2D6 | CYP2D6*4 |
| rs1135840 | 22 | 42126611 | C | G | CYP2D6 | CYP2D6*2 |
| rs16947 | 22 | 42127941 | G | A | CYP2D6 | CYP2D6*2 |
| rs28371706 | 22 | 42129770 | G | A | CYP2D6 | CYP2D6*17 |
| rs28371706 | 22 | 42129770 | G | A | CYP2D6 | CYP2D6*82 |
| rs28371706 | 22 | 42129770 | G | T | CYP2D6 | CYP2D6*17 |
| rs28371706 | 22 | 42129770 | G | T | CYP2D6 | CYP2D6*82 |
| rs28371725 | 22 | 42127803 | C | T | CYP2D6 | CYP2D6*41 |
| rs28371735 | 22 | 42126636 | G | A | CYP2D6 | CYP2D6*4 |
| rs35742686 | 22 | 42128241 | CT | C | CYP2D6 | CYP2D6*3 |
| rs3892097 | 22 | 42128945 | C | T | CYP2D6 | CYP2D6*4 |
| rs5030656 | 22 | 42128173 | CCTT | C | CYP2D6 | CYP2D6*9 |
| rs5030862 | 22 | 42130668 | C | T | CYP2D6 | CYP2D6*12 |
| rs59421388 | 22 | 42127608 | C | T | CYP2D6 | CYP2D6*29 |
| rs61736512 | 22 | 42129132 | C | T | CYP2D6 | CYP2D6*29 |
| rs72549356 | 22 | 42128927 | T | TGGGG | CYP2D6 | CYP2D6*30 |
| | | | | CGAAA | | |
| | | | | GGGGC | | |
| | | | | GAAA | | |
| | | | | (SEQ | | |
| | | | | ID NO: | | |
| | | | | 2218) | | |
| rs72549356 | 22 | 42128927 | T | TGGGG | CYP2D6 | CYP2D6*40 |
| | | | | CGAAA | | |
| | | | | GGGGC | | |
| | | | | GAAA | | |
| | | | | (SEQ | | |
| | | | | ID NO: | | |
| | | | | 2218) | | |
| rs74478221 | 22 | 42126624 | C | T | CYP2D6 | CYP2D6*4 |
| rs75467367 | 22 | 42126623 | G | C | CYP2D6 | CYP2D6*4 |
| rs766507177 | 22 | 42126635 | T | G | CYP2D6 | CYP2D6*4 |
| rs776746 | 7 | 99672916 | T | C | CYP3A, CYP3A5, | |
| | | | | | ZSCAN25 | |
| rs10264272 | 7 | 99665212 | C | T | CYP3A5, | |
| | | | | | ZSCAN25 | |
| rs41303343 | 7 | 99652770 | T | TA | CYP3A5, | |
| | | | | | ZSCAN25 | |
| rs2108622 | 19 | 15879621 | C | T | CYP4F2 | CYP4F2*3 |
| rs3918290 | 1 | 97450058 | C | G | DPYD | |
| rs3918290 | 1 | 97450058 | C | T | DPYD | |
| rs55886062 | 1 | 97515787 | A | T | DPYD | |
| rs55886062 | 1 | 97515787 | A | C | DPYD | |
| rs56038477 | 1 | 97573863 | C | T | DPYD | |
| rs67376798 | 1 | 97082391 | T | A | DPYD | |
| rs6025 | 1 | 169549811 | C | T | F5 | |
| rs2395029 | 6 | 31464003 | T | G | HCP5 | |
| rs1061235 | 6 | 29945521 | A | T | HLA-A | |
| rs17179220 | 6 | 29853458 | G | A | | |
| rs10484555 | 6 | 31313688 | T | C | | |
| rs58102217 | 6 | 31378878 | G | A | | |
| rs12979860 | 19 | 39248147 | C | T | IFNL3, IFNL4 | |
| rs116855232 | 13 | 48045719 | C | T | NUDT15 | NUDT15*2 |
| rs9263726 | 6 | 31138722 | G | A | PSORS1C1, | |
| | | | | | PSORS1C2 | |
| rs111888148 | 19 | 38455463 | G | T | RYR1 | |
| rs111888148 | 19 | 38455463 | G | A | RYR1 | |
| rs112563513 | 19 | 38499223 | G | A | RYR1 | |
| rs118192116 | 19 | 38451850 | C | T | RYR1 | |
| rs118192116 | 19 | 38451850 | C | G | RYR1 | |
| rs118192122 | 19 | 38500643 | G | A | RYR1 | |
| rs118192124 | 19 | 38500636 | C | T | RYR1 | |
| rs118192161 | 19 | 38444211 | C | T | RYR1 | |
| rs118192162 | 19 | 38455359 | A | G | RYR1 | |
| rs118192162 | 19 | 38455359 | A | C | RYR1 | |
| rs118192163 | 19 | 38494565 | G | C | RYR1 | |
| rs118192163 | 19 | 38494565 | G | A | RYR1 | |
| rs118192167 | 19 | 38580004 | A | G | RYR1 | |
| rs118192168 | 19 | 38580403 | G | A | RYR1 | |
| rs118192170 | 19 | 38584989 | T | C | RYR1 | |
| rs118192172 | 19 | 38457545 | C | T | RYR1 | |
| rs118192175 | 19 | 38494564 | C | T | RYR1 | |
| rs118192176 | 19 | 38494579 | G | A | RYR1 | |
| rs118192177 | 19 | 38496283 | C | G | RYR1 | |
| rs118192177 | 19 | 38496283 | C | T | RYR1 | |
| rs118192178 | 19 | 38500898 | C | T | RYR1 | |
| rs118192178 | 19 | 38500898 | C | G | RYR1 | |
| rs121918592 | 19 | 38448712 | G | C | RYR1 | |
| rs121918592 | 19 | 38448712 | G | A | RYR1 | |
| rs121918593 | 19 | 38499993 | G | A | RYR1 | |
| rs121918594 | 19 | 38500655 | G | T | RYR1 | |
| rs121918594 | 19 | 38500655 | G | A | RYR1 | |
| rs121918595 | 19 | 38580094 | C | T | RYR1 | |
| rs121918596 | 19 | 38499645 | GGAG | G | RYR1 | |
| rs144336148 | 19 | 38455472 | G | A | RYR1 | |
| rs1801086 | 19 | 38446710 | G | T | RYR1 | |
| rs1801086 | 19 | 38446710 | G | A | RYR1 | |
| rs1801086 | 19 | 38446710 | G | C | RYR1 | |
| rs193922747 | 19 | 38440802 | T | C | RYR1 | |
| rs193922748 | 19 | 38440829 | C | T | RYR1 | |
| rs193922753 | 19 | 38444212 | G | A | RYR1 | |
| rs193922753 | 19 | 38444212 | G | T | RYR1 | |
| rs193922762 | 19 | 38448673 | C | A | RYR1 | |
| rs193922762 | 19 | 38448673 | C | T | RYR1 | |
| rs193922764 | 19 | 38451842 | C | G | RYR1 | |
| rs193922764 | 19 | 38451842 | C | T | RYR1 | |
| rs193922768 | 19 | 38455471 | C | G | RYR1 | |
| rs193922768 | 19 | 38455471 | C | T | RYR1 | |
| rs193922768 | 19 | 38455471 | C | A | RYR1 | |
| rs193922770 | 19 | 38455528 | C | T | RYR1 | |
| rs193922772 | 19 | 38457546 | G | A | RYR1 | |
| rs193922772 | 19 | 38457546 | G | T | RYR1 | |
| rs193922802 | 19 | 38499655 | G | A | RYR1 | |
| rs193922803 | 19 | 38499670 | C | T | RYR1 | |
| rs193922807 | 19 | 38499731 | G | C | RYR1 | |
| rs193922809 | 19 | 38499975 | G | A | RYR1 | |
| rs193922816 | 19 | 38500642 | C | T | RYR1 | |
| rs193922818 | 19 | 38500899 | G | A | RYR1 | |
| rs193922832 | 19 | 38512321 | G | A | RYR1 | |
| rs193922843 | 19 | 38543832 | G | T | RYR1 | |
| rs193922876 | 19 | 38580114 | C | T | RYR1 | |
| rs193922878 | 19 | 38580370 | C | G | RYR1 | |
| rs28933396 | 19 | 38499997 | G | T | RYR1 | |
| rs28933396 | 19 | 38499997 | G | A | RYR1 | |
| rs28933397 | 19 | 38500654 | C | T | RYR1 | |
| rs63749869 | 19 | 38580440 | G | A | RYR1 | |
| rs4149056 | 12 | 21178615 | T | C | SLCO1B1 | |
| rs1142345 | 6 | 18130687 | T | C | TPMT | |
| rs1142345 | 6 | 18130687 | T | G | TPMT | |
| rs1800460 | 6 | 18138997 | C | T | TPMT | |
| rs1800462 | 6 | 18143724 | C | G | TPMT | |
| rs1800584 | 6 | 18130781 | C | T | TPMT | |
| rs8175347 | 2 | 233760235 | <(TA)5> | <(TA)6> | UGT1A1, | |
| | | | (SEQ | (SEQ | UGT1A10, | |
| | | | ID NO: | ID NO: | UGT1A3, | |
| | | | 2219) | 2220) | UGT1A4, | |
| | | | | | UGT1A5, | |
| | | | | | UGT1A6, | |
| | | | | | UGT1A7, | |
| | | | | | UGT1A8, | |
| | | | | | UGT1A9 | |
| rs8175347 | 2 | 233760235 | <(TA)5> | <(TA)7> | UGT1A1, | |
| | | | (SEQ | (SEQ | UGT1A10, | |
| | | | ID NO: | ID NO: | UGT1A3, | |
| | | | 2219) | 2221) | UGT1A4, | |
| | | | | | UGT1A5, | |
| | | | | | UGT1A6, | |
| | | | | | UGT1A7, | |
| | | | | | UGT1A8, | |
| | | | | | UGT1A9 | |
| rs8175347 | 2 | 233760235 | <(TA)5> | <(TA)8> | UGT1A1, | |
| | | | (SEQ | (SEQ | UGT1A10, | |
| | | | ID NO: | ID NO: | UGT1A3, | |
| | | | 2219) | 2222) | UGT1A4, | |
| | | | | | UGT1A5, | |
| | | | | | UGT1A6, | |
| | | | | | UGT1A7, | |
| | | | | | UGT1A8, | |
| | | | | | UGT1A9 | |
| rs887829 | 2 | 233759924 | C | T | UGT1A1, | UGT1A1*80 |
| | | | | | UGT1A10, | |
| | | | | | UGT1A3, | |
| | | | | | UGT1A4, | |
| | | | | | UGT1A5, | |
| | | | | | UGT1A6, | |
| | | | | | UGT1A7, | |
| | | | | | UGT1A8, | |
| | | | | | UGT1A9 | |
| rs3869066 | 6 | 30035987 | A | G | ZNRD1-AS1 | |
| rs113993958 | 7 | 117530953 | G | T | CFTR | |
| rs113993958 | 7 | 117530953 | G | C | CFTR | |
| rs113993959 | 7 | 117587778 | G | A | CFTR | |
| rs113993959 | 7 | 117587778 | G | T | CFTR | |
| rs115545701 | 7 | 117509089 | C | T | CFTR | |
| rs11971167 | 7 | 117642528 | G | T | CFTR | |
| rs11971167 | 7 | 117642528 | G | A | CFTR | |
| rs121908745 | 7 | 117559589 | CATC | C | CFTR | |
| rs121908746 | 7 | 117592218 | AA | A | CFTR | |
| rs121908747 | 7 | 117627580 | CC | C | CFTR | |
| rs121908748 | 7 | 117590440 | G | A | CFTR | |
| rs121908748 | 7 | 117590440 | G | C | CFTR | |
| rs121908748 | 7 | 117590440 | G | T | CFTR | |
| rs121908751 | 7 | 117530899 | G | A | CFTR | |
| rs121908751 | 7 | 117530899 | G | T | CFTR | |
| rs121908752 | 7 | 117535285 | T | G | CFTR | |
| rs121908753 | 7 | 117540285 | G | A | CFTR | |
| rs121908755 | 7 | 117587800 | G | T | CFTR | |
| rs121908755 | 7 | 117587800 | G | A | CFTR | |
| rs121908757 | 7 | 117587799 | A | C | CFTR | |
| rs121909005 | 7 | 117587801 | T | C | CFTR | |
| rs121909005 | 7 | 117587801 | T | G | CFTR | |
| rs121909011 | 7 | 117540230 | C | T | CFTR | |
| rs121909013 | 7 | 117587805 | G | A | CFTR | |
| rs121909017 | 7 | 117559546 | C | T | CFTR | |
| rs121909019 | 7 | 117611638 | G | T | CFTR | |
| rs121909019 | 7 | 117611638 | G | A | CFTR | |
| rs121909020 | 7 | 117611640 | G | C | CFTR | |
| rs121909020 | 7 | 117611640 | G | A | CFTR | |
| rs121909036 | 7 | 117611635 | T | C | CFTR | |
| rs121909036 | 7 | 117611635 | T | G | CFTR | |
| rs121909041 | 7 | 117642483 | T | A | CFTR | |
| rs121909041 | 7 | 117642483 | T | C | CFTR | |
| rs121909047 | 7 | 117590355 | C | A | CFTR | |
| rs139304906 | 7 | 117611671 | T | C | CFTR | |
| rs139573311 | 7 | 117559471 | T | C | CFTR | |
| rs141033578 | 7 | 117606695 | C | G | CFTR | |
| rs141033578 | 7 | 117606695 | C | T | CFTR | |
| rs150212784 | 7 | 117611595 | T | C | CFTR | |
| rs150212784 | 7 | 117611595 | T | G | CFTR | |
| rs151020603 | 7 | 117504336 | C | A | CFTR | |
| rs151020603 | 7 | 117504336 | C | T | CFTR | |
| rs1800088 | 7 | 117542063 | G | A | CFTR | |
| rs1800088 | 7 | 117542063 | G | T | CFTR | |
| rs1800100 | 7 | 117592169 | C | T | CFTR | |
| rs1800111 | 7 | 117610521 | G | C | CFTR | |
| rs186045772 | 7 | 117611663 | T | A | CFTR | |
| rs193922525 | 7 | 117664770 | G | A | CFTR | |
| rs200321110 | 7 | 117611646 | G | A | CFTR | |
| rs202179988 | 7 | 117611649 | C | T | CFTR | |
| rs267606723 | 7 | 117642451 | G | A | CFTR | |
| rs267606723 | 7 | 117642451 | G | T | CFTR | |
| rs34911792 | 7 | 117627758 | T | G | CFTR | |
| rs36210737 | 7 | 117611743 | T | A | CFTR | |
| rs36210737 | 7 | 117611743 | T | G | CFTR | |
| rs368505753 | 7 | 117509069 | C | T | CFTR | |
| rs397508139 | 7 | 117540237 | T | C | CFTR | |
| rs397508139 | 7 | 117540237 | T | A | CFTR | |
| rs397508144 | 7 | 117540251 | T | C | CFTR | |
| rs397508256 | 7 | 117509035 | G | A | CFTR | |
| rs397508267 | 7 | 117590353 | A | C | CFTR | |
| rs397508276 | 7 | 117590378 | T | C | CFTR | |
| rs397508276 | 7 | 117590378 | T | G | CFTR | |
| rs397508288 | 7 | 117590409 | A | C | CFTR | |
| rs397508288 | 7 | 117590409 | A | G | CFTR | |
| rs397508328 | 7 | 117480095 | A | G | CFTR | |
| rs397508435 | 7 | 117603654 | T | C | CFTR | |
| rs397508435 | 7 | 117603654 | T | A | CFTR | |
| rs397508442 | 7 | 117603708 | C | T | CFTR | |
| rs397508453 | 7 | 117603782 | G | C | CFTR | |
| rs397508453 | 7 | 117603782 | G | A | CFTR | |
| rs397508510 | 7 | 117611601 | C | T | CFTR | |
| rs397508510 | 7 | 117611601 | C | G | CFTR | |
| rs397508513 | 7 | 117611620 | A | C | CFTR | |
| rs397508537 | 7 | 117530955 | C | A | CFTR | |
| rs397508602 | 7 | 117642465 | G | A | CFTR | |
| rs397508759 | 7 | 117534363 | G | T | CFTR | |
| rs397508759 | 7 | 117534363 | G | A | CFTR | |
| rs74503330 | 7 | 117642472 | G | A | CFTR | |
| rs74551128 | 7 | 117548795 | C | T | CFTR | |
| rs74551128 | 7 | 117548795 | C | A | CFTR | |
| rs74597325 | 7 | 117587811 | C | G | CFTR | |
| rs74597325 | 7 | 117587811 | C | T | CFTR | |
| rs74767530 | 7 | 117627537 | C | T | CFTR | |
| rs75039782 | 7 | 117639961 | C | G | CFTR | |
| rs75039782 | 7 | 117639961 | C | T | CFTR | |
| rs75096551 | 7 | 117606754 | G | A | CFTR | |
| rs75096551 | 7 | 117606754 | G | C | CFTR | |
| rs75096551 | 7 | 117606754 | G | T | CFTR | |
| rs75527207 | 7 | 117587806 | G | A | CFTR | |
| rs75541969 | 7 | 117614699 | G | C | CFTR | |
| rs75549581 | 7 | 117587829 | G | A | CFTR | |
| rs75549581 | 7 | 117587829 | G | T | CFTR | |
| rs75961395 | 7 | 117509123 | G | A | CFTR | |
| rs75961395 | 7 | 117509123 | G | T | CFTR | |
| rs76713772 | 7 | 117587738 | G | A | CFTR | |
| rs77010898 | 7 | 117642566 | G | C | CFTR | |
| rs77010898 | 7 | 117642566 | G | A | CFTR | |
| rs77188391 | 7 | 117534366 | G | T | CFTR | |
| rs77409459 | 7 | 117540243 | C | T | CFTR | |
| rs77646904 | 7 | 117559629 | G | A | CFTR | |
| rs77646904 | 7 | 117559629 | G | C | CFTR | |
| rs77646904 | 7 | 117559629 | G | T | CFTR | |
| rs77834169 | 7 | 117530974 | C | G | CFTR | |
| rs77834169 | 7 | 117530974 | C | A | CFTR | |
| rs77834169 | 7 | 117530974 | C | T | CFTR | |
| rs77932196 | 7 | 117540270 | G | T | CFTR | |
| rs77932196 | 7 | 117540270 | G | A | CFTR | |
| rs77932196 | 7 | 117540270 | G | C | CFTR | |
| rs78194216 | 7 | 117611637 | C | A | CFTR | |
| rs78194216 | 7 | 117611637 | C | T | CFTR | |
| rs78655421 | 7 | 117530975 | G | C | CFTR | |
| rs78655421 | 7 | 117530975 | G | A | CFTR | |
| rs78655421 | 7 | 117530975 | G | T | CFTR | |
| rs78756941 | 7 | 117531115 | G | T | CFTR | |
| rs78769542 | 7 | 117611650 | G | C | CFTR | |
| rs78769542 | 7 | 117611650 | G | A | CFTR | |
| rs79635528 | 7 | 117611695 | A | C | CFTR | |
| rs79635528 | 7 | 117611695 | A | G | CFTR | |
| rs80034486 | 7 | 117652877 | C | G | CFTR | |
| rs80055610 | 7 | 117587833 | G | A | CFTR | |
| rs80055610 | 7 | 117587833 | G | C | CFTR | |
| rs80224560 | 7 | 117602868 | G | A | CFTR | |
| rs80282562 | 7 | 117534318 | G | A | CFTR | |
| rs113993960 | 7 | 117559591 | TCTT | T | CFTR | |
| rs199826652 | 7 | 117559591 | TCTT | T | CFTR | |
| rs33926104 | 19 | 40991390 | C | A | CYP2B6 | |
| rs33926104 | 19 | 40991390 | C | T | CYP2B6 | |
| rs33973337 | 19 | 40991381 | A | T | CYP2B6 | |
| rs33980385 | 19 | 40991388 | A | G | CYP2B6 | |
| rs34284776 | 19 | 40991391 | G | A | CYP2B6 | |
| rs34284776 | 19 | 40991391 | G | C | CYP2B6 | |
| rs8192709 | 19 | 40991369 | C | T | CYP2B6 | |
| rs17884712 | 10 | 94775489 | G | A | CYP2C19 | CYP2C19*9 |
| rs28399504 | 10 | 94762706 | A | G | CYP2C19 | CYP2C19*4 |
| rs41291556 | 10 | 94775416 | T | C | CYP2C19 | CYP2C19*8 |
| rs56337013 | 10 | 94852738 | C | T | CYP2C19 | CYP2C19*5 |
| rs6413438 | 10 | 94781858 | C | T | CYP2C19 | CYP2C19*10 |
| rs72552267 | 10 | 94775453 | G | A | CYP2C19 | CYP2C19*6 |
| rs72558186 | 10 | 94781999 | T | A | CYP2C19 | CYP2C19*7 |
| rs72558184 | 10 | 94775453 | G | A | | |
| rs72558187 | 10 | 94941958 | T | C | CYP2C9 | CYP2C9*13 |
| rs72558190 | 10 | 94947782 | C | A | CYP2C9 | CYP2C9*15 |
| rs9332239 | 10 | 94989020 | C | T | CYP2C9 | CYP2C9*12 |
| rs267608309 | 22 | 42129821 | G | A | CYP2D6 | CYP2D6*48 |
| rs28371710 | 22 | 42129075 | C | T | CYP2D6 | CYP2D6*12 |
| rs28371717 | 22 | 42128308 | C | A | CYP2D6 | CYP2D6*33 |
| rs5030655 | 22 | 42129083 | CA | C | CYP2D6 | CYP2D6*6 |
| rs5030865 | 22 | 42129033 | C | A | CYP2D6 | CYP2D6*8 |
| rs5030865 | 22 | 42129033 | C | A | CYP2D6 | CYP2D6*14 |
| rs5030865 | 22 | 42129033 | C | T | CYP2D6 | CYP2D6*8 |
| rs5030865 | 22 | 42129033 | C | T | CYP2D6 | CYP2D6*14 |
| rs769258 | 22 | 42130761 | C | T | CYP2D6 | CYP2D6*35 |
| rs1050829 | X | 154535277 | T | A | G6PD | |
| rs1050829 | X | 154535277 | T | C | G6PD | |
| rs137852313 | X | 154535187 | C | T | G6PD | |
| rs137852314 | X | 154534495 | C | T | G6PD | |
| rs137852315 | X | 154536032 | C | T | G6PD | |
| rs137852316 | X | 154532676 | C | T | G6PD | |
| rs137852317 | X | 154532411 | C | T | G6PD | |
| rs137852318 | X | 154533596 | C | A | G6PD | |
| rs137852318 | X | 154533596 | C | G | G6PD | |
| rs137852318 | X | 154533596 | C | T | G6PD | |
| rs137852319 | X | 154534157 | A | C | G6PD | |
| rs137852319 | X | 154534157 | A | G | G6PD | |
| rs137852320 | X | 154532698 | T | C | G6PD | |
| rs137852321 | X | 154532694 | C | T | G6PD | |
| rs137852322 | X | 154532701 | A | G | G6PD | |
| rs137852323 | X | 154532626 | C | A | G6PD | |
| rs137852324 | X | 154532389 | C | T | G6PD | |
| rs137852325 | X | 154532662 | C | T | G6PD | |
| rs137852326 | X | 154534345 | C | A | G6PD | |
| rs137852326 | X | 154534345 | C | G | G6PD | |
| rs137852327 | X | 154533122 | C | T | G6PD | |
| rs137852328 | X | 154534125 | C | A | G6PD | |
| rs137852328 | X | 154534125 | C | T | G6PD | |
| rs137852329 | X | 154532765 | G | T | G6PD | |
| rs137852330 | X | 154534390 | G | A | G6PD | |
| rs137852331 | X | 154534489 | T | C | G6PD | |
| rs137852332 | X | 154534389 | C | G | G6PD | |
| rs137852332 | X | 154534389 | C | T | G6PD | |
| rs137852333 | X | 154532797 | G | A | G6PD | |
| rs137852334 | X | 154532695 | G | A | G6PD | |
| rs137852335 | X | 154532674 | C | G | G6PD | |
| rs137852336 | X | 154532625 | C | T | G6PD | |
| rs137852337 | X | 154532434 | C | G | G6PD | |
| rs137852337 | X | 154532434 | C | T | G6PD | |
| rs137852339 | X | 154533044 | C | T | G6PD | |
| rs137852342 | X | 154532969 | G | A | G6PD | |
| rs137852343 | X | 154534465 | A | G | G6PD | |
| rs137852344 | X | 154532245 | G | C | G6PD | |
| rs137852345 | X | 154532772 | G | A | G6PD | |
| rs137852346 | X | 154533634 | C | T | G6PD | |
| rs137852347 | X | 154533029 | A | G | G6PD | |
| rs137852348 | X | 154532203 | G | C | G6PD | |
| rs137852349 | X | 154535996 | A | G | G6PD | |
| rs138687036 | X | 154535963 | G | A | G6PD | |
| rs2230037 | X | 154532439 | A | G | G6PD | |
| rs267606835 | X | 154535336 | G | A | G6PD | |
| rs267606835 | X | 154535336 | G | C | G6PD | |
| rs267606836 | X | 154534438 | G | A | G6PD | |
| rs34193178 | X | 154532945 | C | G | G6PD | |
| rs34193178 | X | 154532945 | C | T | G6PD | |
| rs371489738 | X | 154532722 | C | T | G6PD | |
| rs398123544 | X | 154532956 | T | A | G6PD | |
| rs398123546 | X | 154532390 | G | A | G6PD | |
| rs5030869 | X | 154532990 | C | T | G6PD | |
| rs5030870 | X | 154535316 | C | T | G6PD | |
| rs5030872 | X | 154534440 | T | A | G6PD | |
| rs587776730 | X | 154533012 | CGTGG | C | G6PD | |
| | | | GGTCG | | | |
| | | | TCCAG | | | |
| | | | GTACC | | | |
| | | | CTTTG | | | |
| | | | (SEQ | | | |
| | | | ID NO: | | | |
| | | | 2223) | | | |
| rs72554664 | X | 154532257 | C | T | G6PD | |
| rs72554665 | X | 154532269 | C | A | G6PD | |
| rs72554665 | X | 154532269 | C | G | G6PD | |
| rs72554665 | X | 154532269 | C | T | G6PD | |
| rs74575103 | X | 154533586 | C | T | G6PD | |
| rs76645461 | X | 154536156 | A | G | G6PD | |
| rs76723693 | X | 154533025 | A | G | G6PD | |
| rs782090947 | X | 154535995 | T | C | G6PD | |
| rs782098548 | X | 154532459 | C | T | G6PD | |
| rs782308266 | X | 154535962 | C | T | G6PD | |
| rs782487723 | X | 154535180 | C | T | G6PD | |
| rs782754619 | X | 154534348 | T | C | G6PD | |
| rs782757170 | X | 154534102 | G | A | G6PD | |
| rs78365220 | X | 154535270 | A | G | G6PD | |
| rs78478128 | X | 154536168 | G | C | G6PD | |
| . | X | 154534418 | GGAG | G | G6PD, IKBKG | |
| rs1050828 | X | 154536002 | C | T | G6PD, IKBKG | |
| rs137852338 | X | 154546050 | TGAT | T | G6PD, IKBKG | |
| rs137852340 | X | 154546061 | T | C | G6PD, IKBKG | |
| rs5030868 | X | 154534419 | G | A | G6PD, IKBKG | |
| rs746071566 | 13 | 48037782 | AGGAG | A | NUDT15, | NUDT15*2 |
| | | | TC | | SUCLA2 | |
| rs746071566 | 13 | 48037782 | AGGAG | A | NUDT15, | NUDT15*9 |
| | | | TC | | SUCLA2 | |
| rs869320766 | 13 | 48037801 | G | GGAGT | NUDT15, | |
| | | | | CG | SUCLA2 | |
| rs35350960 | 2 | 233760973 | C | A | UGT1A1, | UGT1A1*27 |
| | | | | | UGT1A10, | |
| | | | | | UGT1A3, | |
| | | | | | UGT1A4, | |
| | | | | | UGT1A5, | |
| | | | | | UGT1A6, | |
| | | | | | UGT1A7, | |
| | | | | | UGT1A8, | |
| | | | | | UGT1A9 | |
| rs35350960 | 2 | 233760973 | C | T | UGT1A1, | UGT1A1*27 |
| | | | | | UGT1A10, | |
| | | | | | UGT1A3, | |
| | | | | | UGT1A4, | |
| | | | | | UGT1A5, | |
| | | | | | UGT1A6, | |
| | | | | | UGT1A7, | |
| | | | | | UGT1A8, | |
| | | | | | UGT1A9 | |
| rs4148323 | 2 | 233760498 | G | A | UGT1A1, | UGT1A1*6 |
| | | | | | UGT1A10, | |
| | | | | | UGT1A3, | |
| | | | | | UGT1A4, | |
| | | | | | UGT1A5, | |
| | | | | | UGT1A6, | |
| | | | | | UGT1A7, | |
| | | | | | UGT1A8, | |
| | | | | | UGT1A9 | |
| rs149403002 | 19 | 41004093 | T | G | | |
| rs138100349 | 22 | 42130710 | G | A | CYP2D6 | CYP2D6*22 |
| rs148769737 | 22 | 42128217 | G | T | CYP2D6 | CYP2D6*84 |
| rs199535154 | 22 | 42128812 | A | G | CYP2D6 | CYP2D6*20 |
| rs201377835 | 22 | 42129910 | C | G | CYP2D6 | CYP2D6*11 |
| rs267608276 | 22 | 42129827 | C | G | CYP2D6 | CYP2D6*99 |
| rs267608279 | 22 | 42127962 | TG | T | CYP2D6 | CYP2D6*100 |
| rs267608297 | 22 | 42128235 | G | A | CYP2D6 | CYP2D6*54 |
| rs267608302 | 22 | 42129071 | T | G | CYP2D6 | CYP2D6*50 |
| rs267608311 | 22 | 42129906 | G | A | CYP2D6 | CYP2D6*57 |
| rs267608313 | 22 | 42130719 | G | A | CYP2D6 | CYP2D6*47 |
| rs267608319 | 22 | 42126749 | C | T | CYP2D6 | CYP2D6*31 |
| rs5030867 | 22 | 42127856 | T | G | CYP2D6 | CYP2D6*7 |
| rs72549346 | 22 | 42127531 | C | CAC | CYP2D6 | CYP2D6*42 |
| rs72549347 | 22 | 42127590 | G | A | CYP2D6 | CYP2D6*56 |
| rs72549348 | 22 | 42127619 | T | G | CYP2D6 | CYP2D6*51 |
| rs72549349 | 22 | 42127841 | C | G | CYP2D6 | CYP2D6*44 |
| rs72549351 | 22 | 42128200 | CAGTC | C | CYP2D6 | CYP2D6*38 |
| rs72549352 | 22 | 42128211 | C | CG | CYP2D6 | CYP2D6*21 |
| rs72549353 | 22 | 42128248 | CAGTT | C | CYP2D6 | CYP2D6*19 |
| rs72549354 | 22 | 42128817 | C | CC | CYP2D6 | CYP2D6*20 |
| rs730882170 | 22 | 42127845 | GCACA | G | CYP2D6 | CYP2D6*101 |
| | | | TCCGG | | | |
| | | | ATGTA | | | |
| | | | GGATC | | | |
| | | | (SEQ | | | |
| | | | ID NO: | | | |
| | | | 2224) | | | |
| rs730882251 | 22 | 42126747 | G | A | CYP2D6 | CYP2D6*62 |
| rs75386357 | 22 | 42127473 | C | T | CYP2D6 | CYP2D6*72 |
| rs763964554 | 22 | 42126896 | G | A | CYP2D6 | CYP2D6*96 |
| rs774671100 | 22 | 42130654 | C | CA | CYP2D6 | CYP2D6*15 |
| rs79292917 | 22 | 42127852 | C | T | CYP2D6 | CYP2D6*59 |
| rs147960066 | 22 | 42127590 | G | A | CYP2D6 | CYP2D6*56 |
| rs730882171 | 22 | 42126747 | G | A | CYP2D6 | CYP2D6*62 |
| rs758320086 | 22 | 42128248 | CAGTT | C | CYP2D6 | CYP2D6*19 |
| . | 22 | 42126666 | C | CAGTG | | CYP2D6*18 |
| | | | | GGCAC | | |
| | | | | (SEQ | | |
| | | | | ID NO: | | |
| | | | | 2225) | | |
| . | 22 | 42126666 | C | CAGTG | | |
| | | | | GGCAC | | |
| | | | | (SEQ | | |
| | | | | ID NO: | | |
| | | | | 2225) | | |
| . | 22 | 42128903 | T | TTA | | CYP2D6*60 |
| . | 22 | 42128903 | T | TTA | | |
| . | 22 | 42128795 | AG | A | | CYP2D6*92 |
| . | 22 | 42128795 | AG | A | | |
| . | 22 | 42126956 | T | G | | CYP2D6*55 |
| . | 22 | 42126956 | T | G | | |
| . | 22 | 42129071 | T | A | CYP2D6 | CYP2D6*50 |
| . | 22 | 42129071 | T | A | | CYP2D6*104 |
| . | 22 | 42129071 | T | A | | |
| rs267607275 | 6 | 18149126 | A | G | TPMT | |
| rs72552738 | 6 | 18139689 | C | T | TPMT | |
| rs74423290 | 6 | 18133884 | G | C | TPMT | |
| rs9333569 | 6 | 18149127 | T | C | TPMT | |
| rs9333570 | 6 | 18133890 | C | T | TPMT | |
| rs113934938 | 10 | 94842995 | G | A | CYP2C19 | CYP2C19*28 |
| rs118203756 | 10 | 94775160 | G | C | CYP2C19 | CYP2C19*23 |
| rs138142612 | 10 | 94842861 | G | A | CYP2C19 | CYP2C19*18 |
| rs145328984 | 10 | 94775106 | C | T | CYP2C19 | CYP2C19*30 |
| rs17879685 | 10 | 94849995 | C | T | CYP2C19 | CYP2C19*13 |
| rs17882687 | 10 | 94762760 | A | C | CYP2C19 | CYP2C19*15 |
| rs367543002 | 10 | 94762712 | C | T | CYP2C19 | CYP2C19*34 |
| rs367543003 | 10 | 94762715 | T | C | CYP2C19 | CYP2C19*34 |
| rs370803989 | 10 | 94780579 | G | A | CYP2C19 | CYP2C19*33 |
| rs55752064 | 10 | 94762755 | T | C | CYP2C19 | CYP2C19*14 |
| rs58973490 | 10 | 94775507 | G | A | CYP2C19 | CYP2C19*2 |
| . | 10 | 94762788 | A | T | | CYP2C19*29 |
| . | 10 | 94762788 | A | T | | |
| . | 10 | 94775121 | C | T | | CYP2C19*31 |
| . | 10 | 94775121 | C | T | | |
| . | 10 | 94775185 | A | G | | CYP2C19*32 |
| . | 10 | 94775185 | A | G | | |
| rs114071557 | 10 | 94938683 | A | G | CYP2C9 | CYP2C9*36 |
| rs1237225311 | 10 | 94972233 | C | T | CYP2C9 | CYP2C9*53 |
| rs12414460 | 10 | 94942231 | G | A | CYP2C9 | CYP2C9*42 |
| rs1250577724 | 10 | 94981302 | C | A | CYP2C9 | CYP2C9*55 |
| rs1274535931 | 10 | 94981230 | C | A | CYP2C9 | CYP2C9*58 |
| rs1326630788 | 10 | 94947917 | T | C | CYP2C9 | CYP2C9*48 |
| rs142240658 | 10 | 94938771 | C | T | CYP2C9 | CYP2C9*21 |
| rs182132442 | 10 | 94972119 | C | A | CYP2C9 | CYP2C9*29 |
| rs199523631 | 10 | 94942254 | C | T | CYP2C9 | CYP2C9*45 |
| rs200183364 | 10 | 94942255 | G | A | CYP2C9 | CYP2C9*33 |
| rs200965026 | 10 | 94942249 | C | T | CYP2C9 | CYP2C9*44 |
| rs202201137 | 10 | 94988925 | A | G | CYP2C9 | CYP2C9*61 |
| rs367826293 | 10 | 94981225 | G | A | CYP2C9 | CYP2C9*34 |
| rs371055887 | 10 | 94941897 | G | C | CYP2C9 | CYP2C9*20 |
| rs56165452 | 10 | 94981297 | T | C | CYP2C9 | CYP2C9*4 |
| rs564813580 | 10 | 94938828 | A | G | CYP2C9 | CYP2C9*37 |
| rs57505750 | 10 | 94981201 | T | C | CYP2C9 | CYP2C9*31 |
| rs67807361 | 10 | 94938737 | C | A | CYP2C9 | CYP2C9*7 |
| rs72558189 | 10 | 94942234 | G | A | CYP2C9 | CYP2C9*14 |
| rs72558189 | 10 | 94942234 | G | A | CYP2C9 | CYP2C9*35 |
| rs72558189 | 10 | 94942234 | G | T | CYP2C9 | CYP2C9*14 |
| rs72558189 | 10 | 94942234 | G | T | CYP2C9 | CYP2C9*35 |
| rs72558192 | 10 | 94972179 | A | G | CYP2C9 | CYP2C9*16 |
| rs72558193 | 10 | 94986073 | A | C | CYP2C9 | CYP2C9*18 |
| rs749060448 | 10 | 94981281 | G | A | CYP2C9 | CYP2C9*24 |
| rs750820937 | 10 | 94981250 | C | A | CYP2C9 | CYP2C9*54 |
| rs754487195 | 10 | 94942305 | G | A | CYP2C9 | CYP2C9*46 |
| rs762239445 | 10 | 94941982 | G | T | CYP2C9 | CYP2C9*39 |
| rs764211126 | 10 | 94986042 | A | G | CYP2C9 | CYP2C9*56 |
| rs767284820 | 10 | 94988955 | T | C | CYP2C9 | CYP2C9*60 |
| rs767576260 | 10 | 94942230 | C | T | CYP2C9 | CYP2C9*43 |
| rs769942899 | 10 | 94988917 | G | C | CYP2C9 | CYP2C9*19 |
| rs774550549 | 10 | 94947785 | C | T | CYP2C9 | CYP2C9*47 |
| rs774607211 | 10 | 94942216 | A | G | CYP2C9 | CYP2C9*41 |
| rs781583846 | 10 | 94988984 | G | A | CYP2C9 | CYP2C9*30 |
| rs868182778 | 10 | 94989023 | G | T | CYP2C9 | CYP2C9*32 |
| rs9332130 | 10 | 94949280 | A | G | CYP2C9 | CYP2C9*10 |
| rs988617574 | 10 | 94972180 | C | G | CYP2C9 | CYP2C9*52 |
| . | 10 | 94981365 | C | T | | CYP2C9*17 |
| . | 10 | 94981365 | C | T | | |
| . | 10 | 94938803 | A | G | | CYP2C9*22 |
| . | 10 | 94938803 | A | G | | |
| . | 10 | 94941915 | G | A | | CYP2C9*23 |
| . | 10 | 94941915 | G | A | | |
| . | 10 | 94942249 | C | G | | CYP2C9*26 |
| . | 10 | 94942249 | C | G | CYP2C9 | CYP2C9*44 |
| . | 10 | 94947938 | A | T | | CYP2C9*28 |
| . | 10 | 94947938 | A | T | | |
| . | 10 | 94941976 | G | C | | CYP2C9*38 |
| . | 10 | 94941976 | G | C | | |
| . | 10 | 94942018 | T | C | | CYP2C9*40 |
| . | 10 | 94942018 | T | C | | |
| . | 10 | 94949129 | A | G | | CYP2C9*49 |
| . | 10 | 94949129 | A | G | | |
| . | 10 | 94949144 | C | T | | CYP2C9*50 |
| . | 10 | 94949144 | C | T | | |
| . | 10 | 94972134 | A | G | | CYP2C9*51 |
| . | 10 | 94972134 | A | G | | |
| . | 10 | 94947907 | A | C | | CYP2C9*57 |
| . | 10 | 94947907 | A | C | | |
| . | 10 | 94988855 | A | T | | CYP2C9*59 |
| . | 10 | 94988855 | A | T | | |
| rs1135829 | 22 | 42127973 | T | C | CYP2D6 | CYP2D6*115 |
| rs141009491 | 22 | 42127631 | C | G | CYP2D6 | CYP2D6*116 |
| rs76088846 | 22 | 42127589 | C | T | CYP2D6 | |
| rs78209835 | 22 | 42127611 | C | T | CYP2D6 | CYP2D6*117 |
| rs1135823 | 22 | 42129174 | C | A | CYP2D6 | CYP2D6*53 |
| rs1135824 | 22 | 42129042 | T | C | CYP2D6 | CYP2D6*3 |
| rs1135828 | 22 | 42128181 | A | T | CYP2D6 | CYP2D6*81 |
| rs118203758 | 22 | 42130667 | C | T | CYP2D6 | CYP2D6*71 |
| rs202102799 | 22 | 42127556 | T | C | CYP2D6 | CYP2D6*108 |
| rs267608293 | 22 | 42127515 | T | A | CYP2D6 | |
| rs267608295 | 22 | 42127593 | G | C | CYP2D6 | CYP2D6*25 |
| rs267608308 | 22 | 42129780 | C | T | CYP2D6 | CYP2D6*73 |
| rs267608310 | 22 | 42129836 | G | A | CYP2D6 | CYP2D6*23 |
| rs28371696 | 22 | 42130715 | C | T | CYP2D6 | CYP2D6*15 |
| rs28371703 | 22 | 42129819 | G | T | CYP2D6 | CYP2D6*4 |
| rs28371704 | 22 | 42129809 | T | C | CYP2D6 | CYP2D6*4 |
| rs28371733 | 22 | 42126914 | C | G | CYP2D6 | CYP2D6*4 |
| rs28371733 | 22 | 42126914 | C | G | CYP2D6 | CYP2D6*52 |
| rs28371733 | 22 | 42126914 | C | T | CYP2D6 | CYP2D6*4 |
| rs28371733 | 22 | 42126914 | C | T | CYP2D6 | CYP2D6*52 |
| rs367543000 | 22 | 42128212 | G | A | CYP2D6 | CYP2D6*81 |
| rs374616348 | 22 | 42129183 | C | T | CYP2D6 | CYP2D6*70 |
| rs375135093 | 22 | 42129113 | A | G | CYP2D6 | CYP2D6*89 |
| rs532668079 | 22 | 42126746 | C | T | CYP2D6 | CYP2D6*75 |
| rs535642512 | 22 | 42129759 | C | T | CYP2D6 | CYP2D6*111 |
| rs568495591 | 22 | 42126605 | G | A | CYP2D6 | CYP2D6*112 |
| rs569229126 | 22 | 42129098 | T | C | CYP2D6 | CYP2D6*90 |
| rs569439709 | 22 | 42126752 | C | T | CYP2D6 | CYP2D6*113 |
| rs61736517 | 22 | 42127565 | T | C | CYP2D6 | CYP2D6*108 |
| rs72549358 | 22 | 42130773 | C | T | CYP2D6 | CYP2D6*28 |
| rs745365204 | 22 | 42128848 | C | T | CYP2D6 | CYP2D6*37 |
| rs74802369 | 22 | 42129771 | T | A | CYP2D6 | CYP2D6*82 |
| rs748712690 | 22 | 42127610 | T | C | CYP2D6 | CYP2D6*94 |
| rs751092905 | 22 | 42126735 | C | T | CYP2D6 | CYP2D6*110 |
| rs76187628 | 22 | 42129779 | A | G | CYP2D6 | CYP2D6*82 |
| rs769157652 | 22 | 42126938 | C | T | CYP2D6 | CYP2D6*27 |
| rs77312092 | 22 | 42127457 | C | T | CYP2D6 | CYP2D6*95 |
| rs773790593 | 22 | 42130778 | G | A | CYP2D6 | CYP2D6*87 |
| rs77913725 | 22 | 42128185 | C | T | CYP2D6 | CYP2D6*81 |
| rs78459009 | 22 | 42129765 | T | C | CYP2D6 | CYP2D6*82 |
| rs78482768 | 22 | 42129087 | G | C | CYP2D6 | CYP2D6*28 |
| rs949717872 | 22 | 42127938 | T | G | CYP2D6 | CYP2D6*24 |
| rs1058172 | 22 | 42127526 | C | G | | |
| rs1058172 | 22 | 42127526 | C | T | | |
| rs267608294 | 22 | 42127523 | A | G | CYP2D6 | CYP2D6*105 |
| rs1555888910 | 22 | 42127523 | A | G | CYP2D6 | CYP2D6*105 |
| rs201830078 | 22 | 42128181 | A | T | | |
| rs201830078 | 22 | 42128181 | A | G | | |
| rs774943042 | 22 | 42129056 | C | A | CYP2D6 | |
| rs149157808 | 22 | 42126914 | C | T | CYP2D6 | CYP2D6*4 |
| rs149157808 | 22 | 42126914 | C | T | CYP2D6 | CYP2D6*52 |
| rs149157808 | 22 | 42126914 | C | T | | |
| rs376436494 | 22 | 42127593 | G | A | CYP2D6 | CYP2D6*25 |
| rs376436494 | 22 | 42127593 | G | C | CYP2D6 | CYP2D6*25 |
| rs376436494 | 22 | 42127593 | G | T | CYP2D6 | CYP2D6*25 |
| rs548942748 | 22 | 42128185 | C | T | CYP2D6 | CYP2D6*81 |
| . | 22 | 42127899 | T | C | | CYP2D6*118 |
| . | 22 | 42127514 | A | G | | CYP2D6*26 |
| . | 22 | 42126634 | A | C | | CYP2D6*85 |
| . | 22 | 42126634 | A | C | | |
| . | 22 | 42129056 | C | G | | CYP2D6*91 |
| . | 22 | 42129056 | C | G | CYP2D6 | |
| . | 22 | 42129056 | C | G | | |
| . | 22 | 42128272 | T | G | | CYP2D6*93 |
| . | 22 | 42128272 | T | G | | |
| . | 22 | 42126697 | G | T | | CYP2D6*97 |
| . | 22 | 42126697 | G | T | | |
| . | 22 | 42126681 | G | C | | CYP2D6*98 |
| . | 22 | 42126681 | G | C | | |
| . | 22 | 42129071 | T | A | CYP2D6 | CYP2D6*50 |
| . | 22 | 42129071 | T | A | | CYP2D6*104 |
| . | 22 | 42129071 | T | A | | |
| rs1135825 | 22 | 42129037 | G | T | | |
| rs1135826 | 22 | 42129036 | A | C | | |
| rs61745683 | 22 | 42127512 | C | T | | |
| rs1406719554 | 22 | 42127922 | A | G | | |
| rs369177208 | 22 | 42126719 | C | T | | |
| rs781457579 | 22 | 42129134 | G | A | | |
| rs17002853 | 22 | 42128325 | A | G | | |
| rs747089665 | 22 | 42126926 | G | A | | |
| rs3021084 | 22 | 42126877 | G | A | | |
| . | 22 | 42130727 | AG | A | | |
| . | 22 | 42126981 | G | GGA | | |
| . | 22 | 42129887 | A | G | | |
| . | 22 | 42129155 | C | T | | |
| . | 22 | 42128879 | A | G | | |
| . | 22 | 42128878 | C | A | | |
| . | 22 | 42127602 | C | T | | |
| . | 22 | 42126578 | C | T | | |
| . | 22 | 42126647 | C | T | | |
| rs28365083 | 7 | 99652613 | G | T | CYP3A5, | |
| | | | | | ZSCAN25 | |
| rs28383479 | 7 | 99660516 | C | T | CYP3A5, | |
| | | | | | ZSCAN25 | |
| rs55817950 | 7 | 99676198 | G | A | CYP3A5, | |
| | | | | | ZSCAN25 | |
| rs55965422 | 7 | 99666950 | A | T | CYP3A5, | |
| | | | | | ZSCAN25 | |
| rs55965422 | 7 | 99666950 | A | G | CYP3A5, | |
| | | | | | ZSCAN25 | |
| rs56411402 | 7 | 99665237 | T | C | CYP3A5, | |
| | | | | | ZSCAN25 | |

TABLE 9
PGx Probe Sequences

| | | SEQ | | SEQ | | | |
|---|---|---|---|---|---|---|---|
| | | ID | | ID | | | Ref |
| ID | AlleleA_ProbeSeq | NO: | AlleleB_ProbeSeq | NO: | Chr | Map Info | Strand |
| rs6025 | TTCTCAGAATTTCTG | 22 | | | 1 | 169549811 | + |
| | AAAGGTTACTTCAAG | | | | | | |
| | GACAAAATACCTGTA | | | | | | |
| | TTCCT | | | | | | |
| rs6025 | TTCTCAGAATTTCTG | 23 | | | 1 | 169549811 | + |
| | AAAGGTTACTTCAAG | | | | | | |
| | GACAAAATACCTGTA | | | | | | |
| | TTCCT | | | | | | |
| rs6025 | TTCTCAGAATTTCTG | 24 | | | 1 | 169549811 | + |
| | AAAGGTTACTTCAAG | | | | | | |
| | GACAAAATACCTGTA | | | | | | |
| | TTCCT | | | | | | |
| rs18005 | GAGGGAGGGGCCTCT | 25 | GAGGGAGGGGCCTCT | 1664 | 1 | 201060815 | − |
| 59 | TGGTGCTGACCTGTC | | TGGTGCTGACCTGTC | | | | |
| | CTGTTGTATGTGTCA | | CTGTTGTATGTGTCA | | | | |
| | CAGCA | | CAGCG | | | | |
| rs18005 | GAGGGAGGGGCCTCT | 26 | GAGGGAGGGGCCTCT | 1665 | 1 | 201060815 | − |
| 59 | TGGTGCTGACCTGTC | | TGGTGCTGACCTGTC | | | | |
| | CTGTTGTATGTGTCA | | CTGTTGTATGTGTCA | | | | |
| | CAGCA | | CAGCG | | | | |
| rs18005 | GAGGGAGGGGCCTCT | 27 | GAGGGAGGGGCCTCT | 1666 | 1 | 201060815 | − |
| 59 | TGGTGCTGACCTGTC | | TGGTGCTGACCTGTC | | | | |
| | CTGTTGTATGTGTCA | | CTGTTGTATGTGTCA | | | | |
| | CAGCA | | CAGCG | | | | |
| rs77222 | AAGGGGTCTGCAGGG | 28 | | | 1 | 201091993 | + |
| 6819 | ACACTGCCACCCACT | | | | | | |
| | AGGCACCCCCGACAC | | | | | | |
| | CAGCC | | | | | | |
| rs77222 | CCGGCTTGGATGTCA | 29 | | | 1 | 201091993 | − |
| 6819 | AGGCCCTCAGAGCCT | | | | | | |
| | TCCGAGTGCTCAGAC | | | | | | |
| | CCCTC | | | | | | |
| rs77222 | CCGGCTTGGATGTCA | 30 | | | 1 | 201091993 | − |
| 6819 | AGGCCCTCAGAGCCT | | | | | | |
| | TCCGAGTGCTCAGAC | | | | | | |
| | CCCTC | | | | | | |
| rs67376 | ATCATTACAGGTCAT | 31 | ATCATTACAGGTCAT | 1667 | 1 | 97082391 | + |
| 798 | GTAGCATTTACCACA | | GTAGCATTTACCACA | | | | |
| | GTTGATACACATTTC | | GTTGATACACATTTC | | | | |
| | TTCAA | | TTCAT | | | | |
| rs67376 | GGAACATTTGGTGAA | 32 | GGAACATTTGGTGAA | 1668 | 1 | 97082391 | − |
| 798 | TTGAGCAACGTAGAG | | TTGAGCAACGTAGAG | | | | |
| | CAAGTIGTGGCTATG | | CAAGTIGTGGCTATG | | | | |
| | ATTGT | | ATTGA | | | | |
| rs39182 | ATATTGGTGTCAAAG | 33 | ATATTGGTGTCAAAG | 1669 | 1 | 97450058 | − |
| 90 | TGTCACTGAACTAAA | | TGTCACTGAACTAAA | | | | |
| | GGCTGACTTTCCAGA | | GGCTGACTTTCCAGA | | | | |
| | CAACA | | CAACG | | | | |
| rs39182 | TTCACCAACTTATGC | 34 | TTCACCAACTTATGC | 1670 | 1 | 97450058 | + |
| 90 | CAATTCTCTTGTTTT | | CAATTCTCTTGTTTT | | | | |
| | AGATGTTAAATCACA | | AGATGTTAAATCACA | | | | |
| | CTTAT | | CTTAC | | | | |
| rs39182 | ATTCACCAACTTATG | 35 | | | 1 | 97450058 | + |
| 90 | CCAATTCTCTTGTTT | | | | | | |
| | TAGATGTTAAATCAC | | | | | | |
| | ACTTA | | | | | | |
| rs55886 | GAAAGTTTTGGTGAG | 36 | GAAAGTTTTGGTGAG | 1671 | 1 | 97515787 | + |
| 062 | GGCAAAACCCCATCC | | GGCAAAACCCCATCC | | | | |
| | AGCTTCAAAAGCTCT | | AGCTTCAAAAGCTCT | | | | |
| | TCGAA | | TCGAC | | | | |
| rs55886 | GAAAGTTTTGGTGAG | 37 | GAAAGTTTTGGTGAG | 1672 | 1 | 97515787 | + |
| 062 | GGCAAAACCCCATCC | | GGCAAAACCCCATCC | | | | |
| | AGCTTCAAAAGCTCT | | AGCTTCAAAAGCTCT | | | | |
| | TCGAA | | TCGAC | | | | |
| rs55886 | AATCCTTTTGGTCTT | 38 | AATCCTTTTGGTCTT | 1673 | 1 | 97515787 | − |
| 062 | GCTAGCGCAACTCCA | | GCTAGCGCAACTCCA | | | | |
| | GCCACCAGCACATCA | | GCCACCAGCACATCA | | | | |
| | ATGAT | | ATGAG | | | | |
| rs67376 | GGAACATTTGGTGAA | 39 | GGAACATTTGGTGAA | 1674 | 1 | 97547947 | − |
| 798 | TTGAGCAACGTAGAG | | TTGAGCAACGTAGAG | | | | |
| | CAAGTTGTGGCTATG | | CAAGTTGTGGCTATG | | | | |
| | ATTGT | | ATTGA | | | | |
| rs56038 | GTTATAGTAAAAGGT | 40 | | | 1 | 97573863 | − |
| 477 | GGGAGAATTGTTGCT | | | | | | |
| | ATGCAGTTTGTTCGG | | | | | | |
| | ACAGA | | | | | | |
| rs56038 | GTTATAGTAAAAGGT | 41 | | | 1 | 97573863 | − |
| 477 | GGGAGAATTGTTGCT | | | | | | |
| | ATGCAGTTTGTTCGG | | | | | | |
| | ACAGA | | | | | | |
| rs56038 | GTTATAGTAAAAGGT | 42 | | | 1 | 98039419 | − |
| 477 | GGGAGAATTGTTGCT | | | | | | |
| | ATGCAGTTTGTTCGG | | | | | | |
| | ACAGA | | | | | | |
| rs12248 | GTTTTAGCAAACGAT | 43 | | | 10 | 94761900 | + |
| 560 | TTTTTTTTTCAAATT | | | | | | |
| | TGTGTCTTCTGTTCT | | | | | | |
| | CAAAG | | | | | | |
| rs12248 | GTTTTAGCAAACGAT | 44 | | | 10 | 94761900 | + |
| 560 | TTTTTTTTTCAAATT | | | | | | |
| | TGTGTCTTCTGTTCT | | | | | | |
| | CAAAG | | | | | | |
| rs12248 | GTTTTAGCAAACGAT | 45 | | | 10 | 94761900 | + |
| 560 | TTTTTTTTTCAAATT | | | | | | |
| | TGTGTCTTCTGTTCT | | | | | | |
| | CAAAG | | | | | | |
| rs28399 | AAGGAGAAGCAAACA | 46 | | | 10 | 94762706 | − |
| 504 | TGAGAGACAGAGCAC | | | | | | |
| | AAGGACCACAAAAGG | | | | | | |
| | ATCCA | | | | | | |
| rs28399 | AAGGAGAAGCAAACA | 47 | | | 10 | 94762706 | − |
| 504 | TGAGAGACAGAGCAC | | | | | | |
| | AAGGACCACAAAAGG | | | | | | |
| | ATCCA | | | | | | |
| rs28399 | AAGGAGAAGCAAACA | 48 | | | 10 | 94762706 | − |
| 504 | TGAGAGACAGAGCAC | | | | | | |
| | AAGGACCACAAAAGG | | | | | | |
| | ATCCA | | | | | | |
| rs55752 | TGGGGCCAGGAGGGA | 49 | | | 10 | 94762755 | |
| 064 | GTTTTCCTCTCCCAG | | | | | | |
| | AGCTCTGTCTCCAGA | | | | | | |
| | TTGAA | | | | | | |
| rs55752 | TGGGGCCAGGAGGGA | 50 | | | 10 | 94762755 | − |
| 064 | GTTTTCCTCTCCCAG | | | | | | |
| | AGCTCTGTCTCCAGA | | | | | | |
| | TTGAA | | | | | | |
| rs55752 | TAGGGCCAGGAGGGA | 51 | | | 10 | 94762755 | − |
| 064 | GTTTTCCTCTCCCAG | | | | | | |
| | AGCTCTGTCTCCAGA | | | | | | |
| | TTGAA | | | | | | |
| 10_ | TCATGTTTGCTTCTC | 52 | TCATGTTTGCTTCTC | 1675 | 10 | 94762788 | + |
| 94762788 | CTTTCAATCTGGAGA | | CTTTCAATCTGGAGA | | | | |
| | CAGAGCTCTGGGAGA | | CAGAGCTCTGGGAGA | | | | |
| | GGAAA | | GGAAT | | | | |
| 10_ | TCATGTTTGCTTCTC | 53 | TCATGTTTGCTTCTC | 1676 | 10 | 94762788 | + |
| 94762788 | CTTTCAATCTGGAGA | | CTTTCAATCTGGAGA | | | | |
| | CAGAGCTCTGGGAGA | | CAGAGCTCTGGGAGA | | | | |
| | GGAAA | | GGAAT | | | | |
| 10_ | TCATGTTTGCTTCTC | 54 | TCATGTTTGCTTCTC | 1677 | 10 | 94762788 | + |
| 94762788 | CTTTCAATCTGGAGA | | CTTTCAATCTGGAGA | | | | |
| | CAGAGCTCTGGGAGA | | CAGAGCTCTGGGAGA | | | | |
| | GGAAA | | GGAAT | | | | |
| rs14532 | AGCTCTCAAAAATCT | 55 | | | 10 | 94775106 | + |
| 8984 | ATGGCCCTGTGTTCA | | | | | | |
| | CTCTGTATTTTGGCC | | | | | | |
| | TGGAA | | | | | | |
| rs14532 | AGCTCTCAAAAATCT | 56 | | | 10 | 94775106 | + |
| 8984 | ATGGCCCTGTGTTCA | | | | | | |
| | CTCTGTATTTTGGCC | | | | | | |
| | TGGAA | | | | | | |
| rs14532 | ATCAATCAGGGCTTC | 57 | | | 10 | 94775106 | − |
| 8984 | CTTCACCACTTCATA | | | | | | |
| | TCCATGCAGCACCAC | | | | | | |
| | CATGC | | | | | | |
| 10_ | ATGGCCCTGTGTTCA | 58 | | | 10 | 94775121 | + |
| 94775121 | CTCTGTATTTTGGCC | | | | | | |
| | TGGAACGCATGGTGG | | | | | | |
| | TGCTG | | | | | | |
| 10_ | ATGGCCCTGTGTTCA | 59 | | | 10 | 94775121 | + |
| 94775121 | CTCTGTATTTTGGCC | | | | | | |
| | TGGAACGCATGGTGG | | | | | | |
| | TGCTG | | | | | | |
| 10_ | ATGGCCCTGTGTTCA | 60 | | | 10 | 94775121 | + |
| 94775121 | CTCTGTATTTTGGCC | | | | | | |
| | TGGAACGCATGGTGG | | | | | | |
| | TGCTG | | | | | | |
| rs11820 | GGTGGTGCTGCATGG | 61 | GGTGGTGCTGCATGG | 1678 | 10 | 94775160 | + |
| 3756 | ATATGAAGTGGTGAA | | ATATGAAGTGGTGAA | | | | |
| | GGAAGCCCTGATTGA | | GGAAGCCCTGATTGA | | | | |
| | TCTTG | | TCTTC | | | | |
| rs11820 | GGTGGTGCTGCATGG | 62 | GGTGGTGCTGCATGG | 1679 | 10 | 94775160 | + |
| 3756 | ATATGAAGTGGTGAA | | ATATGAAGTGGTGAA | | | | |
| | GGAAGCCCTGATTGA | | GGAAGCCCTGATTGA | | | | |
| | TCTTG | | TCTTC | | | | |
| rs11820 | TTAGCTCTTTCAGCC | 63 | TTAGCTCTTTCAGCC | 1680 | 10 | 94775160 | − |
| 3756 | AGTGGGAAATGGCCT | | AGTGGGAAATGGCCT | | | | |
| | CTTCCAGAAAACTCC | | CTTCCAGAAAACTCC | | | | |
| | TCTCC | | TCTCG | | | | |
| 10_ | GGTGAAGGAAGCCCT | 64 | | | 10 | 94775185 | + |
| 94775185 | GATTGATCTTGGAGA | | | | | | |
| | GGAGTTTTCTGGAAG | | | | | | |
| | AGGCC | | | | | | |
| 10_ | GGTGAAGGAAGCCCT | 65 | | | 10 | 94775185 | + |
| 94775185 | GATTGATCTTGGAGA | | | | | | |
| | GGAGTTTTCTGGAAG | | | | | | |
| | AGGCC | | | | | | |
| 10_ | GGTGAAGGAAGCCCT | 66 | | | 10 | 94775185 | + |
| 94775185 | GATTGATCTTGGAGA | | | | | | |
| | GGAGTTTTCTGGAAG | | | | | | |
| | AGGCC | | | | | | |
| 10_ | CACTTGCACACCTAC | 67 | | | 10 | 94775185 | − |
| 94775185 | CAAATCCTCTGTTAG | | | | | | |
| | CTCTTTCAGCCAGTG | | | | | | |
| | GGAAA | | | | | | |
| rs12769 | CATCTCTTTCCATTG | 68 | | | 10 | 94775367 | − |
| 205 | CTGAAAACGATTCCT | | | | | | |
| | AACAGGAAGAGAAAC | | | | | | |
| | GAAAC | | | | | | |
| rs12769 | CATCTCTTTCCATTG | 69 | | | 10 | 94775367 | − |
| 205 | CTGAAAACGATTCCT | | | | | | |
| | AACAGGAAGAGAAAC | | | | | | |
| | GAAAC | | | | | | |
| rs41291 | CATCCCAAAATTCCG | 70 | | | 10 | 94775416 | − |
| 556 | CAGCGTCATGAGGGA | | | | | | |
| | GAAACGCCGGATCTC | | | | | | |
| | CTTCC | | | | | | |
| rs41291 | TAGTTTCGTTTCTCT | 71 | | | 10 | 94775416 | + |
| 556 | TCCTGTTAGGAATCR | | | | | | |
| | TTTTCAGCAATGGAA | | | | | | |
| | AGAGA | | | | | | |
| rs41291 | TAGTTTCGTTTCTCT | 72 | | | 10 | 94775416 | + |
| 556 | TCCTGTTAGGAATCR | | | | | | |
| | TTTTCAGCAATGGAA | | | | | | |
| | AGAGA | | | | | | |
| rs72552 | CAATGGAAAGAGATG | 73 | | | 10 | 94775453 | + |
| 267 | GAAGGAGATCCGGCG | | | | | | |
| | TTTCTCCCTCATGAC | | | | | | |
| | GCTGC | | | | | | |
| rs72552 | CTTCCTCTTGAACAC | 74 | | | 10 | 94775453 | − |
| 267 | GGTCCTCAATGCTCC | | | | | | |
| | TCTTCCCCWTCCCAA | | | | | | |
| | AATTC | | | | | | |
| rs72552 | CTTCCTCTTGAACAC | 75 | | | 10 | 94775453 | − |
| 267 | GGTCCTCAATGCTCC | | | | | | |
| | TCTTCCCCATCCCAA | | | | | | |
| | AATTC | | | | | | |
| rs72552 | CTTCCTCTTGAACAC | 76 | | | 10 | 94775453 | − |
| 267 | GGTCCTCAATGCTCC | | | | | | |
| | TCTTCCCCATCCCAA | | | | | | |
| | AATTC | | | | | | |
| rs17884 | CCTCATGACgCTGCG | 77 | | | 10 | 94775489 | + |
| 712 | GAATaTTGGGATGaG | | | | | | |
| | GAAGAGGAGCATtGA | | | | | | |
| | GGACC | | | | | | |
| rs17884 | CAGaAAGGTCAGTGA | 78 | | | 10 | 94775489 | − |
| 712 | TAgaGAGTAtGtTCC | | | | | | |
| | AGCGGGCTTCCTCTT | | | | | | |
| | GAACA | | | | | | |
| rs17884 | CAGaAAGGTCAGTGA | 79 | | | 10 | 94775489 | − |
| 712 | TAgaGAGTAtGtTCC | | | | | | |
| | AGCGGGCTTCCTCTT | | | | | | |
| | GAACA | | | | | | |
| rs58973 | TTTTGGGATGGGGAA | 80 | | | 10 | 94775507 | + |
| 490 | GAGGAGCATTGAGGA | | | | | | |
| | CCGTGTTCAAGAGGA | | | | | | |
| | AGCCC | | | | | | |
| rs58973 | TTTTGGGATGGGGAA | 81 | | | 10 | 94775507 | + |
| 490 | GAGGAGCATTGAGGA | | | | | | |
| | CCGTGTTCAAGAGGA | | | | | | |
| | AGCCC | | | | | | |
| rs58973 | TTTTGGGATGGGGAA | 82 | | | 10 | 94775507 | + |
| 490 | GAGGAGCATTGAGGA | | | | | | |
| | CCGTGTTCAAGAGGA | | | | | | |
| | AGCCC | | | | | | |
| rs37080 | GTTTTCATTCAATTT | 83 | | | 10 | 94780579 | − |
| 3989 | TTCCATCAAGTTAAG | | | | | | |
| | AAATTGCTGATCTTT | | | | | | |
| | ATAAT | | | | | | |
| rs37080 | GTTTTCATTCAATTT | 84 | | | 10 | 94780579 | − |
| 3989 | TTCCATCAAGTTAAG | | | | | | |
| | AAATTGCTGATCTTT | | | | | | |
| | ATAAT | | | | | | |
| rs37080 | GTTTTCATTCAATTT | 85 | | | 10 | 94780579 | − |
| 3989 | TTCCATCAAGTTAAG | | | | | | |
| | AAATTGCTGATCTTT | | | | | | |
| | ATAAT | | | | | | |
| rs49868 | AACTTGATGGAAAAA | 86 | | | 10 | 94780653 | + |
| 93 | TTGAATGAAAACATC | | | | | | |
| | AGGATTGTAAGCACC | | | | | | |
| | CCCTG | | | | | | |
| rs49868 | AACTTGATGGAAAAA | 87 | | | 10 | 94780653 | + |
| 93 | TTGAATGAAAACATC | | | | | | |
| | AGGATTGTAAGCACC | | | | | | |
| | CCCTG | | | | | | |
| rs49868 | AACTTGATGGAAAAA | 88 | | | 10 | 94780653 | + |
| 93 | TTGAATGAAAACATC | | | | | | |
| | AGGATTGTAAGCACC | | | | | | |
| | CCCTG | | | | | | |
| rs64134 | TGTTTTCTCTTAGAT | 89 | | | 10 | 94781858 | + |
| 38 | ATGCAATAATTTTCC | | | | | | |
| | CACTATCATTGATTA | | | | | | |
| | TTTCC | | | | | | |
| rs64134 | TATCACTTTCCATAA | 90 | | | 10 | 94781858 | − |
| 38 | AAGCAAGGTTTTTAA | | | | | | |
| | GTAATTTGTTATGGG | | | | | | |
| | TTCCY | | | | | | |
| rs64134 | TGTTTTCTCTTAGAT | 91 | | | 10 | 94781858 | + |
| 38 | ATGCAATAATTTTCC | | | | | | |
| | CACTATCATTGATTA | | | | | | |
| | TTTCC | | | | | | |
| rs42442 | ATATCACTTTCCATA | 92 | | | 10 | 94781859 | − |
| 85 | AAAGCAAGGTTTTTA | | | | | | |
| | AGTAATTTGTTATGG | | | | | | |
| | GTTCC | | | | | | |
| rs42442 | ATATCACTTTCCATA | 93 | | | 10 | 94781859 | − |
| 85 | AAAGCAAGGTTTTTA | | | | | | |
| | AGTAATTTGTTATGG | | | | | | |
| | GTTCC | | | | | | |
| rs42442 | ATATCACTTTCCATA | 94 | | | 10 | 94781859 | − |
| 85 | AAAGCAAGGTTTTTA | | | | | | |
| | AGTAATTTGTTATGG | | | | | | |
| | GTTCC | | | | | | |
| rs72558 | AACAACCCTCGGGAC | 95 | AACAACCCTCGGGAC | 1681 | 10 | 94781999 | + |
| 186 | TTTATTGATTGCTTC | | TTTATTGATTGCTTC | | | | |
| | CTGATCAAAATGGAG | | CTGATCAAAATGGAG | | | | |
| | AAGGA | | AAGGT | | | | |
| rs72558 | AACAACCCTCGGGAC | 96 | AACAACCCTCGGGAC | 1682 | 10 | 94781999 | + |
| 186 | TTTATTGATTGCTTC | | TTTATTGATTGCTTC | | | | |
| | CTGATCAAAATGGAG | | CTGATCAAAATGGAG | | | | |
| | AAGGA | | AAGGT | | | | |
| rs72558 | CAACAACCCTCGGGA | 97 | | | 10 | 94781999 | + |
| 186 | CTTTATTGATTGCTT | | | | | | |
| | CCTGATCAAAATGGA | | | | | | |
| | GAAGG | | | | | | |
| rs13814 | ATCAGTTCTTACTTG | 98 | | | 10 | 94842861 | + |
| 2612 | TGTCTTGTCAGCTAA | | | | | | |
| | AGTCCAGGAAGAGAT | | | | | | |
| | TGAAC | | | | | | |
| rs37585 | TCTTACTTGTGTCTT | 99 | TCTTACTTGTGTCTT | 1683 | 10 | 94842866 | + |
| 81 | GTCAGCTAAAGTCCA | | GTCAGCTAAAGTCCA | | | | |
| | GGAAGAGATTGAACG | | GGAAGAGATTGAACG | | | | |
| | TGTYA | | TGTYG | | | | |
| rs37585 | TCTTACTTGTGTCTT | 100 | TCTTACTTGTGTCTT | 1684 | 10 | 94842866 | + |
| 81 | GTCAGCTAAAGTCCA | | GTCAGCTAAAGTCCA | | | | |
| | GGAAGAGATTGAACG | | GGAAGAGATTGAACG | | | | |
| | TGTYA | | TGTYG | | | | |
| rs37585 | TTCTTACTTGTGTCT | 101 | | | 10 | 94842866 | + |
| 81 | TGTCAGCTAAAGTCC | | | | | | |
| | AGGAAGAGATTGAAC | | | | | | |
| | GTGTY | | | | | | |
| rs11393 | GATACATCGACCTCA | 102 | | | 10 | 94842995 | + |
| 4938 | TCCCCACCAGCCTGC | | | | | | |
| | CCCATGCAGTGACCT | | | | | | |
| | GTGAC | | | | | | |
| rs11393 | GATACATCGACCTCA | 103 | | | 10 | 94842995 | + |
| 4938 | TCCCCACCAGCCTGC | | | | | | |
| | CCCATGCAGTGACCT | | | | | | |
| | GTGAC | | | | | | |
| rs56337 | AGTTACACATGAGGA | 104 | | | 10 | 94852738 | + |
| 013 | GTAACTTCTCCCTAT | | | | | | |
| | GTTTGTTATTTTCAG | | | | | | |
| | GAAAA | | | | | | |
| rs93320 | TACTGATAGTGTACA | 105 | | | 10 | 94936917 | − |
| 94 | ATGATTCAGGATTTC | | | | | | |
| | GTAATTCAGTCTAGT | | | | | | |
| | AAATT | | | | | | |
| rs11407 | AAGGAGAAGCAAACA | 106 | | | 10 | 94938683 | − |
| 1557 | TGAGAGACAGAGCAC | | | | | | |
| | AAGGACCACAAGAGA | | | | | | |
| | ATCCA | | | | | | |
| rs11407 | AAGGAGAAGCAAACA | 107 | | | 10 | 94938683 | − |
| 1557 | TGAGAGACAGAGCAC | | | | | | |
| | AAGGACCACAAGAGA | | | | | | |
| | ATCCA | | | | | | |
| rs11407 | AAGGAGAAGCAAACA | 108 | | | 10 | 94938683 | − |
| 1557 | TGAGAGACAGAGCAC | | | | | | |
| | AAGGACCACAAGAGA | | | | | | |
| | ATCCA | | | | | | |
| rs67807 | AGGAGTGGGGCCAGG | 109 | | | 10 | 94938737 | − |
| 361 | AGGGAGTTTTCCTCT | | | | | | |
| | CCCAGAGCTCTGTCT | | | | | | |
| | CCAGA | | | | | | |
| rs67807 | AGGAGTGGGGCCAGG | 110 | | | 10 | 94938737 | − |
| 361 | AGGGAGTTTTCCTCT | | | | | | |
| | CCCAGAGCTCTGTCT | | | | | | |
| | CCAGA | | | | | | |
| rs67807 | AGGAGTGGGGCCAGG | 111 | | | 10 | 94938737 | − |
| 361 | AGGGAGTTTTCCTCT | | | | | | |
| | CCCAGAGCTCTGTCT | | | | | | |
| | CCAGA | | | | | | |
| rs14224 | TTTGCTTCTCCTTTC | 112 | | | 10 | 94938771 | + |
| 0658 | ACTCTGGAGACAGAG | | | | | | |
| | CTCTGGGAGAGGAAA | | | | | | |
| | ACTCC | | | | | | |
| rs14224 | TTTGCTTCTCCTTTC | 113 | | | 10 | 94938771 | + |
| 0658 | ACTCTGGAGACAGAG | | | | | | |
| | CTCTGGGAGAGGAAA | | | | | | |
| | ACTCC | | | | | | |
| rs14224 | TACCTATCTGTAGGA | 114 | | | 10 | 94938771 | − |
| 0658 | TATTTCCAATCACTG | | | | | | |
| | GGAGAGGAGTGGGGC | | | | | | |
| | CAGGA | | | | | | |
| 10_ | TACATTGGTTAAGGA | 115 | | | 10 | 94938803 | − |
| 94938803 | TTTGCTGATGTCCTT | | | | | | |
| | AATACCTATCTGTAG | | | | | | |
| | GATAT | | | | | | |
| 10_ | TACATTGGTTAAGGA | 116 | | | 10 | 94938803 | − |
| 94938803 | TTTGCTGATGTCCTT | | | | | | |
| | AATACCTATCTGTAG | | | | | | |
| | GATAT | | | | | | |
| 10_ | TACATTGGTTAAGGA | 117 | | | 10 | 94938803 | − |
| 94938803 | TTTGCTGATGTCCTT | | | | | | |
| | AATACCTATCTGTAG | | | | | | |
| | GATAT | | | | | | |
| 10_ | TACATTGGTTAAGGA | 118 | | | 10 | 94938803 | − |
| 94938803 | TTTGCTGATGTCCTT | | | | | | |
| | AATACCTATCTGTAG | | | | | | |
| | GATAT | | | | | | |
| rs56481 | TTGCAAGCCACTGAA | 119 | | | 10 | 94938828 | − |
| 3580 | GGAGCATACTTACAT | | | | | | |
| | TGGTTAAGGATTTGC | | | | | | |
| | TGATG | | | | | | |
| rs56481 | TTGCAAGCCACTGAA | 120 | | | 10 | 94938828 | − |
| 3580 | GGAGCATACTTACAT | | | | | | |
| | TGGTTAAGGATTTGC | | | | | | |
| | TGATG | | | | | | |
| rs56481 | TTGCAAGCCACTGAA | 121 | | | 10 | 94938828 | − |
| 3580 | GGAGCATACTTACAT | | | | | | |
| | TGGTTAAGGATTTGC | | | | | | |
| | TGATG | | | | | | |
| rs37105 | GCTTCCTTCACTGCT | 122 | GCTTCCTTCACTGCT | 1685 | 10 | 94941897 | − |
| 5887 | TCATATCCATGCAGC | | TCATATCCATGCAGC | | | | |
| | ACCACTATGGGTTTC | | ACCACTATGGGTTTC | | | | |
| | AGGCC | | AGGCG | | | | |
| rs37105 | GCTTCCTTCACTGCT | 123 | GCTTCCTTCACTGCT | 1686 | 10 | 94941897 | − |
| 5887 | TCATATCCATGCAGY | | TCATATCCATGCAGY | | | | |
| | AYCACTATGGGTTTC | | AYCACTATGGGTTTC | | | | |
| | AGGCC | | AGGCG | | | | |
| rs37105 | GCTTCCTTCACTGCT | 124 | GCTTCCTTCACTGCT | 1687 | 10 | 94941897 | − |
| 5887 | TCATATCCATGCAGY | | TCATATCCATGCAGY | | | | |
| | AYCACTATGGGTTTC | | AYCACTATGGGTTTC | | | | |
| | AGGCC | | AGGCG | | | | |
| 10_ | CTCTCCAAGATCAAT | 125 | | | 10 | 94941915 | − |
| 94941915 | CAGGGCTTCCTTCAC | | | | | | |
| | TGCTTCATATCCATG | | | | | | |
| | CAGYA | | | | | | |
| 10_ | TCTCCAAGATCAATC | 126 | TCTCCAAGATCAATC | 1688 | 10 | 94941915 | − |
| 94941915 | AGGGCTTCCTTCACT | | AGGGCTTCCTTCACT | | | | |
| | GCTTCATATCCATGC | | GCTTCATATCCATGC | | | | |
| | AGCAT | | AGCAC | | | | |
| 10_ | TCTCCAAGATCAATC | 127 | TCTCCAAGATCAATC | 1689 | 10 | 94941915 | − |
| 94941915 | AGGGCTTCCTTCACT | | AGGGCTTCCTTCACT | | | | |
| | GCTTCATATCCATGC | | GCTTCATATCCATGC | | | | |
| | AGYAT | | AGYAC | | | | |
| rs72558 | TAGCTCTTTCAGCCA | 128 | | | 10 | 94941958 | − |
| 187 | GTGGGAAAATGCCTC | | | | | | |
| | TTCCAGAAAACTCCT | | | | | | |
| | CTCCA | | | | | | |
| rs72558 | CATaGTGGTGCTGCA | 129 | | | 10 | 94941958 | + |
| 187 | TCGATATGAAGcaGT | | | | | | |
| | GACGGAAGgCCTGAT | | | | | | |
| | TGATC | | | | | | |
| rs72558 | CATaGTGGTGCTGCA | 130 | | | 10 | 94941958 | + |
| 187 | TCGATATGAAGcaGT | | | | | | |
| | GAcGGAAGgCCTGAT | | | | | | |
| | TGATC | | | | | | |
| 10_ | CCTACCAAATCCTCT | 131 | CCTACCAAATCCTCT | 1690 | 10 | 94941976 | − |
| 94941976 | GTTAGCTCTTTCAGC | | GTTAGCTCTTTCAGC | | | | |
| | CAGTGGGAAAATGMC | | CAGTGGGAAAATGMC | | | | |
| | TCTTG | | TCTTC | | | | |
| 10_ | CCTACCAAATCCTCT | 132 | CCTACCAAATCCTCT | 1691 | 10 | 94941976 | − |
| 94941976 | GTTAGCTCTTTCAGC | | GTTAGCTCTTTCAGC | | | | |
| | CAGTGGGAAAATGMC | | CAGTGGGAAAATGMC | | | | |
| | TCTTG | | TCTTC | | | | |
| 10_ | CCTACCAAATCCTCT | 133 | CCTACCAAATCCTCT | 1692 | 10 | 94941976 | − |
| 94941976 | GTTAGCTCTTTCAGC | | GTTAGCTCTTTCAGC | | | | |
| | CAGTGGGAAAATGCC | | CAGTGGGAAAATGCC | | | | |
| | TCTTG | | TCTTC | | | | |
| rs76223 | TGCACACCTACCAAA | 134 | TGCACACCTACCAAA | 1693 | 10 | 94941982 | − |
| 9445 | TCCTCTGTTAGCTCT | | TCCTCTGTTAGCTCT | | | | |
| | TTCAGCCAGTGGGAA | | TTCAGCCAGTGGGAA | | | | |
| | AATGT | | AATGC | | | | |
| rs76223 | TGCACACCTACCAAA | 135 | TGCACACCTACCAAA | 1694 | 10 | 94941982 | − |
| 9445 | TCCTCTGTTAGCTCT | | TCCTCTGTTAGCTCT | | | | |
| | TTCAGCCAGTGGGAA | | TTCAGCCAGTGGGAA | | | | |
| | AATGA | | AATGC | | | | |
| rs76223 | TGCACACCTACCAAA | 136 | TGCACACCTACCAAA | 1695 | 10 | 94941982 | − |
| 9445 | TCCTCTGTTAGCTCT | | TCCTCTGTTAGCTCT | | | | |
| | TTCAGCCAGTGGGAA | | TTCAGCCAGTGGGAA | | | | |
| | AATGA | | AATGC | | | | |
| 10_ | GTTTTCTGGAAGAGG | 137 | | | 10 | 94942018 | + |
| 94942018 | CATTTTCCCACTGGC | | | | | | |
| | TGAAAGAGCTAACAG | | | | | | |
| | AGGAT | | | | | | |
| 10_ | GTTTTCTGGAAGAGG | 138 | | | 10 | 94942018 | + |
| 94942018 | CATTTTCCCACTGGC | | | | | | |
| | TGAAAGAGCTAACAG | | | | | | |
| | AGGAT | | | | | | |
| 10_ | GTTTTCTGGAAGAGG | 139 | | | 10 | 94942018 | + |
| 94942018 | CATTTTCCCACTGGC | | | | | | |
| | TGAAAGAGCTAACAG | | | | | | |
| | AGGAT | | | | | | |
| rs77460 | CCTAGTTTCGTTTCT | 140 | | | 10 | 94942216 | + |
| 7211 | CTTCCTGTTAGGAAT | | | | | | |
| | TGTTTTCAGCAATGG | | | | | | |
| | AAAGA | | | | | | |
| rs77460 | TCCCAAAATTCCGCA | 141 | | | 10 | 94942216 | − |
| 7211 | GCGTCATGAGGGAGA | | | | | | |
| | AACGCCGGATCTCCT | | | | | | |
| | TCCAT | | | | | | |
| rs77460 | TCCCAAAATTCCGCA | 142 | | | 10 | 94942216 | − |
| 7211 | GCGTCATGAGGGAGA | | | | | | |
| | AACGCCGGATCTCCT | | | | | | |
| | TCCAT | | | | | | |
| rs76757 | TCTTCCTGTTAGGAA | 143 | | | 10 | 94942230 | + |
| 6260 | TTGTTTTCAGCAATG | | | | | | |
| | GAAAGARATGGAAGG | | | | | | |
| | AGATC | | | | | | |
| rs76757 | CTTCCTGTTAGGAAT | 144 | CTTCCTGTTAGGAAT | 1696 | 10 | 94942230 | + |
| 6260 | TGTTTTCAGCAATGG | | TGTTTTCAGCAATGG | | | | |
| | AAAGAAATGGAAGGA | | AAAGAAATGGAAGGA | | | | |
| | GATCT | | GATCC | | | | |
| rs76757 | TCTTCCTGTTAGGAA | 145 | | | 10 | 94942230 | + |
| 6260 | TTGTTTTCAGCAATG | | | | | | |
| | GAAAGAAATGGAAGG | | | | | | |
| | AGATC | | | | | | |
| rs12414 | TTCCTGTTAGGAATT | 146 | TTCCTGTTAGGAATT | 1697 | 10 | 94942231 | + |
| 460 | GTTTTCAGCAATGGA | | GTTTTCAGCAATGGA | | | | |
| | AAGAAATGGAAGGAG | | AAGAAATGGAAGGAG | | | | |
| | ATCCA | | ATCCG | | | | |
| rs12414 | TTCCTGTTAGGAATT | 147 | TTCCTGTTAGGAATT | 1698 | 10 | 94942231 | + |
| 460 | GTTTTCAGCAATGGA | | GTTTTCAGCAATGGA | | | | |
| | AAGAAATGGAAGGAG | | AAGAAATGGAAGGAG | | | | |
| | ATCCT | | ATCCG | | | | |
| rs12414 | TTCCTGTTAGGAATT | 148 | TTCCTGTTAGGAATT | 1699 | 10 | 94942231 | + |
| 460 | GTTTTCAGCAATGGA | | GTTTTCAGCAATGGA | | | | |
| | AAGAAATGGAAGGAG | | AAGAAATGGAAGGAG | | | | |
| | ATCCT | | ATCCG | | | | |
| rs12414 | CTTCCTGTTAGGAAT | 149 | | | 10 | 94942231 | + |
| 460 | TGTTTTCAGCAATGG | | | | | | |
| | AAAGAAATGGAAGGA | | | | | | |
| | GATCC | | | | | | |
| rs12414 | CTTCCTGTTAGGAAT | 150 | | | 10 | 94942231 | + |
| 460 | TGTTTTCAGCAATGG | | | | | | |
| | AAAGARATGGAAGGA | | | | | | |
| | GATCC | | | | | | |
| rs12414 | CTTCCTGTTAGGAAT | 151 | | | 10 | 94942231 | + |
| 460 | TGTTTTCAGCAATGG | | | | | | |
| | AAAGARATGGAAGGA | | | | | | |
| | GATCC | | | | | | |
| rs72558 | CCTGTTAGGAATTGT | 152 | | | 10 | 94942234 | + |
| 189 | TTTCAGCAATGGAAA | | | | | | |
| | GAAATGGAAGGAGAT | | | | | | |
| | CCGGC | | | | | | |
| rs72558 | CCTGTTAGGAATTGT | 153 | | | 10 | 94942234 | + |
| 189 | TTTCAGCAATGGAAA | | | | | | |
| | GAAATGGAAGGAGAT | | | | | | |
| | CCGGC | | | | | | |
| rs20096 | TTCAGCAATGGAAAG | 154 | TTCAGCAATGGAAAG | 1700 | 10 | 94942249 | + |
| 5026 | AAATGGAAGGAGATC | | AAATGGAAGGAGATC | | | | |
| | CGGCGTTTCTCCCTC | | CGGCGTTTCTCCCTC | | | | |
| | ATGAC | | ATGAG | | | | |
| rs20096 | TTGAACACGGTCCTC | 155 | TTGAACACGGTCCTC | 1701 | 10 | 94942249 | − |
| 5026 | AATGCTCCTCTTCCC | | AATGCTCCTCTTCCC | | | | |
| | CATCCCAAAATTCCG | | CATCCCAAAATTCCG | | | | |
| | CAGCA | | CAGCG | | | | |
| rs20096 | TTTCAGCAATGGAAA | 156 | | | 10 | 94942249 | + |
| 5026 | GAAATGGAAGGAGAT | | | | | | |
| | CCGGCGTTTCTCCCT | | | | | | |
| | CATGA | | | | | | |
| rs20018 | CTTCCTCTTGAACAC | 157 | | | 10 | 94942255 | − |
| 3364 | GGTCCTCAATGCTCC | | | | | | |
| | TCTTCCCCATCCCAA | | | | | | |
| | AATTC | | | | | | |
| rs20018 | CTTCCTCTTGAACAC | 158 | | | 10 | 94942255 | − |
| 3364 | GGTCCTCAATGCTCC | | | | | | |
| | TCTTCCCCATCCCAA | | | | | | |
| | AATTC | | | | | | |
| rs17998 | AGtAAGGTCAGTGAT | 159 | | | 10 | 94942290 | − |
| 53 | AtgGAGTAgGgTCCA | | | | | | |
| | GCGGGCTTCCTCTTG | | | | | | |
| | AACAC | | | | | | |
| rs17998 | CCCTCATGACGCTGC | 160 | | | 10 | 94942290 | + |
| 53 | GGAATTTTGGGATGG | | | | | | |
| | GGAAGAGGAGCATTG | | | | | | |
| | AGGAC | | | | | | |
| rs75448 | GTAGGGTCACCCACC | 161 | | | 10 | 94942305 | − |
| 7195 | CTTGGTTTTTCTCAA | | | | | | |
| | CTCCTCCACAAGGCA | | | | | | |
| | GHGGG | | | | | | |
| rs75448 | GTAGGGTCACCCACC | 162 | | | 10 | 94942305 | − |
| 7195 | CTTGGTTTTTCTCAA | | | | | | |
| | CTCCTCCACAAGGCA | | | | | | |
| | GHGGG | | | | | | |
| rs75448 | GTAGGGTCACCCACC | 163 | | | 10 | 94942305 | − |
| 7195 | CTTGGTTTTTCTCAA | | | | | | |
| | CTCCTCCACAAGGCA | | | | | | |
| | GCGGG | | | | | | |
| rs79001 | TTTGGGATGGGGAAG | 164 | TTTGGGATGGGGAAG | 1702 | 10 | 94942309 | + |
| 94 | AGGAGCATTGAGGAC | | AGGAGCATTGAGGAC | | | | |
| | CGTGTTCAAGAGGAA | | CGTGTTCAAGAGGAA | | | | |
| | GCCCA | | GCCCG | | | | |
| rs79001 | TTTGGGATGGGGAAG | 165 | TTTGGGATGGGGAAG | 1703 | 10 | 94942309 | + |
| 94 | AGGAGCATTGAGGAC | | AGGAGCATTGAGGAC | | | | |
| | CGTGTTCAAGAGGAA | | CGTGTTCAAGAGGAA | | | | |
| | GCCCA | | GCCCG | | | | |
| rs72558 | TATATCTAAAGTTTA | 166 | TATATCTAAAGTTTA | 1704 | 10 | 94947782 | + |
| 190 | ATAGTATTTTAAATT | | ATAGTATTTTAAATT | | | | |
| | GTTTCTAATTATTTA | | GTTTCTAATTATTTA | | | | |
| | GCCTA | | GCCTC | | | | |
| rs72558 | TATATCTAAAGTTTA | 167 | TATATCTAAAGTTTA | 1705 | 10 | 94947782 | + |
| 190 | ATAGTATTTTAAATT | | ATAGTATTTTAAATT | | | | |
| | GTTTCTAATTATTTA | | GTTTCTAATTATTTA | | | | |
| | GCCTA | | GCCTC | | | | |
| rs72558 | TATATCTAAAGTTTA | 168 | TATATCTAAAGTTTA | 1706 | 10 | 94947782 | + |
| 190 | ATAGTATTTTAAATT | | ATAGTATTTTAAATT | | | | |
| | GTTTCTAATTATTTA | | GTTTCTAATTATTTA | | | | |
| | GCCTA | | GCCTC | | | | |
| rs77455 | TATCTAAAGTTTAAT | 169 | | | 10 | 94947785 | + |
| 0549 | AGTATTTTAAATTGT | | | | | | |
| | TTCTAATTATTTAGC | | | | | | |
| | CTCAC | | | | | | |
| rs77455 | TATCTAAAGTTTAAT | 170 | | | 10 | 94947785 | + |
| 0549 | AGTATTTTAAATTGT | | | | | | |
| | TTCTAATTATTTAGC | | | | | | |
| | CTCAC | | | | | | |
| rs77455 | TATCTAAAGTTTAAT | 171 | | | 10 | 94947785 | + |
| 0549 | AGTATTTTAAATTGT | | | | | | |
| | TTCTAATTATTTAGC | | | | | | |
| | CTCAC | | | | | | |
| 10_ | TTGATTATAAAGATC | 172 | | | 10 | 94947907 | + |
| 94947907 | AGCAATTTCTTAACT | | | | | | |
| | TAATGGAAAAGTTGA | | | | | | |
| | ATGAA | | | | | | |
| 10_ | TGATTATAAAGATCA | 173 | TGATTATAAAGATCA | 1707 | 10 | 94947907 | + |
| 94947907 | GCAATTTCTTAACTT | | GCAATTTCTTAACTT | | | | |
| | AATGGAAAAGTTGAA | | AATGGAAAAGTTGAA | | | | |
| | TGAAA | | TGAAC | | | | |
| 10_ | TGATTATAAAGATCA | 174 | TGATTATAAAGATCA | 1708 | 10 | 94947907 | + |
| 94947907 | GCAATTTCTTAACTT | | GCAATTTCTTAACTT | | | | |
| | AATGGAAAAGTTGAA | | AATGGAAAAGTTGAA | | | | |
| | TGAAA | | TGAAC | | | | |
| rs13266 | TCTCAGGAAGCAAAA | 175 | | | 10 | 94947917 | − |
| 30788 | ATCTTGGCCTTACCT | | | | | | |
| | GGATCCAGGGGCTGC | | | | | | |
| | TCAAA | | | | | | |
| rs13266 | TCTCAGGAAGCAAAA | 176 | | | 10 | 94947917 | − |
| 30788 | ATCTTGGCCTTACCT | | | | | | |
| | GGATCCAGGGGCTGC | | | | | | |
| | TCAAA | | | | | | |
| rs13266 | TCTCAGGAAGCAAAA | 177 | | | 10 | 94947917 | − |
| 30788 | ATCTTGGCCTTACCT | | | | | | |
| | GGATCCAGGGGCTGC | | | | | | |
| | TCAAA | | | | | | |
| 10_ | ATGGAAAAGTTGAAT | 178 | ATGGAAAAGTTGAAT | 1709 | 10 | 94947938 | + |
| 94947938 | GAAAACATCAAGATT | | GAAAACATCAAGATT | | | | |
| | TTGAGCAGCCCCTGG | | TTGAGCAGCCCCTGG | | | | |
| | ATCCA | | ATCCT | | | | |
| 10_ | ATGGAAAAGTTGAAT | 179 | ATGGAAAAGTTGAAT | 1710 | 10 | 94947938 | + |
| 94947938 | GAAAACATCAAGAYT | | GAAAACATCAAGAYT | | | | |
| | TTGAGCAGCCCCTGG | | TTGAGCAGCCCCTGG | | | | |
| | ATCCA | | ATCCT | | | | |
| 10_ | ATGGAAAAGTTGAAT | 180 | ATGGAAAAGTTGAAT | 1711 | 10 | 94947938 | + |
| 94947938 | GAAAACATCAAGATT | | GAAAACATCAAGATT | | | | |
| | TTGAGCAGCCCCTGG | | TTGAGCAGCCCCTGG | | | | |
| | ATCCA | | ATCCT | | | | |
| 10_ | AGCAACGTTTTTAAG | 181 | | | 10 | 94949129 | − |
| 94949129 | TAATTTGTTGTGAGT | | | | | | |
| | TCCCGGGAAGTAATC | | | | | | |
| | AATGA | | | | | | |
| 10_ | AATTTAATAAATTAT | 182 | AATTTAATAAATTAT | 1712 | 10 | 94949129 | + |
| 94949129 | TGTTTTCTCTTAGAT | | TGTTTTCTCTTAGAT | | | | |
| | CTGCAATAATTTTTC | | CTGCAATAATTTTTC | | | | |
| | TCCTA | | TCCTG | | | | |
| 10_ | AATTTAATAAATTAT | 183 | AATTTAATAAATTAT | 1713 | 10 | 94949129 | + |
| 94949129 | TGTTTTCTCTTAGAT | | TGTTTTCTCTTAGAT | | | | |
| | CTGCAATAATTTTTC | | CTGCAATAATTTTTC | | | | |
| | TCCTA | | TCCTG | | | | |
| 10_ | TGTTTTCTCTTAGAT | 184 | TGTTTTCTCTTAGAT | 1714 | 10 | 94949144 | + |
| 94949144 | CTGCAATAATTTTTC | | CTGCAATAATTTTTC | | | | |
| | TCCTATCATTGATTA | | TCCTATCATTGATTA | | | | |
| | CTTCT | | CTTCC | | | | |
| 10_ | TGTTTTCTCTTAGAT | 185 | TGTTTTCTCTTAGAT | 1715 | 10 | 94949144 | + |
| 94949144 | CTGCAATAATTTTTC | | CTGCAATAATTTTTC | | | | |
| | TCCTATCATTGATTA | | TCCTATCATTGATTA | | | | |
| | CTTCT | | CTTCC | | | | |
| 10_ | TTGTTTTCTCTTAGA | 186 | | | 10 | 94949144 | + |
| 94949144 | TCTGCAATAATTTTT | | | | | | |
| | CTCCTATCATTGATT | | | | | | |
| | ACTTC | | | | | | |
| rs93321 | GGACATGAACAACCC | 187 | | | 10 | 94949280 | + |
| 30 | TCAGGACTTTATTGA | | | | | | |
| | TTGCTTCCTGATGAA | | | | | | |
| | AATGG | | | | | | |
| rs93321 | GGACATGAACAACCC | 188 | | | 10 | 94949280 | + |
| 30 | TCAGGACTTTATTGA | | | | | | |
| | TTGCTTCCTGATGAA | | | | | | |
| | AATGG | | | | | | |
| rs93321 | CACAAATTCACAAGC | 189 | | | 10 | 94949282 | − |
| 31 | AGTCACATAACTAAG | | | | | | |
| | CTTTTGTTTACATTT | | | | | | |
| | TACCT | | | | | | |
| rs93321 | CATGAACAACCCTCA | 190 | | | 10 | 94949282 | + |
| 31 | GGACTTTATTGATTG | | | | | | |
| | CTTCCTGATGAAAAT | | | | | | |
| | GGAGA | | | | | | |
| rs93321 | CATGAACAACCCTCA | 191 | | | 10 | 94949282 | + |
| 31 | GGACTTTATTGATTG | | | | | | |
| | CTTCCTGATGAAAAT | | | | | | |
| | GGAGA | | | | | | |
| rs18213 | AAACAAGTCAACTGC | 192 | | | 10 | 94972119 | − |
| 2442 | AGTGTTTTCCAAGCT | | | | | | |
| | TTCAAYAGTAAATTC | | | | | | |
| | AGATG | | | | | | |
| 10_ | CTCTGTCCCAGCTCC | 193 | | | 10 | 94972134 | − |
| 94972134 | AAACAAGTCAACTGC | | | | | | |
| | AGTGTTTTCCAAGCT | | | | | | |
| | TTCAA | | | | | | |
| 10_ | TTGCATCAAATCATT | 194 | | | 10 | 94972134 | + |
| 94972134 | CCTAGGAAAAGCACA | | | | | | |
| | ACCAACCATCTGAAT | | | | | | |
| | TTACT | | | | | | |
| 10_ | TTGCATCAAATCATT | 195 | | | 10 | 94972134 | + |
| 94972134 | CCTAGGAAAAGCACA | | | | | | |
| | ACCAAMCATCTGAAT | | | | | | |
| | TTACT | | | | | | |
| rs72558 | TTACTATTGAAAGCT | 196 | | | 10 | 94972179 | + |
| 192 | TGGAAAACACTGCAG | | | | | | |
| | TTGACTTGTTTGGAG | | | | | | |
| | CTGGG | | | | | | |
| rs72558 | TTACTATTGAAAGCT | 197 | | | 10 | 94972179 | + |
| 192 | TGGAAAACACTGCAG | | | | | | |
| | TTGACTTGTTTGGAG | | | | | | |
| | CTGGG | | | | | | |
| rs72558 | TTACTATTGAAAGCT | 198 | | | 10 | 94972179 | + |
| 192 | TGGAAAACACTGCAG | | | | | | |
| | TTGACTTGTTTGGAG | | | | | | |
| | CTGGG | | | | | | |
| rs98861 | CTTCAGCAGGAGAAG | 199 | CTTCAGCAGGAGAAG | 1716 | 10 | 94972180 | − |
| 7574 | GAGAGCATATYTCAG | | GAGAGCATATYTCAG | | | | |
| | GGTTGTGCTTGTCGT | | GGTTGTGCTTGTCGT | | | | |
| | CTCTG | | CTCTC | | | | |
| rs98861 | CTTCAGCAGGAGAAG | 200 | CTTCAGCAGGAGAAG | 1717 | 10 | 94972180 | − |
| 7574 | GAGAGCATATCTCAG | | GAGAGCATATCTCAG | | | | |
| | GGTTGTGCTTGTCGT | | GGTTGTGCTTGTCGT | | | | |
| | CTCTG | | CTCTC | | | | |
| rs98861 | CTTCAGCAGGAGAAG | 201 | CTTCAGCAGGAGAAG | 1718 | 10 | 94972180 | − |
| 7574 | GAGAGCATATCTCAG | | GAGAGCATATCTCAG | | | | |
| | GGTTGTGCTTGTCGT | | GGTTGTGCTTGTCGT | | | | |
| | CTCTG | | CTCTC | | | | |
| rs12372 | TTCTGAAAATTAATT | 202 | TTCTGAAAATTAATT | 1719 | 10 | 94972233 | − |
| 25311 | AACTTATCATCTCTG | | AACTTATCATCTCTG | | | | |
| | ATCATACCTGTGACC | | ATCATACCTGTGACC | | | | |
| | TCTGA | | TCTGG | | | | |
| rs12372 | TTCTGAAAATTAATT | 203 | TTCTGAAAATTAATT | 1720 | 10 | 94972233 | − |
| 25311 | AACTTATCATCTCTG | | AACTTATCATCTCTG | | | | |
| | ATCATACCTGTGACC | | ATCATACCTGTGACC | | | | |
| | TCTGA | | TCTGG | | | | |
| rs12372 | TTTCTGAAAATTAAT | 204 | | | 10 | 94972233 | − |
| 25311 | TAACTTATCATCTCT | | | | | | |
| | GATCATACCTGTGAC | | | | | | |
| | CTCTG | | | | | | |
| rs57505 | GGCTCCTGTCTTGCA | 205 | | | 10 | 94981201 | − |
| 750 | TGCAGGGGCTCCGGT | | | | | | |
| | TTCTGCCAATCACAC | | | | | | |
| | GTTCA | | | | | | |
| rs57505 | GCTCCTGTCTTGCAT | 206 | GCTCCTGTCTTGCAT | 1721 | 10 | 94981201 | − |
| 750 | GCAGGGGCTCCGGTT | | GCAGGGGCTCCGGTT | | | | |
| | TCTGCCAATCACACG | | TCTGCCAATCACACG | | | | |
| | TTCAA | | TTCAG | | | | |
| rs57505 | GCTCCTGTCTTGCAT | 207 | GCTCCTGTCTTGCAT | 1722 | 10 | 94981201 | − |
| 750 | GCAGGGGCTCCGGTT | | GCAGGGGCTCCGGTT | | | | |
| | TCTGCCAATCACACG | | TCTGCCAATCACACG | | | | |
| | TTCAA | | TTCAG | | | | |
| rs28371 | TCTTATCAGCTAAAG | 208 | | | 10 | 94981224 | + |
| 685 | TCCAGGAAGAGATTG | | | | | | |
| | AACGTGTGATTGGCA | | | | | | |
| | GAAAC | | | | | | |
| rs28371 | TCTTATCAGCTAAAG | 209 | | | 10 | 94981224 | + |
| 685 | TCCAGGAAGAGATTG | | | | | | |
| | AACGTGTGATTGGCA | | | | | | |
| | GAAAC | | | | | | |
| rs36782 | CTTATCAGCTAAAGT | 210 | | | 10 | 94981225 | + |
| 6293 | CCAGGAAGAGATTGA | | | | | | |
| | ACGTGTGATTGGCAG | | | | | | |
| | AAACC | | | | | | |
| rs36782 | CTTATCAGCTAAAGT | 211 | | | 10 | 94981225 | + |
| 6293 | CCAGGAAGAGATTGA | | | | | | |
| | ACGTGTGATTGGCAG | | | | | | |
| | AAACC | | | | | | |
| rs12745 | GTGCACCACAGCATC | 212 | | | 10 | 94981230 | − |
| 35931 | TGTGTAGGGCATGTG | | | | | | |
| | KCTCCTGTCTTGCAT | | | | | | |
| | GCAGG | | | | | | |
| rs12745 | GTGCACCACAGCATC | 213 | | | 10 | 94981230 | − |
| 35931 | TGTGTAGGGCATGTG | | | | | | |
| | KCTCCTGTCTTGCAT | | | | | | |
| | GCAGG | | | | | | |
| rs12745 | GTGCACCACAGCATC | 214 | | | 10 | 94981230 | − |
| 35931 | TGTGTAGGGCATGTG | | | | | | |
| | GCTCCTGTCTTGCAT | | | | | | |
| | GCAGG | | | | | | |
| rs75082 | ATTGAACGTGTGATT | 215 | | | 10 | 94981250 | + |
| 0937 | GGCAGAAACCRGAGC | | | | | | |
| | MCCTGCATGCAAGAC | | | | | | |
| | AGGAG | | | | | | |
| rs75082 | ATTGAACGTGTGATT | 216 | | | 10 | 94981250 | + |
| 0937 | GGCAGAAACCRGAGC | | | | | | |
| | MCCTGCATGCAAGAC | | | | | | |
| | AGGAG | | | | | | |
| rs75082 | TTGAACGTGTGATTG | 217 | TTGAACGTGTGATTG | 1723 | 10 | 94981250 | + |
| 0937 | GCAGAAACCGGAGCC | | GCAGAAACCGGAGCC | | | | |
| | CCTGCATGCAAGACA | | CCTGCATGCAAGACA | | | | |
| | GGAGA | | GGAGC | | | | |
| rs74906 | CACTGCATGGGGCAG | 218 | | | 10 | 94981281 | − |
| 0448 | GCTGGTGGGGAGAAG | | | | | | |
| | GTCAATGTATCTCTG | | | | | | |
| | GACCT | | | | | | |
| rs74906 | ACTGCATGGGGCAGG | 219 | ACTGCATGGGGCAGG | 1724 | 10 | 94981281 | − |
| 0448 | CTGGTGGGGAGAAGG | | CTGGTGGGGAGAAGG | | | | |
| | TCAATGTATCTCTGG | | TCAATGTATCTCTGG | | | | |
| | ACCTT | | ACCTC | | | | |
| rs74906 | CACTGCATGGGGCAG | 220 | | | 10 | 94981281 | − |
| 0448 | GCTGGTGGGGAGAAG | | | | | | |
| | GTCAATGTATCTCTG | | | | | | |
| | GACCT | | | | | | |
| rs10579 | TTAATGTCACAGGTC | 221 | TTAATGTCACAGGTC | 1725 | 10 | 94981296 | − |
| 10 | ACTGCATGGGGCAGG | | ACTGCATGGGGCAGG | | | | |
| | CTGGTGGGGAGAAGG | | CTGGTGGGGAGAAGG | | | | |
| | TCAAT | | TCAAG | | | | |
| rs10579 | TTAATGTCACAGGTC | 222 | TTAATGTCACAGGTC | 1726 | 10 | 94981296 | − |
| 10 | ACTGCATGGGGCAGG | | ACTGCATGGGGCAGG | | | | |
| | CTGGTGGGGAGAAGG | | CTGGTGGGGAGAAGG | | | | |
| | TCAAT | | TCAAG | | | | |
| rs10579 | TTAATGTCACAGGTC | 223 | TTAATGTCACAGGTC | 1727 | 10 | 94981296 | − |
| 10 | ACTGCATGGGGCAGG | | ACTGCATGGGGCAGG | | | | |
| | CTGGTGGGGAGAAGG | | CTGGTGGGGAGAAGG | | | | |
| | TCAAT | | TCAAG | | | | |
| rs56165 | AGCCACATGCCCTAC | 224 | AGCCACATGCCCTAC | 1728 | 10 | 94981297 | + |
| 452 | ACAGATGCTGTGGTG | | ACAGATGCTGTGGTG | | | | |
| | CACGAGGTCCAGAGA | | CACGAGGTCCAGAGA | | | | |
| | TACAT | | TACAC | | | | |
| rs56165 | ATTTAATGTCACAGG | 225 | | | 10 | 94981297 | − |
| 452 | TCACTGCATGGGGCA | | | | | | |
| | GGCTGGTGGGGAGAA | | | | | | |
| | KSTCA | | | | | | |
| rs56165 | ATTTAATGTCACAGG | 226 | | | 10 | 94981297 | − |
| 452 | TCACTGCATGGGGCA | | | | | | |
| | GGCTGGTGGGGAGAA | | | | | | |
| | GGTCA | | | | | | |
| rs28371 | ACATGCCCTAttCAG | 227 | ACATGCCCTAttCAG | 1729 | 10 | 94981301 | + |
| 686 | ATGCTGTaGTGCACG | | ATGCTGTaGTGCACG | | | | |
| | AGGTCCAGAGATACA | | AGGTCCAGAGATACA | | | | |
| | TtGAC | | TtGAG | | | | |
| rs28371 | TGAATTTAAtGTCAC | 228 | TGAATTTAAtGTCAC | 1730 | 10 | 94981301 | − |
| 686 | AGGTCACgGCATaGa | | AGGTCACgGCATaGa | | | | |
| | GCAGGCTGGTGGGGA | | GCAGGCTGGTGGGGA | | | | |
| | gaAGG | | gaAGC | | | | |
| rs28371 | TGAATTTAATGTCAC | 229 | TGAATTTAATGTCAC | 1731 | 10 | 94981301 | − |
| 686 | AGGTCACTGCATGGG | | AGGTCACTGCATGGG | | | | |
| | GCAGGCTGGTGGGGA | | GCAGGCTGGTGGGGA | | | | |
| | GAAKG | | GAAKC | | | | |
| rs12505 | CTGAATTTAATGTCA | 230 | CTGAATTTAATGTCA | 1732 | 10 | 94981302 | − |
| 77724 | CAGGTCACTGCATGG | | CAGGTCACTGCATGG | | | | |
| | GGCAGGCTGGTGGGG | | GGCAGGCTGGTGGGG | | | | |
| | AGAAT | | AGAAG | | | | |
| rs12505 | TCTGAATTTAATGTC | 231 | | | 10 | 94981302 | − |
| 77724 | ACAGGTCACTGCATG | | | | | | |
| | GGGCAGGCTGGTGGG | | | | | | |
| | GAGAA | | | | | | |
| rs12505 | TCTGAATTTAATGTC | 232 | | | 10 | 94981302 | − |
| 77724 | ACAGGTCACTGCATG | | | | | | |
| | GGGCAGGCTGGTGGG | | | | | | |
| | GAGAA | | | | | | |
| 10_ | CCTGCCCCATGCAGT | 233 | CCTGCCCCATGCAGT | 1733 | 10 | 94981365 | + |
| 94981365 | GACCTGTGACATTAA | | GACCTGTGACATTAA | | | | |
| | ATTCAGAAACTATCT | | ATTCAGAAACTATCT | | | | |
| | CATTT | | CATTC | | | | |
| 10_ | CCTGCCCCATGCAGT | 234 | CCTGCCCCATGCAGT | 1734 | 10 | 94981365 | + |
| 94981365 | GACCTGTGACATTAA | | GACCTGTGACATTAA | | | | |
| | ATTCAGAAACTATCT | | ATTCAGAAACTATCT | | | | |
| | CATTT | | CATTC | | | | |
| 10_ | GCCTGCCCCATGCAG | 235 | | | 10 | 94981365 | + |
| 94981365 | TGACCTGTGACATTA | | | | | | |
| | AATTCAGAAACTATC | | | | | | |
| | TCATT | | | | | | |
| rs76421 | GTTGGGAAATTCTTT | 236 | | | 10 | 94986042 | − |
| 1126 | GTTGTCATGTAGCAC | | | | | | |
| | AGAAGTCAGGGAAAT | | | | | | |
| | TAATA | | | | | | |
| rs76421 | GTTGGGAAATTCTTT | 237 | | | 10 | 94986042 | − |
| 1126 | GTTGTCATGTAGCAC | | | | | | |
| | AGAAGTCAGGGAAAT | | | | | | |
| | TAATA | | | | | | |
| rs76421 | GTTGGGAAATTCTTT | 238 | | | 10 | 94986042 | − |
| 1126 | GTTGTCATGTAGCAC | | | | | | |
| | AGAAGTCAGGGAAAT | | | | | | |
| | TAATA | | | | | | |
| rs72558 | TTGTTTCTAGGGCAC | 239 | | | 10 | 94986073 | + |
| 193 | AACCATATTAATTTC | | | | | | |
| | CCTGACTTCTGTGCT | | | | | | |
| | ACATG | | | | | | |
| rs72558 | TGTTTCTAGGGCACA | 240 | TGTTTCTAGGGCACA | 1735 | 10 | 94986073 | + |
| 193 | ACCATATTAATTTCC | | ACCATATTAATTTCC | | | | |
| | CTGACTTCTGTGCTA | | CTGACTTCTGTGCTA | | | | |
| | CATGA | | CATGC | | | | |
| rs72558 | TGTTTCTAGGGCACA | 241 | TGTTTCTAGGGCACA | 1736 | 10 | 94986073 | + |
| 193 | ACCATATTAATTTCC | | ACCATATTAATTTCC | | | | |
| | CTGACTTCTGTGCTA | | CTGACTTCTGTGCTA | | | | |
| | CATGA | | CATGC | | | | |
| 10_ | ACGCATGAGGAGTAA | 242 | ACGCATGAGGAGTAA | 1737 | 10 | 94988855 | + |
| 94988855 | CTGCTCTCTGTGTTT | | CTGCTCTCTGTGTTT | | | | |
| | GCTATTTTCAGGAAA | | GCTATTTTCAGGAAA | | | | |
| | ACGGT | | ACGGA | | | | |
| 10_ | ACGCATGAGGAGTAA | 243 | ACGCATGAGGAGTAA | 1738 | 10 | 94988855 | + |
| 94988855 | CTGCTCTCTGTGTTT | | CTGCTCTCTGTGTTT | | | | |
| | GCTATTTTCAGGAAA | | GCTATTTTCAGGAAA | | | | |
| | ACGGT | | ACGGA | | | | |
| 10_ | ACGCATGAGGAGTAA | 244 | ACGCATGAGGAGTAA | 1739 | 10 | 94988855 | + |
| 94988855 | CTGCTCTCTGTGTTT | | CTGCTCTCTGTGTTT | | | | |
| | GCTATTTTCAGGAAA | | GCTATTTTCAGGAAA | | | | |
| | ACGGT | | ACGGA | | | | |
| rs76994 | AAGCCCTGGCCGGCA | 245 | AAGCCCTGGCCGGCA | 1740 | 10 | 94988917 | + |
| 2899 | TGGAGCTGTTTTTAT | | TGGAGCTGTTTTTAT | | | | |
| | TCCTGACCTCCATTT | | TCCTGACCTCCATTT | | | | |
| | TACAC | | TACAG | | | | |
| rs76994 | AAGCCCTGGCCGGCA | 246 | AAGCCCTGGCCGGCA | 1741 | 10 | 94988917 | + |
| 2899 | TGGAGCTGTTTTTAT | | TGGAGCTGTTTTTAT | | | | |
| | TCCTGACCTCCATTT | | TCCTGACCTCCATTT | | | | |
| | TACAC | | TACAG | | | | |
| rs76994 | AAGCCCTGGCCGGCA | 247 | AAGCCCTGGCCGGCA | 1742 | 10 | 94988917 | + |
| 2899 | TGGAGCTGTTTTTAT | | TGGAGCTGTTTTTAT | | | | |
| | TCCTGACCTCCATTT | | TCCTGACCTCCATTT | | | | |
| | TACAC | | TACAG | | | | |
| rs20220 | TGACAACTGGAGTGG | 248 | | | 10 | 94988925 | − |
| 1137 | TGTCAAGGTTCTTTG | | | | | | |
| | GGTCAACCAGAGATT | | | | | | |
| | TCAGG | | | | | | |
| rs20220 | GACAACTGGAGTGGT | 249 | GACAACTGGAGTGGT | 1743 | 10 | 94988925 | − |
| 1137 | GTCAAGGTTCTTTGG | | GTCAAGGTTCTTTGG | | | | |
| | GTCAACCAGAGATTT | | GTCAACCAGAGATTT | | | | |
| | CAGGT | | CAGGC | | | | |
| rs20220 | TGACAACTGGAGTGG | 250 | | | 10 | 94988925 | − |
| 1137 | TGTCAAGGTTCTTTG | | | | | | |
| | GGTCAACCAGAGATT | | | | | | |
| | TCAGG | | | | | | |
| rs76728 | TCCATTTTACAGAAC | 251 | TCCATTTTACAGAAC | 1744 | 10 | 94988955 | + |
| 4820 | TTTAACCTGAAATCT | | TTTAACCTGAAATCT | | | | |
| | CTGGTTGACCCAAAG | | CTGGTTGACCCAAAG | | | | |
| | AACCT | | AACCC | | | | |
| rs76728 | TCCATTTTACAGAAC | 252 | TCCATTTTACAGAAC | 1745 | 10 | 94988955 | + |
| 4820 | TTTAACCTGAAATCT | | TTTAACCTGAAATCT | | | | |
| | CTGGTTGACCCAAAG | | CTGGTTGACCCAAAG | | | | |
| | AACCT | | AACCC | | | | |
| rs76728 | CTCCATTTTACAGAA | 253 | | | 10 | 94988955 | + |
| 4820 | CTTTAACCTGAAATC | | | | | | |
| | TCTGGTTGACCCAAA | | | | | | |
| | GAACC | | | | | | |
| rs78158 | TCTTCTTCAGACAGG | 254 | | | 10 | 94988984 | − |
| 3846 | AATGAAGCACAGCTG | | | | | | |
| | GTAGAAGGGCGGCAC | | | | | | |
| | AGAGG | | | | | | |
| rs78158 | TCTTCTTCAGACAGG | 255 | | | 10 | 94988984 | − |
| 3846 | AATGAAGCACAGCTG | | | | | | |
| | GTAGAAGGGCGGCAC | | | | | | |
| | AGAGG | | | | | | |
| rs78158 | CTCTGGTTGACCCAA | 256 | | | 10 | 94988984 | + |
| 3846 | AGAACCTTGACACCA | | | | | | |
| | CTCCAGTTGTCAATG | | | | | | |
| | GATTT | | | | | | |
| rs93322 | CTGCAGGGACTGCAC | 257 | | | 10 | 94989020 | − |
| 39 | AGCAGCAGCCAGGCC | | | | | | |
| | ATCTGCTCTTCTTCA | | | | | | |
| | GACAG | | | | | | |
| rs93322 | CTGCAGGGACTGCAC | 258 | | | 10 | 94989020 | − |
| 39 | AGCAGCAGCCAGGCC | | | | | | |
| | ATCTGCTCTTCTTCA | | | | | | |
| | GACAG | | | | | | |
| rs86818 | GAGCTGCAGGGACTG | 259 | | | 10 | 94989023 | − |
| 2778 | CACAGCAGCAGCCAG | | | | | | |
| | GCCATCTGCTCTTCT | | | | | | |
| | TCAGA | | | | | | |
| rs86818 | AGCTGCAGGGACTGC | 260 | AGCTGCAGGGACTGC | 1746 | 10 | 94989023 | − |
| 2778 | ACAGCAGCAGCCAGG | | ACAGCAGCAGCCAGG | | | | |
| | CCATCTGCTCTTCTT | | CCATCTGCTCTTCTT | | | | |
| | CAGAA | | CAGAC | | | | |
| rs86818 | GAGCTGCAGGGACTG | 261 | | | 10 | 94989023 | − |
| 2778 | CACAGCAGCAGCCAG | | | | | | |
| | GCCATCTGCTCTTCT | | | | | | |
| | TCAGA | | | | | | |
| rs12769 | CATCTCTTTCCATTG | 262 | | | 10 | 96535124 | − |
| 205 | CTGAAAACGATTCCT | | | | | | |
| | AACAGGAAGAGAAAC | | | | | | |
| | GAAAC | | | | | | |
| rs13814 | ATCAGTTCTTACTTG | 263 | | | 10 | 96602618 | + |
| 2612 | TGTCTTGTCAGCTAA | | | | | | |
| | AGTCCAGGAAGAGAT | | | | | | |
| | TGAAC | | | | | | |
| rs13814 | ATCAGTTCTTACTTG | 264 | | | 10 | 96602618 | + |
| 2612 | TGTCTTGTCAGCTAA | | | | | | |
| | AGTCCAGGAAGAGAT | | | | | | |
| | TGAAC | | | | | | |
| rs11393 | GATACATCGACCTCA | 265 | | | 10 | 96602752 | + |
| 4938 | TCCCCACCAGCCTGC | | | | | | |
| | CCCATGCAGTGACCT | | | | | | |
| | GTGAC | | | | | | |
| rs56337 | AGTTACACATGAGGA | 266 | | | 10 | 96612495 | + |
| 013 | GTAACTTCTCCCTAT | | | | | | |
| | GTTTGTTATTTTCAG | | | | | | |
| | GAAAA | | | | | | |
| rs56337 | AGTTACACATGAGGA | 267 | | | 10 | 96612495 | + |
| 013 | GTAACTTCTCCCTAT | | | | | | |
| | GTTTGTTATTTTCAG | | | | | | |
| | GAAAA | | | | | | |
| rs93320 | TACTGATAGTGTACA | 268 | | | 10 | 96696674 | − |
| 94 | ATGATTCAGGATTTC | | | | | | |
| | GTAATTCAGTCTAGT | | | | | | |
| | AAATT | | | | | | |
| rs93320 | TACTGATAGTGTACA | 269 | | | 10 | 96696674 | − |
| 94 | ATGATTCAGGATTTC | | | | | | |
| | GTAATTCAGTCTAGT | | | | | | |
| | AAATT | | | | | | |
| rs72558 | CCTGTTAGGAATTGT | 270 | | | 10 | 96701991 | + |
| 189 | TTTCAGCAATGGAAA | | | | | | |
| | GAAATGGAAGGAGAT | | | | | | |
| | CCGGC | | | | | | |
| rs20018 | CAATGGAAAGAAATG | 271 | | | 10 | 96702012 | + |
| 3364 | GAAGGAGATCCGGCG | | | | | | |
| | TTTCTCCCTCATGAC | | | | | | |
| | GCTGC | | | | | | |
| rs17998 | CCCTCATGACGCTGC | 272 | | | 10 | 96702047 | + |
| 53 | GGAATTTTGGGATGG | | | | | | |
| | GGAAGAGGAGCATTG | | | | | | |
| | AGGAC | | | | | | |
| rs79001 | TTTGGGATGGGGAAG | 273 | TTTGGGATGGGGAAG | 1747 | 10 | 96702066 | + |
| 94 | AGGAGCATTGAGGAC | | AGGAGCATTGAGGAC | | | | |
| | CGTGTTCAAGAGGAA | | CGTGTTCAAGAGGAA | | | | |
| | GCCCA | | GCCCG | | | | |
| rs93321 | CAAATTCACAAGCAG | 274 | | | 10 | 96709037 | − |
| 30 | TCACATAACTAAGCT | | | | | | |
| | TTTGTTTACATTTTA | | | | | | |
| | CCTTC | | | | | | |
| rs18213 | AAACAAGTCAACTGC | 275 | | | 10 | 96731876 | − |
| 2442 | AGTGTTTTCCAAGCT | | | | | | |
| | TTCAATAGTAAATTC | | | | | | |
| | AGATG | | | | | | |
| rs18213 | AAACAAGTCAACTGC | 276 | | | 10 | 96731876 | − |
| 2442 | AGTGTTTTCCAAGCT | | | | | | |
| | TTCAATAGTAAATTC | | | | | | |
| | AGATG | | | | | | |
| rs28371 | TCTTATCAGCTAAAG | 277 | | | 10 | 96740981 | + |
| 685 | TCCAGGAAGAGATTG | | | | | | |
| | AACGTGTGATTGGCA | | | | | | |
| | GAAAC | | | | | | |
| rs36782 | CTTATCAGCTAAAGT | 278 | | | 10 | 96740982 | + |
| 6293 | CCAGGAAGAGATTGA | | | | | | |
| | ACGTGTGATTGGCAG | | | | | | |
| | AAACC | | | | | | |
| rs93322 | CTGCAGGGACTGCAC | 279 | | | 10 | 96748777 | − |
| 39 | AGCAGCAGCCAGGCC | | | | | | |
| | ATCTGCTCTTCTTCA | | | | | | |
| | GACAG | | | | | | |
| rs41490 | ATGGTACTATGGGAG | 280 | | | 12 | 21178615 | − |
| 56 | TCTCCCCTATTCCAC | | | | | | |
| | GAAGCATATTACCCA | | | | | | |
| | TGAAC | | | | | | |
| rs41490 | TATCTACATAGGTTG | 281 | | | 12 | 21178615 | + |
| 56 | TTTAAAGGAATCTGG | | | | | | |
| | GTCATACATGTGGAT | | | | | | |
| | ATATG | | | | | | |
| rs41490 | TATCTACATAGGTTG | 282 | | | 12 | 21178615 | + |
| 56 | TTTAAAGGAATCTGG | | | | | | |
| | GTCATACATGTGGAT | | | | | | |
| | ATATG | | | | | | |
| rs74607 | ACGCAACGCGGATGC | 283 | | | 13 | 48037801 | − |
| 1566 | TTGCAGCTGGTCACC | | | | | | |
| | ACGACTCCGACTCCG | | | | | | |
| | ACTCC | | | | | | |
| rs74607 | ACGCAACGCGGATGC | 284 | | | 13 | 48037801 | − |
| 1566 | TTGCAGCTGGTCACC | | | | | | |
| | ACGACTCCGACTCCG | | | | | | |
| | ACTCC | | | | | | |
| rs74607 | ACGCAACGCGGATGC | 285 | | | 13 | 48037801 | − |
| 1566 | TTGCAGCTGGTCACC | | | | | | |
| | ACGACTCCGACTCCG | | | | | | |
| | ACTCC | | | | | | |
| rs11685 | GGGTTCCTTGGGAAG | 286 | | | 13 | 48045719 | + |
| 5232 | AACTACCTCCCCTGG | | | | | | |
| | ACCAGCTTTTCTGGG | | | | | | |
| | GACTG | | | | | | |
| rs11685 | GGGTTCCTTGGGAAG | 287 | | | 13 | 48045719 | + |
| 5232 | AACTACCTCCCCTGG | | | | | | |
| | ACCAGCTTTTCTGGG | | | | | | |
| | GACTG | | | | | | |
| rs11685 | GGGTTCCTTGGGAAG | 288 | | | 13 | 48045719 | + |
| 5232 | AACTACCTCCCCTGG | | | | | | |
| | ACCAGCTTTTCTGGG | | | | | | |
| | GACTG | | | | | | |
| rs99232 | ATCCCTCTGGGAAGT | 289 | | | 16 | 31096368 | − |
| 31 | CAAGCAAGAGAAGAC | | | | | | |
| | CTGAAAAACAACCAT | | | | | | |
| | TGGCC | | | | | | |
| rs99232 | ATCCCTCTGGGAAGT | 290 | | | 16 | 31096368 | − |
| 31 | CAAGCAAGAGAAGAC | | | | | | |
| | CTGAAAAACAACCAT | | | | | | |
| | TGGCC | | | | | | |
| rs99232 | ATCCCTCTGGGAAGT | 291 | | | 16 | 31096368 | − |
| 31 | CAAGCAAGAGAAGAC | | | | | | |
| | CTGAAAAACAACCAT | | | | | | |
| | TGGCC | | | | | | |
| rs21086 | CAGGCATTATCTGCC | 292 | | | 19 | 15879621 | − |
| 22 | TCATCAGTGTTTTCG | | | | | | |
| | GAACCCATCACAACC | | | | | | |
| | CAGCT | | | | | | |
| rs21086 | CAGGCATTATCTGCC | 293 | | | 19 | 15879621 | − |
| 22 | TCATCAGTGTTTTCG | | | | | | |
| | GAACCCATCACAACC | | | | | | |
| | CAGCT | | | | | | |
| rs21086 | CAGGCATTATCTGCC | 294 | | | 19 | 15879621 | − |
| 22 | TCATCAGTGTTTTCG | | | | | | |
| | GAACCCATCACAACC | | | | | | |
| | CAGCT | | | | | | |
| rs19392 | AGGTGGTCCTGCAGT | 295 | | | 19 | 38440802 | + |
| 2747 | GCAGCGCTACCGTGC | | | | | | |
| | TCAAGGAGCAGCTCA | | | | | | |
| | AGCTC | | | | | | |
| rs19392 | AGTGGGCTCCAGGAA | 296 | | | 19 | 38440802 | − |
| 2747 | GCACAGGCGGTTGCC | | | | | | |
| | GAAGCCCTCGGCGGC | | | | | | |
| | CAGGC | | | | | | |
| rs19392 | AGTGGGCTCCAGGAA | 297 | | | 19 | 38440802 | − |
| 2747 | GCACAGGCGGTTGCC | | | | | | |
| | GAAGCCCTCGGCGGC | | | | | | |
| | CAGGC | | | | | | |
| rs19392 | TGCTCAAGGAGCAGC | 298 | | | 19 | 38440829 | + |
| 2748 | TCAAGCTCTGCCTGG | | | | | | |
| | CCGCCGAGGGCTTCG | | | | | | |
| | GCAAC | | | | | | |
| rs19392 | CCCTCCTGCACAGAC | 299 | | | 19 | 38440829 | − |
| 2748 | CTGCGCGTTGCTAGT | | | | | | |
| | GGGCTCCAGGAAGCA | | | | | | |
| | CAGGC | | | | | | |
| rs19392 | CCCTCCTGCACAGAC | 300 | | | 19 | 38440829 | − |
| 2748 | CTGCGCGTTGCTAGT | | | | | | |
| | GGGCTCCAGGAAGCA | | | | | | |
| | CAGGC | | | | | | |
| rs11819 | GGTGGACCATGCACC | 301 | | | 19 | 38444211 | + |
| 2161 | CAGCCTCCAAGCAGA | | | | | | |
| | GGTCTGAAGGAGAAA | | | | | | |
| | AGGTC | | | | | | |
| rs11819 | GGTGGACCATGCACC | 302 | | | 19 | 38444211 | + |
| 2161 | CAGCCTCCAAGCAGA | | | | | | |
| | GGTCTGAAGGAGAAA | | | | | | |
| | AGGTC | | | | | | |
| rs19392 | CAGGTAGCGCTCGGA | 303 | CAGGTAGCGCTCGGA | 1748 | 19 | 38444212 | − |
| 2753 | GGAGACACTGACAAG | | GGAGACACTGACAAG | | | | |
| | GATGATGTCATCCCC | | GATGATGTCATCCCC | | | | |
| | AACGT | | AACGC | | | | |
| rs19392 | CAGGTAGCGCTCGGA | 304 | CAGGTAGCGCTCGGA | 1749 | 19 | 38444212 | − |
| 2753 | GGAGACACTGACAAG | | GGAGACACTGACAAG | | | | |
| | GATGATGTCATCCCC | | GATGATGTCATCCCC | | | | |
| | AACGT | | AACGC | | | | |
| rs19392 | CAGGTAGCGCTCGGA | 305 | CAGGTAGCGCTCGGA | 1750 | 19 | 38444212 | − |
| 2753 | GGAGACACTGACAAG | | GGAGACACTGACAAG | | | | |
| | GATGATGTCATCCCC | | GATGATGTCATCCCC | | | | |
| | AACGT | | AACGC | | | | |
| rs18010 | AACCCTTGACTTCAC | 306 | AACCCTTGACTTCAC | 1751 | 19 | 38446710 | + |
| 86 | TCTCTTCTGTGTCCC | | TCTCTTCTGTGTCCC | | | | |
| | CAGACTTGTCTACTA | | CAGACTTGTCTACTA | | | | |
| | TGAGA | | TGAGG | | | | |
| rs18010 | AACCCTTGACTTCAC | 307 | AACCCTTGACTTCAC | 1752 | 19 | 38446710 | + |
| 86 | TCTCTTCTGTGTCCC | | TCTCTTCTGTGTCCC | | | | |
| | CAGACTTGTCTACTA | | CAGACTTGTCTACTA | | | | |
| | TGAGC | | TGAGG | | | | |
| rs18010 | GAACCCTTGACTTCA | 308 | | | 19 | 38446710 | + |
| 86 | CTCTCTTCTGTGTCC | | | | | | |
| | CCAGACTTGTCTACT | | | | | | |
| | ATGAG | | | | | | |
| rs19392 | AGTGACTCCCCGTAC | 309 | AGTGACTCCCCGTAC | 1753 | 19 | 38448673 | − |
| 2762 | TTGATCTCAGGGGGG | | TTGATCTCAGGGGGG | | | | |
| | CCCATGCCCTCCACA | | CCCATGCCCTCCACA | | | | |
| | TCCCA | | TCCCG | | | | |
| rs19392 | AGTGACTCCCCGTAC | 310 | AGTGACTCCCCGTAC | 1754 | 19 | 38448673 | − |
| 2762 | TTGATCTCAGGGGGG | | TTGATCTCAGGGGGG | | | | |
| | CCCATGCCCTCCACA | | CCCATGCCCTCCACA | | | | |
| | TCCCA | | TCCCG | | | | |
| rs19392 | AGTGACTCCCCGTAC | 311 | AGTGACTCCCCGTAC | 1755 | 19 | 38448673 | − |
| 2762 | TTGATCTCAGGGGGG | | TTGATCTCAGGGGGG | | | | |
| | CCCATGCCCTCCACA | | CCCATGCCCTCCACA | | | | |
| | TCCCA | | TCCCG | | | | |
| rs12191 | GTGAGCCACAGTCCT | 312 | GTGAGCCACAGTCCT | 1756 | 19 | 38448712 | − |
| 8592 | GAGGCCACATGCTGC | | GAGGCCACATGCTGC | | | | |
| | ACGAAGCACAGTGAC | | ACGAAGCACAGTGAC | | | | |
| | TCCCG | | TCCCC | | | | |
| rs12191 | GTGAGCCACAGTCCT | 313 | GTGAGCCACAGTCCT | 1757 | 19 | 38448712 | − |
| 8592 | GAGGCCACATGCTGC | | GAGGCCACATGCTGC | | | | |
| | ACGAAGCACAGTGAC | | ACGAAGCACAGTGAC | | | | |
| | TCCCG | | TCCCC | | | | |
| rs12191 | GTGAGCCACAGTCCT | 314 | GTGAGCCACAGTCCT | 1758 | 19 | 38448712 | − |
| 8592 | GAGGCCACATGCTGC | | GAGGCCACATGCTGC | | | | |
| | ACGAAGCACAGTGAC | | ACGAAGCACAGTGAC | | | | |
| | TCCCG | | TCCCC | | | | |
| rs19392 | GCTCACTTGATGAAC | 315 | GCTCACTTGATGAAC | 1759 | 19 | 38451842 | − |
| 2764 | TGGTTGTATAGGCCA | | TGGTTGTATAGGCCA | | | | |
| | TTGGTGCTGTGGATC | | TTGGTGCTGTGGATC | | | | |
| | ATGCA | | ATGCG | | | | |
| rs19392 | GCTCACTTGATGAAC | 316 | GCTCACTTGATGAAC | 1760 | 19 | 38451842 | − |
| 2764 | TGGTTGTATAGGCCA | | TGGTTGTATAGGCCA | | | | |
| | TTGGTGCTGTGGATC | | TTGGTGCTGTGGATC | | | | |
| | ATGCA | | ATGCG | | | | |
| rs19392 | GCTCACTTGATGAAC | 317 | GCTCACTTGATGAAC | 1761 | 19 | 38451842 | − |
| 2764 | TGGTTGTATAGGCCA | | TGGTTGTATAGGCCA | | | | |
| | TTGGTGCTGTGGATC | | TTGGTGCTGTGGATC | | | | |
| | ATGCA | | ATGCG | | | | |
| rs11819 | GGCAGGTTGCTCACT | 318 | GGCAGGTTGCTCACT | 1762 | 19 | 38451850 | − |
| 2116 | TGATGAACTGGTTGT | | TGATGAACTGGTTGT | | | | |
| | ATAGGCCATTGGTGC | | ATAGGCCATTGGTGC | | | | |
| | TGTGG | | TGTGC | | | | |
| rs11819 | GGCAGGTTGCTCACT | 319 | GGCAGGTTGCTCACT | 1763 | 19 | 38451850 | − |
| 2116 | TGATGAACTGGTTGT | | TGATGAACTGGTTGT | | | | |
| | ATAGGCCATTGGTGC | | ATAGGCCATTGGTGC | | | | |
| | TGTGG | | TGTGC | | | | |
| rs11819 | TGCAGGGGAGGAGGC | 320 | | | 19 | 38455359 | + |
| 2162 | AGCCGAGTCCTGGAA | | | | | | |
| | AGAGATTGTGAATCT | | | | | | |
| | TCTCT | | | | | | |
| rs11819 | GCAGGGGAGGAGGCA | 321 | GCAGGGGAGGAGGCA | 1764 | 19 | 38455359 | + |
| 2162 | GCCGAGTCCTGGAAA | | GCCGAGTCCTGGAAA | | | | |
| | GAGATTGTGAATCTT | | GAGATTGTGAATCTT | | | | |
| | CTCTA | | CTCTC | | | | |
| rs11819 | TGGGGTGTTCTCAGG | 322 | TGGGGTGTTCTCAGG | 1765 | 19 | 38455359 | − |
| 2162 | GGAGTCAGGACTGGG | | GGAGTCAGGACTGGG | | | | |
| | ACCCCTACCTAGGAG | | ACCCCTACCTAGGAG | | | | |
| | TTCAT | | TTCAG | | | | |
| rs11819 | TGGGGTGTTCTCAGG | 323 | TGGGGTGTTCTCAGG | 1766 | 19 | 38455359 | − |
| 2162 | GGAGTCAGGACTGGG | | GGAGTCAGGACTGGG | | | | |
| | ACCCCTACCTAGGAG | | ACCCCTACCTAGGAG | | | | |
| | TTCAT | | TTCAG | | | | |
| rs11188 | CCAGTCCTATTGGAT | 324 | CCAGTCCTATTGGAT | 1767 | 19 | 38455463 | + |
| 8148 | CTGACACCTCTTCCC | | CTGACACCTCTTCCC | | | | |
| | CSCTCAGCTTCTCTA | | CSCTCAGCTTCTCTA | | | | |
| | ATCCA | | ATCCG | | | | |
| rs11188 | CCAGTCCTATTGGAT | 325 | CCAGTCCTATTGGAT | 1768 | 19 | 38455463 | + |
| 8148 | CTGACACCTCTTCCC | | CTGACACCTCTTCCC | | | | |
| | CCCTCAGCTTCTCTA | | CCCTCAGCTTCTCTA | | | | |
| | ATCCA | | ATCCG | | | | |
| rs11188 | CCAGTCCTATTGGAT | 326 | CCAGTCCTATTGGAT | 1769 | 19 | 38455463 | + |
| 8148 | CTGACACCTCTTCCC | | CTGACACCTCTTCCC | | | | |
| | CCCTCAGCTTCTCTA | | CCCTCAGCTTCTCTA | | | | |
| | ATCCA | | ATCCG | | | | |
| rs19392 | TTGCTGACCAGCCAG | 327 | TTGCTGACCAGCCAG | 1770 | 19 | 38455471 | − |
| 2768 | TCCAAGTTTGTGGAG | | TCCAAGTTTGTGGAG | | | | |
| | AAGAGGGCACAGTTG | | AAGAGGGCACAGTTG | | | | |
| | CTAYA | | CTAYG | | | | |
| rs19392 | TTGCTGACCAGCCAG | 328 | TTGCTGACCAGCCAG | 1771 | 19 | 38455471 | − |
| 2768 | TCCAAGTTTGTGGAG | | TCCAAGTTTGTGGAG | | | | |
| | AAGAGGGCACAGTTG | | AAGAGGGCACAGTTG | | | | |
| | CTAYA | | CTAYG | | | | |
| rs19392 | TATTGGATCTGACAC | 329 | | | 19 | 38455471 | + |
| 2768 | CTCTTCCCCCCTCAG | | | | | | |
| | CTTCTCTAATCCGTG | | | | | | |
| | GCAAT | | | | | | |
| rs14433 | ATTGGATCTGACACC | 330 | | | 19 | 38455472 | + |
| 6148 | TCTTCCCCCCTCAGC | | | | | | |
| | TTCTCTAATCCGTGG | | | | | | |
| | CAATC | | | | | | |
| rs14433 | ATTGGATCTGACACC | 331 | | | 19 | 38455472 | + |
| 6148 | TCTTCCCCCCTCAGC | | | | | | |
| | TTCTCTAATCCGTGG | | | | | | |
| | CAATC | | | | | | |
| rs19392 | AGCCTCTGTCCCACT | 332 | | | 19 | 38455528 | − |
| 2770 | CCCCCGGGTTCTCCT | | | | | | |
| | ACCAGACGAGGCCTC | | | | | | |
| | CAGCC | | | | | | |
| rs19392 | AGCCTCTGTCCCACT | 333 | | | 19 | 38455528 | − |
| 2770 | CCCCCGGGTTCTCCT | | | | | | |
| | ACCAGACGAGGCCTC | | | | | | |
| | CAGCC | | | | | | |
| rs19392 | AGCCTCTGTCCCACT | 334 | | | 19 | 38455528 | − |
| 2770 | CCCCCGGGTTCTCCT | | | | | | |
| | ACCAGACGAGGCCTC | | | | | | |
| | CAGCC | | | | | | |
| rs11819 | AAGCTCACGGCCAGG | 335 | | | 19 | 38457545 | − |
| 2172 | CAGCAAGTTCTCAGT | | | | | | |
| | AATAAGATCTTGGTT | | | | | | |
| | GGAGC | | | | | | |
| rs11819 | AAGCTCACGGCCAGG | 336 | | | 19 | 38457545 | − |
| 2172 | CAGCAAGTTCTCAGT | | | | | | |
| | AATAAGATCTTGGTT | | | | | | |
| | GGAGC | | | | | | |
| rs11819 | AAGCTCACGGCCAGG | 337 | | | 19 | 38457545 | − |
| 2172 | CAGCAAGTTCTCAGT | | | | | | |
| | AATAAGATCTTGGTT | | | | | | |
| | GGAGC | | | | | | |
| rs19392 | GTCCTGGACGTGCTA | 338 | GTCCTGGACGTGCTA | 1772 | 19 | 38457546 | + |
| 2772 | TGCTCCCTGTGTGTG | | TGCTCCCTGTGTGTG | | | | |
| | TGTAATGGTGTGGCT | | TGTAATGGTGTGGCT | | | | |
| | GTACT | | GTACG | | | | |
| rs19392 | AAGCTCACGGCCAGG | 339 | AAGCTCACGGCCAGG | 1773 | 19 | 38457546 | − |
| 2772 | CAGCAAGTTCTCAGT | | CAGCAAGTTCTCAGT | | | | |
| | AATAAGATCTTGGTT | | AATAAGATCTTGGTT | | | | |
| | GGAGA | | GGAGC | | | | |
| rs19392 | AAGCTCACGGCCAGG | 340 | AAGCTCACGGCCAGG | 1774 | 19 | 38457546 | − |
| 2772 | CAGCAAGTTCTCAGT | | CAGCAAGTTCTCAGT | | | | |
| | AATAAGATCTTGGTT | | AATAAGATCTTGGTT | | | | |
| | GGAGA | | GGAGC | | | | |
| rs11819 | CGTCCTCCGTGGAAG | 341 | | | 19 | 38494564 | + |
| 2175 | ACACCATGAGCCTGC | | | | | | |
| | TCGAGTGCCTCGGCC | | | | | | |
| | AGATC | | | | | | |
| rs11819 | TCCTCCGTGGAAGAC | 342 | TCCTCCGTGGAAGAC | 1775 | 19 | 38494565 | + |
| 2163 | ACCATGAGCCTGCTC | | ACCATGAGCCTGCTC | | | | |
| | GAGTGCCTCGGCCAG | | GAGTGCCTCGGCCAG | | | | |
| | ATCCA | | ATCCG | | | | |
| rs11819 | TCCTCCGTGGAAGAC | 343 | TCCTCCGTGGAAGAC | 1776 | 19 | 38494565 | + |
| 2163 | ACCATGAGCCTGCTC | | ACCATGAGCCTGCTC | | | | |
| | GAGTGCCTCGGCCAG | | GAGTGCCTCGGCCAG | | | | |
| | ATCCA | | ATCCG | | | | |
| rs11819 | TCCTCCGTGGAAGAC | 344 | TCCTCCGTGGAAGAC | 1777 | 19 | 38494565 | + |
| 2163 | ACCATGAGCCTGCTC | | ACCATGAGCCTGCTC | | | | |
| | GAGTGCCTCGGCCAG | | GAGTGCCTCGGCCAG | | | | |
| | ATCCA | | ATCCG | | | | |
| rs11819 | ACACCATGAGCCTGC | 345 | | | 19 | 38494579 | + |
| 2176 | TCGAGTGCCTCGGCC | | | | | | |
| | AGATCCGCTCGCTGC | | | | | | |
| | TCATC | | | | | | |
| rs11819 | ACACCATGAGCCTGC | 346 | | | 19 | 38494579 | + |
| 2176 | TCGAGTGCCTCGGCC | | | | | | |
| | AGATCCGCTCGCTGC | | | | | | |
| | TCATC | | | | | | |
| rs11819 | ACACCATGAGCCTGC | 347 | | | 19 | 38494579 | + |
| 2176 | TCGAGTGCCTCGGCC | | | | | | |
| | AGATCCGCTCGCTGC | | | | | | |
| | TCATC | | | | | | |
| rs11819 | GTCTTCTACCAACAC | 348 | GTCTTCTACCAACAC | 1778 | 19 | 38496283 | + |
| 2177 | CCGAACCTGATGAGG | | CCGAACCTGATGAGG | | | | |
| | GCGCTGGGCATGCAC | | GCGCTGGGCATGCAC | | | | |
| | GAGAC | | GAGAG | | | | |
| rs11819 | GTCTTCTACCAACAC | 349 | GTCTTCTACCAACAC | 1779 | 19 | 38496283 | + |
| 2177 | CCGAACCTGATGAGG | | CCGAACCTGATGAGG | | | | |
| | GCGCTGGGCATGCAC | | GCGCTGGGCATGCAC | | | | |
| | GAGAC | | GAGAG | | | | |
| rs11819 | AGTCTTCTACCAACA | 350 | | | 19 | 38496283 | + |
| 2177 | CCCGAACCTGATGAG | | | | | | |
| | GGCGCTGGGCATGCA | | | | | | |
| | CGAGA | | | | | | |
| rs11256 | AGACATTGGCTGGAA | 351 | | | 19 | 38499223 | + |
| 3513 | CCCCTGTGGTGGAGA | | | | | | |
| | GCGCTACCTGGACTT | | | | | | |
| | CCTGC | | | | | | |
| rs11256 | AGACATTGGCTGGAA | 352 | | | 19 | 38499223 | + |
| 3513 | CCCCTGTGGTGGAGA | | | | | | |
| | GCGCTACCTGGACTT | | | | | | |
| | CCTGC | | | | | | |
| rs11256 | AGACATTGGCTGGAA | 353 | | | 19 | 38499223 | + |
| 3513 | CCCCTGTGGTGGAGA | | | | | | |
| | GCGCTACCTGGACTT | | | | | | |
| | CCTGC | | | | | | |
| rs19392 | GAGACCCCCTTTCCC | 354 | | | 19 | 38499655 | + |
| 2802 | CATGCGGGTGGCCAG | | | | | | |
| | GCGAGAGCGTGGAGG | | | | | | |
| | AGAAC | | | | | | |
| rs19392 | GAGACCCCCTTTCCC | 355 | | | 19 | 38499655 | + |
| 2802 | CATGCGGGTGGCCAG | | | | | | |
| | GCGAGAGCGTGGAGG | | | | | | |
| | AGAAC | | | | | | |
| rs19392 | GAGACCCCCTTTCCC | 356 | | | 19 | 38499655 | + |
| 2802 | CATGCGGGTGGCCAG | | | | | | |
| | GCGAGAGCGTGGAGG | | | | | | |
| | AGAAC | | | | | | |
| rs19392 | CATGCGGGTGGCCAG | 357 | | | 19 | 38499670 | + |
| 2803 | GCGAGAGCGTGGAGG | | | | | | |
| | AGAACGCCAATGTGG | | | | | | |
| | TGGTG | | | | | | |
| rs19392 | CTCACCCCGCAGGGC | 358 | | | 19 | 38499670 | − |
| 2803 | GGGTCCGAAGCACTC | | | | | | |
| | AGGCTTCCGGATGAG | | | | | | |
| | CAGCC | | | | | | |
| rs19392 | CTCACCCCGCAGGGC | 359 | | | 19 | 38499670 | − |
| 2803 | GGGTCCGAAGCACTC | | | | | | |
| | AGGCTTCCGGATGAG | | | | | | |
| | CAGCC | | | | | | |
| rs19392 | CGCAGGGTCCTCGGA | 360 | CGCAGGGTCCTCGGA | 1780 | 19 | 38499731 | − |
| 2807 | GATGCGGATGGCCTC | | GATGCGGATGGCCTC | | | | |
| | TTCGATGGCAGCCAG | | TTCGATGGCAGCCAG | | | | |
| | CAGCG | | CAGCC | | | | |
| rs19392 | CGCAGGGTCCTCGGA | 361 | CGCAGGGTCCTCGGA | 1781 | 19 | 38499731 | − |
| 2807 | GATGCGGATGGCCTC | | GATGCGGATGGCCTC | | | | |
| | TTCGATGGCAGCCAG | | TTCGATGGCAGCCAG | | | | |
| | CAGCG | | CAGCC | | | | |
| rs19392 | CGGAAGCCTGAGTGC | 362 | CGGAAGCCTGAGTGC | 1782 | 19 | 38499731 | + |
| 2807 | TTCGGACCCGCCCTG | | TTCGGACCCGCCCTG | | | | |
| | CGGGGTGAGGGTGGC | | CGGGGTGAGGGTGGC | | | | |
| | TCAGC | | TCAGG | | | | |
| rs19392 | GGGTCTCACATGCAT | 363 | | | 19 | 38499975 | − |
| 2809 | CTCTGGTGCACAGCG | | | | | | |
| | TCCGAGCAGGTCGAT | | | | | | |
| | CAAGG | | | | | | |
| rs19392 | GGGTCTCACATGCAT | 364 | | | 19 | 38499975 | − |
| 2809 | CTCTGGTGCACAGCG | | | | | | |
| | TCCGAGCAGGTCGAT | | | | | | |
| | CAAGG | | | | | | |
| rs19392 | GGGTCTCACATGCAT | 365 | | | 19 | 38499975 | − |
| 2809 | CTCTGGTGCACAGCG | | | | | | |
| | TCCGAGCAGGTCGAT | | | | | | |
| | CAAGG | | | | | | |
| rs12191 | CCATCCTGCCCTGGC | 366 | | | 19 | 38499993 | − |
| 8593 | TCAGGGTCTCACATG | | | | | | |
| | CATCTCTGGTGCACA | | | | | | |
| | GCGTC | | | | | | |
| rs12191 | ACCTGGGACACGCCA | 367 | | | 19 | 38499993 | + |
| 8593 | TCATGTCCTTCTATG | | | | | | |
| | CCGCCTTGATCGACC | | | | | | |
| | TGCTC | | | | | | |
| rs12191 | ACCTGGGACACGCCA | 368 | | | 19 | 38499993 | + |
| 8593 | TCATGTCCTTCTATG | | | | | | |
| | CCGCCTTGATCGACC | | | | | | |
| | TGCTC | | | | | | |
| rs28933 | GGACACGCCATCATG | 369 | GGACACGCCATCATG | 1783 | 19 | 38499997 | + |
| 396 | TCCTTCTATGCCGCC | | TCCTTCTATGCCGCC | | | | |
| | TTGATCGACCTGCTC | | TTGATCGACCTGCTC | | | | |
| | GGACA | | GGACG | | | | |
| rs28933 | GGACACGCCATCATG | 370 | GGACACGCCATCATG | 1784 | 19 | 38499997 | + |
| 396 | TCCTTCTATGCCGCC | | TCCTTCTATGCCGCC | | | | |
| | TTGATCGACCTGCTC | | TTGATCGACCTGCTC | | | | |
| | GGACA | | GGACG | | | | |
| rs28933 | GGACACGCCATCATG | 371 | GGACACGCCATCATG | 1785 | 19 | 38499997 | + |
| 396 | TCCTTCTATGCCGCC | | TCCTTCTATGCCGCC | | | | |
| | TTGATCGACCTGCTC | | TTGATCGACCTGCTC | | | | |
| | GGACA | | GGACG | | | | |
| rs11819 | TCCCTCCCTCTACTC | 372 | | | 19 | 38500636 | + |
| 2124 | CCCAGCTAATCCAAG | | | | | | |
| | CCGGCAAGGGTGAGG | | | | | | |
| | CCCTG | | | | | | |
| rs11819 | GCCCACAAGGTCCTC | 373 | | | 19 | 38500636 | − |
| 2124 | CAAGGGCACAAGGGA | | | | | | |
| | GCGGAGGATGGCGCG | | | | | | |
| | GATCC | | | | | | |
| rs11819 | GCCCACAAGGTCCTC | 374 | | | 19 | 38500636 | − |
| 2124 | CAAGGGCACAAGGGA | | | | | | |
| | GCGGAGGATGGCGCG | | | | | | |
| | GATCC | | | | | | |
| rs19392 | CCTCTACTCCCCAGC | 375 | | | 19 | 38500642 | + |
| 2816 | TAATCCAAGCCGGCA | | | | | | |
| | AGGGTGAGGCCCTGC | | | | | | |
| | GGATC | | | | | | |
| rs19392 | GATGATGCCCACAAG | 376 | | | 19 | 38500642 | − |
| 2816 | GTCCTCCAAGGGCAC | | | | | | |
| | AAGGGAGCGGAGGAT | | | | | | |
| | GGCGC | | | | | | |
| rs19392 | CCTCTACTCCCCAGC | 377 | | | 19 | 38500642 | + |
| 2816 | TAATCCAAGCCGGCA | | | | | | |
| | AGGGTGAGGCCCTGC | | | | | | |
| | GGATC | | | | | | |
| rs11819 | CTCTACTCCCCAGCT | 378 | | | 19 | 38500643 | + |
| 2122 | AATCCAAGCCGGCAA | | | | | | |
| | GGGTGAGGCCCTGYG | | | | | | |
| | GAYCC | | | | | | |
| rs11819 | CTCTACTCCCCAGCT | 379 | | | 19 | 38500643 | + |
| 2122 | AATCCAAGCCGGCAA | | | | | | |
| | GGGTGAGGCCCTGYG | | | | | | |
| | GAYCC | | | | | | |
| rs11819 | CTCTACTCCCCAGCT | 380 | | | 19 | 38500643 | − |
| 2122 | AATCCAAGCCGGCAA | | | | | | |
| | GGGTGAGGCCCTGCG | | | | | | |
| | GATCC | | | | | | |
| rs28933 | CAGTGGGAGGCTGAT | 381 | | | 19 | 38500654 | − |
| 397 | GATGCCCACAAGGTC | | | | | | |
| | CTCCAAGGGCACAAG | | | | | | |
| | GGAGC | | | | | | |
| rs28933 | CAGTGGGAGGCTGAT | 382 | | | 19 | 38500654 | − |
| 397 | GATGCCCACAAGGTC | | | | | | |
| | CTCCAAGGGCACAAG | | | | | | |
| | GGAGC | | | | | | |
| rs28933 | AGCTAATCCAAGCCG | 383 | | | 19 | 38500654 | + |
| 397 | GCAAGGGTGAGGCCC | | | | | | |
| | TGCGGATCCGCGCCA | | | | | | |
| | TCCTC | | | | | | |
| rs28933 | AGCTAATCCAAGCCG | 384 | | | 19 | 38500654 | + |
| 397 | GCAAGGGTGAGGCCC | | | | | | |
| | TGCGGAYCYRCGCCA | | | | | | |
| | TCCTC | | | | | | |
| rs12191 | GCAGTGGGAGGCTGA | 385 | | | 19 | 38500655 | − |
| 8594 | TGATGCCCACAAGGT | | | | | | |
| | CCTCCAAGGGCACAA | | | | | | |
| | GGGAG | | | | | | |
| rs12191 | GCAGTGGGAGGCTGA | 386 | | | 19 | 38500655 | − |
| 8594 | TGATGCCCACAAGGT | | | | | | |
| | CCTCCAAGGGCACAA | | | | | | |
| | GGGAG | | | | | | |
| rs12191 | CTAATCCAAGCCGGC | 387 | CTAATCCAAGCCGGC | 1786 | 19 | 38500655 | + |
| 8594 | AAGGGTGAGGCCCTG | | AAGGGTGAGGCCCTG | | | | |
| | CGGATCCGCGCCATC | | CGGATCCGCGCCATC | | | | |
| | CTCCA | | CTCCG | | | | |
| rs11819 | ACGTCCAGCACGTGC | 388 | ACGTCCAGCACGTGC | 1787 | 19 | 38500898 | − |
| 2178 | AGCAAGAAGTCCTGG | | AGCAAGAAGTCCTGG | | | | |
| | TTCTCGATGCCATAC | | TTCTCGATGCCATAC | | | | |
| | ACACC | | ACACG | | | | |
| rs11819 | ACGTCCAGCACGTGC | 389 | ACGTCCAGCACGTGC | 1788 | 19 | 38500898 | − |
| 2178 | AGCAAGAAGTCCTGG | | AGCAAGAAGTCCTGG | | | | |
| | TTCTCGATGCCATAC | | TTCTCGATGCCATAC | | | | |
| | ACACC | | ACACG | | | | |
| rs11819 | ACGTCCAGCACGTGC | 390 | ACGTCCAGCACGTGC | 1789 | 19 | 38500898 | − |
| 2178 | AGCAAGAAGTCCTGG | | AGCAAGAAGTCCTGG | | | | |
| | TTCTCGATGCCATAC | | TTCTCGATGCCATAC | | | | |
| | ACACA | | ACACG | | | | |
| rs19392 | AGCATCCTTCGTGCC | 391 | | | 19 | 38500899 | + |
| 2818 | GGACCACAAGGCRTC | | | | | | |
| | CATGGTGCTCTTCCT | | | | | | |
| | GGACC | | | | | | |
| rs19392 | AGCATCCTTCGTGCC | 392 | | | 19 | 38500899 | + |
| 2818 | GGACCACAAGGCGTC | | | | | | |
| | CATGGTGCTCTTCCT | | | | | | |
| | GGACC | | | | | | |
| rs19392 | AGCATCCTTCGTGCC | 393 | | | 19 | 38500899 | + |
| 2818 | GGACCACAAGGCGTC | | | | | | |
| | CATGGTGCTCTTCCT | | | | | | |
| | GGACC | | | | | | |
| rs19392 | TCGTGAAGGCTGGCC | 394 | | | 19 | 38512321 | + |
| 2832 | TCCGCTCCTTCTTCG | | | | | | |
| | AGAGTGCCTCGGAGG | | | | | | |
| | ACATC | | | | | | |
| rs19392 | TCGTGAAGGCTGGCC | 395 | | | 19 | 38512321 | + |
| 2832 | TCCGCTCCTTCTTCG | | | | | | |
| | AGAGTGCCTCGGAGG | | | | | | |
| | ACATC | | | | | | |
| rs19392 | CGGGAACCAGCAGAG | 396 | | | 19 | 38543832 | + |
| 2843 | CCTGGCGCACAGTCG | | | | | | |
| | CCTATGGGACGCAGT | | | | | | |
| | GGTGG | | | | | | |
| rs19392 | CGGGAACCAGCAGAG | 397 | | | 19 | 38543832 | + |
| 2843 | CCTGGCGCACAGTCG | | | | | | |
| | CCTATGGGACGCAGT | | | | | | |
| | GGTGG | | | | | | |
| rs19392 | CTCGAACCTGAGCGA | 398 | | | 19 | 38543832 | − |
| 2843 | GCTTCATCATCATGT | | | | | | |
| | GGGCGAACACGTGCA | | | | | | |
| | GGAAT | | | | | | |
| rs11819 | GGGCAGCAAAGAAGA | 399 | | | 19 | 38580004 | − |
| 2167 | AGTTGTTGTAGTGTC | | | | | | |
| | CCAAGAGGGACATCA | | | | | | |
| | CCATA | | | | | | |
| rs11819 | CCTGACCCCTGGCCC | 400 | | | 19 | 38580004 | + |
| 2167 | TGTGTGCCCACAGTC | | | | | | |
| | CTTCCTGTACCTGGG | | | | | | |
| | CTGGT | | | | | | |
| rs12191 | CAGGTCCTCCCCACA | 401 | | | 19 | 38580094 | − |
| 8595 | CCTGTTTCCCATTGT | | | | | | |
| | GGGTGACAGAGGACA | | | | | | |
| | GGATG | | | | | | |
| rs12191 | CAGGTCCTCCCCACA | 402 | | | 19 | 38580094 | − |
| 8595 | CCTGTTTCCCATTGT | | | | | | |
| | GGGTGACAGAGGACA | | | | | | |
| | GGATG | | | | | | |
| rs19392 | ACATCGCCATGGGGG | 403 | | | 19 | 38580114 | + |
| 2876 | TCAAGACGCTGCGCA | | | | | | |
| | YCATCCTGTCCTCTG | | | | | | |
| | TCACC | | | | | | |
| rs19392 | ACATCGCCATGGGGG | 404 | | | 19 | 38580114 | + |
| 2876 | TCAAGACGCTGCGCA | | | | | | |
| | YCATCCTGTCCTCTG | | | | | | |
| | TCACC | | | | | | |
| rs19392 | ACATCGCCATGGGGG | 405 | | | 19 | 38580114 | + |
| 2876 | TCAAGACGCTGCGCA | | | | | | |
| | CCATCCTGTCCTCTG | | | | | | |
| | TCACC | | | | | | |
| rs19392 | ACGGTGTACAGGTAG | 406 | ACGGTGTACAGGTAG | 1790 | 19 | 38580370 | − |
| 2878 | ACGACCACCGCCAGA | | ACGACCACCGCCAGA | | | | |
| | AGGCCCACGGTCATC | | AGGCCCACGGTCATC | | | | |
| | ACCAC | | ACCAG | | | | |
| rs19392 | ACGGTGTACAGGTAG | 407 | ACGGTGTACAGGTAG | 1791 | 19 | 38580370 | − |
| 2878 | ACGACCACCGCCAGA | | ACGACCACCGCCAGA | | | | |
| | AGGCCCACGGTCATC | | AGGCCCACGGTCATC | | | | |
| | ACCAC | | ACCAG | | | | |
| rs19392 | ACGGTGTACAGGTAG | 408 | ACGGTGTACAGGTAG | 1792 | 19 | 38580370 | − |
| 2878 | ACGACCACCGCCAGA | | ACGACCACCGCCAGA | | | | |
| | AGGCCCACGGTCATC | | AGGCCCACGGTCATC | | | | |
| | ACCAC | | ACCAG | | | | |
| rs11819 | CCCCATCCTGCCCCC | 409 | | | 19 | 38580403 | + |
| 2168 | AGCTGGTGATGACCG | | | | | | |
| | TGGGCCTTCTGGCGG | | | | | | |
| | TGGTC | | | | | | |
| rs11819 | CCCCATCCTGCCCCC | 410 | | | 19 | 38580403 | + |
| 2168 | AGCTGGTGATGACCG | | | | | | |
| | TGGGCCTTCTGGCGG | | | | | | |
| | TGGTC | | | | | | |
| rs11819 | TCAGTGTTACCTGTT | 411 | | | 19 | 38584989 | + |
| 2170 | TCACATGTACGTGGG | | | | | | |
| | TGTCCGGGCTGGCGG | | | | | | |
| | AGGCA | | | | | | |
| rs11819 | TCAGTGTTACCTGTT | 412 | | | 19 | 38584989 | + |
| 2170 | TCACATGTACGTGGG | | | | | | |
| | TGTCCGGGCTGGCGG | | | | | | |
| | AGGCA | | | | | | |
| rs11819 | CCCTGTAGAGCTCGT | 413 | | | 19 | 38584989 | − |
| 2170 | ATTCGTCACCCGCGG | | | | | | |
| | GGTCCTCGATCTCGT | | | | | | |
| | CCCCA | | | | | | |
| rs11819 | GGTGGACCATGCACC | 414 | | | 19 | 38934851 | + |
| 2161 | CAGCCTCCAAGCAGA | | | | | | |
| | GGTCTGAAGGAGAAA | | | | | | |
| | AGGTC | | | | | | |
| rs11819 | GGCAGGTTGCTCACT | 415 | GGCAGGTTGCTCACT | 1793 | 19 | 38942490 | − |
| 2116 | TGATGAACTGGTTGT | | TGATGAACTGGTTGT | | | | |
| | ATAGGCCATTGGTGC | | ATAGGCCATTGGTGC | | | | |
| | TGTGG | | TGTGC | | | | |
| rs14433 | GCTTGCTGACCAGCC | 416 | | | 19 | 38946112 | − |
| 6148 | AGTCCAAGTTTGTGG | | | | | | |
| | AGAAGAGGGCACAGT | | | | | | |
| | TGCTA | | | | | | |
| rs11819 | CGTCCTCCGTGGAAG | 417 | | | 19 | 38985204 | + |
| 2175 | ACACCATGAGCCTGC | | | | | | |
| | TCGAGTGCCTCGGCC | | | | | | |
| | AGATC | | | | | | |
| rs11819 | CGTCCTCCGTGGAAG | 418 | | | 19 | 38985204 | + |
| 2175 | ACACCATGAGCCTGC | | | | | | |
| | TCGAGTGCCTCGGCC | | | | | | |
| | AGATC | | | | | | |
| rs11256 | AGACATTGGCTGGAA | 419 | | | 19 | 38989863 | + |
| 3513 | CCCCTGTGGTGGAGA | | | | | | |
| | GCGCTACCTGGACTT | | | | | | |
| | CCTGC | | | | | | |
| rs12191 | GCTCATGAGACCCCC | 420 | | | 19 | 38990286 | + |
| 8596 | TTTCCCCATGCGGGT | | | | | | |
| | GGCCAGGCGAGAGCG | | | | | | |
| | TGGAG | | | | | | |
| rs12191 | GCTCATGAGACCCCC | 421 | | | 19 | 38990286 | + |
| 8596 | TTTCCCCATGCGGGT | | | | | | |
| | GGCCAGGCGAGAGCG | | | | | | |
| | TGGAG | | | | | | |
| rs12191 | GCTCATGAGACCCCC | 422 | | | 19 | 38990286 | + |
| 8596 | TTTCCCCATGCGGGT | | | | | | |
| | GGCCAGGCGAGAGCG | | | | | | |
| | TGGAG | | | | | | |
| rs11819 | TGATGATGCCCACAA | 423 | | | 19 | 38991283 | − |
| 2122 | GGTCCTCCAAGGGCA | | | | | | |
| | CAAGGGAGCGGAGGA | | | | | | |
| | TGGCG | | | | | | |
| rs19392 | TCGTGAAGGCTGGCC | 424 | | | 19 | 39002961 | + |
| 2832 | TCCGCTCCTTCTTCG | | | | | | |
| | AGAGTGCCTCGGAGG | | | | | | |
| | ACATC | | | | | | |
| rs11819 | GGGCAGCAAAGAAGA | 425 | | | 19 | 39070644 | − |
| 2167 | AGTTGTTGTAGTGTC | | | | | | |
| | CCAAGAGGGACATCA | | | | | | |
| | CCATA | | | | | | |
| rs11819 | GGGCAGCAAAGAAGA | 426 | | | 19 | 39070644 | − |
| 2167 | AGTTGTTGTAGTGTC | | | | | | |
| | CCAAGAGGGACATCA | | | | | | |
| | CCATA | | | | | | |
| rs12191 | CAGGTCCTCCCCACA | 427 | | | 19 | 39070734 | − |
| 8595 | CCTGTTTCCCATTGT | | | | | | |
| | GGGTGACAGAGGACA | | | | | | |
| | GGATG | | | | | | |
| rs11819 | CCCCATCCTGCCCCC | 428 | | | 19 | 39071043 | + |
| 2168 | AGCTGGTGATGACCG | | | | | | |
| | TGGGCCTTCTGGCGG | | | | | | |
| | TGGTC | | | | | | |
| rs63749 | CACACTTCATGTCAG | 429 | | | 19 | 39071080 | − |
| 869 | GTTCATCCTCATCCT | | | | | | |
| | CGCTCTTGTTGTAGA | | | | | | |
| | ACTTG | | | | | | |
| rs63749 | CACACTTCATGTCAG | 430 | | | 19 | 39071080 | − |
| 869 | GTTCATCCTCATCCT | | | | | | |
| | CGCTCTTGTTGTAGA | | | | | | |
| | ACTTG | | | | | | |
| rs63749 | CACACTTCATGTCAG | 431 | | | 19 | 39071080 | − |
| 869 | GTTCATCCTCATCCT | | | | | | |
| | CGCTCTTGTTGTAGA | | | | | | |
| | ACTTG | | | | | | |
| rs12979 | GGGCGAGGGGCTTTG | 432 | | | 19 | 39248147 | − |
| 860 | CTGGGGGAGCGCGGA | | | | | | |
| | GTGCAATTCAACCCT | | | | | | |
| | GGTTC | | | | | | |
| rs12979 | GGGCGAGGGGCTTTG | 433 | | | 19 | 39248147 | − |
| 860 | CTGGGGGAGCGCGGA | | | | | | |
| | GTGCAATTCAACCCT | | | | | | |
| | GGTTC | | | | | | |
| rs12979 | GGGCGAGGGGCTTTG | 434 | | | 19 | 39248147 | − |
| 860 | CTGGGGGAGCGCGGA | | | | | | |
| | GTGCAATTCAACCCT | | | | | | |
| | GGTTC | | | | | | |
| rs34223 | CAGCCTCCAACTGGG | 435 | | | 19 | 40991224 | − |
| 104 | CCTTTTATCCTGACC | | | | | | |
| | CTGCCTCTGCACCCT | | | | | | |
| | GTTAT | | | | | | |
| rs34223 | CAGCCTCCAACTGGG | 436 | | | 19 | 40991224 | − |
| 104 | CCTTTTATCCTGACC | | | | | | |
| | CTGCCTCTGCACCCT | | | | | | |
| | GTTAT | | | | | | |
| rs34223 | CAGCCTCCAACTGGG | 437 | | | 19 | 40991224 | − |
| 104 | CCTTTTATCCTGACC | | | | | | |
| | CTGCCTCTGCACCCT | | | | | | |
| | GTTAT | | | | | | |
| rs81927 | GGGCAGAGGGCGGGG | 438 | | | 19 | 40991369 | − |
| 09 | CCCTGGTGGGAGGCG | | | | | | |
| | GYCATGRGWGTTAGG | | | | | | |
| | GTGGC | | | | | | |
| rs81927 | GGGCAGAGGGCGGGG | 439 | | | 19 | 40991369 | − |
| 09 | CCCTGGTGGGAGGCG | | | | | | |
| | GYCATGRGWGTTAGG | | | | | | |
| | GTGGC | | | | | | |
| rs81927 | GGGCAGAGGGCGGGG | 440 | | | 19 | 40991369 | − |
| 09 | CCCTGGTGGGAGGCG | | | | | | |
| | GTCATGGGTGTTAGG | | | | | | |
| | GTGGC | | | | | | |
| rs33973 | TGCACTCCTCACAGG | 441 | TGCACTCCTCACAGG | 1794 | 19 | 40991381 | + |
| 337 | ACTCTTGCTACTCCT | | ACTCTTGCTACTCCT | | | | |
| | GGTTCAGCGCCACCC | | GGTTCAGCGCCACCC | | | | |
| | TAACA | | TAACT | | | | |
| rs33973 | TGCACTCCTCACAGG | 442 | TGCACTCCTCACAGG | 1795 | 19 | 40991381 | + |
| 337 | ACTCTTGCTACTCCT | | ACTCTTGCTACTCCT | | | | |
| | GGTTCAGCGCCACCC | | GGTTCAGCGCCACCC | | | | |
| | TAACA | | TAACT | | | | |
| rs33973 | TGCACTCCTCACAGG | 443 | TGCACTCCTCACAGG | 1796 | 19 | 40991381 | + |
| 337 | ACTCTTGCTACTCCT | | ACTCTTGCTACTCCT | | | | |
| | GGTTCAGCGCCACCC | | GGTTCAGCGCCACCC | | | | |
| | TAACA | | TAACT | | | | |
| rs33980 | GCAGAAGGTTTCCCA | 444 | | | 19 | 40991388 | − |
| 385 | AAAGGGGCAGAGGGC | | | | | | |
| | GGGGCCCTGGTGGGA | | | | | | |
| | GGSKG | | | | | | |
| rs33980 | CAGAAGGTTTCCCAA | 445 | CAGAAGGTTTCCCAA | 1797 | 19 | 40991388 | − |
| 385 | AAGGGGCAGAGGGCG | | AAGGGGCAGAGGGCG | | | | |
| | GGGCCCTGGTGGGAG | | GGGCCCTGGTGGGAG | | | | |
| | GSKGT | | GSKGC | | | | |
| rs33980 | ACAGGACTCTTGCTA | 446 | ACAGGACTCTTGCTA | 1798 | 19 | 40991388 | + |
| 385 | CTCCTGGTTCAGCGC | | CTCCTGGTTCAGCGC | | | | |
| | CACCCTAACWCYCAT | | CACCCTAACWCYCAT | | | | |
| | GRCCG | | GGCAC | | | | |
| rs33926 | TGCAGAAGGTTTCCC | 447 | TGCAGAAGGTTTCCC | 1799 | 19 | 40991390 | − |
| 104 | AAAAGGGGCAGAGGG | | AAAAGGGGCAGAGGG | | | | |
| | CGGGGCCCTGGTGGG | | CGGGGCCCTGGTGGG | | | | |
| | AGGST | | AGGSG | | | | |
| rs33926 | TGCAGAAGGTTTCCC | 448 | TGCAGAAGGTTTCCC | 1800 | 19 | 40991390 | − |
| 104 | AAAAGGGGCAGAGGG | | AAAAGGGGCAGAGGG | | | | |
| | CGGGGCCCTGGTGGG | | CGGGGCCCTGGTGGG | | | | |
| | AGGBT | | AGGBG | | | | |
| rs33926 | TGCAGAAGGTTTCCC | 449 | TGCAGAAGGTTTCCC | 1801 | 19 | 40991390 | − |
| 104 | AAAAGGGGCAGAGGG | | AAAAGGGGCAGAGGG | | | | |
| | CGGGGCCCTGGTGGG | | CGGGGCCCTGGTGGG | | | | |
| | AGGBT | | AGGBG | | | | |
| rs34284 | ACAGGACTCTTGCTA | 450 | ACAGGACTCTTGCTA | 1802 | 19 | 40991391 | + |
| 776 | CTCCTGGTTCAGCGC | | CTCCTGGTTCAGCGC | | | | |
| | CACCCTAACACCCAT | | CACCCTAACACCCAT | | | | |
| | GACCG | | GACCC | | | | |
| rs34284 | CTGCAGAAGGTTTCC | 451 | CTGCAGAAGGTTTCC | 1803 | 19 | 40991391 | − |
| 776 | CAAAAGGGGCAGAGG | | CAAAAGGGGCAGAGG | | | | |
| | GCGGGGCCCTGGTGG | | GCGGGGCCCTGGTGG | | | | |
| | GAGGC | | GAGGG | | | | |
| rs34284 | CTGCAGAAGGTTTCC | 452 | CTGCAGAAGGTTTCC | 1804 | 19 | 40991391 | − |
| 776 | CAAAAGGGGCAGAGG | | CAAAAGGGGCAGAGG | | | | |
| | GCGGGGCCCTGGTGG | | GCGGGGCCCTGGTGG | | | | |
| | GAGGC | | GAGGG | | | | |
| rs35303 | CGTCTGTGTCTTACC | 453 | CGTCTGTGTCTTACC | 1805 | 19 | 40991441 | − |
| 484 | CTCAGAAAGGATTTG | | CTCAGAAAGGATTTG | | | | |
| | AGTAGGCCTCTTCTA | | AGTAGGCCTCTTCTA | | | | |
| | TCCAT | | TCCAC | | | | |
| rs35303 | CGTCTGTGTCTTACC | 454 | CGTCTGTGTCTTACC | 1806 | 19 | 40991441 | − |
| 484 | CTCAGAAAGGATTTG | | CTCAGAAAGGATTTG | | | | |
| | AGTAGGCCTCTTCTA | | AGTAGGCCTCTTCTA | | | | |
| | TCCAT | | TCCAC | | | | |
| rs35303 | CGTCTGTGTCTTACC | 455 | CGTCTGTGTCTTACC | 1807 | 19 | 40991441 | − |
| 484 | CTCAGAAAGGATTTG | | CTCAGAAAGGATTTG | | | | |
| | AGTAGGCCTCTTCTA | | AGTAGGCCTCTTCTA | | | | |
| | TCCAT | | TCCAC | | | | |
| rs28186 | CTAACAGCCACCCCT | 456 | CTAACAGCCACCCCT | 1808 | 19 | 41004015 | + |
| 4907 | GGTGTGGATGTGATT | | GGTGTGGATGTGATT | | | | |
| | GGCAGTTCCGAGAGA | | GGCAGTTCCGAGAGA | | | | |
| | AATAA | | AATAT | | | | |
| rs28186 | CTAACAGCCACCCCT | 457 | CTAACAGCCACCCCT | 1809 | 19 | 41004015 | + |
| 4907 | GGTGTGGATGTGATT | | GGTGTGGATGTGATT | | | | |
| | GGCAGTTCCGAGAGA | | GGCAGTTCCGAGAGA | | | | |
| | AATAA | | AATAT | | | | |
| rs28186 | CTAACAGCCACCCCT | 458 | CTAACAGCCACCCCT | 1810 | 19 | 41004015 | + |
| 4907 | GGTGTGGATGTGATT | | GGTGTGGATGTGATT | | | | |
| | GGCAGTTCCGAGAGA | | GGCAGTTCCGAGAGA | | | | |
| | AATAA | | AATAT | | | | |
| rs14265 | TGGTGTGGATGTGAT | 459 | | | 19 | 41004030 | + |
| 7251 | TGGCAGTTCCGAGAG | | | | | | |
| | AAATAWGGGGACGTC | | | | | | |
| | TTCAC | | | | | | |
| rs14265 | TGGTGTGGATGTGAT | 460 | | | 19 | 41004030 | + |
| 7251 | TGGCAGTTCCGAGAG | | | | | | |
| | AAATATGGGGACGTC | | | | | | |
| | TTCAC | | | | | | |
| rs14265 | TGGTGTGGATGTGAT | 461 | | | 19 | 41004030 | + |
| 7251 | TGGCAGTTCCGAGAG | | | | | | |
| | AAATATGGGGACGTC | | | | | | |
| | TTCAC | | | | | | |
| rs14940 | TCGACCATGGCGATT | 462 | | | 19 | 41004093 | − |
| 3002 | TTTCCCCGGCCAGAG | | | | | | |
| | AAGGCCTCAGCCTTG | | | | | | |
| | TCCAC | | | | | | |
| rs14940 | TCGACCATGGCGATT | 463 | | | 19 | 41004093 | − |
| 3002 | TTTCCCCGGCCAGAG | | | | | | |
| | AAGGCCTCAGCCTTG | | | | | | |
| | TCCAC | | | | | | |
| rs36060 | TGAGGCTCTCACCAT | 464 | | | 19 | 41004125 | − |
| 847 | ATCCCCGGAAGAATG | | | | | | |
| | GGTCGACCATGGCGA | | | | | | |
| | TTTTT | | | | | | |
| rs36060 | TGAGGCTCTCACCAT | 465 | | | 19 | 41004125 | − |
| 847 | ATCCCCGGAAGAATG | | | | | | |
| | GGTCGACCATGGCGA | | | | | | |
| | TTTTT | | | | | | |
| rs36060 | TGAGGCTCTCACCAT | 466 | | | 19 | 41004125 | − |
| 847 | ATCCCCGGAAGAATG | | | | | | |
| | GGTCGACCATGGCGA | | | | | | |
| | TTTTT | | | | | | |
| rs18633 | GGCCTTCTCTGGCCG | 467 | | | 19 | 41004158 | + |
| 5453 | GGGAAAAATCGCCAT | | | | | | |
| | GGTCGACCCATTCTT | | | | | | |
| | CCGGG | | | | | | |
| rs18633 | GCCTTCTCTGGCCGG | 468 | GCCTTCTCTGGCCGG | 1811 | 19 | 41004158 | + |
| 5453 | GGAAAAATCGCCATG | | GGAAAAATCGCCATG | | | | |
| | GTCGACCCATTCTTC | | GTCGACCCATTCTTC | | | | |
| | CRGGT | | CRGGG | | | | |
| rs18633 | GCCTTCTCTGGCCGG | 469 | GCCTTCTCTGGCCGG | 1812 | 19 | 41004158 | + |
| 5453 | GGAAAAATCGCCATG | | GGAAAAATCGCCATG | | | | |
| | GTCGACCCATTCTTC | | GTCGACCCATTCTTC | | | | |
| | CRGGT | | CRGGG | | | | |
| rs13980 | TCCAACTTCTTCTAC | 470 | | | 19 | 41004303 | + |
| 1276 | AACCAACCCACACCT | | | | | | |
| | SCCCTGCACCCCAGG | | | | | | |
| | TGTGA | | | | | | |
| rs13980 | TCCAACTTCTTCTAC | 471 | | | 19 | 41004303 | − |
| 1276 | AACCAACCCACACCT | | | | | | |
| | SCCCTGCACCCCAGG | | | | | | |
| | TGTGA | | | | | | |
| rs13980 | GGTCACAGAGAATCG | 472 | GGTCACAGAGAATCG | 1813 | 19 | 41004303 | − |
| 1276 | CCGAAGCACCTTCCA | | CCGAAGCACCTTCCA | | | | |
| | GCGGTTTCCATTGGC | | GCGGTTTCCATTGGC | | | | |
| | AAAGA | | AAAGG | | | | |
| rs12721 | AGGTGCTTCGGCGAT | 473 | | | 19 | 41004377 | + |
| 655 | TCTCTGTGACCACTA | | | | | | |
| | TGAGGGACTTCGGGA | | | | | | |
| | TGGGA | | | | | | |
| rs12721 | AGGTGCTTCGGCGAT | 474 | | | 19 | 41004377 | + |
| 655 | TCTCTGTGACCACTA | | | | | | |
| | TGAGGGACTTCGGGA | | | | | | |
| | TGGGA | | | | | | |
| rs12721 | AGGTGCTTCGGCGAT | 475 | | | 19 | 41004377 | + |
| 655 | TCTCTGTGACCACTA | | | | | | |
| | TGAGGGACTTCGGGA | | | | | | |
| | TGGGA | | | | | | |
| rs14588 | AGGACTCACCCTTGG | 476 | AGGACTCACCCTTGG | 1814 | 19 | 41004406 | − |
| 4402 | ATTTCCGAAGCTCCT | | ATTTCCGAAGCTCCT | | | | |
| | CTATCAGACACTGAG | | CTATCAGACACTGAG | | | | |
| | CCTCA | | CCTCC | | | | |
| rs14588 | AGGACTCACCCTTGG | 477 | AGGACTCACCCTTGG | 1815 | 19 | 41004406 | − |
| 4402 | ATTTCCGAAGCTCCT | | ATTTCCGAAGCTCCT | | | | |
| | CTATCAGACACTGAG | | CTATCAGACACTGAG | | | | |
| | CCTCA | | CCTCC | | | | |
| rs14588 | AGGACTCACCCTTGG | 478 | AGGACTCACCCTTGG | 1816 | 19 | 41004406 | − |
| 4402 | ATTTCCGAAGCTCCT | | ATTTCCGAAGCTCCT | | | | |
| | CTATCAGACACTGAG | | CTATCAGACACTGAG | | | | |
| | CCTCA | | CCTCC | | | | |
| rs13865 | CTTTCTTTCCTATTC | 479 | | | 19 | 41004427 | − |
| 2715 | ATCCCCCAGGACTCA | | | | | | |
| | CCCTTGGATTTCCGA | | | | | | |
| | AGYTC | | | | | | |
| rs13865 | CTTTCTTTCCTATTC | 480 | | | 19 | 41004427 | − |
| 2715 | ATCCCCCAGGACTCA | | | | | | |
| | CCCTTGGATTTCCGA | | | | | | |
| | AGCTC | | | | | | |
| rs13865 | CTTTCTTTCCTATTC | 481 | | | 19 | 41004427 | − |
| 2715 | ATCCCCCAGGACTCA | | | | | | |
| | CCCTTGGATTTCCGA | | | | | | |
| | AGCTC | | | | | | |
| rs38267 | TCAGTCTGTGTCCTT | 482 | TCAGTCTGTGTCCTT | 1817 | 19 | 41006919 | + |
| 11 | GACCTGCTGCTTCTT | | GACCTGCTGCTTCTT | | | | |
| | CCTAGGGGCCCTCAT | | CCTAGGGGCCCTCAT | | | | |
| | GGACC | | GGACG | | | | |
| rs38267 | TCAGTCTGTGTCCTT | 483 | TCAGTCTGTGTCCTT | 1818 | 19 | 41006919 | + |
| 11 | GACCTGCTGCTTCTT | | GACCTGCTGCTTCTT | | | | |
| | CCTAGGGGCCCTCAT | | CCTAGGGGCCCTCAT | | | | |
| | GGACC | | GGACG | | | | |
| rs38267 | TCAGTCTGTGTCCTT | 484 | TCAGTCTGTGTCCTT | 1819 | 19 | 41006919 | + |
| 11 | GACCTGCTGCTTCTT | | GACCTGCTGCTTCTT | | | | |
| | CCTAGGGGCCCTCAT | | CCTAGGGGCCCTCAT | | | | |
| | GGACC | | GGACG | | | | |
| rs36056 | GTCTGTGTCCTTGAC | 485 | | | 19 | 41006923 | + |
| 539 | CTGCTGCTTCTTCCT | | | | | | |
| | AGGGGCCCTCATGGA | | | | | | |
| | CCCCA | | | | | | |
| rs36056 | GTCTGTGTCCTTGAC | 486 | | | 19 | 41006923 | + |
| 539 | CTGCTGCTTCTTCCT | | | | | | |
| | AGGGGCCCTCATGGA | | | | | | |
| | CCCCA | | | | | | |
| rs36056 | GTCTGTGTCCTTGAC | 487 | | | 19 | 41006923 | + |
| 539 | CTGCTGCTTCTTCCT | | | | | | |
| | AGGGGCCCTCATGGA | | | | | | |
| | CCCCA | | | | | | |
| rs37452 | ACCTGCTGCTTCTTC | 488 | | | 19 | 41006936 | + |
| 74 | CTAtGgGCCCTCaTG | | | | | | |
| | GACCCCACCTTCCTC | | | | | | |
| | TTCCA | | | | | | |
| rs37452 | ACCTGCaGCTTCTTC | 489 | | | 19 | 41006936 | + |
| 74 | CTAtGgGCCCTCaTG | | | | | | |
| | GACCaCACCTTCCTC | | | | | | |
| | TTCCA | | | | | | |
| rs37452 | GGAATCGTTTTCCAA | 490 | GGAATCGTTTTCCAA | 1820 | 19 | 41006936 | − |
| 74 | AGACGATGGAGCAGA | | AGACGATGGAGCAGA | | | | |
| | TGATGTTGGCGGTAA | | TGATGTTGGCGGTAA | | | | |
| | TGGAA | | TGGAC | | | | |
| rs37348 | TCAGCATCTTCAGGA | 491 | | | 19 | 41006968 | − |
| 9637 | ACTCTTGATCTTGGT | | | | | | |
| | AGTGGAATCGTTTTC | | | | | | |
| | CAAAG | | | | | | |
| rs37348 | CAGCATCTTCAGGAA | 492 | CAGCATCTTCAGGAA | 1821 | 19 | 41006968 | − |
| 9637 | CTCTTGATCTTGGTA | | CTCTTGATCTTGGTA | | | | |
| | GTGGAATCGTTTTCC | | GTGGAATCGTTTTCC | | | | |
| | AAAGA | | AAAGC | | | | |
| rs37348 | CAGCATCTTCAGGAA | 493 | CAGCATCTTCAGGAA | 1822 | 19 | 41006968 | − |
| 9637 | CTCTTGATCTTGGTA | | CTCTTGATCTTGGTA | | | | |
| | GTGGAAKCGTTTTCC | | GTGGAAKCGTTTTCC | | | | |
| | RAAGA | | RAAGC | | | | |
| rs36079 | GGCCGAATACAGAGC | 494 | | | 19 | 41007013 | − |
| 186 | TGATGAGTGAAAAAG | | | | | | |
| | TCTGGTAGAACAAGT | | | | | | |
| | TCAGC | | | | | | |
| rs36079 | GGCCGAATACAGAGC | 495 | | | 19 | 41007013 | − |
| 186 | TGATGAGTGAAAAAG | | | | | | |
| | TCTGGTAGAACAAGT | | | | | | |
| | TCAGC | | | | | | |
| rs36079 | GGCCGAATACAGAGC | 496 | | | 19 | 41007013 | − |
| 186 | TGATGAGTGAAAAAG | | | | | | |
| | TCTGGTAGAACAAGT | | | | | | |
| | TCAGC | | | | | | |
| rs22793 | TCCCAGACCCCACTT | 497 | | | 19 | 41009358 | − |
| 43 | TTTCCATGTGGAGCA | | | | | | |
| | GGTAGGTGTCGATGA | | | | | | |
| | GGTCC | | | | | | |
| rs22793 | TCCCAGACCCCACTT | 498 | | | 19 | 41009358 | − |
| 43 | TTTCCATGTGGAGCA | | | | | | |
| | GGTAGGTGTCGATGA | | | | | | |
| | GGTCC | | | | | | |
| rs22793 | TCCCAGACCCCACTT | 499 | | | 19 | 41009358 | − |
| 43 | TTTCCATGTGGAGCA | | | | | | |
| | GGTAGGTGTCGATGA | | | | | | |
| | GGTCC | | | | | | |
| rs13902 | CCCTGACCCTCCCCT | 500 | CCCTGACCCTCCCCT | 1823 | 19 | 41010006 | + |
| 9625 | TCCTTCCCTACTGTG | | TCCTTCCCTACTGTG | | | | |
| | GACGCAGGAGAAATC | | GACGCAGGAGAAATC | | | | |
| | CAACC | | CAACG | | | | |
| rs34698 | CTCAACACGCTCTCG | 501 | CTCAACACGCTCTCG | 1824 | 19 | 41010088 | − |
| 757 | CTCTTCTTTGCTGGC | | CTCTTCTTTGCTGGC | | | | |
| | ACTGAGACCACCAGC | | ACTGAGACCACCAGC | | | | |
| | ACCAG | | ACCAC | | | | |
| rs34698 | CTCAACACGCTCTCG | 502 | CTCAACACGCTCTCG | 1825 | 19 | 41010088 | + |
| 757 | CTCTTCTTTGCTGGC | | CTCTTCTTTGCTGGC | | | | |
| | ACTGAGACCACCAGC | | ACTGAGACCACCAGC | | | | |
| | ACCAG | | ACCAC | | | | |
| rs34698 | ACCTGCAACATGAGG | 503 | ACCTGCAACATGAGG | 1826 | 19 | 41010088 | − |
| 757 | GTATTTGAGCATGAG | | GTATTTGAGCATGAG | | | | |
| | CAGGAAGCCGTAGCG | | CAGGAAGCCGTAGCG | | | | |
| | GAGAC | | GAGAG | | | | |
| rs28399 | AATGAGATTCATTGG | 504 | | | 19 | 41012316 | + |
| 499 | TCTTCTTTTCTGTAC | | | | | | |
| | AGAGAGAGTCTACAG | | | | | | |
| | GGAGA | | | | | | |
| rs28399 | ATGAGATTCATTGGT | 505 | ATGAGATTCATTGGT | 1827 | 19 | 41012316 | + |
| 499 | CTTCTTTTCTGTACA | | CTTCTTTTCTGTACA | | | | |
| | GAGAGAGTCTACAGG | | GAGAGAGTCTACAGG | | | | |
| | GAGAT | | GAGAC | | | | |
| rs34826 | TCTGTACAGAGAGAG | 506 | | | 19 | 41012339 | + |
| 503 | TCTACAGGGAGATTG | | | | | | |
| | AACAGGTGATTGGCC | | | | | | |
| | CACAT | | | | | | |
| rs34826 | TCTGTACAGAGAGAG | 507 | | | 19 | 41012339 | + |
| 503 | TCTACAGGGAGATTG | | | | | | |
| | AACAGGTGATTGGCC | | | | | | |
| | CACAT | | | | | | |
| rs34826 | TCTGTACAGAGAGAG | 508 | | | 19 | 41012339 | + |
| 503 | TCTACAGGGAGATTG | | | | | | |
| | AACAGGTGATTGGCC | | | | | | |
| | CACAT | | | | | | |
| rs34097 | GGAGGGCTATGGGGT | 509 | | | 19 | 41012465 | − |
| 093 | TCCAGCCGGTCTTAC | | | | | | |
| | CTTGGGGATGATGTA | | | | | | |
| | CCCTC | | | | | | |
| rs34097 | GGAGGGCTATGGGGT | 510 | | | 19 | 41012465 | − |
| 093 | TCCAGCCGGTCTTAC | | | | | | |
| | CTTGGGGATGATGTA | | | | | | |
| | CCCTC | | | | | | |
| rs34097 | GGAGGGCTATGGGGT | 511 | | | 19 | 41012465 | − |
| 093 | TCCAGCCGGTCTTAC | | | | | | |
| | CTTGGGGATGATGTA | | | | | | |
| | CCCTC | | | | | | |
| rs35979 | ATATCTTTTGATCTT | 512 | ATATCTTTTGATCTT | 1828 | 19 | 41012693 | + |
| 566 | GTGATCCTCCCTCAG | | GTGATCCTCCCTCAG | | | | |
| | GACACAGAAGTATTT | | GACACAGAAGTATTT | | | | |
| | CTCAA | | CTCAT | | | | |
| rs35979 | ATATCTTTTGATCTT | 513 | ATATCTTTTGATCTT | 1829 | 19 | 41012693 | + |
| 566 | GTGATCCTCYCTCAG | | GTGATCCTCYCTCAG | | | | |
| | GACACAGAAGTATTT | | GACACAGAAGTATTT | | | | |
| | CTCAA | | CTCAT | | | | |
| rs35979 | ATATCTTTTGATCTT | 514 | ATATCTTTTGATCTT | 1830 | 19 | 41012693 | + |
| 566 | GTGATCCTCYCTCAG | | GTGATCCTCYCTCAG | | | | |
| | GACACAGAAGTATTT | | GACACAGAAGTATTT | | | | |
| | CTCAA | | CTCAT | | | | |
| rs56408 | GCCAGCCCCGTGGCC | 515 | GCCAGCCCCGTGGCC | 1831 | 19 | 41016778 | + |
| 3989 | CCAGAAGACATCGAT | | CCAGAAGACATCGAT | | | | |
| | CTGACACCCCAGGAG | | CTGACACCCCAGGAG | | | | |
| | TGTGA | | TGTGG | | | | |
| rs56408 | GCCAGCCCCGTGGCC | 516 | GCCAGCCCCGTGGCC | 1832 | 19 | 41016778 | + |
| 3989 | CCAGAAGACATCGAT | | CCAGAAGACATCGAT | | | | |
| | CTGACACCCCAGGAG | | CTGACACCCCAGGAG | | | | |
| | TGTGA | | TGTGG | | | | |
| rs56408 | GCCAGCCCCGTGGCC | 517 | GCCAGCCCCGTGGCC | 1833 | 19 | 41016778 | + |
| 3989 | CCAGAAGACATCGAT | | CCAGAAGACATCGAT | | | | |
| | CTGACACCCCAGGAG | | CTGACACCCCAGGAG | | | | |
| | TGTGA | | TGTGG | | | | |
| rs32113 | TGACACCCCAGGAGT | 518 | | | 19 | 41016810 | + |
| 71 | GTGGTGTGGGCAAAA | | | | | | |
| | TACCCCCAACATACC | | | | | | |
| | AGATC | | | | | | |
| rs32113 | TGGAATCCTTTGACC | 519 | TGGAATCCTTTGACC | 1834 | 19 | 41016810 | − |
| 71 | CCCTTCCCTCAGCCC | | CCCTTCCCTCAGCCC | | | | |
| | CTTCAGCGGGGCAGG | | CTTCAGCGGGGCAGG | | | | |
| | AAGCA | | AAGCG | | | | |
| rs32113 | TGGAATCCTTTGACC | 520 | TGGAATCCTTTGACC | 1835 | 19 | 41016810 | − |
| 71 | CCCTTCCCTCAGCCC | | CCCTTCCCTCAGCCC | | | | |
| | CTTCAGCGGGGCAGG | | CTTCAGCGGGGCAGG | | | | |
| | AAGCA | | AAGCG | | | | |
| rs28399 | AATGAGATTCATTGG | 521 | | | 19 | 41518221 | + |
| 499 | TCTTCTTTTCTGTAC | | | | | | |
| | AGAGAGAGTCTACAG | | | | | | |
| | GGAGA | | | | | | |
| rs88782 | TGTGTTCTTTTCTTT | 522 | | | 2 | 233759924 | + |
| 9 | CTAAAAGGCTTTCTA | | | | | | |
| | AAAAGCCTTCTGTTT | | | | | | |
| | AATTT | | | | | | |
| rs88782 | TGTGTTCTTTTCTTT | 523 | | | 2 | 233759924 | + |
| 9 | CTAAAAGGCTTTCTA | | | | | | |
| | AAAAGCCTTCTGTTT | | | | | | |
| | AATTT | | | | | | |
| rs81753 | AACATTAACTTGGTG | 524 | AACATTAACTTGGTG | 1836 | 2 | 233760234 | + |
| 47 | TATCGATTGGTTTTT | | TATCGATTGGTTTTT | | | | |
| | GCCATATATATATAT | | GCCATATATATATAT | | | | |
| | ATAAG | | ATATA | | | | |
| rs81753 | AACATTAACTTGGTG | 525 | AACATTAACTTGGTG | 1837 | 2 | 233760234 | + |
| 47 | TATCGATTGGTTTTT | | TATCGATTGGTTTTT | | | | |
| | GCCATATATATATAT | | GCCATATATATATAT | | | | |
| | ATAAG | | ATATA | | | | |
| rs81753 | CCTTTGCTCCTGCCA | 526 | | | 2 | 233760234 | − |
| 47 | GAGGTTCGCCCTCTC | | | | | | |
| | CTACTTATATATATA | | | | | | |
| | TATAT | | | | | | |
| rs41483 | ATCCTCCCTTTGGAA | 527 | | | 2 | 233760498 | − |
| 23 | TGGCACAGGGTACGT | | | | | | |
| | CTTCAAGGTGTAAAA | | | | | | |
| | TGCTC | | | | | | |
| rs41483 | GACATGAAATAGTTG | 528 | | | 2 | 233760498 | + |
| 23 | TCCTAGCACCTGACG | | | | | | |
| | CCTCGTTGTACATCA | | | | | | |
| | GAGAC | | | | | | |
| rs35350 | ATGCTCATTGCCTTT | 529 | ATGCTCATTGCCTTT | 1838 | 2 | 233760973 | + |
| 960 | TCACAGAACTTTCTG | | TCACAGAACTTTCTG | | | | |
| | TGCGACGTGGTTTAT | | TGCGACGTGGTTTAT | | | | |
| | TCCCA | | TCCCC | | | | |
| rs35350 | CTGGACAGTCACCTC | 530 | CTGGACAGTCACCTC | 1839 | 2 | 233760973 | − |
| 960 | TCTCTGAAGGAATTC | | TCTCTGAAGGAATTC | | | | |
| | TGAGGCAAGGGTTGC | | TGAGGCAAGGGTTGC | | | | |
| | ATACT | | ATACG | | | | |
| rs35350 | ATGCTCATTGCCTTT | 531 | ATGCTCATTGCCTTT | 1840 | 2 | 233760973 | + |
| 960 | TCACAGAACTTTCTG | | TCACAGAACTTTCTG | | | | |
| | TGCGACGTGGTTTAT | | TGCGACGTGGTTTAT | | | | |
| | TCCCA | | TCCCC | | | | |
| rs88782 | AGAGAGGAAGAAGGA | 532 | | | 2 | 234668570 | − |
| 9 | CGACTATGTAGTGAA | | | | | | |
| | CAAGTTAGGCTTCTT | | | | | | |
| | TTCCA | | | | | | |
| rs41483 | GACATGAAATAGTTG | 533 | | | 2 | 234669144 | + |
| 23 | TCCTAGCACCTGACG | | | | | | |
| | CCTCGTTGTACATCA | | | | | | |
| | GAGAC | | | | | | |
| 22_ | TTAGAGCCTCTGGCT | 534 | | | 22 | 42126578 | + |
| 42126578 | AGGGAGCAGGCTGGG | | | | | | |
| | GACTAGGTACCCCAT | | | | | | |
| | TCTAG | | | | | | |
| 22_ | CTTTGCTTTCCTGGT | 535 | | | 22 | 42126578 | − |
| 42126578 | GAGCCCATCCCCCTA | | | | | | |
| | TGAGCTTTGTGCTGT | | | | | | |
| | GCCCC | | | | | | |
| 22_ | CTTTGCTTTCCTGGT | 536 | | | 22 | 42126578 | − |
| 42126578 | GAGCCCATCCCCCTR | | | | | | |
| | YGAGCTTTGTGCTGT | | | | | | |
| | GCCCC | | | | | | |
| 22_ | TTTGCTTTCCTGGTG | 537 | TTTGCTTTCCTGGTG | 1841 | 22 | 42126578 | − |
| 42126578 | AGCCCATCCCCCTAT | | AGCCCATCCCCCTAT | | | | |
| | GAGCTTTGTGCTGTG | | GAGCTTTGTGCTGTG | | | | |
| | CCCCA | | CCCCG | | | | |
| 22_ | TTTGCTTTCCTGGTG | 538 | TTTGCTTTCCTGGTG | 1842 | 22 | 42126578 | − |
| 42126578 | AGCCCATCCCCCTRY | | AGCCCATCCCCCTRY | | | | |
| | GAGCTTTGTGCTGTG | | GAGCTTTGTGCTGTG | | | | |
| | CCCCA | | CCCCG | | | | |
| rs56849 | ACAGCCCCGGCCCAG | 539 | | | 22 | 42126605 | − |
| 5591 | CCACCATGGTGTCTT | | | | | | |
| | TGCYTTYCTGGTGAS | | | | | | |
| | CCCAT | | | | | | |
| rs56849 | CAGCCCCGGCCCAGC | 540 | CAGCCCCGGCCCAGC | 1843 | 22 | 42126605 | − |
| 5591 | CACCATGGTGTCTTT | | CACCATGGTGTCTTT | | | | |
| | GCTTTCCTGGTGAGC | | GCTTTCCTGGTGAGC | | | | |
| | CCATT | | CCATC | | | | |
| rs56849 | CAGCCCCGGCCCAGC | 541 | CAGCCCCGGCCCAGC | 1844 | 22 | 42126605 | − |
| 5591 | CACCATGGTGTCTTT | | CACCATGGTGTCTTT | | | | |
| | GCTTTCCTGGTGAGC | | GCTTTCCTGGTGAGC | | | | |
| | CCATT | | CCATC | | | | |
| rs75467 | TTCTCGGTGCCCACT | 542 | TTCTCGGTGCCCACT | 1845 | 22 | 42126623 | − |
| 367 | GGACAGCCCCGGCCC | | GGACAGCCCCGGCCC | | | | |
| | AGCCACCATGGTGTC | | AGCCACCATGGTGTC | | | | |
| | TTTGG | | TTTGC | | | | |
| rs75467 | TTCTCGGTGCCCACT | 543 | TTCTCGGTGCCCACT | 1846 | 22 | 42126623 | − |
| 367 | GGACAGCCCCGGCCC | | GGACAGCCCCGGCCC | | | | |
| | AGCCACCATGGTGTC | | AGCCACCATGGTGTC | | | | |
| | TTTGG | | TTTGC | | | | |
| rs75467 | TTCTCGGTGCCCACT | 544 | TTCTCGGTGCCCACT | 1847 | 22 | 42126623 | − |
| 367 | GGACAGCCCCGGCCC | | GGACAGCCCCGGCCC | | | | |
| | AGCCACCATGGTGTC | | AGCCACCATGGTGTC | | | | |
| | TTTGG | | TTTGC | | | | |
| rs74478 | GCTTCTCGGTGCCCA | 545 | | | 22 | 42126624 | − |
| 221 | CTGGACAGCCCCGGC | | | | | | |
| | CCAGCCACCATGGTG | | | | | | |
| | TCTTT | | | | | | |
| rs74478 | GCTTCTCGGTGCCCA | 546 | | | 22 | 42126624 | − |
| 221 | CTGGACAGCCCCGGC | | | | | | |
| | CCAGCCACCATGGTG | | | | | | |
| | TCTTT | | | | | | |
| rs74478 | GCTTCTCGGTGCCCA | 547 | | | 22 | 42126624 | − |
| 221 | CTGGACAGCCCCGGC | | | | | | |
| | CCAGCCACCATGGTG | | | | | | |
| | TCTTT | | | | | | |
| rs11358 | CGGGGCACAGCACAA | 548 | CGGGGCACAGCACAA | 1849 | 22 | 42126627 | + |
| 38 | AGCTCATAGGGGGAT | | AGCTCATAGGGGGAT | | | | |
| | GGGCTCACCAGGAAA | | GGGCTCACCAGGAAA | | | | |
| | GCAAA | | GCAAC | | | | |
| rs11358 | GCGGGGCACAGCACA | 549 | | | 22 | 42126627 | + |
| 38 | AAGCTCATAGGGGGA | | | | | | |
| | TGGGCTCACCAGGAA | | | | | | |
| | AGCAA | | | | | | |
| rs11358 | GCGGGGCACAGCACA | 550 | | | 22 | 42126627 | + |
| 38 | AAGCTCATAGGGGGA | | | | | | |
| | TGGGCTCACCAGGAA | | | | | | |
| | AGCAA | | | | | | |
| rs11358 | ACAGCACAAAGCTCA | 551 | ACAGCACAAAGCTCA | 1850 | 22 | 42126633 | + |
| 37 | TAGGGGGATGGGCTC | | TAGGGGGATGGGCTC | | | | |
| | ACCAGGAAAGCAAAG | | ACCAGGAAAGCAAAG | | | | |
| | ACACC | | ACACG | | | | |
| rs11358 | ACAGCACAAAGCTCA | 552 | ACAGCACAAAGCTCA | 1851 | 22 | 42126633 | + |
| 37 | TAGGGGGATGGGCTC | | TAGGGGGATGGGCTC | | | | |
| | ACCAGGAAAGYRAMG | | ACCAGGAAAGYRAMG | | | | |
| | ACACC | | ACACG | | | | |
| rs11358 | GCACTTCAGCTTCTC | 553 | GCACTTCAGCTTCTC | 1852 | 22 | 42126633 | − |
| 37 | GGTGCCCACTGGACA | | GGTGCCCACTGGACA | | | | |
| | GCCCCGGCCCAGCCA | | GCCCCGGCCCAGCCA | | | | |
| | CCATG | | CCATC | | | | |
| 22_ | ACAGCACAAAGCTCA | 554 | | | 22 | 42126634 | + |
| 42126634 | TAGGGGGATGGGCTC | | | | | | |
| | ACCAGGAAAGCAAAG | | | | | | |
| | ACACC | | | | | | |
| 22_ | CAGCACTTCAGCTTC | 555 | | | 22 | 42126634 | − |
| 42126634 | TCGGTGCCCACTGGA | | | | | | |
| | CAGCCCCGGCCCAGC | | | | | | |
| | CACCA | | | | | | |
| 22_ | CAGCACTTCAGCTTC | 556 | | | 22 | 42126634 | − |
| 42126634 | TCGGTGCCCACTGGA | | | | | | |
| | CAGCCCCGGCCCAGC | | | | | | |
| | CACCA | | | | | | |
| rs76650 | CAGCACAAAGCTCAT | 557 | | | 22 | 42126635 | + |
| 7177 | AGGGGGATGGGGTCA | | | | | | |
| | CCAGGAAAGCAAAGA | | | | | | |
| | CACCA | | | | | | |
| rs76650 | GCAGCACTTCAGCTT | 558 | | | 22 | 42126635 | − |
| 7177 | CTCGGTGCCCACTGG | | | | | | |
| | ACAGCCCCGGCCCAG | | | | | | |
| | CCACC | | | | | | |
| rs76650 | GCAGCACTTCAGCTT | 559 | | | 22 | 42126635 | − |
| 7177 | CTCGGTGCCCACTGG | | | | | | |
| | ACAGCCCCGGCCCAG | | | | | | |
| | CCACC | | | | | | |
| rs28371 | AGCACAAAGCTCATA | 560 | | | 22 | 42126636 | + |
| 735 | GGGGGATGGGCTCAC | | | | | | |
| | CAGGAAAGCAAAGAC | | | | | | |
| | ACCAT | | | | | | |
| 22_ | CATAGGGGGATGGGC | 561 | | | 22 | 42126647 | + |
| 42126647 | TCACCAGGAAAGCAA | | | | | | |
| | AGACACCMKRGTGGC | | | | | | |
| | TGGGC | | | | | | |
| 22_ | ATAGGGGGATGGGCT | 562 | ATAGGGGGATGGGCT | 1853 | 22 | 42126647 | + |
| 42126647 | CACCAGGAAAGCAAA | | CACCAGGAAAGCAAA | | | | |
| | GACACCATGGTGGCT | | GACACCATGGTGGCT | | | | |
| | GGGCT | | GGGCC | | | | |
| 22_ | ATAGGGGGATGGGCT | 563 | ATAGGGGGATGGGCT | 1854 | 22 | 42126647 | + |
| 42126647 | CACCAGGAAAGCAAA | | CACCAGGAAAGCAAA | | | | |
| | GACACCMKRGTGGCT | | GACACCMKRGTGGCT | | | | |
| | GGGCT | | GGGCC | | | | |
| 22_ | ACCTCCCTGCTGCAG | 564 | ACCTCCCTGCTGCAG | 1855 | 22 | 42126647 | − |
| 42126647 | CACTTCAGCTTCTCG | | CACTTCAGCTTCTCG | | | | |
| | GTGCCCACTGGACAG | | GTGCCCACTGGACAG | | | | |
| | CCCCA | | CCCCG | | | | |
| 22_ | CATAGaGGGATGGgc | 565 | | | 22 | 42126647 | + |
| 42126647 | TCACCAGgAAagCaA | | | | | | |
| | aGACACcAtgGTGGC | | | | | | |
| | TGGGC | | | | | | |
| rs76577 | AGGAAAGCAAAGACA | 566 | | | 22 | 42126656 | + |
| 6661 | CCATGGTGGCTGGGC | | | | | | |
| | CGGGGCTGTCCAGTG | | | | | | |
| | GGCAC | | | | | | |
| rs11358 | CTCACCAGGAAAGCA | 567 | CTCACCAGGAAAGCA | 1856 | 22 | 42126660 | + |
| 35 | AAGACACCATGGTGG | | AAGACACCATGGTGG | | | | |
| | CTGGGCCGGGGCTGT | | CTGGGCCGGGGCTGT | | | | |
| | CCAGT | | CCAGC | | | | |
| rs76577 | AAAGCAAAGACACCA | 568 | AAAGCAAAGACACCA | 1857 | 22 | 42126666 | + |
| 6661 | TGGTGGCTGGGCCGG | | TGGTGGCTGGGCCGG | | | | |
| | GGCTGTCCAGTGGGC | | GGCTGTCCAGTGGGC | | | | |
| | ACCGA | | ACAGT | | | | |
| rs76577 | GCTCACCAGgAAagc | 569 | GCTCACCAGgAAagc | 1858 | 22 | 42126666 | + |
| 6661 | aAaGACACcAtgGTG | | aAaGACACcAtgGTG | | | | |
| | GCTGGGCCGGGGCTG | | GCTGGGCCGGGGCTG | | | | |
| | TCCaA | | TCCaG | | | | |
| 22_ | CCATGGTGGCTGGGC | 570 | CCATGGTGGCTGGGC | 1859 | 22 | 42126681 | + |
| 42126681 | CGGGGCTGTCCAGTG | | CGGGGCTGTCCAGTG | | | | |
| | GGCACCGAGAAGCTG | | GGCACCGAGAAGCTG | | | | |
| | AAGTG | | AAGTC | | | | |
| 22_ | CCATGGTGGCTGGGC | 571 | CCATGGTGGCTGGGC | 1860 | 22 | 42126681 | + |
| 42126681 | CGGGGCTGTCCAGTG | | CGGGGCTGTCCAGTG | | | | |
| | GGCACCGAGAAGCTG | | GGCACCGAGAAGCTG | | | | |
| | AAGTG | | AAGTC | | | | |
| 22_ | GCCCCTGGCCCGCAT | 572 | GCCCCTGGCCCGCAT | 1861 | 22 | 42126681 | − |
| 42126681 | GGAGCTCTTCCTCTT | | GGAGCTCTTCCTCTT | | | | |
| | CTTCACCTCCCTGCT | | CTTCACCTCCCTGCT | | | | |
| | GCAGC | | GCAGG | | | | |
| 22_ | GGGGCTGTCCAGTGG | 573 | GGGGCTGTCCAGTGG | 1862 | 22 | 42126697 | + |
| 42126697 | GCACCGAGAAGCTGA | | GCACCGAGAAGCTGA | | | | |
| | AGTGCTGCAGCAGGG | | AGTGCTGCAGCAGGG | | | | |
| | AGGTT | | AGGTG | | | | |
| 22_ | CGGGGCTGTCCAGTG | 574 | | | 22 | 42126697 | + |
| 42126697 | GGCACCGAGAAGCTG | | | | | | |
| | AAGTGCTGCAGCAGG | | | | | | |
| | GAGGT | | | | | | |
| 22_ | CGGGGCTGTCCAGTG | 575 | | | 22 | 42126697 | + |
| 42126697 | GGCACCGAGAAGCTG | | | | | | |
| | AAGTGCTGCAGCAGG | | | | | | |
| | GAGGT | | | | | | |
| rs36917 | AGAAGCTGAAGTGCT | 576 | | | 22 | 42126719 | + |
| 7208 | GCAGCAGGGAGGTGA | | | | | | |
| | AGAAGAGGAAGAGCT | | | | | | |
| | CCATG | | | | | | |
| rs36917 | AGAAGCTGAAGTGCT | 577 | | | 22 | 42126719 | + |
| 7208 | GCAGCAGGGAGGTKA | | | | | | |
| | AGAAGAGGAAGAGCT | | | | | | |
| | CCATG | | | | | | |
| rs36917 | GAAGCTGAAGTGCTG | 578 | GAAGCTGAAGTGCTG | 1863 | 22 | 42126719 | + |
| 7208 | CAGCAGGGAGGTKAA | | CAGCAGGGAGGTKAA | | | | |
| | GAAGAGGAAGAGCTC | | GAAGAGGAAGAGCTC | | | | |
| | CATGT | | CATGC | | | | |
| rs75109 | CAGCAGGGAGGTGAA | 579 | | | 22 | 42126735 | + |
| 2905 | GAAGAGGAAGAGCTC | | | | | | |
| | CATGCGGGCCAGGGG | | | | | | |
| | CTCCC | | | | | | |
| rs75109 | CAGCAGGGAGGTGAA | 580 | | | 22 | 42126735 | + |
| 2905 | GAAGAGGAAGAGCTC | | | | | | |
| | CATGCGGGCCAGGGG | | | | | | |
| | CTCCC | | | | | | |
| rs75109 | CAGCAGGGAGGTGAA | 581 | | | 22 | 42126735 | + |
| 2905 | GAAGAGGAAGAGCTC | | | | | | |
| | CATGCGGGCCAGGGG | | | | | | |
| | CTCCC | | | | | | |
| rs53266 | TGAAGAAGAGGAAGA | 582 | | | 22 | 42126746 | + |
| 8079 | GCTCCATGCGGGCCA | | | | | | |
| | GGGGCTCCCCGAGGC | | | | | | |
| | ATGCA | | | | | | |
| rs53266 | TGAAGAAGAGGAAGA | 583 | | | 22 | 42126746 | + |
| 8079 | GCTCCATGCGGGCCA | | | | | | |
| | GGGGCTCCCCGAGGC | | | | | | |
| | ATGCA | | | | | | |
| rs53266 | TGAAGAAGAGGAAGA | 584 | | | 22 | 42126746 | + |
| 8079 | GCTCCATGCGGGCCA | | | | | | |
| | GGGGCTCCCCGAGGC | | | | | | |
| | ATGCA | | | | | | |
| rs73088 | GAAGAAGAGGAAGAG | 585 | | | 22 | 42126747 | + |
| 2251 | CTCCATGCGGGCCAG | | | | | | |
| | GGGCTCCCCGAGGCA | | | | | | |
| | TGCAC | | | | | | |
| rs73088 | GAAGAAGAGGAAGAG | 586 | | | 22 | 42126747 | + |
| 2251 | CTCCATGCGGGCCAG | | | | | | |
| | GGGCTCCCCGAGGCA | | | | | | |
| | TGCAC | | | | | | |
| rs73088 | GAAGAAGAGGAAGAG | 587 | | | 22 | 42126747 | + |
| 2251 | CTCCATGCGGGaCAG | | | | | | |
| | aGGCTCaCCGAGGCA | | | | | | |
| | TGCAC | | | | | | |
| rs26760 | AGAAGAGGAAGAGCT | 588 | | | 22 | 42126749 | + |
| 8319 | CCATGCGGGCCAGGG | | | | | | |
| | GCTCCCCGAGGCATG | | | | | | |
| | CACGG | | | | | | |
| rs26760 | AGAAGAGtAAGAGCT | 589 | | | 22 | 42126749 | + |
| 8319 | CcATGCtGGtCAGcG | | | | | | |
| | GCTCCCCGAGGCATG | | | | | | |
| | CACGG | | | | | | |
| rs56943 | GGGGTATCACCCAGG | 590 | | | 22 | 42126752 | − |
| 9709 | AGCCAGGCTCACTGA | | | | | | |
| | CGCCCCTCCCCTCCC | | | | | | |
| | CACAG | | | | | | |
| rs56943 | GGGGTATCACCCAGG | 591 | | | 22 | 42126752 | − |
| 9709 | AGCCAGGCTCACTGA | | | | | | |
| | CGCCCCTCCCCTCCC | | | | | | |
| | CACAG | | | | | | |
| rs30210 | CCGCTTCCACCCCGA | 592 | | | 22 | 42126877 | − |
| 84 | ACACTTCCTGGATGC | | | | | | |
| | CYAGGGCCACTTTGT | | | | | | |
| | GAAGC | | | | | | |
| rs30210 | GGGAGCCGGGCTCCC | 593 | | | 22 | 42126877 | − |
| 84 | CACAGGCACCTGCTG | | | | | | |
| | AGAAAGGCAGGAAGG | | | | | | |
| | CCTCC | | | | | | |
| rs76396 | CCGTCTGGGAGAAGC | 594 | | | 22 | 42126896 | − |
| 4554 | CCTTCCGCTTCCACC | | | | | | |
| | CCGAACACTTCCTGG | | | | | | |
| | ATGCC | | | | | | |
| rs76396 | CCGTCTGGGAGAAGC | 595 | | | 22 | 42126896 | − |
| 4554 | CCTTCCGCTTCCACC | | | | | | |
| | CCGAACACTTCCTGG | | | | | | |
| | ATGCC | | | | | | |
| rs76396 | CCGTCTGGGAGAAGC | 596 | | | 22 | 42126896 | − |
| 4554 | CCTTCCGCTTCCACC | | | | | | |
| | CCGAACACTTCCTGG | | | | | | |
| | ATGCC | | | | | | |
| rs28371 | GGTGCTGAAGGATGA | 597 | GGTGCTGAAGGATGA | 1864 | 22 | 42126914 | − |
| 733 | GGCCGTCTGGGAGAA | | GGCCGTCTGGGAGAA | | | | |
| | GCCCTTCCGCTTCCA | | GCCCTTCCGCTTCCA | | | | |
| | CCCCC | | CCCCG | | | | |
| rs74708 | CGGCTTCACAAAGTG | 598 | | | 22 | 42126926 | + |
| 9665 | GCCCTGGGCATCCRG | | | | | | |
| | GAAGTGTTBGGGGTG | | | | | | |
| | GAAGC | | | | | | |
| rs74708 | CGGCTTCACAAAGTG | 599 | | | 22 | 42126926 | + |
| 9665 | GCCCTGGGCATCCAG | | | | | | |
| | GAAGTGTTCGGGGTG | | | | | | |
| | GAAGC | | | | | | |
| rs74708 | CGGCTTCACAAAGTG | 600 | | | 22 | 42126926 | + |
| 9665 | GCCCTGGGCATCCAG | | | | | | |
| | GAAGTGTTCGGGGTG | | | | | | |
| | GAAGC | | | | | | |
| 22_ | CATCTTCTGCCCAGG | 601 | | | 22 | 42126956 | − |
| 42126956 | GAACGACACTCATCA | | | | | | |
| | tCAAtCTGTCATCGG | | | | | | |
| | TGCTG | | | | | | |
| 22_ | CATCTTCTGCCCAGG | 602 | | | 22 | 42126956 | − |
| 42126956 | GAACGACACTCATCA | | | | | | |
| | tCAAtCTGTtATCGG | | | | | | |
| | TGCTG | | | | | | |
| 22_ | GCTTCTCCCAGACGG | 603 | | | 22 | 42126981 | + |
| 42126981 | CCTCATCCTTCAGCA | | | | | | |
| | CCGATGACAGGTTGG | | | | | | |
| | TGATG | | | | | | |
| 22_ | GCTTCTCCCAGACGG | 604 | | | 22 | 42126981 | + |
| 42126981 | CCTCATCCTTCAGCA | | | | | | |
| | CYGATGACAGGTTGG | | | | | | |
| | TGATG | | | | | | |
| 22_ | GAGTCCAGTCCCCAC | 605 | | | 22 | 42126981 | − |
| 42126981 | TCTCACCCTGCATCT | | | | | | |
| | CCTGCCCAGGGAACG | | | | | | |
| | ACACT | | | | | | |
| rs77312 | CTGGGTGTGACCCAT | 606 | CTGGGTGTGACCCAT | 1865 | 22 | 42127457 | − |
| 092 | ATGACATCCCGTGAC | | ATGACATCCCGTGAC | | | | |
| | ATCGAAGTACAGGGC | | ATCGAAGTACAGGGC | | | | |
| | TTCCA | | TTCCG | | | | |
| rs77312 | TGCTGGTGCTGAGCT | 607 | | | 22 | 42127457 | + |
| 092 | GGGGTGAGGAGGGCG | | | | | | |
| | CCAGGCCTACCTTAG | | | | | | |
| | GGATG | | | | | | |
| rs26760 | CATGCCCTACACCAC | 608 | | | 22 | 42127514 | − |
| 8293 | TGCCGTGATTCATGA | | | | | | |
| | GGTGCAGCGCTTTGG | | | | | | |
| | GGACA | | | | | | |
| rs26760 | CATGCCCTACACCAC | 609 | | | 22 | 42127514 | − |
| 8293 | TGCCGTGATTCATGA | | | | | | |
| | GGTGCAGCGCTTTGG | | | | | | |
| | GGACA | | | | | | |
| rs26760 | CATGCCCTACACCAC | 610 | | | 22 | 42127514 | − |
| 8293 | TGCCGTGATTCATGA | | | | | | |
| | GGTGCAGCGCTTTGG | | | | | | |
| | GGACA | | | | | | |
| rs15558 | CCAGGCTCACATGCC | 611 | | | 22 | 42127523 | − |
| 88910 | CTACACCACTGCCGT | | | | | | |
| | GATTCAYGAGGTGCA | | | | | | |
| | GCVCT | | | | | | |
| rs15558 | CAGGCTCACATGCCC | 612 | CAGGCTCACATGCCC | 1866 | 22 | 42127523 | − |
| 88910 | TACACCACTGCCGTG | | TACACCACTGCCGTG | | | | |
| | ATTCATGAGGTGCAG | | ATTCATGAGGTGCAG | | | | |
| | CGCTT | | CGCTC | | | | |
| rs15558 | CAGGCTCACATGCCC | 613 | CAGGCTCACATGCCC | 1867 | 22 | 42127523 | − |
| 88910 | TACACCACTGCCGTG | | TACACCACTGCCGTG | | | | |
| | ATTCATGAGGTGCAG | | ATTCATGAGGTGCAG | | | | |
| | CGCTT | | CGCTC | | | | |
| rs20210 | GATAGGGCAGGTGCG | 614 | | | 22 | 42127556 | − |
| 2799 | GCGACCAGAGATGGG | | | | | | |
| | TGACCAGGCTCRCAT | | | | | | |
| | GCCCT | | | | | | |
| rs61736 | TGTCCCCAAAGCGCT | 615 | | | 22 | 42127565 | + |
| 517 | GCACCTCATGAATCA | | | | | | |
| | CGGCAGTGGTGYAGG | | | | | | |
| | GCATG | | | | | | |
| rs61736 | TGTCCCCAAAGCGCT | 616 | | | 22 | 42127565 | + |
| 517 | GCACCTCATGAATCA | | | | | | |
| | CGGCAGTGGTGYAGG | | | | | | |
| | GCATG | | | | | | |
| rs61736 | GTCCCCAAAGCGCTG | 617 | GTCCCCAAAGCGCTG | 1868 | 22 | 42127565 | + |
| 517 | CACCTCATGAATCAC | | CACCTCATGAATCAC | | | | |
| | GGCAGTGGTGTAGGG | | GGCAGTGGTGTAGGG | | | | |
| | CATGT | | CATGC | | | | |
| rs76088 | TCCAGGCCGTGTCCA | 618 | | | 22 | 42127589 | − |
| 846 | ACAGGAGATCGACGA | | | | | | |
| | CGTGATAGGGCAGDT | | | | | | |
| | GNGGY | | | | | | |
| rs76088 | TCCAGGCCGTGTCCA | 619 | | | 22 | 42127589 | − |
| 846 | ACAGGAGATCGACGA | | | | | | |
| | CGTGATAGGGCAGDT | | | | | | |
| | GNGGY | | | | | | |
| rs72549 | GTCCAGGCCGTGTCC | 620 | | | 22 | 42127590 | − |
| 347 | AACAGGAGATCGACG | | | | | | |
| | ACGTGATAGGGCAGG | | | | | | |
| | TGCGG | | | | | | |
| rs26760 | ACGGCAGTGGTGTAG | 621 | ACGGCAGTGGTGTAG | 1869 | 22 | 42127593 | + |
| 8295 | GGCATGTGAGCCTGG | | GGCATGTGAGCCTGG | | | | |
| | TCACCCATCTCTGGT | | TCACCCATCTCTGGT | | | | |
| | YRCCA | | YRCCG | | | | |
| rs26760 | ACGGCAGTGGTGTAG | 622 | ACGGCAGTGGTGTAG | 1870 | 22 | 42127593 | + |
| 8295 | GGCATGTGAGCCTGG | | GGCATGTGAGCCTGG | | | | |
| | TCACCCATCTCTGGT | | TCACCCATCTCTGGT | | | | |
| | YRCCA | | YRCCG | | | | |
| 22_ | GGTGTAGGGCATGTG | 623 | | | 22 | 42127602 | + |
| 42127602 | AGCCTGGTCACCCAT | | | | | | |
| | CTCTGGTCGCCGCAC | | | | | | |
| | CTGCC | | | | | | |
| 22_ | GGTGTAGGGCATGTG | 624 | | | 22 | 42127602 | + |
| 42127602 | AGCCTGGTCACCCAT | | | | | | |
| | CTCTGGTCRCCNCAH | | | | | | |
| | CTGCC | | | | | | |
| 22_ | GACGCATGTCTGTCC | 625 | GACGCATGTCTGTCC | 1871 | 22 | 42127602 | − |
| 42127602 | AGGCCGTGTCCAACA | | AGGCCGTGTCCAACA | | | | |
| | GGAGATCGACGACGT | | GGAGATCGACGACGT | | | | |
| | GATAA | | GATAG | | | | |
| rs59421 | TTGTGGGGACGCATG | 626 | | | 22 | 42127608 | − |
| 388 | TCTGTCCAGGCCGTG | | | | | | |
| | TCCAACAGGAGATCR | | | | | | |
| | ACRRC | | | | | | |
| rs59421 | TTGTGGGGACGCATG | 627 | | | 22 | 42127608 | − |
| 388 | TCTGTCCAGGCCGTG | | | | | | |
| | TCCAACAGGAGATCR | | | | | | |
| | ACRRC | | | | | | |
| rs59421 | TTGTGGGGACGCATG | 628 | | | 22 | 42127608 | − |
| 388 | TCTGTCCAGGCCGTG | | | | | | |
| | TCCAACAGGAGATCR | | | | | | |
| | ACRRC | | | | | | |
| rs74871 | CATTGTGGGGACGCA | 629 | | | 22 | 42127610 | − |
| 2690 | TGTCTGTCCAGGCCG | | | | | | |
| | TGTCCAACAGGMGAT | | | | | | |
| | CRACR | | | | | | |
| rs74871 | CATTGTGGGGACGCA | 630 | | | 22 | 42127610 | − |
| 2690 | TGTCTGTCCAGGCCG | | | | | | |
| | TGTCCAACAGGMGAT | | | | | | |
| | CRACR | | | | | | |
| rs78209 | CCATTGTGGGGACGC | 631 | | | 22 | 42127611 | − |
| 835 | ATGTCTGTCCAGGCC | | | | | | |
| | STGTCCAACAGGMGA | | | | | | |
| | TCRAC | | | | | | |
| rs78209 | CCATTGTGGGGACGC | 632 | | | 22 | 42127611 | − |
| 835 | ATGTCTGTCCAGGCC | | | | | | |
| | STGTCCAACAGGMGA | | | | | | |
| | TCRAC | | | | | | |
| rs78209 | CATTGTGGGGACGCA | 633 | CATTGTGGGGACGCA | 1872 | 22 | 42127611 | − |
| 835 | TGTCTGTCCAGGCCG | | TGTCTGTCCAGGCCG | | | | |
| | TGTCCAACAGGAGAT | | TGTCCAACAGGAGAT | | | | |
| | CGACA | | CGACG | | | | |
| rs14100 | GGACCCCCTGGGTGC | 634 | GGACCCCCTGGGTGC | 1873 | 22 | 42127631 | − |
| 9491 | TGACCCATTGTGGGG | | TGACCCATTGTGGGG | | | | |
| | ACGCATGTCTGTCCA | | ACGCATGTCTGTCCA | | | | |
| | GGCCG | | GGCCC | | | | |
| rs14100 | GGACCCCCTGGGTGC | 635 | GGACCCCCTGGGTGC | 1874 | 22 | 42127631 | − |
| 9491 | TGACCCATTGTGGGG | | TGACCCATTGTGGGG | | | | |
| | ACGCATGTCTGTCCA | | ACGCATGTCTGTCCA | | | | |
| | GGCCG | | GGCCC | | | | |
| rs14100 | GGACCCCCTGGGTGC | 636 | GGACCCCCTGGGTGC | 1875 | 22 | 42127631 | − |
| 9491 | TGACCCATTGTGGGG | | TGACCCATTGTGGGG | | | | |
| | ACGCATGTCTGTCCA | | ACGCATGTCTGTCCA | | | | |
| | GGCCG | | GGCCC | | | | |
| rs28371 | CGGATGTGCAGCGTG | 637 | | | 22 | 42127803 | + |
| 725 | AGaCaATCTGGGAAA | | | | | | |
| | CAGTGCAtGtGCCGA | | | | | | |
| | GGGAG | | | | | | |
| rs28371 | CGtATGTGCAGCGTG | 638 | | | 22 | 42127803 | + |
| 725 | AGaCaATCTGGGAAA | | | | | | |
| | CAGTGCAtGtGCCGA | | | | | | |
| | GGGAG | | | | | | |
| rs28371 | CGtATGTGCAGCGTG | 639 | | | 22 | 42127803 | + |
| 725 | AGaCaATCTGGGAAA | | | | | | |
| | CAGTGCAtGtGCCGA | | | | | | |
| | GGGAG | | | | | | |
| rs72549 | CCTGTACCCTTCCTC | 640 | CCTGTACCCTTCCTC | 1876 | 22 | 42127841 | + |
| 349 | CCTCGGCCCCTGCAC | | CCTCGGCCCCTGCAC | | | | |
| | TGTTTCCCAGATGGG | | TGTTTCCCAGATGGG | | | | |
| | CTCAC | | CTCAG | | | | |
| rs72549 | CCTGTACCCTTCCTC | 641 | CCTGTACCCTTCCTC | 1877 | 22 | 42127841 | + |
| 349 | CCTCGGCCCCTGCAC | | CCTCGGCCCCTGCAC | | | | |
| | TGTTTCCCAGATGGG | | TGTTTCCCAGATGGG | | | | |
| | CTCAC | | CTCAG | | | | |
| rs72549 | CCTGTACCCTTCCTC | 642 | CCTGTACCCTTCCTC | 1878 | 22 | 42127841 | + |
| 349 | CCTCGGCCCCTGCAC | | CCTCGGCCCCTGCAC | | | | |
| | TGTTTCCCAGATGGG | | TGTTTCCCAGATGGG | | | | |
| | CTCAC | | CTCAG | | | | |
| rs73088 | TACCCTTCCTCCCTC | 643 | | | 22 | 42127845 | + |
| 2170 | GGCCCCTGCACTGTT | | | | | | |
| | TCCCAGATGGGCTCA | | | | | | |
| | CGCTG | | | | | | |
| rs73088 | TACCCTTCCTCCCTC | 644 | | | 22 | 42127845 | + |
| 2170 | GGCCCCTGCACTGTT | | | | | | |
| | TCCCAGATGGGCTCA | | | | | | |
| | CGCTG | | | | | | |
| rs73088 | TACCCTTCCTCCCTC | 645 | | | 22 | 42127846 | + |
| 2170 | GGCCCCTGCACTGTT | | | | | | |
| | TCCCAGATGGGCTCA | | | | | | |
| | CGCTG | | | | | | |
| rs79292 | TCCTCCCTCgGcCaC | 646 | | | 22 | 42127852 | + |
| 917 | TGCacTGtttCCCAG | | | | | | |
| | acaGGCTCACGCTGC | | | | | | |
| | ACATC | | | | | | |
| rs79292 | ACCACCTCGACCACG | 647 | | | 22 | 42127852 | − |
| 917 | CTGGCCTGGtGCCTC | | | | | | |
| | CTGCTCATGATCCTA | | | | | | |
| | CAtCc | | | | | | |
| rs79292 | ACCACCTCGACCACa | 648 | | | 22 | 42127852 | − |
| 917 | CTGaCCTGGcGCCTC | | | | | | |
| | CTGCTCATGATCCTA | | | | | | |
| | CAtCc | | | | | | |
| rs50308 | CCCTCGGCCCCTGCA | 649 | | | 22 | 42127856 | + |
| 67 | CTGTTTCCCAGATGG | | | | | | |
| | GCTCACGCTGCACAT | | | | | | |
| | CCGGA | | | | | | |
| rs50308 | CCCTCGGCCCCTGCA | 650 | | | 22 | 42127856 | + |
| 67 | CTGTTTCCCAGATGG | | | | | | |
| | GCTCACGCTGCACAT | | | | | | |
| | CCGGA | | | | | | |
| rs50308 | CCCTCGGCCCCTGCA | 651 | | | 22 | 42127856 | + |
| 67 | CTGTTTCCCAGATGG | | | | | | |
| | GCTCACGCTGCACAT | | | | | | |
| | CCGGA | | | | | | |
| 22_ | ATCCGGATGTAGGAT | 652 | | | 22 | 42127899 | + |
| 42127899 | CATGAGCAGGAGGCC | | | | | | |
| | CCAGGCCAGCGTGGT | | | | | | |
| | CGAGG | | | | | | |
| 22_ | ATCCGGATGTAGGAT | 653 | | | 22 | 42127899 | + |
| 42127899 | CATGAGCAGGAGGCC | | | | | | |
| | CCAGGCCAGCGTGGT | | | | | | |
| | CGAGG | | | | | | |
| 22_ | ATCCGGATGTAGGAT | 654 | | | 22 | 42127899 | + |
| 42127899 | CATGAGCAGGAGGCC | | | | | | |
| | CCAGGCCAGCGTGGT | | | | | | |
| | CGAGG | | | | | | |
| rs14067 | GAGGCCCCAGGCCAG | 655 | GAGGCCCCAGGCCAG | 1879 | 22 | 42127922 | + |
| 19554 | CGTGGTCGAGGYGGT | | CGTGGTCGAGGYGGT | | | | |
| | CACCATCCCGGCAGA | | CACCATCCCGGCAGA | | | | |
| | GAACA | | GAACG | | | | |
| rs14067 | CCCTGAGAGCAGCTT | 656 | | | 22 | 42127922 | − |
| 19554 | CAATGATGAGAACCT | | | | | | |
| | GCGCATAGTGGTGGC | | | | | | |
| | TGACC | | | | | | |
| rs14067 | CCCTGAGAGCAGCTT | 657 | | | 22 | 42127922 | − |
| 19554 | CAATGATGAGAACCT | | | | | | |
| | GYGCMTAGTGGTGGC | | | | | | |
| | TGACC | | | | | | |
| rs94971 | GTGGTCGAGGTGGTC | 658 | GTGGTCGAGGTGGTC | 1880 | 22 | 42127938 | + |
| 7872 | ACCATCCCGGCAGAG | | ACCATCCCGGCAGAG | | | | |
| | AACAGGTCAGCCACC | | AACAGGTCAGCCACC | | | | |
| | ACTAT | | ACTAG | | | | |
| rs94971 | GTGGTCGAGGTGGTC | 659 | GTGGTCGAGGTGGTC | 1881 | 22 | 42127938 | + |
| 7872 | ACCATCCCGGCAGAG | | ACCATCCCGGCAGAG | | | | |
| | AACAGGTCAGCCACC | | AACAGGTCAGCCACC | | | | |
| | ACTAT | | ACTAG | | | | |
| rs94971 | CGTGGTCGAGGTGGT | 660 | | | 22 | 42127938 | + |
| 7872 | CACCATCCCGGCAGA | | | | | | |
| | GAACAGGTCAGCCAC | | | | | | |
| | CACTA | | | | | | |
| rs16947 | CTGCTCAGGCCAAGG | 661 | | | 22 | 42127941 | − |
| | GGAACCCTGAGAGCA | | | | | | |
| | GCTTCAATGATGAGA | | | | | | |
| | ACCTG | | | | | | |
| rs26760 | TGTCCCGAGTATGCT | 662 | | | 22 | 42127962 | − |
| 8279 | CTCGGCCCTGCTCAG | | | | | | |
| | GCCAAGGGGARCCCT | | | | | | |
| | GAGAG | | | | | | |
| rs26760 | TGTCCCGAGTATGCT | 663 | | | 22 | 42127962 | − |
| 8279 | CTCGGCCCTGCTCAG | | | | | | |
| | GCCAAGGGGARCCCT | | | | | | |
| | GAGAG | | | | | | |
| rs11358 | TACCCCGTTCTGTCC | 664 | | | 22 | 42127973 | − |
| 29 | CGAGTATGCTCTCGG | | | | | | |
| | CCCTGCTCAGGCCAA | | | | | | |
| | GGGGA | | | | | | |
| rs11358 | ACCCCGTTCTGTCCC | 665 | ACCCCGTTCTGTCCC | 1882 | 22 | 42127973 | − |
| 29 | GAGTATGCTCTCGGC | | GAGTATGCTCTCGGC | | | | |
| | CCTGCTCAGGCCAAG | | CCTGCTCAGGCCAAG | | | | |
| | GGGAA | | GGGAG | | | | |
| rs11358 | ACCCCGTTCTGTCCC | 666 | ACCCCGTTCTGTCCC | 1883 | 22 | 42127973 | − |
| 29 | GAGTATGCTCTCGGC | | GAGTATGCTCTCGGC | | | | |
| | CCTGCTCAGGCCAAG | | CCTGCTCAGGCCAAG | | | | |
| | GGGAA | | GGGAG | | | | |
| rs11358 | TACCCCGTTCTGTCC | 667 | | | 22 | 42127973 | − |
| 29 | CGAGTATGCTCTCGG | | | | | | |
| | CCCTGCTCAGGCCAA | | | | | | |
| | GGGGA | | | | | | |
| rs11358 | TACCCCGTTCTGTCC | 668 | | | 22 | 42127973 | − |
| 29 | CGAGTATGCTCTCGG | | | | | | |
| | CCCTGCTCAGGCCAA | | | | | | |
| | GGGGA | | | | | | |
| rs50306 | CAGCCCAGCCCCCCt | 669 | | | 22 | 42128174 | − |
| 56 | GAGACtTGACTGAGt | | | | | | |
| | CCTTCCTGGCAGAGA | | | | | | |
| | TGGAG | | | | | | |
| rs50306 | CAGCCCAGCCCCCCt | 670 | | | 22 | 42128174 | − |
| 56 | GAGACtTGACTGAGt | | | | | | |
| | CCTTCCTGGCAGAGA | | | | | | |
| | TGGAG | | | | | | |
| rs50306 | CAGCCCAGCCCCCCt | 671 | | | 22 | 42128174 | − |
| 56 | GAGACtTGACTGAGt | | | | | | |
| | CCTTCCTGGCAGAGA | | | | | | |
| | TGGAG | | | | | | |
| rs11358 | GACCCAGCCCAGCCC | 672 | GACCCAGCCCAGCCC | 1884 | 22 | 42128181 | − |
| 28 | CCCCGAGACCTGACT | | CCCCGAGACCTGACT | | | | |
| | GAGGCCTTCCTKGCA | | GAGGCCTTCCTKGCA | | | | |
| | RAGAA | | RAGAT | | | | |
| rs11358 | GACCCAGCCCAGCCC | 673 | GACCCAGCCCAGCCC | 1885 | 22 | 42128181 | − |
| 28 | CCCCGAGACCTGACT | | CCCCGAGACCTGACT | | | | |
| | GAGGCCTTCCTKGCA | | GAGGCCTTCCTKGCA | | | | |
| | RAGAA | | RAGAT | | | | |
| rs77913 | CCTGGGACCCAGCCC | 674 | | | 22 | 42128185 | − |
| 725 | AGCCCCCCCGAGACC | | | | | | |
| | TGACTGAGGCCTTCC | | | | | | |
| | TKGCA | | | | | | |
| rs72549 | CACCGTGGCAGCCAC | 675 | | | 22 | 42128201 | + |
| 351 | TCTCACCTTCTCCAT | | | | | | |
| | CTCTGCCAGGAAGGC | | | | | | |
| | CTCAG | | | | | | |
| rs72549 | CACCGTGGCAGCCAC | 676 | | | 22 | 42128201 | + |
| 351 | TCTCACCTTCTCCAT | | | | | | |
| | CTCTGCCAGGAAGGC | | | | | | |
| | CTCAG | | | | | | |
| rs36754 | ATGAGCTGCTAACTG | 677 | | | 22 | 42128212 | − |
| 3000 | AGCACAGGATGACCT | | | | | | |
| | GGGACCCAGCCCAGC | | | | | | |
| | CCCCC | | | | | | |
| rs36754 | ATGAGCTGCTAACTG | 678 | | | 22 | 42128212 | − |
| 3000 | AGCACAGGATGACCT | | | | | | |
| | GGGACCCAGCCCAGC | | | | | | |
| | MMYCC | | | | | | |
| rs36754 | ATGAGCTGCTAACTG | 679 | | | 22 | 42128212 | − |
| 3000 | AGCACAGGATGACCT | | | | | | |
| | GGGACCCAGCCCAGC | | | | | | |
| | CCCCC | | | | | | |
| rs72549 | TGCTAACTGAGCACA | 680 | TGCTAACTGAGCACA | 1886 | 22 | 42128218 | − |
| 352 | GGATGACCTGGGACC | | GGATGACCTGGGACC | | | | |
| | CAGCCCAGCCCYCCY | | CAGCCCAGCCCCYCC | | | | |
| | RAGAC | | YRAGA | | | | |
| rs72549 | TGCTAACTGAGCACA | 681 | TGCTAACTGAGCACA | 1887 | 22 | 42128218 | − |
| 352 | GGATGACCTGGGACC | | GGATGACCTGGGACC | | | | |
| | CAGCCCAGCCCYCCY | | CAGCCCAGCCCCYCC | | | | |
| | RAGAC | | YRAGA | | | | |
| rs72549 | TGCTAACTGAGCACA | 682 | TGCTAACTGAGCACA | 1888 | 22 | 42128218 | − |
| 352 | GGATGACCTGGGACC | | GGATGACCTGGGACC | | | | |
| | CAGCCCAGCCCCCCC | | CAGCCCAGCCCCCCC | | | | |
| | GAGAC | | CGAGA | | | | |
| rs26760 | CTGCCAGGAAGGCCT | 683 | | | 22 | 42128235 | + |
| 8297 | CAGTCAGGTCTCGGG | | | | | | |
| | GGGGCTGGGCTGGGT | | | | | | |
| | CCCAG | | | | | | |
| rs35742 | GAAGGCCTCAGTCAG | 684 | | | 22 | 42128241 | + |
| 686 | GTCTCGGGGGGGCTG | | | | | | |
| | GGCTGGGTCCCAGGT | | | | | | |
| | CATCC | | | | | | |
| rs35742 | GAAGGCCTCAGTCAG | 685 | | | 22 | 42128241 | + |
| 686 | GTCTCGGGGGKGCTG | | | | | | |
| | GGCTGGGTCCCAGRT | | | | | | |
| | CATCC | | | | | | |
| rs72549 | AGTCAGGTCTCGGGG | 686 | | | 22 | 42128248 | + |
| 353 | GGGCTGGGCTGGGTC | | | | | | |
| | CCAGGTCATCCTGTG | | | | | | |
| | CTCAG | | | | | | |
| rs72549 | AGTCAGGTCTCGGGG | 687 | | | 22 | 42128248 | + |
| 353 | GGGCTGGGCTGGGTC | | | | | | |
| | CCAGRTCATCCTGTG | | | | | | |
| | CTCAG | | | | | | |
| rs72549 | AAGGTCCTACGCTTC | 688 | | | 22 | 42128248 | − |
| 353 | CAAAAGGCTTTCCTG | | | | | | |
| | ACCCAGCTGGATGAG | | | | | | |
| | CTGCT | | | | | | |
| 22_ | GGCTGGGTCCCAGGT | 689 | | | 22 | 42128272 | + |
| 42128272 | CATCCTGTGCTCAGT | | | | | | |
| | TAGCAGCTCATCCAG | | | | | | |
| | CTGGG | | | | | | |
| 22_ | GGCTGGGTCCCAGGT | 690 | | | 22 | 42128272 | + |
| 42128272 | CATCCTGTGCTCAGT | | | | | | |
| | TAGCAGCTCATCCAG | | | | | | |
| | CTGGG | | | | | | |
| 22_ | GGCTGGGTCCCAGGT | 691 | | | 22 | 42128272 | + |
| 42128272 | CATCCTGTGCTCAGT | | | | | | |
| | TAGCAGCTCATCCAG | | | | | | |
| | CTGGG | | | | | | |
| rs28371 | TGGTGTAGGTGCTGA | 692 | | | 22 | 42128308 | − |
| 717 | ATGCTGTCCCCGTCC | | | | | | |
| | TCCTGCATATCCCAG | | | | | | |
| | CGCTG | | | | | | |
| rs28371 | TGGTGTAGGTGCTGA | 693 | | | 22 | 42128308 | − |
| 717 | ATGCTGTCCCCGTCC | | | | | | |
| | TCCTGCATATCCCAG | | | | | | |
| | CGCTG | | | | | | |
| rs28371 | TGGTGTAGGTGCTGA | 694 | | | 22 | 42128308 | − |
| 717 | ATGCTGTCCCCGTCC | | | | | | |
| | TCCTGCATATCCCAG | | | | | | |
| | CGCTG | | | | | | |
| rs17002 | GGAAAGCCTTTTGGA | 695 | | | 22 | 42128325 | + |
| 853 | AGCGTAGGACCTTGC | | | | | | |
| | CAGMCARCGCTGGGA | | | | | | |
| | TRTGC | | | | | | |
| rs17002 | GAAAGCCTTTTGGAA | 696 | GAAAGCCTTTTGGAA | 1889 | 22 | 42128325 | + |
| 853 | GCGTAGGACCTTGCC | | GCGTAGGACCTTGCC | | | | |
| | AGMCARCGCTGGGAT | | AGMCARCGCTGGGAT | | | | |
| | RTGCA | | RTGCG | | | | |
| rs17002 | GAAAGCCTTTTGGAA | 697 | GAAAGCCTTTTGGAA | 1890 | 22 | 42128325 | + |
| 853 | GCGTAGGACCTTGCC | | GCGTAGGACCTTGCC | | | | |
| | AGMCARCGCTGGGAT | | AGMCARCGCTGGGAT | | | | |
| | RTGCA | | RTGCG | | | | |
| 22_ | TTCCTCAGGCTGCTG | 698 | | | 22 | 42128795 | − |
| 42128795 | GACCTAGCTCAGGAG | | | | | | |
| | GGACTGAAGGAGGAG | | | | | | |
| | TCGGG | | | | | | |
| 22_ | TTCCTCAGGCTGCTG | 699 | | | 22 | 42128795 | − |
| 42128795 | GACCTAGCTCAGGAG | | | | | | |
| | GGACTGAAGGAGGAG | | | | | | |
| | TCGGG | | | | | | |
| rs19953 | GTACGACGACCCTCG | 700 | | | 22 | 42128812 | − |
| 5154 | CTTCCTCAGGCTGCT | | | | | | |
| | GGACCTAGCTCAGGA | | | | | | |
| | GGVAY | | | | | | |
| rs72549 | GTCTCTCGCTCCGCA | 701 | | | 22 | 42128817 | + |
| 354 | CCTCGCGCAGAAAGC | | | | | | |
| | CCGACTCCTCCTTCR | | | | | | |
| | RTBCC | | | | | | |
| rs72549 | GTCTCTCGCTCCGCA | 702 | | | 22 | 42128817 | + |
| 354 | CCTCGCGCAGAAAGC | | | | | | |
| | CCGACTCCTCCTTCA | | | | | | |
| | GTCCC | | | | | | |
| rs72549 | GTCTCTCGCTCCGCA | 703 | | | 22 | 42128817 | + |
| 354 | CCTCGCGCAGAAAGC | | | | | | |
| | CCGACTCCTCCTTCA | | | | | | |
| | GTCCC | | | | | | |
| rs74536 | CCGACTCCTCCTTCA | 704 | | | 22 | 42128848 | + |
| 5204 | GTCCCTCCTGAGCTA | | | | | | |
| | GGTCCAGCAGCCTGA | | | | | | |
| | GGAAG | | | | | | |
| rs74536 | CCGACTCCTCCTTCA | 705 | | | 22 | 42128848 | + |
| 5204 | GTCCCTCCTGAGCTA | | | | | | |
| | GGTCCAGCAGCCTGA | | | | | | |
| | GGAAG | | | | | | |
| rs74536 | CCGACTCCTCCTTCA | 706 | | | 22 | 42128848 | + |
| 5204 | GTCCCTCCTGAGCTA | | | | | | |
| | GGTCCAGCAGCCTGA | | | | | | |
| | GGAAG | | | | | | |
| 22_ | GGTCCAGCAGCCTGA | 707 | | | 22 | 42128878 | + |
| 42128878 | GGAAGCGAGGGTCGT | | | | | | |
| | CGTACTCGAAGCGGC | | | | | | |
| | GCCCG | | | | | | |
| 22_ | GTCCAGCAGCCTGAG | 708 | GTCCAGCAGCCTGAG | 1891 | 22 | 42128878 | + |
| 42128878 | GAAGCGAGGGTCGTC | | GAAGCGAGGGTCGTC | | | | |
| | GTACTCGAAGCGGCG | | GTACTCGAAGCGGCG | | | | |
| | CCCGA | | CCCGC | | | | |
| 22_ | AACGGTCTCTTGGAC | 709 | AACGGTCTCTTGGAC | 1892 | 22 | 42128878 | − |
| 42128878 | AAAGCCGTGAGCAAC | | AAAGCCGTGAGCAAC | | | | |
| | GTGATCGCCTCCCTC | | GTGATCGCCTCCCTC | | | | |
| | ACCTT | | ACCTG | | | | |
| 22_ | TCCAGCAGCCTGAGG | 710 | TCCAGCAGCCTGAGG | 1893 | 22 | 42128879 | + |
| 42128879 | AAGCGAGGGTCGTCG | | AAGCGAGGGTCGTCG | | | | |
| | TACTCGAAGCGGCGC | | TACTCGAAGCGGCGC | | | | |
| | CCGCA | | CCGCG | | | | |
| 22_ | CCAACGGTCTCTTGG | 711 | | | 22 | 42128879 | − |
| 42128879 | ACAAAGCCGTGAGCA | | | | | | |
| | ACGTGATCGCCTCCC | | | | | | |
| | TCACC | | | | | | |
| 22_ | CAACGGTCTCTTGGA | 712 | CAACGGTCTCTTGGA | 1894 | 22 | 42128879 | − |
| 42128879 | CAAAGCCGTGAGCAA | | CAAAGCCGTGAGCAA | | | | |
| | CGTGATCGCCTCCCT | | CGTGATCGCCTCCCT | | | | |
| | CACCT | | CACCC | | | | |
| rs72549 | GGCGACCCCTTACCC | 713 | GGCGACCCCTTACCC | 1895 | 22 | 42128927 | − |
| 356 | GCATCTCCCACCCCC | | GCATCTCCCACCCCC | | | | |
| | ARGACGCCCCTTTYG | | ARGACGCCCCTTTYG | | | | |
| | CCCCA | | CCCCT | | | | |
| rs72549 | GGCGACCCCTTACCC | 714 | GGCGACCCCTTACCC | 1896 | 22 | 42128927 | − |
| 356 | GCATCTCCCACCCCC | | GCATCTCCCACCCCC | | | | |
| | ARGACGCCCCTTTYG | | ARGACGCCCCTTTYG | | | | |
| | CCCCA | | CCCCT | | | | |
| rs38920 | GAAGGCGGGGGACGG | 715 | | | 22 | 42128945 | − |
| 97 | GGAAGGCGACCCCTT | | | | | | |
| | ACCCGCATCTCCCAC | | | | | | |
| | CCCCA | | | | | | |
| rs38920 | GAAGGCGGGGGACGG | 716 | | | 22 | 42128945 | − |
| 97 | GGAAGGCGACCCCTT | | | | | | |
| | ACCCGCATCTCCCAC | | | | | | |
| | CCCCA | | | | | | |
| rs50308 | GGTGACCGAGGAGGC | 717 | GGTGACCGAGGAGGC | 1897 | 22 | 42129033 | − |
| 65 | CGCCTGCCTTTGTGC | | CGCCTGCCTTTGTGC | | | | |
| | CGCCTTCGCCAACCA | | CGCCTTCGCCAACCA | | | | |
| | CTCCA | | CTCCG | | | | |
| rs50308 | GGTGACCGAGGAGGC | 718 | GGTGACCGAGGAGGC | 1898 | 22 | 42129033 | − |
| 65 | CGCCTGCCTTTGTGC | | CGCCTGCCTTTGTGC | | | | |
| | CGCCTTCGCCAACCA | | CGCCTTCGCCAACCA | | | | |
| | CTCCA | | CTCCG | | | | |
| rs50308 | GGGTGACCGAGGAGG | 719 | | | 22 | 42129033 | − |
| 65 | CCGCCTGCCTTTGTG | | | | | | |
| | CCGCCTTCGCCAACC | | | | | | |
| | ACTCC | | | | | | |
| rs11358 | CCCCGCCTTCCCAGT | 720 | | | 22 | 42129036 | + |
| 26 | TCCCGCTTTGTGCCC | | | | | | |
| | TTCTGCCCATCACCC | | | | | | |
| | ACHRG | | | | | | |
| rs11358 | AGTGGGTGACCGAGG | 721 | | | 22 | 42129036 | − |
| 26 | AGGCCGCCTGCCTTT | | | | | | |
| | GTGCCGCCTTCGCYR | | | | | | |
| | ACCAM | | | | | | |
| rs11358 | GTGGGTGACCGAGGA | 722 | GTGGGTGACCGAGGA | 1899 | 22 | 42129036 | − |
| 26 | GGCCGCCTGCCTTTG | | GGCCGCCTGCCTTTG | | | | |
| | TGCCGCCTTCGCYRA | | TGCCGCCTTCGCYRA | | | | |
| | CCAMT | | CCAMG | | | | |
| rs11358 | CAGTGGGTGACCGAG | 723 | | | 22 | 42129037 | − |
| 25 | GAGGCCGCCTGCCTT | | | | | | |
| | TGTGCCGCCTTCGCC | | | | | | |
| | AACCA | | | | | | |
| rs11358 | CAGTGGGTGACCGAG | 724 | | | 22 | 42129037 | − |
| 25 | GAGGCCGCCTGCCTT | | | | | | |
| | TSTGCCGCCTTCGCY | | | | | | |
| | RACCA | | | | | | |
| rs11358 | CTTCCCAGTTCCCGC | 725 | | | 22 | 42129042 | + |
| 24 | TTTGTGCCCTTCTGC | | | | | | |
| | CCATCACCCACCRGM | | | | | | |
| | KTGGT | | | | | | |
| rs11358 | TTCCCAGTTCCCGCT | 726 | TTCCCAGTTCCCGCT | 2208 | 22 | 42129042 | + |
| 24 | TTGTGCCCTTCTGCC | | TTGTGCCCTTCTGCC | | | | |
| | CATCACCCACCRGMK | | CATCACCCACCRGMK | | | | |
| | TGGTT | | TGGTC | | | | |
| rs11358 | TGGAGCAGTGGGTGA | 727 | | | 22 | 42129042 | − |
| 24 | CCGAGGAGGCCGCCT | | | | | | |
| | GCCTTTSTGCCGCCT | | | | | | |
| | TCGCY | | | | | | |
| 22_ | TTTGTGCCCTTCTGC | 728 | TTTGTGCCCTTCTGC | 2209 | 22 | 42129056 | + |
| 42129056 | CCATCACCCACCGGA | | CCATCACCCACCGGA | | | | |
| | GTGGTTGGCGAAGGC | | GTGGTTGGCGAAGGC | | | | |
| | GGCAC | | GGCAG | | | | |
| 22_ | GGCAAGAAGTCGCTG | 729 | GGCAAGAAGTCGCTG | 2210 | 22 | 42129056 | − |
| 42129056 | GAGCAGTGGGTGACC | | GAGCAGTGGGTGACC | | | | |
| | GAGGAGGCCGCCTGC | | GAGGAGGCCGCCTGC | | | | |
| | CTTTG | | CTTTC | | | | |
| 22_ | GGCAAGAAGTCGCTG | 730 | GGCAAGAAGTCGCTG | 2211 | 22 | 42129056 | − |
| 42129056 | GAGCAGTGGGTGACC | | GAGCAGTGGGTGACC | | | | |
| | RAGGHGGCYGCCTGC | | RAGGHGGCYGCCTGC | | | | |
| | CTTTG | | CTTTC | | | | |
| 22_ | GGCAAGAAGTCGCTG | 731 | GGCAAGAAGTCGCTG | 2212 | 22 | 42129056 | − |
| 42129056 | GAGCAGTGGGTGACC | | GAGCAGTGGGTGACC | | | | |
| | GAGGAGGCCGCCTGC | | GAGGAGGCCGCCTGC | | | | |
| | CTTTG | | CTTTC | | | | |
| rs26760 | CCCATCACCCACCGG | 732 | | | 22 | 42129071 | + |
| 8302 | AGTGGTTGGCGAAGG | | | | | | |
| | CGGCACAAAGGCAGG | | | | | | |
| | CGGCC | | | | | | |
| rs28371 | TCACCCACCGGAGTG | 733 | | | 22 | 42129075 | + |
| 710 | GTTGGCGAAGGCGGC | | | | | | |
| | ACAAAGGCAGGCGGC | | | | | | |
| | CTCCT | | | | | | |
| rs28371 | TCACCCACCGGAGTG | 734 | | | 22 | 42129075 | + |
| 710 | GTTGGCGAAGGCGGC | | | | | | |
| | ACAAAGGCAGGCGGC | | | | | | |
| | CTCCT | | | | | | |
| rs50306 | CCGTGTCCACCTTGC | 735 | | | 22 | 42129083 | − |
| 55 | GCAACTTGGGCCTGG | | | | | | |
| | GCAAGAAGTCGCTGG | | | | | | |
| | AGCAG | | | | | | |
| rs50306 | CCGTGTCCACCTTGC | 736 | | | 22 | 42129083 | − |
| 55 | GCAACTTGGGCCTGG | | | | | | |
| | GCAAGAAGTCGCTGG | | | | | | |
| | AGCAG | | | | | | |
| rs78482 | TGGTTGGCGAAGGCG | 737 | TGGTTGGCGAAGGCG | 2213 | 22 | 42129087 | + |
| 768 | GCACAAAGGCAGGCG | | GCACAAAGGCAGGCG | | | | |
| | GCCTCCTCGGTCACC | | GCCTCCTCGGTCACC | | | | |
| | CACTC | | CACTG | | | | |
| rs56922 | AGGCGGCACAAAGGC | 738 | | | 22 | 42129098 | + |
| 9126 | AGGCGGCCTCCTYGG | | | | | | |
| | TCACCCACTSCTCCA | | | | | | |
| | GCGAC | | | | | | |
| rs56922 | AGGCGGCACAAAGGC | 739 | | | 22 | 42129098 | + |
| 9126 | AGGCGGCCTCCTYGG | | | | | | |
| | TCACCCACTSCTCCA | | | | | | |
| | GCGAC | | | | | | |
| rs56922 | GGCGGCACAAAGGCA | 740 | GGCGGCACAAAGGCA | 2214 | 22 | 42129098 | + |
| 9126 | GGCGGCCTCCTCGGT | | GGCGGCCTCCTCGGT | | | | |
| | CACCCACTGCTCCAG | | CACCCACTGCTCCAG | | | | |
| | CGACT | | CGACC | | | | |
| rs37513 | GCCCGCGTGGCGCGA | 741 | | | 22 | 42129113 | − |
| 5093 | GCAGAGGCGCTTCTC | | | | | | |
| | CGTGTCCACCTTGCG | | | | | | |
| | CAACT | | | | | | |
| rs37513 | GCCCGCGTGGCGCGA | 742 | | | 22 | 42129113 | − |
| 5093 | GCAGAGGCGCTTCTC | | | | | | |
| | CGTGTCCACCTTGCG | | | | | | |
| | CAACT | | | | | | |
| rs37513 | GCCCGCGTGGCGCGA | 743 | | | 22 | 42129113 | − |
| 5093 | GCAGAGGCGCTTCTC | | | | | | |
| | CGTGTCCACCTTGCG | | | | | | |
| | CAACT | | | | | | |
| rs61736 | GTTCCTGGCGCGCTA | 744 | GTTCCTGGCGCGCTA | 2215 | 22 | 42129132 | − |
| 512 | TGGGCCCGCGTGGCG | | TGGGCCCGCGTGGCG | | | | |
| | CGAGCAGAGGCGCTT | | CGAGCAGAGGCGCTT | | | | |
| | CTCCA | | CTCCG | | | | |
| rs61736 | GTTCCTGGCGCGCTA | 745 | GTTCCTGGCGCGCTA | 2216 | 22 | 42129132 | − |
| 512 | TGGGCCCGCGTGGCG | | TGGGCCCGCGTGGCG | | | | |
| | CGAGCAGAGGCGCTT | | CGAGCAGAGGCGCTT | | | | |
| | CTCCA | | CTCCG | | | | |
| rs61736 | GTTCCTGGCGCGCTA | 746 | GTTCCTGGCGCGCTA | 2217 | 22 | 42129132 | − |
| 512 | TGGGCCCGCGTGGCG | | TGGGCCCGCGTGGCG | | | | |
| | CGAGCAGAGGCGCTT | | CGAGCAGAGGCGCTT | | | | |
| | CTCCA | | CTCCG | | | | |
| rs78145 | ACTGCTCCAGCGACT | 747 | | | 22 | 42129134 | + |
| 7579 | TCTTGCCCAGGCCCR | | | | | | |
| | AGTTGCGCAAGGTGG | | | | | | |
| | ASAYG | | | | | | |
| rs78145 | GGTGTTCCTGGCGCG | 748 | | | 22 | 42129134 | − |
| 7579 | CTATGGGCCCGCGTG | | | | | | |
| | GCGCGAGCAGAGGCG | | | | | | |
| | CTTCT | | | | | | |
| rs78145 | GTGTTCCTGGCGCGC | 749 | GTGTTCCTGGCGCGC | 1900 | 22 | 42129134 | − |
| 7579 | TATGGGCCCGCGTGG | | TATGGGCCCGCGTGG | | | | |
| | CGCGAGCAGAGGCGC | | CGCGAGCAGAGGCGC | | | | |
| | TTCTT | | TTCTC | | | | |
| 22_ | CCAGGCCCAAGTTGC | 750 | | | 22 | 42129155 | + |
| 42129155 | GCAAGGTGGACACGG | | | | | | |
| | AGAAGCGCCTCTGCT | | | | | | |
| | CGCGC | | | | | | |
| 22_ | CAGGCCCAAGTTGCG | 751 | CAGGCCCAAGTTGCG | 1901 | 22 | 42129155 | + |
| 42129155 | CAAGGTGGACACGGA | | CAAGGTGGACACGGA | | | | |
| | GAAGCGCCTCTGCTC | | GAAGCGCCTCTGCTC | | | | |
| | GCGCT | | GCGCC | | | | |
| 22_ | CGTGCCCGTCCCACC | 752 | | | 22 | 42129155 | − |
| 42129155 | CCCAGGGGTGTTYCT | | | | | | |
| | GKCGCGCTATGGGCC | | | | | | |
| | CGCGT | | | | | | |
| rs11358 | GCCTTCATGGCCACG | 753 | | | 22 | 42129174 | − |
| 23 | CGCACGTGCCCGTCC | | | | | | |
| | CACCCCCAGGGGTGT | | | | | | |
| | TCCTG | | | | | | |
| rs11358 | ACGGAGAAGCGCCTC | 754 | ACGGAGAAGCGCCTC | 1902 | 22 | 42129180 | + |
| 22 | TGCTCGCGCCACGCG | | TGCTCGCGCCACGCG | | | | |
| | GGCCCATAGCGCGCC | | GGCCCATAGCGCGCC | | | | |
| | AGGAA | | AGGAT | | | | |
| rs37461 | GGAGAAGCGCCTCTG | 755 | | | 22 | 42129183 | + |
| 6348 | CTCGCGCCACGCGGG | | | | | | |
| | CCCATAGCGCGCCAG | | | | | | |
| | GAACA | | | | | | |
| rs37461 | GGAGAAGCGCCTCTG | 756 | | | 22 | 42129183 | + |
| 6348 | CTCGCGCCACGCGGG | | | | | | |
| | CCCATAGCGCGCCAG | | | | | | |
| | GAACA | | | | | | |
| rs37461 | GGAGAAGCGCCTCTG | 757 | | | 22 | 42129183 | + |
| 6348 | CTCGCGCCACGCGGG | | | | | | |
| | CCCATAGCGCGCCAG | | | | | | |
| | GAACA | | | | | | |
| rs53564 | AATCTGTCTCTGTCC | 758 | | | 22 | 42129759 | + |
| 2512 | CCACCGCTGCTTGCC | | | | | | |
| | TTGGGAACGCGGCCC | | | | | | |
| | RAARC | | | | | | |
| rs53564 | AATCTGTCTCTGTCC | 759 | | | 22 | 42129759 | + |
| 2512 | CCACCGCTGCTTGCC | | | | | | |
| | TTGGGAACGCGGCCC | | | | | | |
| | GAAAC | | | | | | |
| rs53564 | AATCTGTCTCTGTCC | 760 | | | 22 | 42129759 | + |
| 2512 | CCACCGCTGCTTGCC | | | | | | |
| | TTGGGAACGCGGCCC | | | | | | |
| | GAAAC | | | | | | |
| rs78459 | TCTCTGTCCCCACCG | 761 | | | 22 | 42129765 | + |
| 009 | CTGCTTGCCTTGGGA | | | | | | |
| | ACGCGGCCCRAARCY | | | | | | |
| | CAGGA | | | | | | |
| rs78459 | CTCTGTCCCCACCGC | 762 | CTCTGTCCCCACCGC | 1903 | 22 | 42129765 | + |
| 009 | TGCTTGCCTTGGGAA | | TGCTTGCCTTGGGAA | | | | |
| | CGCGGCCCGAAACCC | | CGCGGCCCGAAACCC | | | | |
| | AGGAT | | AGGAC | | | | |
| rs78459 | CTCTGTCCCCACCGC | 763 | CTCTGTCCCCACCGC | 1904 | 22 | 42129765 | + |
| 009 | TGCTTGCCTTGGGAA | | TGCTTGCCTTGGGAA | | | | |
| | CGCGGCCCRAARCYC | | CGCGGCCCRAARCYC | | | | |
| | AGGAT | | AGGAC | | | | |
| rs28371 | CTGGTGACCCACGGC | 764 | CTGGTGACCCACGGC | 1905 | 22 | 42129770 | − |
| 706 | GAGGACACCGCCGAC | | GAGGACACCGCCGAC | | | | |
| | CGCCCGCCTGTGCCC | | CGCCCGCCTGTGCCC | | | | |
| | ATCAT | | ATCAC | | | | |
| rs28371 | CTGGTGACCCACGGC | 765 | CTGGTGACCCACGGC | 1906 | 22 | 42129770 | − |
| 706 | GAGGACACCGCCGAC | | GAGGACACCGCCGAC | | | | |
| | CGCCCGCCTGTGCCC | | CGCCCGCCTGTGCCC | | | | |
| | ATCAT | | ATCAC | | | | |
| rs76187 | GCTGCTTGCCTTGGG | 766 | | | 22 | 42129779 | + |
| 628 | AACGCGGCCCGAAAC | | | | | | |
| | CCAGGAYCTGGDWGA | | | | | | |
| | TGGGC | | | | | | |
| rs76187 | CTGCTTGCCTTGGGA | 767 | CTGCTTGCCTTGGGA | 1907 | 22 | 42129779 | + |
| 628 | ACGCGGCCCGAAACC | | ACGCGGCCCGAAACC | | | | |
| | CAGGAYCTGGDWGAT | | CAGGAYCTGGDWGAT | | | | |
| | GGGCA | | GGGCG | | | | |
| rs76187 | GCGCGAGGCGCTGGT | 768 | | | 22 | 42129779 | − |
| 628 | GACCCACGGCGAGGA | | | | | | |
| | SACSGCCGACCGCCC | | | | | | |
| | GCCTR | | | | | | |
| rs28371 | CAGGATCTGGGTGAT | 769 | CAGGATCTGGGTGAT | 1908 | 22 | 42129809 | + |
| 704 | GGGCACAGGCGGGCG | | GGGCACAGGCGGGCG | | | | |
| | GTCGGCGGTGTCCTC | | GTCGGCGGTGTCCTC | | | | |
| | GCCGT | | GCCGC | | | | |
| rs28371 | CAGGATCTGGGTGAT | 770 | CAGGATCTGGGTGAT | 1909 | 22 | 42129809 | + |
| 704 | GGGCACAGGCGGGCG | | GGGCACAGGCGGGCG | | | | |
| | GTCGGCGGTGTCCTC | | GTCGGCGGTGTCCTC | | | | |
| | GCCGT | | GCCGC | | | | |
| rs28371 | CAGGATCTGGGTGAT | 771 | CAGGATCTGGGTGAT | 1910 | 22 | 42129809 | + |
| 704 | GGGCACAGGCGGGCG | | GGGCACAGGCGGGCG | | | | |
| | GTCGGCGGTGTCCTC | | GTCGGCGGTGTCCTC | | | | |
| | GCCGT | | GCCGC | | | | |
| rs28371 | CCTGGACGCCGGTGG | 772 | | | 22 | 42129819 | − |
| 703 | TCGTGCTCAATGGGC | | | | | | |
| | TGGCGGCCGTGCGCG | | | | | | |
| | AGGCG | | | | | | |
| rs28371 | CCTGGACGCCGGTGG | 773 | | | 22 | 42129819 | − |
| 703 | TCGTGCTCAATGGGC | | | | | | |
| | TGGCGGCCGTGCGCG | | | | | | |
| | AGGCG | | | | | | |
| rs26760 | TGATGGGCACAGGCG | 774 | | | 22 | 42129821 | + |
| 8309 | GGCGGTCGGCGGTST | | | | | | |
| | CCTCGCCGYGGGTCA | | | | | | |
| | CCAKC | | | | | | |
| rs26760 | TGATGGGCACAGGCG | 775 | | | 22 | 42129821 | + |
| 8309 | GGCGGTCGGCGGTST | | | | | | |
| | CCTCGCCGYGGGTCA | | | | | | |
| | CCAKC | | | | | | |
| rs26760 | TGATGGGCACAGGCG | 776 | | | 22 | 42129821 | + |
| 8309 | GGCGGTCGGCGGTST | | | | | | |
| | CCTCGCCGYGGGTCA | | | | | | |
| | CCAKC | | | | | | |
| rs26760 | CACAGGCGGGCGGTC | 777 | CACAGGCGGGCGGTC | 1911 | 22 | 42129827 | + |
| 8276 | GGCGGTGTCCTCGCC | | GGCGGTGTCCTCGCC | | | | |
| | GYGGGTCACCAKCRC | | GYGGGTCACCAKCRC | | | | |
| | CTCGC | | CTCGG | | | | |
| rs26760 | CACAGGCGGGCGGTC | 778 | CACAGGCGGGCGGTC | 1912 | 22 | 42129827 | + |
| 8276 | GGCGGTGTCCTCGCC | | GGCGGTGTCCTCGCC | | | | |
| | GTGGGTCACCAGCGC | | GTGGGTCACCAGCGC | | | | |
| | CTCGC | | CTCGG | | | | |
| rs26760 | CAGCTGGCCTGGACG | 779 | CAGCTGGCCTGGACG | 1913 | 22 | 42129827 | − |
| 8276 | CCGGTGGTCGTGCTC | | CCGGTGGTCGTGCTC | | | | |
| | AATGGGCTGGCGGCC | | AATGGGCTGGCGGCC | | | | |
| | GTGCG | | GTGCC | | | | |
| rs26760 | GGCGGTCGGCGGTGT | 780 | | | 22 | 42129836 | + |
| 8310 | CCTCGCCGTGGGTCA | | | | | | |
| | CCAGCGCCTCGCGCA | | | | | | |
| | CGGCC | | | | | | |
| 22_ | CAGCCCATTGAGCAC | 781 | CAGCCCATTGAGCAC | 1914 | 22 | 42129887 | + |
| 42129887 | GACCACCGGCGTCCA | | GACCACCGGCGTCCA | | | | |
| | GGCCAGCTGCAGGCT | | GGCCAGCTGCAGGCT | | | | |
| | GAACA | | GAACG | | | | |
| 22_ | ACAAGAGGCCCTGAC | 782 | | | 22 | 42129887 | − |
| 42129887 | CCTCCCTCTGCAGTT | | | | | | |
| | GCGGCGCCGCTTCGG | | | | | | |
| | GGACG | | | | | | |
| 22_ | ACAAGAGGCCCTGAC | 783 | | | 22 | 42129887 | − |
| 42129887 | CCTCCCTCTGCASTT | | | | | | |
| | GYGGCGCCGCTTCGG | | | | | | |
| | GGACG | | | | | | |
| rs26760 | CTGGGGTGATCCTGG | 784 | | | 22 | 42129906 | − |
| 8311 | CTTGACAAGAGGCCC | | | | | | |
| | TGACCCTCCCTCTGC | | | | | | |
| | ASTTG | | | | | | |
| rs26760 | CTGGGGTGATCCTGG | 785 | | | 22 | 42129906 | − |
| 8311 | CTTGACAAGAGGCCC | | | | | | |
| | TGACCCTCCCTCTGC | | | | | | |
| | AGTTG | | | | | | |
| rs26760 | CTGGGGTGATCCTGG | 786 | | | 22 | 42129906 | − |
| 8311 | CTTGACAAGAGGCCC | | | | | | |
| | TGACCCTCCCTCTGC | | | | | | |
| | AGTTG | | | | | | |
| rs20137 | AGTCTGGGGTGATCC | 787 | AGTCTGGGGTGATCC | 1915 | 22 | 42129910 | − |
| 7835 | TGGCTTGACAAGAGG | | TGGCTTGACAAGAGG | | | | |
| | CCCTGACCCTCCCTC | | CCCTGACCCTCCCTC | | | | |
| | TGCAG | | TGCAC | | | | |
| rs20137 | AGTCTGGGGTGATCC | 788 | AGTCTGGGGTGATCC | 1916 | 22 | 42129910 | − |
| 7835 | TGGCTTGACAAGAGG | | TGGCTTGACAAGAGG | | | | |
| | CCCTGACCCTCCCTC | | CCCTGACCCTCCCTC | | | | |
| | TGCAG | | TGCAC | | | | |
| rs11820 | CGAAGCAGTATGGTG | 789 | | | 22 | 42130667 | + |
| 3758 | TGTTCTGGAAGTCCA | | | | | | |
| | CATGCAGCAGGTTGC | | | | | | |
| | CCAGC | | | | | | |
| rs11820 | GAAGCAGTATGGTGT | 790 | GAAGCAGTATGGTGT | 1917 | 22 | 42130667 | + |
| 3758 | GTTCTGGAAGTCCAC | | GTTCTGGAAGTCCAC | | | | |
| | ATGCAGCAGGTTGCC | | ATGCAGCAGGTTGCC | | | | |
| | CAGCT | | CAGCC | | | | |
| rs50308 | GGCGCCAACGCTGGG | 791 | | | 22 | 42130668 | − |
| 62 | CTGCACGCTACCCAC | | | | | | |
| | CAGGCCCCCTGCCAC | | | | | | |
| | TGCCC | | | | | | |
| rs50308 | GGCGCCAACGCTGGG | 792 | | | 22 | 42130668 | − |
| 62 | CTGCACGCTACCCAC | | | | | | |
| | CAGGCCCCCTGCCAC | | | | | | |
| | TGCCC | | | | | | |
| rs10658 | GTCCACATGCAGCAG | 793 | | | 22 | 42130692 | + |
| 52 | GTTGCCCAGCCYGGG | | | | | | |
| | CAGTGGCAGGGGRCC | | | | | | |
| | TGGYG | | | | | | |
| rs10658 | GTCCACATGCAGCAG | 794 | | | 22 | 42130692 | + |
| 52 | GTTGCCCAGCCCGGG | | | | | | |
| | CAGTGGCAGGGGGCC | | | | | | |
| | TGGTG | | | | | | |
| rs13810 | TGATAGTGGCCATCT | 795 | | | 22 | 42130710 | − |
| 0349 | TCCTGCTCCTGGTGG | | | | | | |
| | ACCTGATGCACCGGC | | | | | | |
| | GCCAA | | | | | | |
| rs13810 | TGATAGTGGCCATCT | 796 | | | 22 | 42130710 | − |
| 0349 | TCCTGCTCCTGGTGG | | | | | | |
| | ACCTGATGCACCGGC | | | | | | |
| | GCCAA | | | | | | |
| rs13810 | TGATAGTGGCCATCT | 797 | | | 22 | 42130710 | − |
| 0349 | TCCTGCTCCTGGTGG | | | | | | |
| | ACCTGATGCACYRGC | | | | | | |
| | RCCAA | | | | | | |
| rs28371 | GCCGTGATAGTGGCC | 798 | GCCGTGATAGTGGCC | 1918 | 22 | 42130715 | − |
| 696 | ATCTTCCTGCTCCTG | | ATCTTCCTGCTCCTG | | | | |
| | GTGGACCTGATGCAC | | GTGGACCTGATGCAC | | | | |
| | CGGCA | | CGGCG | | | | |
| rs26760 | GGGCAGTGGCAGGGG | 799 | | | 22 | 42130719 | + |
| 8313 | GCCTGGTGGGTAGCG | | | | | | |
| | TGCAGCCCAGCRTTG | | | | | | |
| | GYGCC | | | | | | |
| rs26760 | GGGCAGTGGCAGGGG | 800 | | | 22 | 42130719 | + |
| 8313 | GCCTGGTGGGTAGCG | | | | | | |
| | TGCAGCCCAGCRTTG | | | | | | |
| | GYGCC | | | | | | |
| rs26760 | GGGCAGTGGCAGGGG | 801 | | | 22 | 42130719 | + |
| 8313 | GCCTGGTGGGTAGCG | | | | | | |
| | TGCAGCCCAGCRTTG | | | | | | |
| | GYGCC | | | | | | |
| 22_ | AGGGGGCCTGGTGGG | 802 | | | 22 | 42130727 | + |
| 42130727 | TAGCGTGCAGCCCAG | | | | | | |
| | CGTTGGCGCCGGTGC | | | | | | |
| | ATCAG | | | | | | |
| 22_ | AGGGGGCCTGGTGGG | 803 | | | 22 | 42130727 | + |
| 42130727 | TAGCGTGCAGCCCAG | | | | | | |
| | CGTTGGYGCYRGTGC | | | | | | |
| | ATCAG | | | | | | |
| 22_ | CACTGGTGCCCCTGG | 804 | | | 22 | 42130727 | − |
| 42130727 | CCGTGATAGTGGCCA | | | | | | |
| | TCTTCCTGCTCCTGG | | | | | | |
| | TGGAC | | | | | | |
| rs76925 | CATTTGGTAGTGAGG | 805 | | | 22 | 42130761 | − |
| 8 | CAGGTATGGGGCTAG | | | | | | |
| | AAGCACTGGTGCCCC | | | | | | |
| | TGGCC | | | | | | |
| rs76925 | CATTTGGTAGTGAGG | 806 | | | 22 | 42130761 | − |
| 8 | CAGGTATGGGGCTAG | | | | | | |
| | AAGCACTGGTGCCCC | | | | | | |
| | TGGCC | | | | | | |
| rs72549 | TCCAGAGGAGCCCAT | 807 | | | 22 | 42130773 | − |
| 358 | TTGGTAGTGAGGCAG | | | | | | |
| | GTATGGGGCTAGAAG | | | | | | |
| | CACTG | | | | | | |
| rs72549 | CCAGAGGAGCCCATT | 808 | CCAGAGGAGCCCATT | 1919 | 22 | 42130773 | − |
| 358 | TGGTAGTGAGGCAGG | | TGGTAGTGAGGCAGG | | | | |
| | TATGGGGCTAGAAGC | | TATGGGGCTAGAAGC | | | | |
| | ACTGA | | ACTGG | | | | |
| rs72549 | TCCAGAGGAGCCCAT | 809 | | | 22 | 42130773 | − |
| 358 | TTGGTAGTGAGGCAG | | | | | | |
| | GTATGGGGCTAGAAG | | | | | | |
| | CACTG | | | | | | |
| rs77379 | GTGTGTCCAGAGGAG | 810 | | | 22 | 42130778 | − |
| 0593 | CCCATTTGGTAGTGA | | | | | | |
| | GGCAGGTATGGGGCT | | | | | | |
| | AGAAG | | | | | | |
| rs11358 | ACTGGACAGCCCCGG | 811 | ACTGGACAGCCCCGG | 1920 | 22 | 42522613 | − |
| 40 | CCCAGCCACCATGGT | | CCCAGCCACCATGGT | | | | |
| | GTCTTTGCTTTCCTG | | GTCTTTGCTTTCCTG | | | | |
| | GTGAC | | GTGAG | | | | |
| rs11358 | ACTGGACAGCCCCGG | 812 | ACTGGACAGCCCCGG | 1921 | 22 | 42522613 | − |
| 40 | CCCAGCCACCATGGT | | CCCAGCCACCATGGT | | | | |
| | GTCTTTGCTTTCCTG | | GTCTTTGCTTTCCTG | | | | |
| | GTGAC | | GTGAG | | | | |
| rs11358 | ACTGGACAGCCCCGG | 813 | ACTGGACAGCCCCGG | 1922 | 22 | 42522613 | − |
| 40 | CCCAGCCACCATGGT | | CCCAGCCACCATGGT | | | | |
| | GTCTTTGCTTTCCTG | | GTCTTTGCTTTCCTG | | | | |
| | GTGAC | | GTGAG | | | | |
| rs11358 | GCGGGGCACAGCACA | 814 | | | 22 | 42522629 | + |
| 38 | AAGCTCATAGGGGGA | | | | | | |
| | TGGGGTCACCAGGAA | | | | | | |
| | AGCAA | | | | | | |
| rs28371 | AGCACAAAGCTCATA | 815 | | | 22 | 42522638 | + |
| 735 | GGGGGATGGGGTCAC | | | | | | |
| | CAGGAAAGCAAAGAC | | | | | | |
| | ACCAT | | | | | | |
| rs28371 | AGCACAAAGCTCATA | 816 | | | 22 | 42522638 | + |
| 735 | GGGGGATGGGGTCAC | | | | | | |
| | CAGGAAAGCAAAGAC | | | | | | |
| | ACCAT | | | | | | |
| rs26760 | AGAAGAGGAAGAGCT | 817 | | | 22 | 42522751 | + |
| 8319 | CCATGCGGGCCAGGG | | | | | | |
| | GCTCCCCGAGGCATG | | | | | | |
| | CACGG | | | | | | |
| rs28371 | CGGTGCTGAAGGATG | 818 | | | 22 | 42522916 | − |
| 733 | AGGCCGTCTGGGAGA | | | | | | |
| | AGCCCTTCCGCTTCC | | | | | | |
| | ACCCC | | | | | | |
| rs28371 | CAGGAAGGCCTCCGG | 819 | | | 22 | 42522916 | + |
| 733 | CTTCACAAAGTGGCC | | | | | | |
| | CTGGGCATCCAGGAA | | | | | | |
| | GTGTT | | | | | | |
| 22_ | CATCTTCTGCCCAGG | 820 | | | 22 | 42522958 | − |
| 42126956 | GAACGACACTCATCA | | | | | | |
| | CCAACCTGTCATCGG | | | | | | |
| | TGCTG | | | | | | |
| rs77312 | CTGGGTGTGACCCAT | 821 | CTGGGTGTGACCCAT | 1923 | 22 | 42523459 | − |
| 092 | ATGACATCCCGTGAC | | ATGACATCCCGTGAC | | | | |
| | ATCGAAGTACAGGGC | | ATCGAAGTACAGGGC | | | | |
| | TTCCG | | TTCCC | | | | |
| rs77312 | CCTGGGTGTGACCCA | 822 | | | 22 | 42523459 | − |
| 092 | TATGACATCCCGTGA | | | | | | |
| | CATCGAAGTACAGGG | | | | | | |
| | CTTCC | | | | | | |
| rs72549 | TGGGTGACCAGGCTC | 823 | | | 22 | 42523533 | − |
| 346 | ACATGCCCTACACCA | | | | | | |
| | CTGCCGTGATTCATG | | | | | | |
| | AGGTG | | | | | | |
| rs72549 | TGGGTGACCAGGCTC | 824 | | | 22 | 42523533 | − |
| 346 | ACATGCCCTACACCA | | | | | | |
| | CTGCCGTGATTCATG | | | | | | |
| | AGGTG | | | | | | |
| rs72549 | TGGGTGACCAGGCTC | 825 | | | 22 | 42523533 | − |
| 346 | ACATGCCCTACACCA | | | | | | |
| | CTGCCGTGATTCATG | | | | | | |
| | AGGTG | | | | | | |
| rs20210 | GGGGGACGATGTCCC | 826 | | | 22 | 42523558 | + |
| 2799 | CAAAGCGCTGCACCT | | | | | | |
| | CATGAATCACGGCAG | | | | | | |
| | TGGTG | | | | | | |
| rs20210 | GGGGGACGATGTCCC | 827 | | | 22 | 42523558 | + |
| 2799 | CAAAGCGCTGCACCT | | | | | | |
| | CATGAATCACGGCAG | | | | | | |
| | TGGTG | | | | | | |
| rs76088 | GAATCACGGCAGTGG | 828 | | | 22 | 42523591 | + |
| 846 | TGTAGGGCATGTGAG | | | | | | |
| | CCTGGTCACCCATCT | | | | | | |
| | CTGGT | | | | | | |
| rs74871 | CATTGTGGGGACGCA | 829 | | | 22 | 42523612 | − |
| 2690 | TGTCTGTCCAGGCCG | | | | | | |
| | TGTCCAACAGGAGAT | | | | | | |
| | CGACG | | | | | | |
| rs72549 | GTGCTGACCCATTGT | 830 | | | 22 | 42523621 | − |
| 348 | GGGGACGCATGTCTG | | | | | | |
| | TCCAGGCCGTGTCCA | | | | | | |
| | ACAGG | | | | | | |
| rs72549 | GTGCTGACCCATTGT | 831 | | | 22 | 42523621 | − |
| 348 | GGGGACGCATGTCTG | | | | | | |
| | TCCAGGCCGTGTCCA | | | | | | |
| | ACAGG | | | | | | |
| rs72549 | GTGCTGACCCATTGT | 832 | | | 22 | 42523621 | − |
| 348 | GGGGACGCATGTCTG | | | | | | |
| | TCCAGGCCGTGTCCA | | | | | | |
| | ACAGG | | | | | | |
| rs16947 | CTGCTCAGGCCAAGG | 833 | | | 22 | 42523943 | − |
| | GGAACCCTGAGAGCA | | | | | | |
| | GCTTCAATGATGAGA | | | | | | |
| | ACCTG | | | | | | |
| rs16947 | CTGCTCAGGCCAAGG | 834 | | | 22 | 42523943 | − |
| | GGAACCCTGAGAGCA | | | | | | |
| | GCTTCAATGATGAGA | | | | | | |
| | ACCTG | | | | | | |
| rs26760 | TGTCCCGAGTATGCT | 835 | | | 22 | 42523965 | − |
| 8279 | CTCGGCCCTGCTCAG | | | | | | |
| | GCCAAGGGGAACCCT | | | | | | |
| | GAGAG | | | | | | |
| rs11358 | GACCCAGCCCAGCCC | 836 | GACCCAGCCCAGCCC | 1924 | 22 | 42524183 | − |
| 28 | CCCCGAGACCTGACT | | CCCCGAGACCTGACT | | | | |
| | GAGGCCTTCCTGGCA | | GAGGCCTTCCTGGCA | | | | |
| | GAGAA | | GAGAT | | | | |
| rs77913 | CCCACCACCCTTGCC | 837 | | | 22 | 42524187 | + |
| 725 | CCCCACCGTGGCAGC | | | | | | |
| | CACTCTCACCTTCTC | | | | | | |
| | CATCT | | | | | | |
| rs77913 | CCCACCACCCTTGCC | 838 | | | 22 | 42524187 | + |
| 725 | CCCCACCGTGGCAGC | | | | | | |
| | CACTCTCACCTTCTC | | | | | | |
| | CATCT | | | | | | |
| rs72549 | CACCGTGGCAGCCAC | 839 | | | 22 | 42524203 | + |
| 351 | TCTCACCTTCTCCAT | | | | | | |
| | CTCTGCCAGGAAGGC | | | | | | |
| | CTCAG | | | | | | |
| rs26760 | CTGCCAGGAAGGCCT | 840 | | | 22 | 42524237 | + |
| 8297 | CAGTCAGGTCTCGGG | | | | | | |
| | GGGGCTGGGCTGGGT | | | | | | |
| | CCCAG | | | | | | |
| rs26760 | CTGCCAGGAAGGCCT | 841 | | | 22 | 42524237 | + |
| 8297 | CAGTCAGGTCTCGGG | | | | | | |
| | GGGGCTGGGCTGGGT | | | | | | |
| | CCCAG | | | | | | |
| rs19953 | TCCTCGGTCTCTCGC | 842 | | | 22 | 42524814 | + |
| 5154 | TCCGCACCTCGCGCA | | | | | | |
| | GAAAGCCCGACTCCT | | | | | | |
| | CCTTC | | | | | | |
| rs38920 | CACGTTGCTCACGGC | 843 | | | 22 | 42524947 | + |
| 97 | TTTGTCCAAGAGACC | | | | | | |
| | GTTGGGGCGAAAGGG | | | | | | |
| | GCGTC | | | | | | |
| rs26760 | CCCATCACCCACCGG | 844 | | | 22 | 42525073 | + |
| 8302 | AGTGGTTGGCGAAGG | | | | | | |
| | CGGCACAAAGGCAGG | | | | | | |
| | CGGCC | | | | | | |
| rs26760 | CCCATCACCCACCGG | 845 | | | 22 | 42525073 | + |
| 8302 | AGTGGTTGGCGAAGG | | | | | | |
| | CGGCACAAAGGCAGG | | | | | | |
| | CGGCC | | | | | | |
| rs28371 | TCACCCACCGGAGTG | 846 | | | 22 | 42525077 | + |
| 710 | GTTGGCGAAGGCGGC | | | | | | |
| | ACAAAGGCAGGCGGC | | | | | | |
| | CTCCT | | | | | | |
| rs78482 | TGGTTGGCGAAGGCG | 847 | TGGTTGGCGAAGGCG | 1925 | 22 | 42525089 | + |
| 768 | GCACAAAGGCAGGCG | | GCACAAAGGCAGGCG | | | | |
| | GCCTCCTCGGTCACC | | GCCTCCTCGGTCACC | | | | |
| | CACTC | | CACTG | | | | |
| rs78482 | TGGTTGGCGAAGGCG | 848 | TGGTTGGCGAAGGCG | 1926 | 22 | 42525089 | + |
| 768 | GCACAAAGGCAGGCG | | GCACAAAGGCAGGCG | | | | |
| | GCCTCCTCGGTCACC | | GCCTCCTCGGTCACC | | | | |
| | CACTC | | CACTG | | | | |
| rs11358 | GGTGGAGACGGAGAA | 849 | | | 22 | 42525176 | + |
| 23 | GCGCCTCTGCTCGCG | | | | | | |
| | CCACGCGGGCCCATA | | | | | | |
| | GCGCG | | | | | | |
| rs11358 | GGTGGAGACGGAGAA | 850 | | | 22 | 42525176 | + |
| 23 | GCGCCTCTGCTCGCG | | | | | | |
| | CCACGCGGGCCCATA | | | | | | |
| | GCGCG | | | | | | |
| rs11358 | ACGGAGAAGCGCCTC | 851 | ACGGAGAAGCGCCTC | 1927 | 22 | 42525182 | + |
| 22 | TGCTCGCGCCACGCG | | TGCTCGCGCCACGCG | | | | |
| | GGCCCATAGCGCGCC | | GGCCCATAGCGCGCC | | | | |
| | AGGAA | | AGGAT | | | | |
| rs11358 | CTAATGCCTTCATGG | 852 | CTAATGCCTTCATGG | 1928 | 22 | 42525182 | − |
| 22 | CCACGCGCACGTGCC | | CCACGCGCACGTGCC | | | | |
| | CGTCCCACCCCCAGG | | CGTCCCACCCCCAGG | | | | |
| | GGTGT | | GGTGA | | | | |
| rs28371 | CTGGTGACCCACGGC | 853 | CTGGTGACCCACGGC | 1929 | 22 | 42525772 | − |
| 706 | GAGGACACCGCCGAC | | GAGGACACCGCCGAC | | | | |
| | CGCCCGCCTGTGCCC | | CGCCCGCCTGTGCCC | | | | |
| | ATCAT | | ATCAC | | | | |
| rs26760 | CTGCTTGCCTTGGGA | 854 | | | 22 | 42525782 | + |
| 8308 | ACGCGGCCCGAAACC | | | | | | |
| | CAGGATCTGGGTGAT | | | | | | |
| | GGGCA | | | | | | |
| rs26760 | CTGCTTGCCTTGGGA | 855 | | | 22 | 42525782 | + |
| 8308 | ACGCGGCCCGAAACC | | | | | | |
| | CAGGATCTGGGTGAT | | | | | | |
| | GGGCA | | | | | | |
| rs26760 | CTGCTTGCCTTGGGA | 856 | | | 22 | 42525782 | + |
| 8308 | ACGCGGCCCGAAACC | | | | | | |
| | CAGGATCTGGGTGAT | | | | | | |
| | GGGCA | | | | | | |
| rs28371 | CCTGGACGCCGGTGG | 857 | | | 22 | 42525821 | − |
| 703 | TCGTGCTCAATGGGC | | | | | | |
| | TGGCGGCCGTGCGCG | | | | | | |
| | AGGCG | | | | | | |
| rs26760 | GGCGGTCGGCGGTGT | 858 | | | 22 | 42525838 | + |
| 8310 | CCTCGCCGTGGGTCA | | | | | | |
| | CCAGCGCCTCGCGCA | | | | | | |
| | CGGCC | | | | | | |
| rs26760 | GGCGGTCGGCGGTGT | 859 | | | 22 | 42525838 | + |
| 8310 | CCTCGCCGTGGGTCA | | | | | | |
| | CCAGCGCCTCGCGCA | | | | | | |
| | CGGCC | | | | | | |
| rs20137 | AGTCTGGGGGGATCC | 860 | AGTCTGGGGGGATCC | 1930 | 22 | 42525912 | − |
| 7835 | TGGCTTGACAAGAGG | | TGGCTTGACAAGAGG | | | | |
| | CCCTGACCCTCCCTC | | CCCTGACCCTCCCTC | | | | |
| | TGCAG | | TGCAC | | | | |
| rs77467 | CCTCACCTGGTCGAA | 861 | | | 22 | 42526656 | + |
| 1100 | GCAGTATGGTGTGTT | | | | | | |
| | CTGGAAGTCCACATG | | | | | | |
| | CAGCA | | | | | | |
| rs77467 | CCTCACCTGGTCGAA | 862 | | | 22 | 42526656 | + |
| 1100 | GCAGTATGGTGTGTT | | | | | | |
| | CTGGAAGTCCACATG | | | | | | |
| | CAGCA | | | | | | |
| rs77467 | CCTCACCTGGTCGAA | 863 | | | 22 | 42526656 | + |
| 1100 | GCAGTATGGTGTGTT | | | | | | |
| | CTGGAAGTCCACATG | | | | | | |
| | CAGCA | | | | | | |
| rs50308 | GAAGCAGTATGGTGT | 864 | | | 22 | 42526670 | + |
| 62 | GTTCTGGAAGTCCAC | | | | | | |
| | ATGCAGCAGGTTGCC | | | | | | |
| | CAGCC | | | | | | |
| rs10658 | GTCCACATGCAGCAG | 865 | | | 22 | 42526694 | + |
| 52 | GTTGCCCAGCCCGGG | | | | | | |
| | CAGTGGCAGGGGGCC | | | | | | |
| | TGGTG | | | | | | |
| rs28371 | GGCCGTGATAGTGGC | 866 | | | 22 | 42526717 | − |
| 696 | CATCTTCCTGCTCCT | | | | | | |
| | GGTGGACCTGATGCA | | | | | | |
| | CCGGC | | | | | | |
| rs28371 | GGCCGTGATAGTGGC | 867 | | | 22 | 42526717 | − |
| 696 | CATCTTCCTGCTCCT | | | | | | |
| | GGTGGACCTGATGCA | | | | | | |
| | CCGGC | | | | | | |
| rs76925 | CATTTGGTAGTGAGG | 868 | | | 22 | 42526763 | − |
| 8 | CAGGTATGGGGCTAG | | | | | | |
| | AAGCACTGGTGCCCC | | | | | | |
| | TGGCC | | | | | | |
| rs77379 | GTGTGTCCAGAGGAG | 869 | | | 22 | 42526780 | − |
| 0593 | CCCATTTGGTAGTGA | | | | | | |
| | GGCAGGTATGGGGCT | | | | | | |
| | AGAAG | | | | | | |
| rs77379 | GTGTGTCCAGAGGAG | 870 | | | 22 | 42526780 | − |
| 0593 | CCCATTTGGTAGTGA | | | | | | |
| | GGCAGGTATGGGGCT | | | | | | |
| | AGAAG | | | | | | |
| rs11423 | TCAGTGTGATTTTAT | 871 | | | 6 | 18130687 | + |
| 45 | TTTATCTATGTCTCA | | | | | | |
| | TTTACTTTTCTGTAA | | | | | | |
| | GTAGA | | | | | | |
| rs11423 | TGAAGAACGACATAA | 872 | | | 6 | 18130687 | − |
| 45 | AAGTTGGGGAATTGA | | | | | | |
| | CTGTCTTTTTGAAAA | | | | | | |
| | GTTAT | | | | | | |
| rs11423 | GAAGAACGACATAAA | 873 | GAAGAACGACATAAA | 1931 | 6 | 18130687 | − |
| 45 | AGTTGGGGAATTGAC | | AGTTGGGGAATTGAC | | | | |
| | TGTCTTTTTGAAAAG | | TGTCTTTTTGAAAAG | | | | |
| | TTATA | | TTATG | | | | |
| rs18005 | CCTGATGTCATTCTT | 874 | | | 6 | 18130781 | − |
| 84 | CATAGTATTTTAACA | | | | | | |
| | TGTTACTCTTTCTTG | | | | | | |
| | TTTCA | | | | | | |
| rs18005 | CCTGATGTCATTCTT | 875 | | | 6 | 18130781 | − |
| 84 | CATAGTATTTTAACA | | | | | | |
| | TGTTACTCTTTCTTG | | | | | | |
| | TTTCA | | | | | | |
| rs18005 | CCTGATGTCATTCTT | 876 | | | 6 | 18130781 | − |
| 84 | CATAGTATTTTAACA | | | | | | |
| | TGTTACTCTTTCTTG | | | | | | |
| | TTTCA | | | | | | |
| rs74423 | CTGTGTAGAGAAATG | 877 | CTGTGTAGAGAAATG | 1932 | 6 | 18133884 | − |
| 290 | TAACAAATACCTTCT | | TAACAAATACCTTCT | | | | |
| | TGTGTTCTTTGTAGC | | TGTGTTCTTTGTAGC | | | | |
| | TATGC | | TATGG | | | | |
| rs74423 | CTGTGTAGAGAAATG | 878 | CTGTGTAGAGAAATG | 1933 | 6 | 18133884 | − |
| 290 | TAACAAATACCTTCT | | TAACAAATACCTTCT | | | | |
| | TGTGTTCTTTGTAGC | | TGTGTTCTTTGTAGC | | | | |
| | TATGC | | TATGG | | | | |
| rs74423 | CTGTGTAGAGAAATG | 879 | CTGTGTAGAGAAATG | 1934 | 6 | 18133884 | − |
| 290 | TAACAAATACCTTCT | | TAACAAATACCTTCT | | | | |
| | TGTGTTCTTTGTAGC | | TGTGTTCTTTGTAGC | | | | |
| | TATGG | | TATGC | | | | |
| rs93335 | GGAGATACTGAAACT | 880 | | | 6 | 18133890 | + |
| 70 | TCTTTCCCAGGAGGG | | | | | | |
| | AAAACATTGTATCTG | | | | | | |
| | CATAG | | | | | | |
| rs93335 | GGAGATACTGAAACT | 881 | | | 6 | 18133890 | + |
| 70 | TCTTTCCCAGGAGGG | | | | | | |
| | AAAACATTGTATCTG | | | | | | |
| | CATAG | | | | | | |
| rs93335 | CCTTTGCCTGTGTAG | 882 | | | 6 | 18133890 | − |
| 70 | AGAAATGTAACAAAT | | | | | | |
| | ACCTTCTTGTGTTCT | | | | | | |
| | TTGTA | | | | | | |
| rs18004 | TTTCTGGTAGGACAA | 883 | | | 6 | 18138997 | − |
| 60 | ATATTGGCAAATTTG | | | | | | |
| | ACATGATTTGGGATA | | | | | | |
| | GAGGA | | | | | | |
| rs18004 | TTTCTGGTAGGACAA | 884 | | | 6 | 18138997 | − |
| 60 | ATATTGGCAAATTTG | | | | | | |
| | ACATGATTTGGGATA | | | | | | |
| | GAGGA | | | | | | |
| rs18004 | TTTCTGGTAGGACAA | 885 | | | 6 | 18138997 | − |
| 60 | ATATTGGCAAATTTG | | | | | | |
| | ACATGATTTGGGATA | | | | | | |
| | GAGGA | | | | | | |
| rs72552 | AAGTGCAGATGTAGT | 886 | | | 6 | 18139689 | + |
| 738 | ATTCAACCTACCTGG | | | | | | |
| | GAAGATCAAAAATAC | | | | | | |
| | TGCAA | | | | | | |
| rs72552 | AAGTGCAGATGTAGT | 887 | | | 6 | 18139689 | + |
| 738 | ATTCAACCTACCTGG | | | | | | |
| | GAAGATCAAAAATAC | | | | | | |
| | TGCAA | | | | | | |
| rs72552 | AAGTGCAGATGTAGT | 888 | | | 6 | 18139689 | + |
| 738 | ATTCAACCTACCTGG | | | | | | |
| | GAAGATCAAAAATAC | | | | | | |
| | TGCAA | | | | | | |
| rs18004 | ATCCCAAGTTCACTG | 889 | ATCCCAAGTTCACTG | 1935 | 6 | 18143724 | + |
| 62 | ATTTCCACACCAACT | | ATTTCCACACCAACT | | | | |
| | ACACTGTGTCCCCGG | | ACACTGTGTCCCCGG | | | | |
| | TCTGG | | TCTGC | | | | |
| rs18004 | TCATTTGAAAACATA | 890 | TCATTTGAAAACATA | 1936 | 6 | 18143724 | − |
| 62 | ATTTAAGTGTAAATG | | ATTTAAGTGTAAATG | | | | |
| | TATGATTTTATGCAG | | TATGATTTTATGCAG | | | | |
| | GTTTC | | GTTTG | | | | |
| rs18004 | ATCCCAAGTTCACTG | 891 | ATCCCAAGTTCACTG | 1937 | 6 | 18143724 | + |
| 62 | ATTTCCACACCAACT | | ATTTCCACACCAACT | | | | |
| | ACACTGTGTCCCCGG | | ACACTGTGTCCCCGG | | | | |
| | TCTGG | | TCTGC | | | | |
| rs26760 | CGTAGGCACGGAAGA | 892 | | | 6 | 18149126 | − |
| 7275 | CATATGCTTGTGAGA | | | | | | |
| | CAAAGGTGTCTCTGA | | | | | | |
| | AACTA | | | | | | |
| rs26760 | CGTAGGCACGGAAGA | 893 | | | 6 | 18149126 | − |
| 7275 | CATATGCTTGTGAGA | | | | | | |
| | CAAAGGTGTCTCTGA | | | | | | |
| | AACTA | | | | | | |
| rs26760 | CGTAGGCACGGAAGA | 894 | | | 6 | 18149126 | − |
| 7275 | CATATGCTTGTGAGA | | | | | | |
| | CAAAGGTGTCTCTGA | | | | | | |
| | AACTA | | | | | | |
| rs93335 | ACGTAGGCACGGAAG | 895 | | | 6 | 18149127 | − |
| 69 | ACATATGCTTGTGAG | | | | | | |
| | ACAAAGGTGTCTCTG | | | | | | |
| | AAACT | | | | | | |
| rs93335 | ACGTAGGCACGGAAG | 896 | | | 6 | 18149127 | − |
| 69 | ACATATGCTTGTGAG | | | | | | |
| | ACAAAGGTGTCTCTG | | | | | | |
| | AAACT | | | | | | |
| rs93335 | ACGTAGGCACGGAAG | 897 | | | 6 | 18149127 | − |
| 69 | ACATATGCTTGTGAG | | | | | | |
| | ACAAAGGTGTCTCTG | | | | | | |
| | AAACT | | | | | | |
| rs38690 | CAGGGTCTAGTAGAT | 898 | | | 6 | 30035987 | + |
| 66 | CACATGATTCATCAG | | | | | | |
| | AGGACTTGAAATACA | | | | | | |
| | TGCTG | | | | | | |
| rs38690 | CAGGGTCTAGTAGAT | 899 | | | 6 | 30035987 | + |
| 66 | CACATGATTCATCAG | | | | | | |
| | AGGACTTGAAATACA | | | | | | |
| | TGCTG | | | | | | |
| rs38690 | CAGGGTCTAGTAGAT | 2198 | | | 6 | 30035987 | + |
| 66 | CACATGATTCATCAG | | | | | | |
| | AGGACTTGAAATACA | | | | | | |
| | TGCTG | | | | | | |
| rs92637 | TTTACAAGGACCCCA | 2199 | | | 6 | 31138722 | + |
| 26 | GCTCCTTAACACAGA | | | | | | |
| | TCCCAGCTCCGAGGA | | | | | | |
| | AACTC | | | | | | |
| rs92637 | TTTACAAGGACCCCA | 2200 | | | 6 | 31138722 | + |
| 26 | GCTCCTTAACACAGA | | | | | | |
| | TCCCAGCTCCGAGGA | | | | | | |
| | AACTC | | | | | | |
| rs92637 | TTTACAAGGACCCCA | 2201 | | | 6 | 31138722 | + |
| 26 | GCTCCTTAACACAGA | | | | | | |
| | TCCCAGCTCCGAGGA | | | | | | |
| | AACTC | | | | | | |
| rs23950 | CCTTAAAAGGGGACA | 2202 | | | 6 | 31464003 | − |
| 29 | AATGAGTGCCCATTG | | | | | | |
| | AACTACACATTACAG | | | | | | |
| | CTGCC | | | | | | |
| rs23950 | CCTTAAAAGGGGACA | 2203 | | | 6 | 31464003 | − |
| 29 | AATGAGTGCCCATTG | | | | | | |
| | AACTACACATTACAG | | | | | | |
| | CTGCC | | | | | | |
| rs23950 | CCTTAAAAGGGGACA | 2204 | | | 6 | 31464003 | − |
| 29 | AATGAGTGCCCATTG | | | | | | |
| | AACTACACATTACAG | | | | | | |
| | CTGCC | | | | | | |
| rs80282 | TACTTCATCAAATTT | 2205 | | | 7 | 117174372 | − |
| 562 | GTTCAGGTTGTTGGA | | | | | | |
| | AAGGAGACTAACAAG | | | | | | |
| | TTGTC | | | | | | |
| rs11399 | TTTGGTAATAGGACA | 2206 | | | 7 | 117227832 | + |
| 3959 | TCTCCAAGTTTGCAG | | | | | | |
| | AGAAAGACAATATAG | | | | | | |
| | TTCTT | | | | | | |
| rs12190 | CAGAGAAAGACAATA | 2207 | | | 7 | 117227859 | + |
| 9013 | TAGTTCTTGGAGAAG | | | | | | |
| | GTGGAATCACACTGA | | | | | | |
| | GTGGA | | | | | | |
| rs12190 | CAGAGAAAGACAATA | 900 | | | 7 | 117227859 | + |
| 9013 | TAGTTCTTGGAGAAG | | | | | | |
| | GTGGAATCACACTGA | | | | | | |
| | GTGGA | | | | | | |
| rs20032 | TCACTCATCTTGTTA | 901 | | | 7 | 117251700 | + |
| 1110 | CAAGCTTAAAAGGAC | | | | | | |
| | TATGGACACTTCGTG | | | | | | |
| | CCTTC | | | | | | |
| rs74503 | CACTTTTACCTTATA | 902 | | | 7 | 117282526 | + |
| 330 | GGTGGGCCTCTTGGG | | | | | | |
| | AAGAACTGGATCAGG | | | | | | |
| | GAAGA | | | | | | |
| rs12190 | TATAGGTGGGCCTCT | 903 | | | 7 | 117282537 | + |
| 9041 | TGGGAAGAACTGGAT | | | | | | |
| | CAGGGAAGAGTACTT | | | | | | |
| | TGTTA | | | | | | |
| rs19392 | TCTTCGCCTTACTGA | 904 | | | 7 | 117304824 | − |
| 2525 | GAACAGATCTAGCCA | | | | | | |
| | AGCACATCAACTGCT | | | | | | |
| | TGTGG | | | | | | |
| rs39750 | GAAAAAAAGTTTGGA | 905 | | | 7 | 117480095 | − |
| 8328 | GACAACGCTGGCCTT | | | | | | |
| | TTCCAGAGGCGACCT | | | | | | |
| | CTGCA | | | | | | |
| rs39750 | GGAGCTTGAGCCCAG | 906 | | | 7 | 117480095 | + |
| 8328 | ACGGCCCTAGCAGGG | | | | | | |
| | ACCCCAGCGCCCGAG | | | | | | |
| | AGACC | | | | | | |
| rs39750 | GAAAAAAAGTTTGGA | 907 | | | 7 | 117480095 | − |
| 8328 | GACAACGCTGGCCTT | | | | | | |
| | TTCCAGAGGCGACCT | | | | | | |
| | CTGCA | | | | | | |
| rs15102 | TAAACAATGTACATG | 908 | TAAACAATGTACATG | 1938 | 7 | 117504336 | − |
| 0603 | AACATACCTTTCCAA | | AACATACCTTTCCAA | | | | |
| | TTTTTCAGATAGATT | | TTTTTCAGATAGATT | | | | |
| | GTCAA | | GTCAG | | | | |
| rs15102 | TAAACAATGTACATG | 909 | TAAACAATGTACATG | 1939 | 7 | 117504336 | − |
| 0603 | AACATACCTTTCCAA | | AACATACCTTTCCAA | | | | |
| | TTTTTCAGATAGATT | | TTTTTCAGATAGATT | | | | |
| | GTCAA | | GTCAG | | | | |
| rs15102 | TAAACAATGTACATG | 910 | TAAACAATGTACATG | 1940 | 7 | 117504336 | − |
| 0603 | AACATACCTTTCCAA | | AACATACCTTTCCAA | | | | |
| | TTTTTCAGATAGATT | | TTTTTCAGATAGATT | | | | |
| | GTCAA | | GTCAG | | | | |
| rs39750 | GGCATTAATGAGTTT | 911 | | | 7 | 117509035 | − |
| 8256 | AGGATTTTTCTTTGA | | | | | | |
| | AGCCAGCTCTCTATC | | | | | | |
| | CCATT | | | | | | |
| rs39750 | GGCATTAATGAGTTT | 912 | | | 7 | 117509035 | − |
| 8256 | AGGATTTTTCTTTGA | | | | | | |
| | AGCCAGCTCTCTATC | | | | | | |
| | CCATT | | | | | | |
| rs39750 | GGCATTAATGAGTTT | 913 | | | 7 | 117509035 | − |
| 8256 | AGGATTTTTCTTTGA | | | | | | |
| | AGCCAGCTCTCTATC | | | | | | |
| | CCATT | | | | | | |
| rs36850 | TTTATTCTTTTGCAG | 914 | | | 7 | 117509069 | + |
| 5753 | AGAATGGGATAGAGA | | | | | | |
| | GCTGGCTTCAAAGAA | | | | | | |
| | AAATC | | | | | | |
| rs36850 | TTTATTCTTTTGCAG | 915 | | | 7 | 117509069 | + |
| 5753 | AGAATGGGATAGAGA | | | | | | |
| | GCTGGCTTCAAAGAA | | | | | | |
| | AAATC | | | | | | |
| rs36850 | TTTATTCTTTTGCAG | 916 | | | 7 | 117509069 | + |
| 5753 | AGAATGGGATAGAGA | | | | | | |
| | GCTGGCTTCAAAGAA | | | | | | |
| | AAATC | | | | | | |
| rs11554 | GGGATAGAGAGCTGG | 917 | | | 7 | 117509089 | + |
| 5701 | CTTCAAAGAAAAATC | | | | | | |
| | YTAAACTCATTAATG | | | | | | |
| | CCCTT | | | | | | |
| rs11554 | GGGATAGAGAGCTGG | 918 | | | 7 | 117509089 | + |
| 5701 | CTTCAAAGAAAAATC | | | | | | |
| | YTAAACTCATTAATG | | | | | | |
| | CCCTT | | | | | | |
| rs11554 | GGGATAGAGAGCTGG | 919 | | | 7 | 117509089 | + |
| 5701 | CTTCAAAGAAAAATC | | | | | | |
| | CTAAACTCATTAATG | | | | | | |
| | CCCTT | | | | | | |
| rs75961 | ACATAATGAATGTAC | 920 | ACATAATGAATGTAC | 1941 | 7 | 117509123 | − |
| 395 | AAATGAGATCCTTAC | | AAATGAGATCCTTAC | | | | |
| | CCCTAAATATAAAAA | | CCCTAAATATAAAAA | | | | |
| | GATTT | | GATTC | | | | |
| rs75961 | ACATAATGAATGTAC | 921 | ACATAATGAATGTAC | 1942 | 7 | 117509123 | − |
| 395 | AAATGAGATCCTTAC | | AAATGAGATCCTTAC | | | | |
| | CCCTAAATATAAAAA | | CCCTAAATATAAAAA | | | | |
| | GATTT | | GATTC | | | | |
| rs75961 | ACATAATGAATGTAC | 922 | ACATAATGAATGTAC | 1943 | 7 | 117509123 | − |
| 395 | AAATGAGATCCTTAC | | AAATGAGATCCTTAC | | | | |
| | CCCTAAATATAAAAA | | CCCTAAATATAAAAA | | | | |
| | GATTT | | GATTC | | | | |
| rs12190 | GAAGCTATGATTCTT | 923 | GAAGCTATGATTCTT | 1944 | 7 | 117530899 | − |
| 8751 | CCCAGTAAGAGAGGC | | CCCAGTAAGAGAGGC | | | | |
| | TGTACTGCTTTGGTG | | TGTACTGCTTTGGTG | | | | |
| | ACTTA | | ACTTC | | | | |
| rs12190 | GAAGCTATGATTCTT | 924 | GAAGCTATGATTCTT | 1945 | 7 | 117530899 | − |
| 8751 | CCCAGTAAGAGAGGC | | CCCAGTAAGAGAGGC | | | | |
| | TGTACTGCTTTGGTG | | TGTACTGCTTTGGTG | | | | |
| | ACTTA | | ACTTC | | | | |
| rs12190 | GAAGCTATGATTCTT | 925 | GAAGCTATGATTCTT | 1946 | 7 | 117530899 | − |
| 8751 | CCCAGTAAGAGAGGC | | CCCAGTAAGAGAGGC | | | | |
| | TGTACTGCTTTGGTG | | TGTACTGCTTTGGTG | | | | |
| | ACTTA | | ACTTC | | | | |
| rs11399 | CACCAAAGCAGTACA | 926 | CACCAAAGCAGTACA | 1947 | 7 | 117530953 | + |
| 3958 | GCCTCTCTTACTGGG | | GCCTCTCTTACTGGG | | | | |
| | AAGAATCATAGCTTC | | AAGAATCATAGCTTC | | | | |
| | CTATC | | CTATG | | | | |
| rs11399 | CACCAAAGCAGTACA | 927 | CACCAAAGCAGTACA | 1948 | 7 | 117530953 | + |
| 3958 | GCCTCTCTTACTGGG | | GCCTCTCTTACTGGG | | | | |
| | AAGAATCATAGCTTC | | AAGAATCATAGCTTC | | | | |
| | CTATC | | CTATG | | | | |
| rs11399 | CACCAAAGCAGTACA | 928 | CACCAAAGCAGTACA | 1949 | 7 | 117530953 | + |
| 3958 | GCCTCTCTTACTGGG | | GCCTCTCTTACTGGG | | | | |
| | AAGAATCATAGCTTC | | AAGAATCATAGCTTC | | | | |
| | CTATC | | CTATG | | | | |
| rs39750 | ACCAAAGCAGTACAG | 929 | | | 7 | 117530955 | − |
| 8537 | CCTCTCTTACTGGGA | | | | | | |
| | AGAATCATAGCTTCC | | | | | | |
| | TATGA | | | | | | |
| rs39750 | ACCAAAGCAGTACAG | 930 | | | 7 | 117530955 | + |
| 8537 | CCTCTCTTACTGGGA | | | | | | |
| | AGAATCATAGCTTCC | | | | | | |
| | TATGA | | | | | | |
| rs39750 | ACCAAAGCAGTACAG | 931 | | | 7 | 117530955 | + |
| 8537 | CCTCTCTTACTGGGA | | | | | | |
| | AGAATCATAGCTTCC | | | | | | |
| | TATGA | | | | | | |
| rs77834 | CACAATAAAGAGAAG | 932 | | | 7 | 117530974 | − |
| 169 | GCATAAGCCTATGCC | | | | | | |
| | TAGATAAATCGCGAT | | | | | | |
| | AGAGC | | | | | | |
| rs77834 | CTTACTGGGAAGAAT | 933 | CTTACTGGGAAGAAT | 1950 | 7 | 117530974 | + |
| 169 | CATAGCTTCCTATGA | | CATAGCTTCCTATGA | | | | |
| | CCCGGATAACAAGGA | | CCCGGATAACAAGGA | | | | |
| | GGAAT | | GGAAC | | | | |
| rs77834 | TCTTACTGGGAAGAA | 934 | | | 7 | 117530974 | + |
| 169 | TCATAGCTTCCTATG | | | | | | |
| | ACCCGGATAACAAGG | | | | | | |
| | AGGAA | | | | | | |
| rs78655 | TTACTGGGAAGAATC | 935 | TTACTGGGAAGAATC | 1951 | 7 | 117530975 | + |
| 421 | ATAGCTTCCTATGAC | | ATAGCTTCCTATGAC | | | | |
| | CCGGATAACAAGGAG | | CCGGATAACAAGGAG | | | | |
| | GAACA | | GAACG | | | | |
| rs78655 | TTACTGGGAAGAATC | 936 | TTACTGGGAAGAATC | 1952 | 7 | 117530975 | + |
| 421 | ATAGCTTCCTATGAC | | ATAGCTTCCTATGAC | | | | |
| | CCGGATAACAAGGAG | | CCGGATAACAAGGAG | | | | |
| | GAACA | | GAACG | | | | |
| rs78655 | TCACAATAAAGAGAA | 937 | | | 7 | 117530975 | − |
| 421 | GGCATAAGCCTATGC | | | | | | |
| | CTAGATAAATCGCGA | | | | | | |
| | TAGAG | | | | | | |
| rs80282 | TACTTCATCAAATTT | 938 | | | 7 | 117534318 | − |
| 562 | GTTCAGGTTGTTGGA | | | | | | |
| | AAGGAGACTAACAAG | | | | | | |
| | TTGTC | | | | | | |
| rs80282 | TACTTCATCAAATTT | 939 | | | 7 | 117534318 | − |
| 562 | GTTCAGGTTGTTGGA | | | | | | |
| | AAGGAGACTAACAAG | | | | | | |
| | TTGTC | | | | | | |
| rs39750 | GTATTGGACAACTTG | 940 | | | 7 | 117534363 | + |
| 8759 | TTAGTCTCCTTTCCA | | | | | | |
| | ACAACCTGAACAAAT | | | | | | |
| | TTGAT | | | | | | |
| rs39750 | TATTGGACAACTTGT | 941 | TATTGGACAACTTGT | 1953 | 7 | 117534363 | + |
| 8759 | TAGTCTCCTTTCCAA | | TAGTCTCCTTTCCAA | | | | |
| | CAACCTGAACAAATT | | CAACCTGAACAAATT | | | | |
| | TGATA | | TGATG | | | | |
| rs39750 | GTATTGGACAACTTG | 942 | | | 7 | 117534363 | + |
| 8759 | TTAGTCTCCTTTCCA | | | | | | |
| | ACAACCTGAACAAAT | | | | | | |
| | TTGAT | | | | | | |
| rs77188 | TTGGACAACTTGTTA | 943 | | | 7 | 117534366 | + |
| 391 | GTCTCCTTTCCAACA | | | | | | |
| | ACCTGAACAAATTTG | | | | | | |
| | ATGAA | | | | | | |
| rs77188 | TTGGACAACTTGTTA | 944 | | | 7 | 117534366 | + |
| 391 | GTCTCCTTTCCAACA | | | | | | |
| | ACCTGAACAAATTTG | | | | | | |
| | ATGAA | | | | | | |
| rs77188 | TTGGACAACTTGTTA | 945 | | | 7 | 117534366 | + |
| 391 | GTCTCCTTTCCAACA | | | | | | |
| | ACCTGAACAAATTTG | | | | | | |
| | ATGAA | | | | | | |
| rs12190 | TTTTATTTTCCAGGG | 946 | | | 7 | 117535285 | + |
| 8752 | ACTTGCATTGGCACA | | | | | | |
| | TTTCGTGTGGATCGC | | | | | | |
| | TCCTT | | | | | | |
| rs12190 | TTTTATTTTCCAGGG | 947 | | | 7 | 117535285 | + |
| 8752 | ACTTGCATTGGCACA | | | | | | |
| | TTTCGTGTGGATCGC | | | | | | |
| | TCCTT | | | | | | |
| rs12190 | TTTTATTTTCCAGGG | 948 | | | 7 | 117535285 | + |
| 8752 | ACTTGCATTGGCACA | | | | | | |
| | TTTCGTGTGGATCGC | | | | | | |
| | TCCTT | | | | | | |
| rs12190 | TGGTGTTTTTATCTG | 949 | | | 7 | 117540230 | + |
| 9011 | TGCTTCCCTATGCAC | | | | | | |
| | TAATCAAAGGAATCA | | | | | | |
| | TCCTC | | | | | | |
| rs12190 | TGGTGTTTTTATCTG | 950 | | | 7 | 117540230 | + |
| 9011 | TGCTTCCCTATGCAC | | | | | | |
| | TAATCAAAGGAATCA | | | | | | |
| | TCCTC | | | | | | |
| rs12190 | TGGTGTTTTTATCTG | 951 | | | 7 | 117540230 | + |
| 9011 | TGCTTCCCTATGCAC | | | | | | |
| | TAATCAAAGGAATCA | | | | | | |
| | TCCTC | | | | | | |
| rs39750 | TTATCTGTGCTTCCC | 952 | TTATCTGTGCTTCCC | 1954 | 7 | 117540237 | + |
| 8139 | TATGCACTAATCAAA | | TATGCACTAATCAAA | | | | |
| | GGAATCATCCTCYGG | | GGAATCATCCTCYGG | | | | |
| | AAAAT | | AAAAC | | | | |
| rs39750 | TTATCTGTGCTTCCC | 953 | TTATCTGTGCTTCCC | 1955 | 7 | 117540237 | + |
| 8139 | TATGCACTAATCAAA | | TATGCACTAATCAAA | | | | |
| | GGAATCATCCTCYGG | | GGAATCATCCTCYGG | | | | |
| | AAAAT | | AAAAC | | | | |
| rs39750 | TTATCTGTGCTTCCC | 954 | TTATCTGTGCTTCCC | 1956 | 7 | 117540237 | + |
| 8139 | TATGCACTAATCAAA | | TATGCACTAATCAAA | | | | |
| | GGAATCATCCTCCGG | | GGAATCATCCTCCGG | | | | |
| | AAAAT | | AAAAC | | | | |
| rs77409 | TGTGCTTCCCTATGC | 955 | | | 7 | 117540243 | + |
| 459 | ACTAATCAAAGGAAT | | | | | | |
| | CATCCTCCGGAAAAT | | | | | | |
| | ATTCA | | | | | | |
| rs77409 | GAAATTGCCGAGTGA | 956 | | | 7 | 117540243 | − |
| 459 | CCGCCATGCGCAGAA | | | | | | |
| | CAATGCAGAATGRGA | | | | | | |
| | TGGTG | | | | | | |
| rs77409 | TGTGCTTCCCTATGC | 957 | | | 7 | 117540243 | + |
| 459 | ACTAATCAAAGGAAT | | | | | | |
| | CATCCTCCGGAAAAT | | | | | | |
| | ATTCA | | | | | | |
| rs39750 | CTATGCACTAATCAA | 958 | CTATGCACTAATCAA | 1957 | 7 | 117540251 | + |
| 8144 | AGGAATCATCCTCYG | | AGGAATCATCCTCYG | | | | |
| | GAAAAHATTCAYCAC | | GAAAAHATTCAYCAC | | | | |
| | CATCT | | CATCC | | | | |
| rs39750 | AGCCCAGGGAAATTG | 959 | | | 7 | 117540251 | − |
| 8144 | CCGAGTGACCGCCAT | | | | | | |
| | GYGCAGAACAATGCA | | | | | | |
| | GAATG | | | | | | |
| rs39750 | GCCCAGGGAAATTGC | 960 | GCCCAGGGAAATTGC | 1958 | 7 | 117540251 | − |
| 8144 | CGAGTGACCGCCATG | | CGAGTGACCGCCATG | | | | |
| | YGCAGAACAATGCAG | | YGCAGAACAATGCAG | | | | |
| | AATGA | | AATGG | | | | |
| rs77932 | GTCATACCATGTTTG | 961 | GTCATACCATGTTTG | 1959 | 7 | 117540270 | − |
| 196 | TACAGCCCAGGGAAA | | TACAGCCCAGGGAAA | | | | |
| | TTGCCGAGTGACCGC | | TTGCCGAGTGACCGC | | | | |
| | CATGC | | CATGG | | | | |
| rs77932 | GTCATACCATGTTTG | 962 | GTCATACCATGTTTG | 1960 | 7 | 117540270 | − |
| 196 | TACAGCCCAGGGAAA | | TACAGCCCAGGGAAA | | | | |
| | TTGCYGAGTGACCGC | | TTGCYGAGTGACCGC | | | | |
| | CATGT | | CATGC | | | | |
| rs77932 | GTCATACCATGTTTG | 963 | GTCATACCATGTTTG | 1961 | 7 | 117540270 | − |
| 196 | TACAGCCCAGGGAAA | | TACAGCCCAGGGAAA | | | | |
| | TTGCYGAGTGACCGC | | TTGCYGAGTGACCGC | | | | |
| | CATGT | | CATGC | | | | |
| rs12190 | TTATTGCTCCAAGAG | 964 | | | 7 | 117540285 | − |
| 8753 | AGTCATACCATGTTT | | | | | | |
| | GTACAGCCCAGGGAA | | | | | | |
| | ATTGC | | | | | | |
| rs12190 | TTATTGCTCCAAGAG | 965 | | | 7 | 117540285 | − |
| 8753 | AGTCATACCATGTTT | | | | | | |
| | GTACAGCCCAGGGAA | | | | | | |
| | ATTGC | | | | | | |
| rs12190 | TTATTGCTCCAAGAG | 966 | | | 7 | 117540285 | − |
| 8753 | AGTCATACCATGTTT | | | | | | |
| | GTACAGCCCAGGGAA | | | | | | |
| | ATTGC | | | | | | |
| rs74551 | AGTGAAGAACAAAAG | 967 | AGTGAAGAACAAAAG | 1962 | 7 | 117548795 | − |
| 128 | AACTACCTTGCCTGC | | AACTACCTTGCCTGC | | | | |
| | TCCAGTGGATCCAGC | | TCCAGTGGATCCAGC | | | | |
| | AACCT | | AACCG | | | | |
| rs74551 | AGTGAAGAACAAAAG | 968 | AGTGAAGAACAAAAG | 1963 | 7 | 117548795 | − |
| 128 | AACTACCTTGCCTGC | | AACTACCTTGCCTGC | | | | |
| | TCCAGTGGATCCAGC | | TCCAGTGGATCCAGC | | | | |
| | AACCT | | AACCG | | | | |
| rs74551 | AGTGAAGAACAAAAG | 969 | AGTGAAGAACAAAAG | 1964 | 7 | 117548795 | − |
| 128 | AACTACCTTGCCTGC | | AACTACCTTGCCTGC | | | | |
| | TCCAGTGGATCCAGC | | TCCAGTGGATCCAGC | | | | |
| | AACCT | | AACCG | | | | |
| rs13957 | CGTGATTTGATAATG | 970 | | | 7 | 117559471 | + |
| 3311 | ACCTAATAATGATGG | | | | | | |
| | GTTTTATTTCCAGAC | | | | | | |
| | TTCAC | | | | | | |
| rs13957 | GCTTAATTTTACCCT | 971 | | | 7 | 117559471 | − |
| 3311 | CTGAAGGCTCCAGTT | | | | | | |
| | CTCCCATAATCACCA | | | | | | |
| | TTAGA | | | | | | |
| rs13957 | GCTTAATTTTACCCT | 972 | | | 7 | 117559471 | − |
| 3311 | CTGAAGGCTCCAGTT | | | | | | |
| | CTCCCATAATCACCA | | | | | | |
| | TTAGA | | | | | | |
| rs12190 | GGAGCCTTCAGAGGG | 973 | | | 7 | 117559546 | + |
| 9017 | TAAAATTAAGCACAG | | | | | | |
| | TGGAAGAATTTCATT | | | | | | |
| | CTGTT | | | | | | |
| rs12190 | GGAGCCTTCAGAGGG | 974 | | | 7 | 117559546 | + |
| 9017 | TAAAATTAAGCACAG | | | | | | |
| | TGGAAGAATTTCATT | | | | | | |
| | CTGTT | | | | | | |
| rs12190 | GGAGCCTTCAGAGGG | 975 | | | 7 | 117559546 | + |
| 9017 | TAAAATTAAGCACAG | | | | | | |
| | TGGAAGAATTTCATT | | | | | | |
| | CTGTT | | | | | | |
| rs11399 | TGTTCTCAGTTTTCC | 976 | | | 7 | 117559591 | + |
| 3960 | TGGATTATGCCTGGC | | | | | | |
| | ACCATTAAAGAAAAT | | | | | | |
| | ATCAT | | | | | | |
| rs11399 | TGTTCTCAGTTTTCC | 977 | | | 7 | 117559591 | + |
| 3960 | TGGATTATGCCTGGC | | | | | | |
| | ACCATTAAAGAAAAT | | | | | | |
| | ATCAT | | | | | | |
| rs11399 | TGTTCTCAGTTTTCC | 978 | | | 7 | 117559591 | + |
| 3960 | TGGATTATGCCTGGC | | | | | | |
| | ACCATTAAAGAAAAT | | | | | | |
| | ATCAT | | | | | | |
| rs77646 | AGAAAATATCATCTT | 979 | AGAAAATATCATCTT | 1965 | 7 | 117559629 | + |
| 904 | TGGTGTTTCCTATGA | | TGGTGTTTCCTATGA | | | | |
| | TGAATATAGATACAG | | TGAATATAGATACAG | | | | |
| | AAGCA | | AAGCG | | | | |
| rs77646 | AGAAAATATCATCTT | 980 | AGAAAATATCATCTT | 1966 | 7 | 117559629 | + |
| 904 | TGGTGTTTCCTATGA | | TGGTGTTTCCTATGA | | | | |
| | TGAATATAGATACAG | | TGAATATAGATACAG | | | | |
| | AAGCA | | AAGCG | | | | |
| rs77646 | AGAAAATATCATCTT | 981 | AGAAAATATCATCTT | 1967 | 7 | 117559629 | + |
| 904 | TGGTGTTTCCTATGA | | TGGTGTTTCCTATGA | | | | |
| | TGAATATAGATACAG | | TGAATATAGATACAG | | | | |
| | AAGCA | | AAGCG | | | | |
| rs11399 | TTTGGTAATAGGACA | 982 | | | 7 | 117587778 | + |
| 3959 | TCTCCAAGTTTGCAG | | | | | | |
| | AGAAAGACAATATAG | | | | | | |
| | TTCTT | | | | | | |
| rs11399 | TTGGTAATAGGACAT | 983 | TTGGTAATAGGACAT | 1968 | 7 | 117587778 | + |
| 3959 | CTCCAAGTTTGCAGA | | CTCCAAGTTTGCAGA | | | | |
| | GAAAGACAATATAGT | | GAAAGACAATATAGT | | | | |
| | TCTTT | | TCTTG | | | | |
| rs12190 | AGTTTGCAGAGAAAG | 984 | | | 7 | 117587799 | + |
| 8757 | ACAATATAGTTCTTG | | | | | | |
| | GAGAAGGTGGAATCA | | | | | | |
| | CACTG | | | | | | |
| rs12190 | AGTTTGCAGAGAAAG | 985 | | | 7 | 117587799 | + |
| 8757 | ACAATATAGTTCTTG | | | | | | |
| | GAGAAGGTGGAATCA | | | | | | |
| | CACTG | | | | | | |
| rs12190 | AGTTTGCAGAGAAAG | 986 | | | 7 | 117587799 | + |
| 8757 | ACAATATAGTTCTTG | | | | | | |
| | GAGAAGGTGGAATCA | | | | | | |
| | CACTG | | | | | | |
| rs12190 | GTTTGCAGAGAAAGA | 987 | | | 7 | 117587800 | + |
| 8755 | CAATATAGTTCTTGG | | | | | | |
| | AGAAGGTGGAATCAC | | | | | | |
| | ACTGA | | | | | | |
| rs12190 | TTTGCAGAGAAAGAC | 988 | TTTGCAGAGAAAGAC | 1969 | 7 | 117587800 | + |
| 8755 | AATATAGTTCTTGGA | | AATATAGTTCTTGGA | | | | |
| | GAAGGTGGAATCACA | | GAAGGTGGAATCACA | | | | |
| | CTGAA | | CTGAG | | | | |
| rs12190 | GTTTGCAGAGAAAGA | 989 | | | 7 | 117587800 | + |
| 8755 | CAATATAGTTCTTGG | | | | | | |
| | AGAAGGTGGAATCAC | | | | | | |
| | ACTGA | | | | | | |
| rs12190 | TTGCAGAGAAAGACA | 990 | TTGCAGAGAAAGACA | 1970 | 7 | 117587801 | + |
| 9005 | ATATAGTTCTTGGAG | | ATATAGTTCTTGGAG | | | | |
| | AAGGTGGAATCACAC | | AAGGTGGAATCACAC | | | | |
| | TGAGT | | TGAGG | | | | |
| rs12190 | TTGCAGAGAAAGACA | 991 | TTGCAGAGAAAGACA | 1971 | 7 | 117587801 | + |
| 9005 | ATATAGTTCTTGGAG | | ATATAGTTCTTGGAG | | | | |
| | AAGGTGGAATCACAC | | AAGGTGGAATCACAC | | | | |
| | TGAGT | | TGAGG | | | | |
| rs12190 | TTGCAGAGAAAGACA | 992 | TTGCAGAGAAAGACA | 1972 | 7 | 117587801 | + |
| 9005 | ATATAGTTCTTGGAG | | ATATAGTTCTTGGAG | | | | |
| | AAGGTGGAATCACAC | | AAGGTGGAATCACAC | | | | |
| | TGAGT | | TGAGG | | | | |
| rs12190 | CAGAGAAAGACAATA | 993 | | | 7 | 117587805 | + |
| 9013 | TAGTTCTTGGAGAAG | | | | | | |
| | GTGGAATCACACTGA | | | | | | |
| | GTGGA | | | | | | |
| rs75527 | TAGACCAATAATTAG | 994 | | | 7 | 117587806 | − |
| 207 | TTATTCACCTTGCTA | | | | | | |
| | AAGAAATTCTTGCTC | | | | | | |
| | GTTGA | | | | | | |
| rs75527 | TAGACCAATAATTAG | 995 | | | 7 | 117587806 | − |
| 207 | TTATTCACCTTGCTA | | | | | | |
| | AAGAAATTCTTGCTC | | | | | | |
| | GTTGA | | | | | | |
| rs75527 | TAGACCAATAATTAG | 996 | | | 7 | 117587806 | − |
| 207 | TTATTCACCTTGCTA | | | | | | |
| | AAGAAATTCTTGCTC | | | | | | |
| | GTTGA | | | | | | |
| rs74597 | AGACAATATAGTTCT | 997 | AGACAATATAGTTCT | 1973 | 7 | 117587811 | + |
| 325 | TGGAGAAGGTGGAAT | | TGGAGAAGGTGGAAT | | | | |
| | CACACTGAGTGGAGG | | CACACTGAGTGGAGG | | | | |
| | TCAAT | | TCAAC | | | | |
| rs74597 | AGACAATATAGTTCT | 998 | AGACAATATAGTTCT | 1974 | 7 | 117587811 | + |
| 325 | TGGAGAAGGTGGAAT | | TGGAGAAGGTGGAAT | | | | |
| | CACACTGAGTGGAGG | | CACACTGAGTGGAGG | | | | |
| | TCAAT | | TCAAC | | | | |
| rs74597 | AAGACAATATAGTTC | 999 | | | 7 | 117587811 | + |
| 325 | TTGGAGAAGGTGGAA | | | | | | |
| | TCACACTGAGTGGAG | | | | | | |
| | GTCAA | | | | | | |
| rs75549 | AGAAGGTGGAATCAC | 1000 | AGAAGGTGGAATCAC | 1975 | 7 | 117587829 | + |
| 581 | ACTGAGTGGAGGTCA | | ACTGAGTGGAGGTCA | | | | |
| | ACGAGCAAGAATTTC | | ACGAGCAAGAATTTC | | | | |
| | TTTAA | | TTTAG | | | | |
| rs75549 | AGAAGGTGGAATCAC | 1001 | AGAAGGTGGAATCAC | 1976 | 7 | 117587829 | + |
| 581 | ACTGAGTGGAGGTCA | | ACTGAGTGGAGGTCA | | | | |
| | ACGAGCAAGAATTTC | | ACGAGCAAGAATTTC | | | | |
| | TTTAA | | TTTAG | | | | |
| rs75549 | TGACATTTACAGCAA | 1002 | | | 7 | 117587829 | − |
| 581 | ATGCTTGCTAGACCA | | | | | | |
| | ATAATTAGTTATTCA | | | | | | |
| | CCTTG | | | | | | |
| rs80055 | GAATGACATTTACAG | 1003 | GAATGACATTTACAG | 1977 | 7 | 117587833 | − |
| 610 | CAAATGCTTGCTAGA | | CAAATGCTTGCTAGA | | | | |
| | CCAATAATTAGTTAT | | CCAATAATTAGTTAT | | | | |
| | TCACG | | TCACC | | | | |
| rs80055 | GGTGGAATCACACTG | 1004 | GGTGGAATCACACTG | 1978 | 7 | 117587833 | + |
| 610 | AGTGGAGGTCAACGA | | AGTGGAGGTCAACGA | | | | |
| | GCAAGAATTTCTTTA | | GCAAGAATTTCTTTA | | | | |
| | GCAAC | | GCAAG | | | | |
| rs80055 | GGTGGAATCACACTG | 1005 | GGTGGAATCACACTG | 1979 | 7 | 117587833 | + |
| 610 | AGTGGAGGTCAACGA | | AGTGGAGGTCAACGA | | | | |
| | GCAAGAATTTCTTTA | | GCAAGAATTTCTTTA | | | | |
| | GCAAA | | GCAAG | | | | |
| rs39750 | TATCCAAAAGGAGAG | 1006 | | | 7 | 117590353 | − |
| 8267 | TCTAATAAATACAAA | | | | | | |
| | TCAGCATCTTTGTAT | | | | | | |
| | ACTKC | | | | | | |
| rs39750 | TATCCAAAAGGAGAG | 1007 | | | 7 | 117590353 | − |
| 8267 | TCTAATAAATACAAA | | | | | | |
| | TCAGCATCTTTGTAT | | | | | | |
| | ACTGC | | | | | | |
| rs39750 | TATCCAAAAGGAGAG | 1008 | | | 7 | 117590353 | − |
| 8267 | TCTAATAAATACAAA | | | | | | |
| | TCAGCATCTTTGTAT | | | | | | |
| | ACTGC | | | | | | |
| rs12190 | GGTATCCAAAAGGAG | 1009 | | | 7 | 117590355 | − |
| 9047 | AGTCTAATAAATACA | | | | | | |
| | AATCAGCATCTTTGT | | | | | | |
| | ATACT | | | | | | |
| rs12190 | GGTATCCAAAAGGAG | 1010 | | | 7 | 117590355 | − |
| 9047 | AGTCTAATAAATACA | | | | | | |
| | AATCAGCATCTTTGT | | | | | | |
| | ATACT | | | | | | |
| rs12190 | GGTATCCAAAAGGAG | 1011 | | | 7 | 117590355 | − |
| 9047 | AGTCTAATAAATACA | | | | | | |
| | AATCAGCATCTTTGT | | | | | | |
| | ATACT | | | | | | |
| rs39750 | TCTTTTTCTGTTAAA | 1012 | TCTTTTTCTGTTAAA | 1980 | 7 | 117590378 | − |
| 8276 | ACATCTAGGTATCCA | | ACATCTAGGTATCCA | | | | |
| | AAAGGAGAGTCTAAT | | AAAGGAGAGTCTAAT | | | | |
| | AAATA | | AAATC | | | | |
| rs39750 | TCTTTTTCTGTTAAA | 1013 | TCTTTTTCTGTTAAA | 1981 | 7 | 117590378 | − |
| 8276 | ACATCTAGGTATCCA | | ACATCTAGGTATCCA | | | | |
| | AAAGGAGAGTCTAAT | | AAAGGAGAGTCTAAT | | | | |
| | AAATA | | AAATC | | | | |
| rs39750 | TTCTTTTTCTGTTAA | 1014 | | | 7 | 117590378 | − |
| 8276 | AACATCTAGGTATCC | | | | | | |
| | AAAAGGAGAGTCTAA | | | | | | |
| | TAAAT | | | | | | |
| rs39750 | TACAAAGATGCTGAT | 1015 | TACAAAGATGCTGAT | 1982 | 7 | 117590409 | + |
| 8288 | TTGTATTTATTAGAC | | TTGTATTTATTAGAC | | | | |
| | TCTCCTTTTGGATAC | | TCTCCTTTTGGATAC | | | | |
| | CTAGA | | CTAGG | | | | |
| rs39750 | TACAAAGATGCTGAT | 1016 | TACAAAGATGCTGAT | 1983 | 7 | 117590409 | + |
| 8288 | TTGTATTTATTAGAC | | TTGTATTTATTAGAC | | | | |
| | TCTCCTTTTGGATAC | | TCTCCTTTTGGATAC | | | | |
| | CTAGA | | CTAGG | | | | |
| rs39750 | TACAAAGATGCTGAT | 1017 | TACAAAGATGCTGAT | 1984 | 7 | 117590409 | + |
| 8288 | TTGTATTTATTAGAC | | TTGTATTTATTAGAC | | | | |
| | TCTCCTTTTGGATAC | | TCTCCTTTTGGATAC | | | | |
| | CTAGA | | CTAGG | | | | |
| rs18001 | ACCAATTTAGTGCAG | 1018 | | | 7 | 117592169 | + |
| 00 | AAAGAAGAAATTCAA | | | | | | |
| | TCCTAACTGAGACCT | | | | | | |
| | TACAC | | | | | | |
| rs18001 | ACCAATTTAGTGCAG | 1019 | | | 7 | 117592169 | + |
| 00 | AAAGAAGAAATTCAA | | | | | | |
| | TCCTAACTGAGACCT | | | | | | |
| | TACAC | | | | | | |
| rs18001 | ACCAATTTAGTGCAG | 1020 | | | 7 | 117592169 | + |
| 00 | AAAGAAGAAATTCAA | | | | | | |
| | TCCTAACTGAGACCT | | | | | | |
| | TACAC | | | | | | |
| rs39750 | GTTCCATGTAGTCAC | 1021 | | | 7 | 117594930 | − |
| 8387 | TGCTGGTATGCTCTC | | | | | | |
| | CATATCATCAAAAAA | | | | | | |
| | GCACT | | | | | | |
| rs39750 | GCATGAAACTGTACT | 1022 | GCATGAAACTGTACT | 1985 | 7 | 117594930 | + |
| 8387 | GTCTTATTGTAATAG | | GTCTTATTGTAATAG | | | | |
| | CCATAATTCTTTTAT | | CCATAATTCTTTTAT | | | | |
| | TCAGT | | TCAGG | | | | |
| rs39750 | TTCCATGTAGTCACT | 1023 | TTCCATGTAGTCACT | 1986 | 7 | 117594930 | − |
| 8387 | GCTGGTATGCTCTCC | | GCTGGTATGCTCTCC | | | | |
| | ATATCATCAAAAAAG | | ATATCATCAAAAAAG | | | | |
| | CACTA | | CACTC | | | | |
| rs80224 | ATTTAATCAACAGAA | 1024 | ATTTAATCAACAGAA | 1987 | 7 | 117602868 | − |
| 560 | ATAAAACACAATCTA | | ATAAAACACAATCTA | | | | |
| | CACAATAGGACATGG | | CACAATAGGACATGG | | | | |
| | AATAT | | AATAC | | | | |
| rs80224 | ATTTAATCAACAGAA | 1025 | ATTTAATCAACAGAA | 1988 | 7 | 117602868 | − |
| 560 | ATAAAACACAATCTA | | ATAAAACACAATCTA | | | | |
| | CACAATAGGACATGG | | CACAATAGGACATGG | | | | |
| | AATAT | | AATAC | | | | |
| rs80224 | TATTTAATCAACAGA | 1026 | | | 7 | 117602868 | − |
| 560 | AATAAAACACAATCT | | | | | | |
| | ACACAATAGGACATG | | | | | | |
| | GAATA | | | | | | |
| rs39750 | TGTGATTAGAGTATG | 1027 | TGTGATTAGAGTATG | 1989 | 7 | 117603654 | − |
| 8435 | CACCAGTGGTAGACC | | CACCAGTGGTAGACC | | | | |
| | TCTGAAGAATCCCAT | | TCTGAAGAATCCCAT | | | | |
| | AGCAA | | AGCAT | | | | |
| rs39750 | TGTGATTAGAGTATG | 1028 | TGTGATTAGAGTATG | 1990 | 7 | 117603654 | − |
| 8435 | CACCAGTGGTAGACC | | CACCAGTGGTAGACC | | | | |
| | TCTGAAGAATCCCAT | | TCTGAAGAATCCCAT | | | | |
| | AGCAA | | AGCAG | | | | |
| rs39750 | CAGTTCGTATTATGT | 1029 | | | 7 | 117603654 | + |
| 8435 | GTTTTACATTTACGT | | | | | | |
| | GGGAGTAGCCGACAC | | | | | | |
| | TTTGC | | | | | | |
| rs39750 | TATGGGATTCTTCAG | 1030 | | | 7 | 117603708 | + |
| 8442 | AGGTCTACCACTGGT | | | | | | |
| | GCATACTCTAATCAC | | | | | | |
| | AGTGT | | | | | | |
| rs39750 | TATGGGATTCTTCAG | 1031 | | | 7 | 117603708 | + |
| 8442 | AGGTCTACCACTGGT | | | | | | |
| | GCATACTCTAATCAC | | | | | | |
| | AGTGT | | | | | | |
| rs39750 | TATGGGATTCTTCAG | 1032 | | | 7 | 117603708 | + |
| 8442 | AGGTCTACCACTGGT | | | | | | |
| | GCATACTCTAATCAC | | | | | | |
| | AGTGT | | | | | | |
| rs39750 | CCTATTGATGGTGGA | 1033 | CCTATTGATGGTGGA | 1991 | 7 | 117603782 | − |
| 8453 | TCAGCAGTTTCATTT | | TCAGCAGTTTCATTT | | | | |
| | CTTAGACCTAGTAAA | | CTTAGACCTAGTAAA | | | | |
| | GTACG | | GTACC | | | | |
| rs39750 | CCTATTGATGGTGGA | 1034 | CCTATTGATGGTGGA | 1992 | 7 | 117603782 | − |
| 8453 | TCAGCAGTTTCATTT | | TCAGCAGTTTCATTT | | | | |
| | CTTAGACCTAGTAAA | | CTTAGACCTAGTAAA | | | | |
| | GTACG | | GTACC | | | | |
| rs39750 | CCTATTGATGGTGGA | 1035 | CCTATTGATGGTGGA | 1993 | 7 | 117603782 | − |
| 8453 | TCAGCAGTTTCATTT | | TCAGCAGTTTCATTT | | | | |
| | CTTAGACCTAGTAAA | | CTTAGACCTAGTAAA | | | | |
| | GTACG | | GTACC | | | | |
| rs14103 | GTCAAATATGGTAAG | 1036 | GTCAAATATGGTAAG | 1994 | 7 | 117606695 | − |
| 3578 | AGGCAGAAGGTCATC | | AGGCAGAAGGTCATC | | | | |
| | CAAAATTGCTATATC | | CAAAATTGCTATATC | | | | |
| | TTTGA | | TTTGG | | | | |
| rs14103 | GTCAAATATGGTAAG | 1037 | GTCAAATATGGTAAG | 1995 | 7 | 117606695 | − |
| 3578 | AGGCAGAAGGTCATC | | AGGCAGAAGGTCATC | | | | |
| | CAAAATTGCTATATC | | CAAAATTGCTATATC | | | | |
| | TTTGA | | TTTGG | | | | |
| rs14103 | GTCAAATATGGTAAG | 1038 | GTCAAATATGGTAAG | 1996 | 7 | 117606695 | − |
| 3578 | AGGCAGAAGGTCATC | | AGGCAGAAGGTCATC | | | | |
| | CAAAATTGCTATATC | | CAAAATTGCTATATC | | | | |
| | TTTGA | | TTTGG | | | | |
| rs18001 | AGGGTTGTAAAACTG | 1039 | AGGGTTGTAAAACTG | 1997 | 7 | 117610521 | − |
| 11 | CGACAACTGCTATAG | | CGACAACTGCTATAG | | | | |
| | CTCCAATCACAATTA | | CTCCAATCACAATTA | | | | |
| | ATAAC | | ATAAG | | | | |
| rs18001 | AGGGTTGTAAAACTG | 1040 | AGGGTTGTAAAACTG | 1998 | 7 | 117610521 | − |
| 11 | CGACAACTGCTATAG | | CGACAACTGCTATAG | | | | |
| | CTCCAATCACAATTA | | CTCCAATCACAATTA | | | | |
| | ATAAG | | ATAAC | | | | |
| rs18001 | AGGGTTGTAAAACTG | 1041 | AGGGTTGTAAAACTG | 1999 | 7 | 117610521 | − |
| 11 | CGACAACTGCTATAG | | CGACAACTGCTATAG | | | | |
| | CTCCAATCACAATTA | | CTCCAATCACAATTA | | | | |
| | ATAAC | | ATAAG | | | | |
| rs76151 | AGATGAGTGAAAATT | 1042 | | | 7 | 117611555 | − |
| 804 | GGACTCCTGCCTGTG | | | | | | |
| | AAATATTTCCATAGA | | | | | | |
| | AAACA | | | | | | |
| rs76151 | TTCAGGTACAAGATA | 1043 | | | 7 | 117611555 | + |
| 804 | TTATGAAATTACATT | | | | | | |
| | TTGTGTTTATGTTAT | | | | | | |
| | TTGCA | | | | | | |
| rs76151 | AGATGAGTGAAAATT | 1044 | | | 7 | 117611555 | − |
| 804 | GGACTCCTGCCTGTG | | | | | | |
| | AAATATTTCCATAGA | | | | | | |
| | AAACA | | | | | | |
| rs15021 | AAGGCACGAAGTGTC | 1045 | AAGGCACGAAGTGTC | 2000 | 7 | 117611595 | − |
| 2784 | CATAGTCCTTTTAAG | | CATAGTCCTTTTAAG | | | | |
| | CTTGTAACAAGATGA | | CTTGTAACAAGATGA | | | | |
| | GTGAA | | GTGAC | | | | |
| rs15021 | AAGGCACGAAGTGTC | 1046 | AAGGCACGAAGTGTC | 2001 | 7 | 117611595 | − |
| 2784 | CATAGTCCTTTTAAG | | CATAGTCCTTTTAAG | | | | |
| | CTTGTAACAAGATGA | | CTTGTAACAAGATGA | | | | |
| | GTGAA | | GTGAC | | | | |
| rs15021 | GTTATTTGCAATGTT | 1047 | | | 7 | 117611595 | + |
| 2784 | TTCTATGGAAATATT | | | | | | |
| | TCACAGGCAGGAGTC | | | | | | |
| | CAATT | | | | | | |
| rs39750 | CGTCCGAAGGCACGA | 1048 | CGTCCGAAGGCACGA | 2002 | 7 | 117611601 | − |
| 8510 | AGTGTCCATAGTCCT | | AGTGTCCATAGTCCT | | | | |
| | TTTAAGCTTGTAACA | | TTTAAGCTTGTAACA | | | | |
| | AGATA | | AGATG | | | | |
| rs39750 | CGTCCGAAGGCACGA | 1049 | CGTCCGAAGGCACGA | 2003 | 7 | 117611601 | − |
| 8510 | AGTGTCCATAGTCCT | | AGTGTCCATAGTCCT | | | | |
| | TTTAAGCTTGTAACA | | TTTAAGCTTGTAACA | | | | |
| | AGATA | | AGATG | | | | |
| rs39750 | CGTCCGAAGGCACGA | 1050 | CGTCCGAAGGCACGA | 2004 | 7 | 117611601 | − |
| 8510 | AGTGTCCATAGTCCT | | AGTGTCCATAGTCCT | | | | |
| | TTTAAGCTTGTAACA | | TTTAAGCTTGTAACA | | | | |
| | AGATA | | AGATG | | | | |
| rs39750 | ATATTTCACAGGCAG | 1051 | | | 7 | 117611620 | + |
| 8513 | GAGTCCAATTTTCAC | | | | | | |
| | TCATCTTGTTACAAG | | | | | | |
| | CTTAA | | | | | | |
| rs39750 | ATATTTCACAGGCAG | 1052 | | | 7 | 117611620 | + |
| 8513 | GAGTCCAATTTTCAC | | | | | | |
| | TCATCTTGTTACAAG | | | | | | |
| | CTTAA | | | | | | |
| rs39750 | ATATTTCACAGGCAG | 1053 | | | 7 | 117611620 | + |
| 8513 | GAGTCCAATTTTCAC | | | | | | |
| | TCATCTTGTTACAAG | | | | | | |
| | CTTAA | | | | | | |
| rs12190 | AGTCCAATTTTCACT | 1054 | AGTCCAATTTTCACT | 2005 | 7 | 117611635 | + |
| 9036 | CATCTTGTTACAAGC | | CATCTTGTTACAAGC | | | | |
| | TTAAMAGGACTATGG | | TTAAMAGGACTATGG | | | | |
| | ACACT | | ACACC | | | | |
| rs12190 | AGTCCAATTTTCACT | 1055 | AGTCCAATTTTCACT | 2006 | 7 | 117611635 | + |
| 9036 | CATCTTGTTACAAGC | | CATCTTGTTACAAGC | | | | |
| | TTAAMAGGACTATGG | | TTAAMAGGACTATGG | | | | |
| | ACACT | | ACACC | | | | |
| rs12190 | AGTCCAATTTTCACT | 1056 | AGTCCAATTTTCACT | 2007 | 7 | 117611635 | + |
| 9036 | CATCTTGTTACAAGC | | CATCTTGTTACAAGC | | | | |
| | TTAAAAGGACTATGG | | TTAAAAGGACTATGG | | | | |
| | ACACT | | ACACC | | | | |
| rs78194 | TCCAATTTTCACTCA | 1057 | TCCAATTTTCACTCA | 2008 | 7 | 117611637 | + |
| 216 | TCTTGTTACAAGCTT | | TCTTGTTACAAGCTT | | | | |
| | AAAAGGACTATGGAC | | AAAAGGACTATGGAC | | | | |
| | ACTTA | | ACTTC | | | | |
| rs78194 | TCCAATTTTCACTCA | 1058 | TCCAATTTTCACTCA | 2009 | 7 | 117611637 | + |
| 216 | TCTTGTTACAAGCTT | | TCTTGTTACAAGCTT | | | | |
| | AAAAGGACTATGGAC | | AAAAGGACTATGGAC | | | | |
| | ACTTA | | ACTTC | | | | |
| rs78194 | TCCAATTTTCACTCA | 1059 | TCCAATTTTCACTCA | 2010 | 7 | 117611637 | + |
| 216 | TCTTGTTACAAGCTT | | TCTTGTTACAAGCTT | | | | |
| | AAAAGGACTATGGAC | | AAAAGGACTATGGAC | | | | |
| | ACTTA | | ACTTC | | | | |
| rs12190 | CCAATTTTCACTCAT | 1060 | CCAATTTTCACTCAT | 2011 | 7 | 117611638 | + |
| 9019 | CTTGTTACAAGCTTA | | CTTGTTACAAGCTTA | | | | |
| | AAAGGACTATGGACA | | AAAGGACTATGGACA | | | | |
| | CTTCA | | CTTCG | | | | |
| rs12190 | CCAATTTTCACTCAT | 1061 | CCAATTTTCACTCAT | 2012 | 7 | 117611638 | + |
| 9019 | CTTGTTACAAGCTTA | | CTTGTTACAAGCTTA | | | | |
| | AAAGGACTATGGACA | | AAAGGACTATGGACA | | | | |
| | CTTCA | | CTTCG | | | | |
| rs12190 | CCAATTTTCACTCAT | 1062 | CCAATTTTCACTCAT | 2013 | 7 | 117611638 | + |
| 9019 | CTTGTTACAAGCTTA | | CTTGTTACAAGCTTA | | | | |
| | AAAGGACTATGGACA | | AAAGGACTATGGACA | | | | |
| | CTTCA | | CTTCG | | | | |
| rs12190 | AATTTTCACTCATCT | 1063 | AATTTTCACTCATCT | 2014 | 7 | 117611640 | + |
| 9020 | TGTTACAAGCTTAAA | | TGTTACAAGCTTAAA | | | | |
| | AGGACTATGGACACT | | AGGACTATGGACACT | | | | |
| | TCGTA | | TCGTG | | | | |
| rs12190 | AATTTTCACTCATCT | 1064 | AATTTTCACTCATCT | 2015 | 7 | 117611640 | + |
| 9020 | TGTTACAAGCTTAAA | | TGTTACAAGCTTAAA | | | | |
| | AGGACTATGGACACT | | AGGACTATGGACACT | | | | |
| | TCGTA | | TCGTG | | | | |
| rs12190 | ATTCAGAGCTTTGTG | 1065 | | | 7 | 117611640 | − |
| 9020 | GAACAGAGTTTCAAA | | | | | | |
| | GTAAGGCTGCCGTCC | | | | | | |
| | GAAGG | | | | | | |
| rs20032 | TCACTCATCTTGTTA | 1066 | | | 7 | 117611646 | + |
| 1110 | CAAGCTTAAAAGGAC | | | | | | |
| | TATGGACACTTCGTG | | | | | | |
| | CCTTC | | | | | | |
| rs20032 | TCACTCATCTTGTTA | 1067 | | | 7 | 117611646 | + |
| 1110 | CAAGCTTAAAAGGAC | | | | | | |
| | TATGGACACTTCGTG | | | | | | |
| | CCTTC | | | | | | |
| rs20217 | CTCATCTTGTTACAA | 1068 | | | 7 | 117611649 | + |
| 9988 | GCTTAAAAGGACTAT | | | | | | |
| | GGACACTTCGTGCCT | | | | | | |
| | TCGGA | | | | | | |
| rs20217 | CTCATCTTGTTACAA | 1069 | | | 7 | 117611649 | + |
| 9988 | GCTTAAAAGGACTAT | | | | | | |
| | GGACACTTCGTGCCT | | | | | | |
| | TCGGA | | | | | | |
| rs20217 | CTCATCTTGTTACAA | 1070 | | | 7 | 117611649 | + |
| 9988 | GCTTAAAAGGACTAT | | | | | | |
| | GGACACTTCGTGCCT | | | | | | |
| | TCGGA | | | | | | |
| rs78769 | AGTATGTAAATTCAG | 1071 | AGTATGTAAATTCAG | 2016 | 7 | 117611650 | − |
| 542 | AGCTTTGTGGAACRG | | AGCTTTGTGGAACRG | | | | |
| | AGTTTCWAAGTAAGG | | AGTTTCWAAGTAAGG | | | | |
| | CTGCT | | CTGCC | | | | |
| rs78769 | AGTATGTAAATTCAG | 1072 | AGTATGTAAATTCAG | 2017 | 7 | 117611650 | − |
| 542 | AGCTTTGTGGAACRG | | AGCTTTGTGGAACRG | | | | |
| | AGTTTCWAAGTAAGG | | AGTTTCWAAGTAAGG | | | | |
| | CTGCT | | CTGCC | | | | |
| rs78769 | AGTATGTAAATTCAG | 1073 | AGTATGTAAATTCAG | 2018 | 7 | 117611650 | − |
| 542 | AGCTTTGTGGAACRG | | AGCTTTGTGGAACRG | | | | |
| | AGTTTCWAAGTAAGG | | AGTTTCWAAGTAAGG | | | | |
| | CTGCT | | CTGCC | | | | |
| rs18604 | AGAACCAGTTGGCAG | 1074 | AGAACCAGTTGGCAG | 2019 | 7 | 117611663 | − |
| 5772 | TATGTAAATTCAGAG | | TATGTAAATTCAGAG | | | | |
| | CTTTGTGGAACAGAG | | CTTTGTGGAACAGAG | | | | |
| | TTTCA | | TTTCT | | | | |
| rs18604 | AGAACCAGTTGGCAG | 1075 | AGAACCAGTTGGCAG | 2020 | 7 | 117611663 | − |
| 5772 | TATGTAAATTCAGAG | | TATGTAAATTCAGAG | | | | |
| | CTTTGTGGAACAGAG | | CTTTGTGGAACAGAG | | | | |
| | TTTCT | | TTTCA | | | | |
| rs18604 | AGAACCAGTTGGCAG | 1076 | AGAACCAGTTGGCAG | 2021 | 7 | 117611663 | − |
| 5772 | TATGTAAATTCAGAG | | TATGTAAATTCAGAG | | | | |
| | CTTTGTGGAACAGAG | | CTTTGTGGAACAGAG | | | | |
| | TTTCT | | TTTCA | | | | |
| rs13930 | ACAGGTACAAGAACC | 1077 | | | 7 | 117611671 | − |
| 4906 | AGTTGGCAGTATGTA | | | | | | |
| | AATTCAGAGCTTTGT | | | | | | |
| | GGAAC | | | | | | |
| rs13930 | ACAGGTACAAGAACC | 1078 | | | 7 | 117611671 | − |
| 4906 | AGTTGGCAGTATGTA | | | | | | |
| | AATTCAGAGCTTTGT | | | | | | |
| | GGAAC | | | | | | |
| rs13930 | ACAGGTACAAGAACC | 1079 | | | 7 | 117611671 | − |
| 4906 | AGTTGGCAGTATGTA | | | | | | |
| | AATTCAGAGCTTTGT | | | | | | |
| | GGAAC | | | | | | |
| rs79635 | GGACGGCAGCCTTAC | 1080 | GGACGGCAGCCTTAC | 2022 | 7 | 117611695 | + |
| 528 | TTTGAAACTCYGTTC | | TTTGAAACTCYGTTC | | | | |
| | CACAAAGCTCTGAAT | | CACAAAGCTCTGAAT | | | | |
| | TTACA | | TTACC | | | | |
| rs79635 | GGACGGCAGCCTTAC | 1081 | GGACGGCAGCCTTAC | 2023 | 7 | 117611695 | + |
| 528 | TTTGAAACTCYGTTC | | TTTGAAACTCYGTTC | | | | |
| | CACAAAGCTCTGAAT | | CACAAAGCTCTGAAT | | | | |
| | TTACA | | TTACC | | | | |
| rs79635 | GGACGGCAGCCTTAC | 1082 | GGACGGCAGCCTTAC | 2024 | 7 | 117611695 | + |
| 528 | TTTGAAACTCYGTTC | | TTTGAAACTCYGTTC | | | | |
| | CACAAAGCTCTGAAT | | CACAAAGCTCTGAAT | | | | |
| | TTACA | | TTACC | | | | |
| rs36210 | CATACTGCCAACTGG | 1083 | CATACTGCCAACTGG | 2025 | 7 | 117611743 | + |
| 737 | TTCTTGTACCTGTCA | | TTCTTGTACCTGTCA | | | | |
| | ACACTGCGCTGGTTC | | ACACTGCGCTGGTTC | | | | |
| | CAAAA | | CAAAT | | | | |
| rs36210 | AATGAAGGTAACAGC | 1084 | AATGAAGGTAACAGC | 2026 | 7 | 117611743 | − |
| 737 | AATGAAGAAGATGAC | | AATGAAGAAGATGAC | | | | |
| | AAAAATCATTTCTAT | | AAAAATCATTTCTAT | | | | |
| | TCTCT | | TCTCA | | | | |
| rs36210 | AATGAAGGTAACAGC | 1085 | AATGAAGGTAACAGC | 2027 | 7 | 117611743 | − |
| 737 | AATGAAGAAGATGAC | | AATGAAGAAGATGAC | | | | |
| | AAAAATCATTTCTAT | | AAAAATCATTTCTAT | | | | |
| | TCTCT | | TCTCA | | | | |
| rs75541 | AGCCATGAATATCAT | 1086 | AGCCATGAATATCAT | 2028 | 7 | 117614699 | + |
| 969 | GAGTACATTGCAGTG | | GAGTACATTGCAGTG | | | | |
| | GGCTGTAAACTCCAG | | GGCTGTAAACTCCAG | | | | |
| | CATAC | | CATAG | | | | |
| rs75541 | AGCCATGAATATCAT | 1087 | AGCCATGAATATCAT | 2029 | 7 | 117614699 | + |
| 969 | GAGTACATTGCAGTG | | GAGTACATTGCAGTG | | | | |
| | GGCTGTAAACTCCAG | | GGCTGTAAACTCCAG | | | | |
| | CATAC | | CATAG | | | | |
| rs75541 | AGCCATGAATATCAT | 1088 | AGCCATGAATATCAT | 2030 | 7 | 117614699 | + |
| 969 | GAGTACATTGCAGTG | | GAGTACATTGCAGTG | | | | |
| | GGCTGTAAACTCCAG | | GGCTGTAAACTCCAG | | | | |
| | CATAC | | CATAG | | | | |
| rs74767 | TGACTTGGTAGGTTT | 1089 | | | 7 | 117627537 | − |
| 530 | ACCTTCTGTTGGCAT | | | | | | |
| | GTCAATGAACTTAAA | | | | | | |
| | GACTC | | | | | | |
| rs74767 | TGACTTGGTAGGTTT | 1090 | | | 7 | 117627537 | − |
| 530 | ACCTTCTGTTGGCAT | | | | | | |
| | GTCAATGAACTTAAA | | | | | | |
| | GACTC | | | | | | |
| rs74767 | TGACTTGGTAGGTTT | 1091 | | | 7 | 117627537 | − |
| 530 | ACCTTCTGTTGGCAT | | | | | | |
| | GTCAATGAACTTAAA | | | | | | |
| | GACTC | | | | | | |
| rs74767 | TGACTTGGTAGGTTT | 1092 | | | 7 | 117627537 | − |
| 530 | ACCTTCTGTTGGCAT | | | | | | |
| | GTCAATGAACTTAAA | | | | | | |
| | GACTC | | | | | | |
| rs34911 | TACACAGAAGGTGGA | 1093 | | | 7 | 117627758 | + |
| 792 | AATGCCATATTAGAG | | | | | | |
| | AACATTTCCTTCTCA | | | | | | |
| | ATAAG | | | | | | |
| rs34911 | TACACAGAAGGTGGA | 1094 | | | 7 | 117627758 | + |
| 792 | AATGCCATATTAGAG | | | | | | |
| | AACATTTCCTTCTCA | | | | | | |
| | ATAAG | | | | | | |
| rs34911 | TACACAGAAGGTGGA | 1095 | | | 7 | 117627758 | + |
| 792 | AATGCCATATTAGAG | | | | | | |
| | AACATTTCCTTCTCA | | | | | | |
| | ATAAG | | | | | | |
| rs75039 | AATTGCATTGTACCA | 1096 | AATTGCATTGTACCA | 2031 | 7 | 117639961 | − |
| 782 | TGAATAGAACATTTC | | TGAATAGAACATTTC | | | | |
| | CTTTCAGGGTGTCTT | | CTTTCAGGGTGTCTT | | | | |
| | ACTCA | | ACTCG | | | | |
| rs75039 | AATTGCATTGTACCA | 1097 | AATTGCATTGTACCA | 2032 | 7 | 117639961 | − |
| 782 | TGAATAGAACATTTC | | TGAATAGAACATTTC | | | | |
| | CTTTCAGGGTGTCTT | | CTTTCAGGGTGTCTT | | | | |
| | ACTCA | | ACTCG | | | | |
| rs75039 | AATTGCATTGTACCA | 1098 | AATTGCATTGTACCA | 2033 | 7 | 117639961 | − |
| 782 | TGAATAGAACATTTC | | TGAATAGAACATTTC | | | | |
| | CTTTCAGGGTGTCTT | | CTTTCAGGGTGTCTT | | | | |
| | ACTCA | | ACTCG | | | | |
| rs26760 | TGTCACAGAAGTGAT | 1099 | TGTCACAGAAGTGAT | 2034 | 7 | 117642451 | + |
| 6723 | CCCATCACTTTTACC | | CCCATCACTTTTACC | | | | |
| | TTATAGGTGGGCCTC | | TTATAGGTGGGCCTC | | | | |
| | TTGGA | | TTGGG | | | | |
| rs26760 | TGTCACAGAAGTGAT | 1100 | TGTCACAGAAGTGAT | 2035 | 7 | 117642451 | + |
| 6723 | CCCATCACTTTTACC | | CCCATCACTTTTACC | | | | |
| | TTATAGGTGGGCCTC | | TTATAGGTGGGCCTC | | | | |
| | TTGGA | | TTGGG | | | | |
| rs26760 | TGTCACAGAAGTGAT | 1101 | TGTCACAGAAGTGAT | 2036 | 7 | 117642451 | + |
| 6723 | CCCATCACTTTTACC | | CCCATCACTTTTACC | | | | |
| | TTATAGGTGGGCCTC | | TTATAGGTGGGCCTC | | | | |
| | TTGGA | | TTGGG | | | | |
| rs39750 | ATCCCATCACTTTTA | 1102 | | | 7 | 117642465 | + |
| 8602 | CCTTATAGGTGGGCC | | | | | | |
| | TCTTGGGAAGAACTG | | | | | | |
| | GATCA | | | | | | |
| rs39750 | TCCTTCAGTGTTCAG | 1103 | | | 7 | 117642465 | − |
| 8602 | TAGTCTCAAAAAAGC | | | | | | |
| | TGATAACAAAGTACT | | | | | | |
| | CTTCC | | | | | | |
| rs39750 | ATCCCATCACTTTTA | 1104 | | | 7 | 117642465 | + |
| 8602 | CCTTATAGGTGGGCC | | | | | | |
| | TCTTGGGAAGAACTG | | | | | | |
| | GATCA | | | | | | |
| rs74503 | CACTTTTACCTTATA | 1105 | | | 7 | 117642472 | + |
| 330 | GGTGGGCCTCTTGGG | | | | | | |
| | AAGAACTGGATCAGG | | | | | | |
| | GAAGA | | | | | | |
| rs74503 | CACTTTTACCTTATA | 1106 | | | 7 | 117642472 | + |
| 330 | GGTGGGCCTCTTGGG | | | | | | |
| | AAGAACTGGATCAGG | | | | | | |
| | GAAGA | | | | | | |
| rs12190 | ATAGGTGGGCCTCTT | 1107 | ATAGGTGGGCCTCTT | 2037 | 7 | 117642483 | + |
| 9041 | GGGAAGAACTGGATC | | GGGAAGAACTGGATC | | | | |
| | AGGGAAGAGTACTTT | | AGGGAAGAGTACTTT | | | | |
| | GTTAT | | GTTAC | | | | |
| rs12190 | TATAGGTGGGCCTCT | 1108 | | | 7 | 117642483 | + |
| 9041 | TGGGAAGAACTGGAT | | | | | | |
| | CAGGGAAGAGTACTT | | | | | | |
| | TGTTA | | | | | | |
| rs11971 | AAGGCTTTCCTCCAC | 1109 | AAGGCTTTCCTCCAC | 2038 | 7 | 117642528 | − |
| 167 | TGTTGCAAAGTTATT | | TGTTGCAAAGTTATT | | | | |
| | GAATCCCAAGACACA | | GAATCCCAAGACACA | | | | |
| | CCATT | | CCATC | | | | |
| rs11971 | AAAGGCTTTCCTCCA | 1110 | | | 7 | 117642528 | − |
| 167 | CTGTTGCAAAGTTAT | | | | | | |
| | TGAATCCCAAGACAC | | | | | | |
| | ACCAT | | | | | | |
| rs11971 | AAAGGCTTTCCTCCA | 1111 | | | 7 | 117642528 | − |
| 167 | CTGTTGCAAAGTTAT | | | | | | |
| | TGAATCCCAAGACAC | | | | | | |
| | ACCAT | | | | | | |
| rs77010 | AAATCCAGATCGATG | 1112 | AAATCCAGATCGATG | 2039 | 7 | 117642566 | + |
| 898 | GTGTGTCTTGGGATT | | GTGTGTCTTGGGATT | | | | |
| | CAATAACTTTGCAAC | | CAATAACTTTGCAAC | | | | |
| | AGTGA | | AGTGG | | | | |
| rs77010 | AAATCCAGATCGATG | 1113 | AAATCCAGATCGATG | 2040 | 7 | 117642566 | + |
| 898 | GTGTGTCTTGGGATT | | GTGTGTCTTGGGATT | | | | |
| | CAATAACTTTGCAAC | | CAATAACTTTGCAAC | | | | |
| | AGTGA | | AGTGG | | | | |
| rs77010 | AAATCCAGATCGATG | 1114 | AAATCCAGATCGATG | 2041 | 7 | 117642566 | + |
| 898 | GTGTGTCTTGGGATT | | GTGTGTCTTGGGATT | | | | |
| | CAATAACTTTGCAAC | | CAATAACTTTGCAAC | | | | |
| | AGTGA | | AGTGG | | | | |
| rs80034 | CTGCAACTTTCCATA | 1115 | CTGCAACTTTCCATA | 2042 | 7 | 117652877 | − |
| 486 | TTTCTTGATCACTCC | | TTTCTTGATCACTCC | | | | |
| | ACTGTTCATAGGGAT | | ACTGTTCATAGGGAT | | | | |
| | CCAAG | | CCAAC | | | | |
| rs80034 | CTGCAACTTTCCATA | 1116 | CTGCAACTTTCCATA | 2043 | 7 | 117652877 | − |
| 486 | TTTCTTGATCACTCC | | TTTCTTGATCACTCC | | | | |
| | ACTGTTCATAGGGAT | | ACTGTTCATAGGGAT | | | | |
| | CCAAG | | CCAAC | | | | |
| rs80034 | CTGCAACTTTCCATA | 1117 | CTGCAACTTTCCATA | 2044 | 7 | 117652877 | − |
| 486 | TTTCTTGATCACTCC | | TTTCTTGATCACTCC | | | | |
| | ACTGTTCATAGGGAT | | ACTGTTCATAGGGAT | | | | |
| | CCAAG | | CCAAC | | | | |
| rs19392 | TCTTCGCCTTACTGA | 1118 | | | 7 | 117664770 | − |
| 2525 | GAACAGATCTAGCCA | | | | | | |
| | AGCACATCAACTGCT | | | | | | |
| | TGTGG | | | | | | |
| rs19392 | TCTTCGCCTTACTGA | 1119 | | | 7 | 117664770 | − |
| 2525 | GAACAGATCTAGCCA | | | | | | |
| | AGCACATCAACTGCT | | | | | | |
| | TGTGG | | | | | | |
| rs41303 | CATTCACCACCATGT | 1120 | | | 7 | 99250393 | + |
| 343 | CAAGGTACTCCATCT | | | | | | |
| | GTACCACGGCATCAT | | | | | | |
| | AGGTA | | | | | | |
| rs41303 | CATTCACCACCATGT | 1121 | | | 7 | 99250393 | + |
| 343 | CAAGGTACTCCATCT | | | | | | |
| | GTACCACGGCATCAT | | | | | | |
| | AGGTA | | | | | | |
| rs77674 | CCAAGGCTTCATATG | 1122 | | | 7 | 99270539 | + |
| 6 | ATGAAGGGTAATGTG | | | | | | |
| | GTCCAAACAGGGAAG | | | | | | |
| | AGATA | | | | | | |
| rs77674 | CCAAGGCTTCATATG | 1123 | | | 7 | 99270539 | + |
| 6 | ATGAAGGGTAATGTG | | | | | | |
| | GTCCAAACAGGGAAG | | | | | | |
| | AGATA | | | | | | |
| rs28365 | TGAAATCAATGGGGT | 1124 | | | 7 | 99652613 | − |
| 083 | ATTCATTCCCAAAGG | | | | | | |
| | GTCAATGGTGGTGAT | | | | | | |
| | TCCAA | | | | | | |
| rs28365 | TGAAATCAATGGGGT | 1125 | | | 7 | 99652613 | |
| 083 | ATTCATTCCCAAAGG | | | | | | |
| | GTCAATGGTGGTGAT | | | | | | |
| | TCCAA | | | | | | |
| rs28365 | TGAAATCAATGGGGT | 1126 | | | 7 | 99652613 | − |
| 083 | ATTCATTCCCAAAGG | | | | | | |
| | GTCAATGGTGGTGAT | | | | | | |
| | TCCAA | | | | | | |
| rs41303 | CATTCACCACCATGT | 1127 | | | 7 | 99652770 | + |
| 343 | CAAGGTACTCCATCT | | | | | | |
| | GTACCACGGCATCAT | | | | | | |
| | AGGTA | | | | | | |
| rs28383 | TCTTCCCTTCATCTC | 1128 | | | 7 | 99660516 | + |
| 479 | CAGGGGTCATCCCCT | | | | | | |
| | CACCTTATTGGGCAA | | | | | | |
| | AACTG | | | | | | |
| rs28383 | TCTTCCCTTCATCTC | 1129 | | | 7 | 99660516 | + |
| 479 | CAGGGGTCATCCCCT | | | | | | |
| | CACCTTATTGGGCAA | | | | | | |
| | AACTG | | | | | | |
| rs28383 | TCTTCCCTTCATCTC | 1130 | | | 7 | 99660516 | + |
| 479 | CAGGGGTCATCCCCT | | | | | | |
| | CACCTTATTGGGCAA | | | | | | |
| | AACTG | | | | | | |
| rs10264 | ATACTTATTGAGAGA | 1131 | | | 7 | 99665212 | + |
| 272 | AATAATGGATCTAAG | | | | | | |
| | AAACCAAATTTTAGG | | | | | | |
| | AACTT | | | | | | |
| rs10264 | ATACTTATTGAGAGA | 1132 | | | 7 | 99665212 | + |
| 272 | AATAATGGATCTAAG | | | | | | |
| | AAACCAAATTTTAGG | | | | | | |
| | AACTT | | | | | | |
| rs10264 | AACATCGACTCTCTC | 1133 | | | 7 | 99665212 | − |
| 272 | AACAATCCACAAGAC | | | | | | |
| | CCCTTTGTGGAGAGC | | | | | | |
| | ACTAA | | | | | | |
| rs56411 | CTAAGAAACCAAATT | 1134 | | | 7 | 99665237 | + |
| 402 | TTAGGAACTTCTTAG | | | | | | |
| | TGCTCTCCACAAAGG | | | | | | |
| | GGTCT | | | | | | |
| rs56411 | CTAAGAAACCAAATT | 1135 | | | 7 | 99665237 | + |
| 402 | TTAGGAACTTCTTAG | | | | | | |
| | TGCTCTCCACAAAGG | | | | | | |
| | GGTCT | | | | | | |
| rs56411 | CTAAGAAACCAAATT | 1136 | | | 7 | 99665237 | + |
| 402 | TTAGGAACTTCTTAG | | | | | | |
| | TGCTCTCCACAAAGG | | | | | | |
| | GGTCT | | | | | | |
| rs55965 | TCCCCAGATTCATTC | 1137 | | | 7 | 99666950 | + |
| 422 | TTTACATTTCTAATT | | | | | | |
| | AAGACTCATCTTATT | | | | | | |
| | TTCAT | | | | | | |
| rs55965 | CCCCAGATTCATTCT | 1138 | CCCCAGATTCATTCT | 2045 | 7 | 99666950 | + |
| 422 | TTACATTTCTAATTA | | TTACATTTCTAATTA | | | | |
| | AGACTCATCTTATTT | | AGACTCATCTTATTT | | | | |
| | TCATA | | TCATG | | | | |
| rs55965 | CCCCAGATTCATTCT | 1139 | CCCCAGATTCATTCT | 2046 | 7 | 99666950 | + |
| 422 | TTACATTTCTAATTA | | TTACATTTCTAATTA | | | | |
| | AGACTCATCTTATTT | | AGACTCATCTTATTT | | | | |
| | TCATA | | TCATG | | | | |
| rs77674 | CCAAGGCTTCATATG | 1140 | | | 7 | 99672916 | + |
| 6 | ATGAAGGGTAATGTG | | | | | | |
| | GTCCAAACAGGGAAG | | | | | | |
| | AGATA | | | | | | |
| rs55817 | GTCACAATCCCTGTG | 1141 | | | 7 | 99676198 | − |
| 950 | ACCTGATTTCTGTTT | | | | | | |
| | CACTTTGTAGATATG | | | | | | |
| | GGACC | | | | | | |
| rs55817 | GTCACAATCCCTGTG | 1142 | | | 7 | 99676198 | − |
| 950 | ACCTGATTTCTGTTT | | | | | | |
| | CACTTTGTAGATATG | | | | | | |
| | GGACC | | | | | | |
| rs55817 | GTCACAATCCCTGTG | 1143 | | | 7 | 99676198 | − |
| 950 | ACCTGATTTCTGTTT | | | | | | |
| | CACTTTGTAGATATG | | | | | | |
| | GGACC | | | | | | |
| rs22300 | ATCAGCAAGACACTC | 1144 | | | X | 153760654 | − |
| 37 | TCTCCCTCACAGAAC | | | | | | |
| | GTGAAGCTCCCTGAC | | | | | | |
| | GCCTA | | | | | | |
| rs13785 | CGGCCACATCATGGA | 1145 | | | X | 153760987 | + |
| 2345 | ACTGCAGCCTCACCT | | | | | | |
| | CGGCCTTGCGCTCGT | | | | | | |
| | TCAGG | | | | | | |
| rs76723 | CCAGTACGTGGGGAA | 1146 | | | X | 153761240 | − |
| 693 | CCCCGATGGAGAGGG | | | | | | |
| | CGAGGCCACCAAAGG | | | | | | |
| | GTACC | | | | | | |
| rs50308 | TGCCCAGGTAGTGGT | 1147 | | | X | 153762634 | + |
| 68 | CGATGCGGTAGATCT | | | | | | |
| | GGTCCTCACGGAACA | | | | | | |
| | GGGAG | | | | | | |
| rs50308 | TGCCCAGGTAGTGGT | 1148 | | | X | 153762634 | + |
| 68 | CGATGCGGTAGATCT | | | | | | |
| | GGTCCTCACGGAACA | | | | | | |
| | GGGAG | | | | | | |
| rs78478 | GGCAGTGGTGGGACA | 1149 | GGCAGTGGTGGGACA | 2047 | X | 153764383 | + |
| 128 | CACTTACCAGATGGT | | CACTTACCAGATGGT | | | | |
| | GGGGTAGATCTTCTT | | GGGGTAGATCTTCTT | | | | |
| | CTTGC | | CTTGG | | | | |
| rs78478 | GGCAGTGGTGGGACA | 1150 | GGCAGTGGTGGGACA | 2048 | X | 153764383 | + |
| 128 | CACTTACCAGATGGT | | CACTTACCAGATGGT | | | | |
| | GGGGTAGATCTTCTT | | GGGGTAGATCTTCTT | | | | |
| | CTTGC | | CTTGG | | | | |
| X_ | GGGCTCAGAGCTTGT | 1151 | | | X | 154532046 | + |
| 154532046 | GGGGGTTCACCCACT | | | | | | |
| | TGTAGGTGCCCTCAT | | | | | | |
| | ACTGG | | | | | | |
| X_ | GGGCTCAGAGCTTGT | 1152 | | | X | 154532046 | + |
| 154532046 | GGGGGTTCACCCACT | | | | | | |
| | TGTAGGTGCCCTCAT | | | | | | |
| | ACTGG | | | | | | |
| X_ | GGGCTCAGAGCTTGT | 1153 | | | X | 154532046 | + |
| 154532046 | GGGGGTTCACCCACT | | | | | | |
| | TGTAGGTGCCCTCAT | | | | | | |
| | ACTGG | | | | | | |
| X_ | CCACCCGTCACTCTC | 1154 | | | X | 154532057 | − |
| 154532057 | CAGCCGAGGCCCCAC | | | | | | |
| | GGAGGCAGACGAGCT | | | | | | |
| | GATGA | | | | | | |
| X_ | CCACCCGTCACTCTC | 1155 | | | X | 154532057 | − |
| 154532057 | CAGCCGAGGCCCCAC | | | | | | |
| | GGAGGCAGACGAGCT | | | | | | |
| | GATGA | | | | | | |
| X_ | CCACCCGTCACTCTC | 1156 | | | X | 154532057 | − |
| 154532057 | CAGCCGAGGCCCCAC | | | | | | |
| | GGAGGCAGACGAGCT | | | | | | |
| | GATGA | | | | | | |
| X_ | TGCCCTCATACTGGA | 1157 | | | X | 154532082 | + |
| 154532082 | AACCCACTCTCTTCA | | | | | | |
| | TCAGCTCGTCTGCCT | | | | | | |
| | CCGTG | | | | | | |
| X_ | TGCCCTCATACTGGA | 1158 | | | X | 154532082 | + |
| 154532082 | AACCCACTCTCTTCA | | | | | | |
| | TCAGCTCGTCTGCCT | | | | | | |
| | CCGTG | | | | | | |
| X_ | TGCCCTCATACTGGA | 1159 | | | X | 154532082 | + |
| 154532082 | AACCCACTCTCTTCA | | | | | | |
| | TCAGCTCGTCTGCCT | | | | | | |
| | CCGTG | | | | | | |
| X_ | GCCCTCATACTGGAA | 1160 | | | X | 154532083 | + |
| 154532083 | ACCCACTCTCTTCAT | | | | | | |
| | CAGCTCGTCTGCCTC | | | | | | |
| | CGTGG | | | | | | |
| X_ | GCCCTCATACTGGAA | 1161 | | | X | 154532083 | + |
| 154532083 | ACCCACTCTCTTCAT | | | | | | |
| | CAGCTCGTCTGCCTC | | | | | | |
| | CGTGG | | | | | | |
| X_ | GCCCTCATACTGGAA | 1162 | | | X | 154532083 | + |
| 154532083 | ACCCACTCTCTTCAT | | | | | | |
| | CAGCTCGTCTGCCTC | | | | | | |
| | CGTGG | | | | | | |
| X_ | CCTCATACTGGAAAC | 1163 | | | X | 154532085 | + |
| 154532085 | CCACTCTCTTCATCA | | | | | | |
| | GCTCGTCTGCCTCCG | | | | | | |
| | TGRRG | | | | | | |
| X_ | CCTCATACTGGAAAC | 1164 | | | X | 154532085 | + |
| 154532085 | CCACTCTCTTCATCA | | | | | | |
| | GCTCGTCTGCCTCCG | | | | | | |
| | TGRRG | | | | | | |
| X_ | CCTCATACTGGAAAC | 1165 | | | X | 154532085 | + |
| 154532085 | CCACTCTCTTCATCA | | | | | | |
| | GCTCGTCTGCCTCCG | | | | | | |
| | TGRRG | | | | | | |
| X_ | CTCATACTGGAAACC | 1166 | | | X | 154532086 | + |
| 154532086 | CACTCTCTTCATCAG | | | | | | |
| | CTCGTCTGCCTCCGT | | | | | | |
| | GGGGC | | | | | | |
| X_ | CTCATACTGGAAACC | 1167 | | | X | 154532086 | + |
| 154532086 | CACTCTCTTCATCAG | | | | | | |
| | CTCGTCTGCCTCCGT | | | | | | |
| | GGGGC | | | | | | |
| X_ | CTCATACTGGAAACC | 1168 | | | X | 154532086 | + |
| 154532086 | CACTCTCTTCATCAG | | | | | | |
| | CTCGTCTGCCTCCGT | | | | | | |
| | GGGGC | | | | | | |
| rs13785 | TTCACCCCACTGCTG | 1169 | TTCACCCCACTGCTG | 2049 | X | 154532203 | − |
| 2348 | CACCAGATTGAGCTG | | CACCAGATTGAGCTG | | | | |
| | GAGAAGCCCAAGCCC | | GAGAAGCCCAAGCCC | | | | |
| | ATCCG | | ATCCC | | | | |
| rs13785 | TTCACCCCACTGCTG | 1170 | TTCACCCCACTGCTG | 2050 | X | 154532203 | − |
| 2348 | CACCAGATTGAGCTG | | CACCAGATTGAGCTG | | | | |
| | GAGAAGCCCAAGCCC | | GAGAAGCCCAAGCCC | | | | |
| | ATCCG | | ATCCC | | | | |
| X_ | GCGACGAGCTCCGTG | 1171 | | | X | 154532231 | − |
| 154532231 | AGGCCTGGCGTATTT | | | | | | |
| | TCACCCCACTGCTGC | | | | | | |
| | ACCAG | | | | | | |
| X_ | GCGACGAGCTCCGTG | 1172 | | | X | 154532231 | − |
| 154532231 | AGGCCTGGCGTATTT | | | | | | |
| | TCACCCCACTGCTGC | | | | | | |
| | ACCAG | | | | | | |
| X_ | GCGACGAGCTCCGTG | 1173 | | | X | 154532231 | − |
| 154532231 | AGGCCTGGCGTATTT | | | | | | |
| | TCACCCCACTGCTGC | | | | | | |
| | ACCAG | | | | | | |
| rs13785 | CCATGTCCCCTCAGC | 1174 | CCATGTCCCCTCAGC | 2051 | X | 154532245 | − |
| 2344 | GACGAGCTCCGTGAG | | GACGAGCTCCGTGAG | | | | |
| | GCCTGGCGTATTTTC | | GCCTGGCGTATTTTC | | | | |
| | ACCCC | | ACCCG | | | | |
| rs13785 | CCATGTCCCCTCAGC | 1175 | CCATGTCCCCTCAGC | 2052 | X | 154532245 | − |
| 2344 | GACGAGCTCCGTGAG | | GACGAGCTCCGTGAG | | | | |
| | GCCTGGCGTATTTTC | | GCCTGGCGTATTTTC | | | | |
| | ACCCC | | ACCCG | | | | |
| rs13785 | CCATGTCCCCTCAGC | 1176 | CCATGTCCCCTCAGC | 2053 | X | 154532245 | − |
| 2344 | GACGAGCTCCGTGAG | | GACGAGCTCCGTGAG | | | | |
| | GCCTGGCGTATTTTC | | GCCTGGCGTATTTTC | | | | |
| | ACCCC | | ACCCG | | | | |
| rs72554 | CTCCCAAGCCATACC | 1177 | | | X | 154532257 | − |
| 664 | ATGTCCCCTCAGCGA | | | | | | |
| | CGAGCTCCGTGAGGC | | | | | | |
| | CTGGC | | | | | | |
| rs72554 | CTCCCAAGCCATACC | 1178 | | | X | 154532257 | − |
| 664 | ATGTCCCCTCAGCGA | | | | | | |
| | CGAGCTCCGTGAGGC | | | | | | |
| | CTGGC | | | | | | |
| rs72554 | CTCCCAAGCCATACC | 1179 | | | X | 154532257 | − |
| 664 | ATGTCCCCTCAGCGA | | | | | | |
| | CGAGCTCCGTGAGGC | | | | | | |
| | CTGGC | | | | | | |
| X_ | CTCCCAAGCCATACC | 1180 | CTCCCAAGCCATACC | 2054 | X | 154532258 | − |
| 154532258 | ATGTCCCCTCAGCGA | | ATGTCCCCTCAGCGA | | | | |
| | CGAGCTCCGTGAGGC | | CGAGCTCCGTGAGGC | | | | |
| | CTGGT | | CTGGC | | | | |
| X_ | GGCTTGGGCTTCTCC | 1181 | GGCTTGGGCTTCTCC | 2055 | X | 154532258 | + |
| 154532258 | AGCTCAATCTGGTGC | | AGCTCAATCTGGTGC | | | | |
| | AGCAGTSGGGTGAAA | | AGCAGTSGGGTGAAA | | | | |
| | ATACT | | ATACG | | | | |
| X_ | CTCCCAAGCCATACC | 1182 | CTCCCAAGCCATACC | 2056 | X | 154532258 | − |
| 154532258 | ATGTCCCCTCAGCGA | | ATGTCCCCTCAGCGA | | | | |
| | CGAGCTCCGTGAGGC | | CGAGCTCCGTGAGGC | | | | |
| | CTGGA | | CTGGC | | | | |
| rs78260 | GGGCTTCTCCAGCTC | 1183 | | | X | 154532264 | + |
| 8284 | AATCTGGTGCAGCAG | | | | | | |
| | TGGGGTGAAAATACG | | | | | | |
| | CCAGG | | | | | | |
| rs78260 | GGGCTTCTCCAGCTC | 1184 | | | X | 154532264 | + |
| 8284 | AATCTGGTGCAGCAG | | | | | | |
| | TGGGGTGAAAATACG | | | | | | |
| | CCAGG | | | | | | |
| rs78260 | GGGCTTCTCCAGCTC | 1185 | | | X | 154532264 | + |
| 8284 | AATCTGGTGCAGCAG | | | | | | |
| | TGGGGTGAAAATACG | | | | | | |
| | CCAGG | | | | | | |
| X_ | GCTTCTCCAGCTCAA | 1186 | GCTTCTCCAGCTCAA | 2057 | X | 154532265 | + |
| 154532265 | TCTGGTGCAGCAGTS | | TCTGGTGCAGCAGTS | | | | |
| | GGGTGAAAATAYDCC | | GGGTGAAAATAYDCC | | | | |
| | AGGCG | | AGGCC | | | | |
| X_ | GCTTCTCCAGCTCAA | 1187 | GCTTCTCCAGCTCAA | 2058 | X | 154532265 | + |
| 154532265 | TCTGGTGCAGCAGTS | | TCTGGTGCAGCAGTS | | | | |
| | GGGTGAAAATAYDCC | | GGGTGAAAATAYDCC | | | | |
| | AGGCG | | AGGCC | | | | |
| X_ | GCTTCTCCAGCTCAA | 1188 | GCTTCTCCAGCTCAA | 2059 | X | 154532265 | + |
| 154532265 | TCTGGTGCAGCAGTS | | TCTGGTGCAGCAGTS | | | | |
| | GGGTGAAAATAYDCC | | GGGTGAAAATAYDCC | | | | |
| | AGGCG | | AGGCC | | | | |
| rs72554 | GTGCCACCGGCCTCC | 1189 | GTGCCACCGGCCTCC | 2060 | X | 154532269 | − |
| 665 | CAAGCCATACCATGT | | CAAGCCATACCATGT | | | | |
| | CCCCTCAGCGACGAG | | CCCCTCAGCGACGAG | | | | |
| | CTCCT | | CTCCG | | | | |
| rs72554 | GTGCCACCGGCCTCC | 1190 | GTGCCACCGGCCTCC | 2061 | X | 154532269 | − |
| 665 | CAAGCCATACCATGT | | CAAGCCATACCATGT | | | | |
| | CCCCTCAGCGACGAG | | CCCCTCAGCGACGAG | | | | |
| | CTCCT | | CTCCG | | | | |
| rs72554 | CTCCAGCTCAATCTG | 1191 | CTCCAGCTCAATCTG | 2062 | X | 154532269 | + |
| 665 | GTGCAGCAGTGGGGT | | GTGCAGCAGTGGGGT | | | | |
| | GAAAATACDCCAGGY | | GAAAATACDCCAGGY | | | | |
| | STCAA | | STCAC | | | | |
| X_ | AATCTGGTGCAGCAG | 1192 | AATCTGGTGCAGCAG | 2063 | X | 154532278 | + |
| 154532278 | TGGGGTGAAAATACG | | TGGGGTGAAAATACG | | | | |
| | CCAGGCCTCACGGAG | | CCAGGCCTCACGGAG | | | | |
| | CTCGT | | CTCGA | | | | |
| X_ | AATCTGGTGCAGCAG | 1193 | AATCTGGTGCAGCAG | 2064 | X | 154532278 | + |
| 154532278 | TGGGGTGAAAATACG | | TGGGGTGAAAATACG | | | | |
| | CCAGGCCTCACGGAG | | CCAGGCCTCACGGAG | | | | |
| | CTCGT | | CTCGA | | | | |
| X_ | AATCTGGTGCAGCAG | 1194 | AATCTGGTGCAGCAG | 2065 | X | 154532278 | + |
| 154532278 | TGGGGTGAAAATACG | | TGGGGTGAAAATACG | | | | |
| | CCAGGCCTCACGGAG | | CCAGGCCTCACGGAG | | | | |
| | CTCGT | | CTCGA | | | | |
| X_ | ATCTGGTGCAGCAGT | 1195 | ATCTGGTGCAGCAGT | 2066 | X | 154532279 | + |
| 154532279 | GGGGTGAAAATACGC | | GGGGTGAAAATACGC | | | | |
| | CAGGCCTCACGGAGC | | CAGGCCTCACGGAGC | | | | |
| | TCGTG | | TCGTC | | | | |
| X_ | ATCTGGTGCAGCAGT | 1196 | ATCTGGTGCAGCAGT | 2067 | X | 154532279 | + |
| 154532279 | GGGGTGAAAATACGC | | GGGGTGAAAATACGC | | | | |
| | CAGGCCTCACGGAGC | | CAGGCCTCACGGAGC | | | | |
| | TCGTG | | TCGTC | | | | |
| X_ | ATCTGGTGCAGCAGT | 1197 | ATCTGGTGCAGCAGT | 2068 | X | 154532279 | + |
| 154532279 | GGGGTGAAAATACGC | | GGGGTGAAAATACGC | | | | |
| | CAGGCCTCACGGAGC | | CAGGCCTCACGGAGC | | | | |
| | TCGTG | | TCGTC | | | | |
| rs13785 | CCACCCCATAGCCCA | 1198 | | | X | 154532389 | + |
| 2324 | CAGGTATGCAGGGGC | | | | | | |
| | CGGCAGCTGGGCCTC | | | | | | |
| | ACCTG | | | | | | |
| rs13785 | CCACCCCATAGCCCA | 1199 | | | X | 154532389 | + |
| 2324 | CAGGTATGCAGGGGC | | | | | | |
| | CGGCAGCTGGGCCTC | | | | | | |
| | ACCTG | | | | | | |
| rs13785 | TGAGCGCCTCATCCT | 1200 | | | X | 154532389 | |
| 2324 | GGACGTCTTCTGCGG | | | | | | |
| | GAGCCAGATGCACTT | | | | | | |
| | CGTGC | | | | | | |
| rs39812 | ATGAGCGCCTCATCC | 1201 | | | X | 154532390 | − |
| 3546 | TGGACGTCTTCTGCG | | | | | | |
| | GGAGCCAGATGCACT | | | | | | |
| | TCGTG | | | | | | |
| rs39812 | ATGAGCGCCTCATCC | 1202 | | | X | 154532390 | − |
| 3546 | TGGACGTCTTCTGCG | | | | | | |
| | GGAGCCAGATGCACT | | | | | | |
| | TCGTG | | | | | | |
| rs39812 | ACGAGCGCCTCATCC | 1203 | | | X | 154532390 | − |
| 3546 | TGGACGTCTTCTGCG | | | | | | |
| | GGAGCCAGATGCACT | | | | | | |
| | TCGTG | | | | | | |
| X_ | TATGAGCGCCTCATC | 1204 | TATGAGCGCCTCATC | 2069 | X | 154532392 | − |
| 154532392 | CTGGACGTCTTCTGC | | CTGGACGTCTTCTGC | | | | |
| | GGGAGCCAGATGCAC | | GGGAGCCAGATGCAC | | | | |
| | TTCGT | | TTCGA | | | | |
| X_ | TATGAGCGCCTCATC | 1205 | TATGAGCGCCTCATC | 2070 | X | 154532392 | − |
| 154532392 | CTGGACGTCTTCTGC | | CTGGACGTCTTCTGC | | | | |
| | GGGAGCCAGATGCAC | | GGGAGCCAGATGCAC | | | | |
| | TTCGT | | TTCGA | | | | |
| X_ | TATGAGCGCCTCATC | 1206 | TATGAGCGCCTCATC | 2071 | X | 154532392 | − |
| 154532392 | CTGGACGTCTTCTGC | | CTGGACGTCTTCTGC | | | | |
| | GGGAGCCAGATGCAC | | GGGAGCCAGATGCAC | | | | |
| | TTCGT | | TTCGA | | | | |
| X_ | TCCCTGACGCCTATG | 1207 | TCCCTGACGCCTATG | 2072 | X | 154532403 | − |
| 154532403 | AGCGCCTCATCCTGG | | AGCGCCTCATCCTGG | | | | |
| | ACGTCTTCTGCRGGR | | ACGTCTTCTGCRGGR | | | | |
| | GCCAG | | GCCAC | | | | |
| X_ | TCCCTGACGCCTATG | 1208 | TCCCTGACGCCTATG | 2073 | X | 154532403 | − |
| 154532403 | AGCGCCTCATCCTGG | | AGCGCCTCATCCTGG | | | | |
| | ACGTCTTCTGCRGGR | | ACGTCTTCTGCRGGR | | | | |
| | GCCAG | | GCCAC | | | | |
| X_ | TCCCTGACGCCTATG | 1209 | TCCCTGACGCCTATG | 2074 | X | 154532403 | − |
| 154532403 | AGCGCCTCATCCTGG | | AGCGCCTCATCCTGG | | | | |
| | ACGTCTTCTGCRGGR | | ACGTCTTCTGCRGGR | | | | |
| | GCCAG | | GCCAC | | | | |
| X_ | TGAAGCTCCCTGACG | 1210 | | | X | 154532408 | − |
| 154532408 | CCTATGAGCGCYTCA | | | | | | |
| | TCCTGGACGTCTTCT | | | | | | |
| | GCRGG | | | | | | |
| X_ | TGAAGCTCCCTGACG | 1211 | | | X | 154532408 | − |
| 154532408 | CCTATGAGCGCYTCA | | | | | | |
| | TCCTGGACGTCTTCT | | | | | | |
| | GCRGG | | | | | | |
| X_ | TGAAGCTCCCTGACG | 1212 | | | X | 154532408 | − |
| 154532408 | CCTATGAGCGCYTCA | | | | | | |
| | TCCTGGACGTCTTCT | | | | | | |
| | GCRGG | | | | | | |
| rs13785 | GCAGGGGCCGGCAGC | 1213 | | | X | 154532411 | + |
| 2317 | TGGGCCTCACCTGCG | | | | | | |
| | CACGAAGTGCATCTG | | | | | | |
| | GCTCC | | | | | | |
| rs13785 | GCAGGGGCCGGCAGC | 1214 | | | X | 154532411 | + |
| 2317 | TGGGCCTCACCTGCG | | | | | | |
| | CACGAAGTGCATCTG | | | | | | |
| | GCTCC | | | | | | |
| rs13785 | ACGTGAAGCTCCCTG | 1215 | | | X | 154532411 | − |
| 2317 | ACGCCTACGAGCGCC | | | | | | |
| | TCATCCTGGACGTCT | | | | | | |
| | TCTGC | | | | | | |
| X_ | TCACCTGCGCACGAA | 1216 | | | X | 154532432 | + |
| 154532432 | GTGCATCTGGCYCCY | | | | | | |
| | GCAGAAGACGTCCAG | | | | | | |
| | GATGA | | | | | | |
| X_ | TCACCTGCGCACGAA | 1217 | | | X | 154532432 | + |
| 154532432 | GTGCATCTGGCTCCC | | | | | | |
| | GCAGAAGACGTCCAG | | | | | | |
| | GATGA | | | | | | |
| X_ | TCACCTGCGCACGAA | 1218 | | | X | 154532432 | + |
| 154532432 | GTGCATCTGGCTCCC | | | | | | |
| | GCAGAAGACGTCCAG | | | | | | |
| | GATGA | | | | | | |
| rs13785 | CCTGCGCACGAAGTG | 1219 | CCTGCGCACGAAGTG | 2075 | X | 154532434 | + |
| 2337 | CATCTGGCTCCYGCA | | CATCTGGCTCCYGCA | | | | |
| | GAAGACGTCCAGGAT | | GAAGACGTCCAGGAT | | | | |
| | GARGC | | GARGG | | | | |
| rs13785 | CCTGCGCACGAAGTG | 1220 | CCTGCGCACGAAGTG | 2076 | X | 154532434 | + |
| 2337 | CATCTGGCTCCCGCA | | CATCTGGCTCCCGCA | | | | |
| | GAAGACGTCCAGGAT | | GAAGACGTCCAGGAT | | | | |
| | GAGGC | | GAGGG | | | | |
| rs13785 | CCTGCGCACGAAGTG | 1221 | CCTGCGCACGAAGTG | 2077 | X | 154532434 | + |
| 2337 | CATCTGGCTCCCGCA | | CATCTGGCTCCCGCA | | | | |
| | GAAGACGTCCAGGAT | | GAAGACGTCCAGGAT | | | | |
| | GAGGC | | GAGGG | | | | |
| rs22300 | ATCAGCAAGACACTC | 1222 | | | X | 154532439 | − |
| 37 | TCTCCCTCACAGAAC | | | | | | |
| | GTGAAGCTCCCTGAC | | | | | | |
| | GCCTA | | | | | | |
| rs22300 | ATCAGCAAGACACTC | 1223 | | | X | 154532439 | − |
| 37 | TCTCCCTCACAGAAC | | | | | | |
| | GTGAAGCTCCCTGAC | | | | | | |
| | GCCTA | | | | | | |
| X_ | TCCCGCAGAAGACGT | 1224 | | | X | 154532458 | + |
| 154532458 | CCAGGATGAGGCGCT | | | | | | |
| | CATAGGCGTCAGGGA | | | | | | |
| | GCTTC | | | | | | |
| X_ | CATGGTGGCAGGCAG | 1225 | | | X | 154532458 | − |
| 154532458 | TGGCATCAGCAAGAC | | | | | | |
| | ACTCTCTCCCTCACA | | | | | | |
| | GAACR | | | | | | |
| X_ | CATGGTGGCAGGCAG | 1226 | | | X | 154532458 | − |
| 154532458 | TGGCATCAGCAAGAC | | | | | | |
| | ACTCTCTCCCTCACA | | | | | | |
| | GAACR | | | | | | |
| rs78209 | ACATGGTGGCAGGCA | 1227 | | | X | 154532459 | − |
| 8548 | GTGGCATCAGCAAGA | | | | | | |
| | CACTCTCTCCCTCAC | | | | | | |
| | AGAAC | | | | | | |
| rs78209 | ACATGGTGGCAGGCA | 1228 | | | X | 154532459 | − |
| 8548 | GTGGCATCAGCAAGA | | | | | | |
| | CACTCTCTCCCTCAC | | | | | | |
| | AGAAC | | | | | | |
| rs78209 | ACATGGTGGCAGGCA | 1229 | | | X | 154532459 | − |
| 8548 | GTGGCATCAGCAAGA | | | | | | |
| | CACTCTCTCCCTCAC | | | | | | |
| | AGAAC | | | | | | |
| X_ | GGGCCCAGGCCGCCC | 1230 | | | X | 154532570 | + |
| 154532570 | ACCCTCCACACTGCT | | | | | | |
| | CCTTCTCTGTAGGGC | | | | | | |
| | ACCTT | | | | | | |
| X_ | GGGCCCAGGCCGCCC | 1231 | | | X | 154532570 | + |
| 154532570 | ACCCTCCACACTGCT | | | | | | |
| | CCTTCTCTGTAGGGC | | | | | | |
| | ACCTT | | | | | | |
| X_ | GGGCCCAGGCCGCCC | 1232 | | | X | 154532570 | + |
| 154532570 | ACCCTCCACACTGCT | | | | | | |
| | CCTTCTCTGTAGGGC | | | | | | |
| | ACCTT | | | | | | |
| X_ | GACCAAGAAGCCGGG | 1233 | GACCAAGAAGCCGGG | 2078 | X | 154532590 | − |
| 154532590 | CATGTTCTTCAACCC | | CATGTTCTTCAACCC | | | | |
| | CGAGGAGTCGGAGCT | | CGAGGAGTCGGAGCT | | | | |
| | GGACG | | GGACC | | | | |
| X_ | GACCAAGAAGCCGGG | 1234 | GACCAAGAAGCCGGG | 2079 | X | 154532590 | − |
| 154532590 | CATGTTCTTCAACCC | | CATGTTCTTCAACCC | | | | |
| | CRAGGAGTCGGAGCT | | CRAGGAGTCGGAGCT | | | | |
| | GGACG | | GGACC | | | | |
| X_ | GACCAAGAAGCCGGG | 1235 | GACCAAGAAGCCGGG | 2080 | X | 154532590 | − |
| 154532590 | CATGTTCTTCAACCC | | CATGTTCTTCAACCC | | | | |
| | CRAGGAGTCGGAGCT | | CRAGGAGTCGGAGCT | | | | |
| | GGACG | | GGACC | | | | |
| X_ | GTAGGGCACCTTGTA | 1236 | | | X | 154532608 | + |
| 154532608 | TCTGTTGCCGTAGGT | | | | | | |
| | CASGTCCAGCTCCGA | | | | | | |
| | CTCCT | | | | | | |
| X_ | GTAGGGCACCTTGTA | 1237 | | | X | 154532608 | + |
| 154532608 | TCTGTTGCCGTAGGT | | | | | | |
| | CAGGTCCAGCTCCGA | | | | | | |
| | CTCCT | | | | | | |
| X_ | GTAGGGCACCTTGTA | 1238 | | | X | 154532608 | + |
| 154532608 | TCTGTTGCCGTAGGT | | | | | | |
| | CAGGTCCAGCTCCGA | | | | | | |
| | CTCCT | | | | | | |
| X_ | TCTGTTGCCGTAGGT | 1239 | | | X | 154532623 | + |
| 154532623 | CAGGTCCAGCTCCGA | | | | | | |
| | CTCCTYGGGGTTGAA | | | | | | |
| | GAACA | | | | | | |
| X_ | TCTGTTGCCGTAGGT | 1240 | | | X | 154532623 | + |
| 154532623 | CAGGTCCAGCTCCGA | | | | | | |
| | CTCCTYGGGGTTGAA | | | | | | |
| | GAACA | | | | | | |
| X_ | TCTGTTGCCGTAGGT | 1241 | | | X | 154532623 | + |
| 154532623 | CAGGTCCAGCTCCGA | | | | | | |
| | CTCCTCGGGGTTGAA | | | | | | |
| | GAACA | | | | | | |
| rs13785 | GTTGCCGTAGGTCAG | 1242 | GTTGCCGTAGGTCAG | 2081 | X | 154532625 | + |
| 2336 | GTCCAGCTCCGACTC | | GTCCAGCTCCGACTC | | | | |
| | CTYGGGGTTGAAGAA | | CTYGGGGTTGAAGAA | | | | |
| | CAYGG | | CAYGC | | | | |
| rs13785 | GTGCAGCCCAACGAG | 1243 | GTGCAGCCCAACGAG | 2082 | X | 154532625 | − |
| 2336 | GCCGTGTACACCAAG | | GCCGTGTACACCAAG | | | | |
| | ATGATGACCAAGAAG | | ATGATGACCAAGAAG | | | | |
| | YSGKC | | YSGKG | | | | |
| rs13785 | TGTTGCCGTAGGTCA | 1244 | | | X | 154532625 | + |
| 2336 | GGTCCAGCTCCGACT | | | | | | |
| | CCTCGGGGTTGAAGA | | | | | | |
| | ACATG | | | | | | |
| rs13785 | GTTGCCGTAGGTCAG | 1245 | | | X | 154532626 | + |
| 2323 | GTCCAGCTCCGACTC | | | | | | |
| | CTCGGGGTTGAAGAA | | | | | | |
| | CATGC | | | | | | |
| rs13785 | GTTGCCGTAGGTCAG | 1246 | | | X | 154532626 | + |
| 2323 | GTCCAGCTCCGACTC | | | | | | |
| | CTYGGGGTTGAAGAA | | | | | | |
| | CAYGC | | | | | | |
| rs13785 | GTTGCCGTAGGTCAG | 1247 | | | X | 154532626 | + |
| 2323 | GTCCAGCTCCGACTC | | | | | | |
| | CTCGGGGTTGAAGAA | | | | | | |
| | CATGC | | | | | | |
| X_ | GCCGTAGGTCAGGTC | 1248 | GCCGTAGGTCAGGTC | 2083 | X | 154532628 | + |
| 154532628 | CAGCTCCGACTCCTC | | CAGCTCCGACTCCTC | | | | |
| | GGGGTTGAAGAACAT | | GGGGTTGAAGAACAT | | | | |
| | GCCCG | | GCCCC | | | | |
| X_ | GCCGTAGGTCAGGTC | 1249 | GCCGTAGGTCAGGTC | 2084 | X | 154532628 | + |
| 154532628 | CAGCTCCGACTCCTC | | CAGCTCCGACTCCTC | | | | |
| | GGGGTTGAAGAACAT | | GGGGTTGAAGAACAT | | | | |
| | GCCCG | | GCCCC | | | | |
| X_ | GCCGTAGGTCAGGTC | 1250 | GCCGTAGGTCAGGTC | 2085 | X | 154532628 | + |
| 154532628 | CAGCTCCGACTCCTC | | CAGCTCCGACTCCTC | | | | |
| | GGGGTTGAAGAACAT | | GGGGTTGAAGAACAT | | | | |
| | GCCCG | | GCCCC | | | | |
| X_ | GCCGTAGGTCAGGTC | 1251 | | | X | 154532629 | + |
| 154532629 | CAGCTCCGACTCCTC | | | | | | |
| | GGGGTTGAAGAACAT | | | | | | |
| | GCCCG | | | | | | |
| X_ | GCCGTAGGTCAGGTC | 1252 | | | X | 154532629 | + |
| 154532629 | CAGCTCCGACTCCTC | | | | | | |
| | GGGGTTGAAGAACAT | | | | | | |
| | GCCCG | | | | | | |
| X_ | GCCGTAGGTCAGGTC | 1253 | | | X | 154532629 | + |
| 154532629 | CAGCTCCGACTCCTC | | | | | | |
| | GGGGTTGAAGAACAT | | | | | | |
| | GCCCG | | | | | | |
| X_ | AGGTCAGGTCCAGCT | 1254 | | | X | 154532634 | + |
| 154532634 | CCGACTCCTCGGGGT | | | | | | |
| | TGAAGAACATGCCCG | | | | | | |
| | GCTTC | | | | | | |
| X_ | AGGTCAGGTCCAGCT | 1255 | | | X | 154532634 | + |
| 154532634 | CCGACTCCTCGGGGT | | | | | | |
| | TGAAGAACATGCMCS | | | | | | |
| | RCTTC | | | | | | |
| X_ | AGGTCAGGTCCAGCT | 1256 | | | X | 154532634 | + |
| 154532634 | CCGACTCCTCGGGGT | | | | | | |
| | TGAAGAACATGCCCG | | | | | | |
| | GCTTC | | | | | | |
| X_ | AGGTCCAGCTCCGAC | 1257 | | | X | 154532639 | + |
| 154532639 | TCCTCGGGGTTGAAG | | | | | | |
| | AACATGCCCSRCTTC | | | | | | |
| | KTGGT | | | | | | |
| X_ | AGGTCCAGCTCCGAC | 1258 | | | X | 154532639 | + |
| 154532639 | TCCTCGGGGTTGAAG | | | | | | |
| | AACATGCCCGGCTTC | | | | | | |
| | TTGGT | | | | | | |
| X_ | AGGTCCAGCTCCGAC | 1259 | | | X | 154532639 | + |
| 154532639 | TCCTCGGGGTTGAAG | | | | | | |
| | AACATGCCCGGCTTC | | | | | | |
| | TTGGT | | | | | | |
| X_ | CCGACTCCTCGGGGT | 1260 | | | X | 154532649 | + |
| 154532649 | TGAAGAACATGCCCG | | | | | | |
| | GCTTCTTGGTCATCA | | | | | | |
| | TCTTG | | | | | | |
| X_ | CCGACTCCTCGGGGT | 1261 | | | X | 154532649 | + |
| 154532649 | TGAAGAACATGCCCG | | | | | | |
| | GCTTCTTGGTCATCA | | | | | | |
| | TCTTG | | | | | | |
| X_ | CCGACTCCTCGGGGT | 1262 | | | X | 154532649 | + |
| 154532649 | TGAAGAACATGCCCG | | | | | | |
| | GCTTCTTGGTCATCA | | | | | | |
| | TCTTG | | | | | | |
| X_ | CCACCAGCAGTGCAA | 1263 | | | X | 154532661 | − |
| 154532661 | GCGCAACGAGCTGGT | | | | | | |
| | GATCCGCSTGCAGCY | | | | | | |
| | CAACR | | | | | | |
| X_ | CCACCAGCAGTGCAA | 1264 | | | X | 154532661 | − |
| 154532661 | GCGCAACGAGCTGGT | | | | | | |
| | GATCCGCGTGCAGCC | | | | | | |
| | CAACG | | | | | | |
| X_ | CCACCAGCAGTGCAA | 1265 | | | X | 154532661 | − |
| 154532661 | GCGCAACGAGCTGGT | | | | | | |
| | GATCCGCGTGCAGCC | | | | | | |
| | CAACG | | | | | | |
| rs13785 | GTTGAAGAACATGCC | 1266 | | | X | 154532662 | + |
| 2325 | CGGCTTCTTGGTYAT | | | | | | |
| | CATCTTGKTGTACAC | | | | | | |
| | GGCCT | | | | | | |
| rs13785 | TCCACCAGCAGTGCA | 1267 | | | X | 154532662 | − |
| 2325 | AGCGCAACGAGCTGG | | | | | | |
| | TGATCCGCGTGCAGC | | | | | | |
| | CCAAC | | | | | | |
| rs13785 | GTTGAAGAACATGCC | 1268 | | | X | 154532662 | + |
| 2325 | CGGCTTCTTGGTCAT | | | | | | |
| | CATCTTGGTGTACAC | | | | | | |
| | GGCCT | | | | | | |
| X_ | CATCTTCCACCAGCA | 1269 | | | X | 154532667 | − |
| 154532667 | GTGCAAGCGCAACGA | | | | | | |
| | GCTGGTGATCSRCST | | | | | | |
| | GCAGC | | | | | | |
| X_ | CATCTTCCACCAGCA | 1270 | | | X | 154532667 | − |
| 154532667 | GTGCAAGCGCAACGA | | | | | | |
| | GCTGGTGATCSRCST | | | | | | |
| | GCAGC | | | | | | |
| X_ | CATCTTCCACCAGCA | 1271 | | | X | 154532667 | − |
| 154532667 | GTGCAAGCGCAACGA | | | | | | |
| | GCTGGTGATCCGCGT | | | | | | |
| | GCAGC | | | | | | |
| rs13785 | CCCGGCTTCTTGGTC | 1272 | CCCGGCTTCTTGGTC | 2086 | X | 154532674 | + |
| 2335 | ATCATCTTGGTGTAC | | ATCATCTTGGTGTAC | | | | |
| | ACGGCCYYGTTGRGC | | ACGGCCYYGTTGRGC | | | | |
| | TGCAC | | TGCAG | | | | |
| rs13785 | CCCGGCTTCTTGGTC | 1273 | CCCGGCTTCTTGGTC | 2087 | X | 154532674 | + |
| 2335 | ATCATCTTGGTGTAC | | ATCATCTTGGTGTAC | | | | |
| | ACGGCCYYGTTGRGC | | ACGGCCYYGTTGRGC | | | | |
| | TGCAC | | TGCAG | | | | |
| rs13785 | CGGCGACATCTTCCA | 1274 | CGGCGACATCTTCCA | 2088 | X | 154532674 | − |
| 2335 | CCAGCAGTGCAAGCG | | CCAGCAGTGCAAGCG | | | | |
| | CAACGAGCTGGTGAT | | CAACGAGCTGGTGAT | | | | |
| | CCGCG | | CCGCC | | | | |
| rs13785 | GGCCGGCGACATCTT | 1275 | | | X | 154532676 | − |
| 2316 | CCACCAGCAGTGCAA | | | | | | |
| | GCGCAACGAGCTGGT | | | | | | |
| | GATCC | | | | | | |
| rs13785 | GGCCGGCGACATCTT | 1276 | | | X | 154532676 | − |
| 2316 | CCACCAGCAGTGCAA | | | | | | |
| | GCGCAACGAGCTGGT | | | | | | |
| | GATCC | | | | | | |
| rs13785 | GGCCGGCGACATCTT | 1277 | | | X | 154532676 | − |
| 2316 | CCACCAGCAGTGCAA | | | | | | |
| | GCGCAACGAGCTGGT | | | | | | |
| | GATCC | | | | | | |
| X_ | GGCCGGCGACATCTT | 1278 | GGCCGGCGACATCTT | 2089 | X | 154532677 | − |
| 154532677 | CCACCAGCAGTGCAA | | CCACCAGCAGTGCAA | | | | |
| | GCGCRACGRGCTGGT | | GCGCRACGRGCTGGT | | | | |
| | GAYCG | | GAYCC | | | | |
| X_ | GGCCGGCGACATCTT | 1279 | GGCCGGCGACATCTT | 2090 | X | 154532677 | − |
| 154532677 | CCACCAGCAGTGCAA | | CCACCAGCAGTGCAA | | | | |
| | GCGCRACGRGCTGGT | | GCGCRACGRGCTGGT | | | | |
| | GAYCG | | GAYCC | | | | |
| X_ | GGCCGGCGACATCTT | 1280 | GGCCGGCGACATCTT | 2091 | X | 154532677 | − |
| 154532677 | CCACCAGCAGTGCAA | | CCACCAGCAGTGCAA | | | | |
| | GCGCRACGRGCTGGT | | GCGCRACGRGCTGGT | | | | |
| | GAYCG | | GAYCC | | | | |
| X_ | TGTGGCCGGCGACAT | 1281 | | | X | 154532679 | − |
| 154532679 | CTTCCACCAGCAGTG | | | | | | |
| | CAAGCGCAACGAGCT | | | | | | |
| | GGTGA | | | | | | |
| X_ | TGTGGCCGGCGACAT | 1282 | | | X | 154532679 | − |
| 154532679 | CTTCCACCAGCAGTG | | | | | | |
| | CAAGCGCAACGAGCT | | | | | | |
| | GGTGA | | | | | | |
| X_ | TGTGGCCGGCGACAT | 1283 | | | X | 154532679 | − |
| 154532679 | CTTCCACCAGCAGTG | | | | | | |
| | CAAGCGCAACGAGCT | | | | | | |
| | GGTGA | | | | | | |
| X_ | GTTCCATGATGTGGC | 1284 | | | X | 154532688 | − |
| 154532688 | CGGCGACATCTTCCA | | | | | | |
| | CCAGCAGTGCAAGYR | | | | | | |
| | CRACG | | | | | | |
| X_ | GTTCCATGATGTGGC | 1285 | | | X | 154532688 | − |
| 154532688 | CGGCGACATCTTCCA | | | | | | |
| | CCAGCAGTGCAAGYR | | | | | | |
| | CRACG | | | | | | |
| X_ | GTTCCATGATGTGGC | 1286 | | | X | 154532688 | − |
| 154532688 | CGGCGACATCTTCCA | | | | | | |
| | CCAGCAGTGCAAGYR | | | | | | |
| | CRACG | | | | | | |
| X_ | CATCTTGGTGTACAC | 1287 | | | X | 154532692 | + |
| 154532692 | GGCCTCGTTGGGCTG | | | | | | |
| | CACGCGGATCACCAG | | | | | | |
| | CTCGT | | | | | | |
| X_ | TGCAGTTCCATGATG | 1288 | | | X | 154532692 | − |
| 154532692 | TGGCCGGCGACATCT | | | | | | |
| | TCCACCAGCAGTGCR | | | | | | |
| | AGYRC | | | | | | |
| X_ | TGCAGTTCCATGATG | 1289 | | | X | 154532692 | − |
| 154532692 | TGGCCGGCGACATCT | | | | | | |
| | TCCACCAGCAGTGCR | | | | | | |
| | AGYRC | | | | | | |
| rs13785 | GCTGCAGTTCCATGA | 1290 | | | X | 154532694 | − |
| 2321 | TGTGGCCGGCGACAT | | | | | | |
| | CTTCCACCAGCAGTG | | | | | | |
| | CAAGC | | | | | | |
| rs13785 | GCTGCAGTTCCATGA | 1291 | | | X | 154532694 | − |
| 2321 | TGTGGCCGGCGACAT | | | | | | |
| | CTTCCACCAGCAGTG | | | | | | |
| | CAAGC | | | | | | |
| rs13785 | GCTGCAGTTCCATGA | 1292 | | | X | 154532694 | − |
| 2321 | TGTGGCCGGCGACAT | | | | | | |
| | CTTCCACCAGCAGTG | | | | | | |
| | CAAGC | | | | | | |
| rs13785 | GGCTGCAGTTCCATG | 1293 | | | X | 154532695 | − |
| 2334 | ATGTGGCCGGCGACA | | | | | | |
| | TCTTCCACCAGCAGT | | | | | | |
| | GCAAG | | | | | | |
| rs13785 | GGCTGCAGTTCCATG | 1294 | | | X | 154532695 | − |
| 2334 | ATGTGGCCGGCGACA | | | | | | |
| | TCTTCCACCAGCAGT | | | | | | |
| | GCAAG | | | | | | |
| rs13785 | GGCTGCAGTTCCATG | 1295 | | | X | 154532695 | − |
| 2334 | ATGTGGCCGGCGACA | | | | | | |
| | TCTTCCACCAGCAGT | | | | | | |
| | GCAAG | | | | | | |
| rs13785 | GGCTGCAGTTCCATG | 1296 | | | X | 154532695 | − |
| 2334 | ATGTGGCCGGCGACA | | | | | | |
| | TCTTCCACCAGCAGT | | | | | | |
| | GCAAG | | | | | | |
| rs13785 | TGAGGCTGCAGTTCC | 1297 | | | X | 154532698 | − |
| 2320 | ATGATGTGGCCGGCG | | | | | | |
| | ACATCTTCCACCAGC | | | | | | |
| | AGTGC | | | | | | |
| rs13785 | TGAGGCTGCAGTTCC | 1298 | | | X | 154532698 | − |
| 2320 | ATGATGTGGCCGGCG | | | | | | |
| | ACATCTTCCACCAGC | | | | | | |
| | AGTGC | | | | | | |
| rs13785 | TGAGGCTGCAGTTCC | 1299 | | | X | 154532698 | − |
| 2320 | ATGATGTGGCCGGCG | | | | | | |
| | ACATCTTCCACCAGC | | | | | | |
| | AGTGC | | | | | | |
| X_ | TGAGGCTGCAGTTCC | 1300 | TGAGGCTGCAGTTCC | 2092 | X | 154532699 | − |
| 154532699 | ATGATGTGGCCGGCG | | ATGATGTGGCCGGCG | | | | |
| | ACATCYTCCACCAGC | | ACATCYTCCACCAGC | | | | |
| | AGYKC | | AGYKG | | | | |
| X_ | TGAGGCTGCAGTTCC | 1301 | TGAGGCTGCAGTTCC | 2093 | X | 154532699 | − |
| 154532699 | ATGATGTGGCCGGCG | | ATGATGTGGCCGGCG | | | | |
| | ACATCYTCCACCAGC | | ACATCYTCCACCAGC | | | | |
| | AGYKC | | AGYKG | | | | |
| X_ | TGAGGCTGCAGTTCC | 1302 | TGAGGCTGCAGTTCC | 2094 | X | 154532699 | − |
| 154532699 | ATGATGTGGCCGGCG | | ATGATGTGGCCGGCG | | | | |
| | ACATCTTCCACCAGC | | ACATCTTCCACCAGC | | | | |
| | AGTGC | | AGTGG | | | | |
| X_ | GGTGAGGCTGCAGTT | 1303 | | | X | 154532700 | − |
| 154532700 | CCATGATGTGGCCGG | | | | | | |
| | CGACATCTTCCACCA | | | | | | |
| | GCAGT | | | | | | |
| X_ | GGTGAGGCTGCAGTT | 1304 | | | X | 154532700 | − |
| 154532700 | CCATGATGTGGCCGG | | | | | | |
| | CGACATCTTCCACCA | | | | | | |
| | GCAGT | | | | | | |
| X_ | GGTGAGGCTGCAGTT | 1305 | | | X | 154532700 | − |
| 154532700 | CCATGATGTGGCCGG | | | | | | |
| | CGACATCTTCCACCA | | | | | | |
| | GCAGT | | | | | | |
| rs13785 | AGGTGAGGCTGCAGT | 1306 | | | X | 154532701 | − |
| 2322 | TCCATGATGTGGCCG | | | | | | |
| | GCGACRYCYTCCACC | | | | | | |
| | AGCAG | | | | | | |
| rs13785 | AGGTGAGGCTGCAGT | 1307 | | | X | 154532701 | − |
| 2322 | TCCATGATGTGGCCG | | | | | | |
| | GCGACRYCYTCCACC | | | | | | |
| | AGCAG | | | | | | |
| rs13785 | AGGTGAGGCTGCAGT | 1308 | | | X | 154532701 | − |
| 2322 | TCCATGATGTGGCCG | | | | | | |
| | GCGACATCTTCCACC | | | | | | |
| | AGCAG | | | | | | |
| X_ | GTTGGGCTGCACGCG | 1309 | | | X | 154532713 | + |
| 154532713 | GATCACCAGCTCGTT | | | | | | |
| | GCGCTTGCACTGCTG | | | | | | |
| | GTGGA | | | | | | |
| X_ | GTTGGGCTGCACGCG | 1310 | | | X | 154532713 | + |
| 154532713 | GATCACCAGCTCGTT | | | | | | |
| | GCGCTTGCACTGCTG | | | | | | |
| | GTGGA | | | | | | |
| X_ | GTTGGGCTGCACGCG | 1311 | | | X | 154532713 | + |
| 154532713 | GATCACCAGCTCGTT | | | | | | |
| | GCGCTTGCACTGCTG | | | | | | |
| | GTGGA | | | | | | |
| X_ | TGGGCTGCACGCGGA | 1312 | | | X | 154532715 | + |
| 154532715 | TCACCAGCTCGTTGC | | | | | | |
| | GCTTGCACTGCTGGT | | | | | | |
| | GGAAG | | | | | | |
| X_ | TGGGCTGCACGCGGA | 1313 | | | X | 154532715 | + |
| 154532715 | TCACCAGCTCGTTGC | | | | | | |
| | GCTTGCACTGCTGGT | | | | | | |
| | GGAAG | | | | | | |
| X_ | TGGGCTGCACGCGGA | 1314 | | | X | 154532715 | + |
| 154532715 | TCACCAGCTCGTTGC | | | | | | |
| | GCTTGCACTGCTGGT | | | | | | |
| | GGAAG | | | | | | |
| X_ | GGGCTGCACGCGGAT | 1315 | | | X | 154532716 | + |
| 154532716 | CACCAGCTCGTTGCG | | | | | | |
| | CTTGCACTGCTGGTG | | | | | | |
| | GAAGA | | | | | | |
| X_ | GGGCTGCACGCGGAT | 1316 | | | X | 154532716 | + |
| 154532716 | CACCAGCTCGTTGCG | | | | | | |
| | CTTGCACTGCTGGTG | | | | | | |
| | GAAGA | | | | | | |
| X_ | GGGCTGCACGCGGAT | 1317 | | | X | 154532716 | + |
| 154532716 | CACCAGCTCGTTGCG | | | | | | |
| | CTTGCACTGCTGGTG | | | | | | |
| | GAAGA | | | | | | |
| rs37148 | CACGCGGATCACCAG | 1318 | | | X | 154532722 | + |
| 9738 | CTCGTTGCGCTTGCA | | | | | | |
| | CTGCTGGTGGAAGAT | | | | | | |
| | GTCGC | | | | | | |
| rs37148 | CACGCGGATCACCAG | 1319 | | | X | 154532722 | + |
| 9738 | CTCGTTGCGCTTGCA | | | | | | |
| | CTGCTGGTGGAAGAT | | | | | | |
| | GTCGC | | | | | | |
| rs37148 | CACGCGGATCACCAG | 1320 | | | X | 154532722 | + |
| 9738 | CTCGTTGCGCTTGCA | | | | | | |
| | CTGCTGGTGGAAGAT | | | | | | |
| | GTCGC | | | | | | |
| X_ | AAGATGTCGCCGGCC | 1321 | AAGATGTCGCCGGCC | 2095 | X | 154532752 | + |
| 154532752 | ACATCATGGAAYTGC | | ACATCATGGAAYTGC | | | | |
| | AGCCTCACCTCGGCC | | AGCCTCACCTCGGCC | | | | |
| | TYGCG | | TTGCC | | | | |
| X_ | CTGCTGGTGGAAGAT | 1322 | | | X | 154532752 | + |
| 154532752 | GTCGCCGGCCACATC | | | | | | |
| | ATGGAACTGCAGCCT | | | | | | |
| | CACCT | | | | | | |
| X_ | GTGGAAGATGTCGCC | 1323 | | | X | 154532758 | + |
| 154532758 | GGCCACATCATGGAA | | | | | | |
| | CTGCAGCCTCACCTC | | | | | | |
| | GGCCT | | | | | | |
| X_ | GTGGAAGATGTCGCC | 1324 | | | X | 154532758 | + |
| 154532758 | GGCCACATCATGGAA | | | | | | |
| | CTGCAGCCTCACCTC | | | | | | |
| | GGCCT | | | | | | |
| X_ | GTGGAAGATGTCGCC | 1325 | | | X | 154532758 | + |
| 154532758 | GGCCACATCATGGAA | | | | | | |
| | CTGCAGCCTCACCTC | | | | | | |
| | GGCCT | | | | | | |
| rs13785 | TGTCGCCGGCCACAT | 1326 | TGTCGCCGGCCACAT | 2096 | X | 154532765 | + |
| 2329 | CATGGAACTGCAGCC | | CATGGAACTGCAGCC | | | | |
| | TCACCTCGGCCTYGC | | TCACCTCGGCCTYGC | | | | |
| | GCTCT | | GCTCG | | | | |
| rs13785 | TCCGCCACGTAGGGG | 1327 | TCCGCCACGTAGGGG | 2097 | X | 154532765 | − |
| 2329 | TGCCCTTCATCCTGC | | TGCCCTTCATCCTGC | | | | |
| | GCTGCGGCAAGGCCC | | GCTGCGGCAAGGCCC | | | | |
| | TGAAA | | TGAAC | | | | |
| rs13785 | TCCGCCACGTAGGGG | 1328 | TCCGCCACGTAGGGG | 2098 | X | 154532765 | − |
| 2329 | TGCCCTTCATCCTGC | | TGCCCTTCATCCTGC | | | | |
| | GCTGCGGCAAGGCCC | | GCTGCGGCAAGGCCC | | | | |
| | TGAAC | | TGAAG | | | | |
| rs13785 | ATGTCGCCGGCCACA | 1329 | | | X | 154532765 | + |
| 2329 | TCATGGAACTGCAGC | | | | | | |
| | CTCACCTCGGCCTTG | | | | | | |
| | CGCTC | | | | | | |
| rs13785 | CGGCCACATCATGGA | 1330 | | | X | 154532772 | + |
| 2345 | ACTGCAGCCTCACCT | | | | | | |
| | CGGCCTTGCGCTCGT | | | | | | |
| | TCAGG | | | | | | |
| rs13785 | CGGCCACATCATGGA | 1331 | | | X | 154532772 | + |
| 2345 | ACTGCAGCCTCACCT | | | | | | |
| | CGGCCTTGCGCTCGT | | | | | | |
| | TCAGG | | | | | | |
| X_ | GCTGCCCTTTCCGCC | 1332 | | | X | 154532773 | − |
| 154532773 | ACGTAGGGGTGYCCT | | | | | | |
| | TCATCCTGCGCTGCG | | | | | | |
| | GCAAG | | | | | | |
| X_ | GCTGCCCTTTCCGCC | 1333 | | | X | 154532773 | − |
| 154532773 | ACGTAGGGGTGYCCT | | | | | | |
| | TCATCCTGCGCTGCG | | | | | | |
| | GCAAG | | | | | | |
| X_ | GCTGCCCTTTCCGCC | 1334 | | | X | 154532773 | − |
| 154532773 | ACGTAGGGGTGYCCT | | | | | | |
| | TCATCCTGCGCTGCG | | | | | | |
| | GCAAG | | | | | | |
| rs13785 | CACCCTGAGAGAGCT | 1335 | | | X | 154532797 | − |
| 2333 | GGTGCTGAGGCTGCC | | | | | | |
| | CTTTCCGCCACGTAG | | | | | | |
| | GGGTG | | | | | | |
| rs13785 | CACCTCGGCCTTGCG | 1336 | | | X | 154532797 | + |
| 2333 | CTCGTTCAGGGCCTT | | | | | | |
| | GCCGCAGCGCAGGAT | | | | | | |
| | GAAGG | | | | | | |
| rs13785 | CACCTCGGCCTTGCG | 1337 | | | X | 154532797 | + |
| 2333 | CTCGTTCAGGGCCTT | | | | | | |
| | GCCGCAGCGCAGGAT | | | | | | |
| | GAAGG | | | | | | |
| X_ | GTCCACACCCTGAGA | 1338 | | | X | 154532802 | − |
| 154532802 | GAGCTGGTGCTGAGG | | | | | | |
| | CTGCCCTTTCCGCCA | | | | | | |
| | CGTAG | | | | | | |
| X_ | GTCCACACCCTGAGA | 1339 | | | X | 154532802 | − |
| 154532802 | GAGCTGGTGCTGAGG | | | | | | |
| | CTGCCCTTTCCGCCA | | | | | | |
| | CGTAG | | | | | | |
| X_ | GTCCACACCCTGAGA | 1340 | | | X | 154532802 | − |
| 154532802 | GAGCTGGTGCTGAGG | | | | | | |
| | CTGCCCTTTCCGCCA | | | | | | |
| | CGTAG | | | | | | |
| rs34193 | GGGCATGCCCAGTTC | 1341 | GGGCATGCCCAGTTC | 2099 | X | 154532945 | + |
| 178 | TGCCTTGCTGGGCCT | | TGCCTTGCTGGGCCT | | | | |
| | CGAAGGCATCACCTA | | CGAAGGCATCACCTA | | | | |
| | CCATG | | CCATC | | | | |
| rs34193 | GGGCATGCCCAGTTC | 1342 | GGGCATGCCCAGTTC | 2100 | X | 154532945 | + |
| 178 | TGCCTTGCTGGGCCT | | TGCCTTGCTGGGCCT | | | | |
| | CGAAGGCATCACCTA | | CGAAGGCATCACCTA | | | | |
| | CCATG | | CCATC | | | | |
| rs34193 | GGGCATGCCCAGTTC | 1343 | GGGCATGCCCAGTTC | 2101 | X | 154532945 | + |
| 178 | TGCCTTGCTGGGCCT | | TGCCTTGCTGGGCCT | | | | |
| | CGAAGGCATCACCTA | | CGAAGGCATCACCTA | | | | |
| | CCATG | | CCATC | | | | |
| rs39812 | GTTCTGCCTTGCTGG | 1344 | GTTCTGCCTTGCTGG | 2102 | X | 154532956 | + |
| 3544 | GCCTCGAAGGCATCA | | GCCTCGAAGGCATCA | | | | |
| | CCTACCATCCCACCT | | CCTACCATCCCACCT | | | | |
| | CTCAT | | CTCAA | | | | |
| rs39812 | GTTCTGCCTTGCTGG | 1345 | GTTCTGCCTTGCTGG | 2103 | X | 154532956 | + |
| 3544 | GCCTCGAAGGCATCA | | GCCTCGAAGGCATCA | | | | |
| | CCTACCATCCCACCT | | CCTACCATCCCACCT | | | | |
| | CTCAT | | CTCAA | | | | |
| rs39812 | GTTCTGCCTTGCTGG | 1346 | GTTCTGCCTTGCTGG | 2104 | X | 154532956 | + |
| 3544 | GCCTCGAAGGCATCA | | GCCTCGAAGGCATCA | | | | |
| | CCTACCATCCCACCT | | CCTACCATCCCACCT | | | | |
| | CTCAT | | CTCAA | | | | |
| rs13785 | TGGGCCTCGAAGGCA | 1347 | | | X | 154532969 | + |
| 2342 | TCACCTACCATCCCA | | | | | | |
| | CCTCTCATTCTCCAC | | | | | | |
| | ATAGA | | | | | | |
| rs13785 | TGGGCCTCGAAGGCA | 1348 | | | X | 154532969 | + |
| 2342 | TCACCTACCATCCCA | | | | | | |
| | CCTCTCATTCTCCAC | | | | | | |
| | ATAGA | | | | | | |
| rs13785 | TGGGCCTCGAAGGCA | 1349 | | | X | 154532969 | + |
| 2342 | TCACCTACCATCCCA | | | | | | |
| | CCTCTCATTCTCCAC | | | | | | |
| | ATAGA | | | | | | |
| X_ | CCTACCATCCCACCT | 1350 | | | X | 154532987 | + |
| 154532987 | CTCATTCTCCACATA | | | | | | |
| | GARGACGACGGCTGC | | | | | | |
| | AAAAG | | | | | | |
| X_ | CCTACCATCCCACCT | 1351 | | | X | 154532987 | + |
| 154532987 | CTCATTCTCCACATA | | | | | | |
| | GARGACGACGGCTGC | | | | | | |
| | AAAAG | | | | | | |
| X_ | CCTACCATCCCACCT | 1352 | | | X | 154532987 | + |
| 154532987 | CTCATTCTCCACATA | | | | | | |
| | GAGGACGACGGCTGC | | | | | | |
| | AAAAG | | | | | | |
| X_ | TACCATCCCACCTCT | 1353 | | | X | 154532989 | + |
| 154532989 | CATTCTCCACATAGA | | | | | | |
| | RGACGACGGCTGCAA | | | | | | |
| | AAGYG | | | | | | |
| X_ | TACCATCCCACCTCT | 1354 | | | X | 154532989 | + |
| 154532989 | CATTCTCCACATAGA | | | | | | |
| | GGACGACGGCTGCAA | | | | | | |
| | AAGTG | | | | | | |
| X_ | TACCATCCCACCTCT | 1355 | | | X | 154532989 | + |
| 154532989 | CATTCTCCACATAGA | | | | | | |
| | GGACGACGGCTGCAA | | | | | | |
| | AAGTG | | | | | | |
| rs50308 | ACCATCCCACCTCTC | 1356 | | | X | 154532990 | + |
| 69 | ATTCTCCACATAGAG | | | | | | |
| | GACGACGGCTGCAAA | | | | | | |
| | AGTGG | | | | | | |
| rs50308 | ACCATCCCACCTCTC | 1357 | | | X | 154532990 | + |
| 69 | ATTCTCCACATAGAR | | | | | | |
| | GACGACGGCTGCAAA | | | | | | |
| | AGYGG | | | | | | |
| rs50308 | ACCATCCCACCTCTC | 1358 | | | X | 154532990 | + |
| 69 | ATTCTCCACATAGAG | | | | | | |
| | GACGACGGCTGCAAA | | | | | | |
| | AGTGG | | | | | | |
| X_ | CATTCTCCACATAGA | 1359 | | | X | 154533004 | + |
| 154533004 | GGACGACGGCTGCAA | | | | | | |
| | AAGYGKYGGTGGTGG | | | | | | |
| | ACCCG | | | | | | |
| X_ | CATTCTCCACATAGA | 1360 | | | X | 154533004 | + |
| 154533004 | GGACGACGGCTGCAA | | | | | | |
| | AAGYGKYGGTGGTGG | | | | | | |
| | ACCCG | | | | | | |
| X_ | CATTCTCCACATAGA | 1361 | | | X | 154533004 | + |
| 154533004 | GGACGACGGCTGCAA | | | | | | |
| | AAGYGKYGGTGGTGG | | | | | | |
| | ACCCG | | | | | | |
| X_ | GGTCCTGGGCCAGTA | 1362 | GGTCCTGGGCCAGTA | 2105 | X | 154533014 | − |
| 154533014 | CGTGGGGAACCCCGA | | CGTGGGGAACCCCGA | | | | |
| | TGGAGAGGGCRAGGC | | TGGAGAGGGCRAGGC | | | | |
| | CACCA | | CACGG | | | | |
| X_ | GGTCCTGGGCCAGTA | 1363 | GGTCCTGGGCCAGTA | 2106 | X | 154533014 | − |
| 154533014 | CGTGGGGAACCCCGA | | CGTGGGGAACCCCGA | | | | |
| | TGGAGAGGGCRAGGC | | TGGAGAGGGCRAGGC | | | | |
| | CACCA | | CACGG | | | | |
| X_ | GGTCCTGGGCCAGTA | 1364 | GGTCCTGGGCCAGTA | 2107 | X | 154533014 | − |
| 154533014 | CGTGGGGAACCCCGA | | CGTGGGGAACCCCGA | | | | |
| | TGGAGAGGGCGAGGC | | TGGAGAGGGCGAGGC | | | | |
| | CACCA | | CACGG | | | | |
| X_ | AGAGGACGACGGCTG | 1365 | | | X | 154533016 | + |
| 154533016 | CAAAAGTGGCGGTGG | | | | | | |
| | TGGACCCGYGGGGCA | | | | | | |
| | CCGTG | | | | | | |
| X_ | AGAGGACGACGGCTG | 1366 | | | X | 154533016 | + |
| 154533016 | CAAAAGTGGCGGTGG | | | | | | |
| | TGGACCCGYGGGGCA | | | | | | |
| | CCGTG | | | | | | |
| X_ | AGAGGACGACGGCTG | 1367 | | | X | 154533016 | + |
| 154533016 | CAAAAGTGGCGGTGG | | | | | | |
| | TGGACCCGYGGGGCA | | | | | | |
| | CCGTG | | | | | | |
| rs76723 | CCAGTACGTGGGGAA | 1368 | | | X | 154533025 | − |
| 693 | CCCCGATGGAGAGGG | | | | | | |
| | CGAGGCCACCAAAGG | | | | | | |
| | GTACC | | | | | | |
| rs13785 | TGCAAAAGTGGCGGT | 1369 | | | X | 154533029 | + |
| 2347 | GGTGGACCCGCGGGG | | | | | | |
| | CACCGTGGGGTCGTC | | | | | | |
| | CAGGT | | | | | | |
| rs13785 | TGCAAAAGTGGCGGT | 1370 | | | X | 154533029 | + |
| 2347 | GGTGGACCCGCGGGG | | | | | | |
| | CACCGTGGGGTCGTC | | | | | | |
| | CAGGT | | | | | | |
| rs13785 | TGCAAAAGTGGCGGT | 1371 | | | X | 154533029 | + |
| 2347 | GGTGGACCCGCGGGG | | | | | | |
| | CACCGTGGGGTCGTC | | | | | | |
| | CAGGT | | | | | | |
| X_ | CCTGGGCCAGTACGT | 1372 | | | X | 154533031 | − |
| 154533031 | GGGGAACCCCGATGG | | | | | | |
| | AGAGGGCRAGGCCAC | | | | | | |
| | CAAAG | | | | | | |
| X_ | CCTGGGCCAGTACGT | 1373 | | | X | 154533031 | − |
| 154533031 | GGGGAACCCCGATGG | | | | | | |
| | AGAGGGCRAGGCCAC | | | | | | |
| | CAAAG | | | | | | |
| X_ | CCTGGGCCAGTACGT | 1374 | | | X | 154533031 | − |
| 154533031 | GGGGAACCCCGATGG | | | | | | |
| | AGAGGGCRAGGCCAC | | | | | | |
| | CAAAG | | | | | | |
| rs13785 | CCAACAATGTGGTCC | 1375 | | | X | 154533044 | − |
| 2339 | TGGGCCAGTACGTGG | | | | | | |
| | RGAACCCCGATGGAG | | | | | | |
| | AGGGC | | | | | | |
| rs13785 | CCAACAATGTGGTCC | 1376 | | | X | 154533044 | − |
| 2339 | TGGGCCAGTACGTGG | | | | | | |
| | GGAACCCCGATGGAG | | | | | | |
| | AGGGC | | | | | | |
| rs13785 | CCAACAATGTGGTCC | 1377 | | | X | 154533044 | − |
| 2339 | TGGGCCAGTACGTGG | | | | | | |
| | GGAACCCCGATGGAG | | | | | | |
| | AGGGC | | | | | | |
| X_ | TGGGGTCGTCCAGGT | 1378 | | | X | 154533064 | + |
| 154533064 | ACCCTTTGGTGGCCT | | | | | | |
| | YGCCCTCTCCATCGG | | | | | | |
| | GGTTC | | | | | | |
| X_ | CCAGGTACCCTTTGG | 1379 | CCAGGTACCCTTTGG | 2108 | X | 154533072 | + |
| 154533072 | TGGCCTCGCCCTCTC | | TGGCCTCGCCCTCTC | | | | |
| | CATCGGGGTTCYCCA | | CATCGGGGTTCYCCA | | | | |
| | CGTAG | | CGTAC | | | | |
| X_ | CCAGGTACCCTTTGG | 1380 | CCAGGTACCCTTTGG | 2109 | X | 154533072 | + |
| 154533072 | TGGCCTCGCCCTCTC | | TGGCCTCGCCCTCTC | | | | |
| | CATCGGGGTTCYCCA | | CATCGGGGTTCYCCA | | | | |
| | CGTAG | | CGTAC | | | | |
| X_ | CCAGGTACCCTTTGG | 1381 | CCAGGTACCCTTTGG | 2110 | X | 154533072 | + |
| 154533072 | TGGCCTCGCCCTCTC | | TGGCCTCGCCCTCTC | | | | |
| | CATCGGGGTTCYCCA | | CATCGGGGTTCYCCA | | | | |
| | CGTAG | | CGTAC | | | | |
| X_ | GTACCCTTTGGTGGC | 1382 | | | X | 154533077 | + |
| 154533077 | CTCGCCCTCTCCATC | | | | | | |
| | GGGGTTCCCCACGTA | | | | | | |
| | CTGGC | | | | | | |
| X_ | TCAAGGTGTTGAAAT | 1383 | | | X | 154533077 | − |
| 154533077 | GCATCTCAGAGGTGC | | | | | | |
| | AGGCCAACAATGTGK | | | | | | |
| | TCCTG | | | | | | |
| X_ | TCAAGGTGTTGAAAT | 1384 | | | X | 154533077 | − |
| 154533077 | GCATCTCAGAGGTGC | | | | | | |
| | AGGCCAACAATGTGK | | | | | | |
| | TCCTG | | | | | | |
| X_ | CTCAGGTCAAGGTGT | 1385 | | | X | 154533083 | − |
| 154533083 | TGAAATGCATCTCAG | | | | | | |
| | AGGTGCAGGCCAACA | | | | | | |
| | ATGTG | | | | | | |
| X_ | CTCAGGTCAAGGTGT | 1386 | | | X | 154533083 | − |
| 154533083 | TGAAATGCATCTCAG | | | | | | |
| | AGGTGCAGGCCAACA | | | | | | |
| | ATGTG | | | | | | |
| X_ | CTCAGGTCAAGGTGT | 1387 | | | X | 154533083 | − |
| 154533083 | TGAAATGCATCTCAG | | | | | | |
| | AGGTGCAGGCCAACA | | | | | | |
| | ATGTG | | | | | | |
| rs13785 | CTCAACACCCAAGGA | 1388 | | | X | 154533122 | − |
| 2327 | GCCCATTCTCTCCCT | | | | | | |
| | TGGCTTTCTCTCAGG | | | | | | |
| | TCAAG | | | | | | |
| rs13785 | CTCAACACCCAAGGA | 1389 | | | X | 154533122 | − |
| 2327 | GCCCATTCTCTCCCT | | | | | | |
| | TGGCTTTCTCTCAGG | | | | | | |
| | TCAAG | | | | | | |
| rs13785 | CTCAACACCCAAGGA | 1390 | | | X | 154533122 | − |
| 2327 | GCCCATTCTCTCCCT | | | | | | |
| | TGGCTTTCTCTCAGG | | | | | | |
| | TCAAG | | | | | | |
| rs74575 | GTGTCTGGTGGCCAT | 1391 | | | X | 154533586 | − |
| 103 | GGAGAAGCCCGCCTC | | | | | | |
| | CACCAACTCAGATGA | | | | | | |
| | CGTCC | | | | | | |
| rs74575 | GTGTCTGGTGGCCAT | 1392 | | | X | 154533586 | − |
| 103 | GGAGAAGCCCGCCTC | | | | | | |
| | CACCAACTCAGATGA | | | | | | |
| | CGTCC | | | | | | |
| rs74575 | GTGTCTGGTGGCCAT | 1393 | | | X | 154533586 | − |
| 103 | GGAGAAGCCCGCCTC | | | | | | |
| | CACCAACTCAGATGA | | | | | | |
| | CGTCC | | | | | | |
| X_ | TGTGTCTGGTGGCCA | 1394 | | | X | 154533587 | − |
| 154533587 | TGGAGAAGCCCGCCT | | | | | | |
| | CCACCAACTCAGATG | | | | | | |
| | ACGTC | | | | | | |
| X_ | TGTGTCTGGTGGCCA | 1395 | | | X | 154533587 | − |
| 154533587 | TGGAGAAGCCCGCCT | | | | | | |
| | CCACCAACTCAGATG | | | | | | |
| | ACGTC | | | | | | |
| X_ | TGTGTCTGGTGGCCA | 1396 | | | X | 154533587 | − |
| 154533587 | TGGAGAAGCCCGCCT | | | | | | |
| | CCACCAACTCAGATG | | | | | | |
| | ACGTC | | | | | | |
| X_ | GCTGTGTCTGGTGGC | 1397 | | | X | 154533589 | − |
| 154533589 | CATGGAGAAGCCCGC | | | | | | |
| | CTCCACCAACTCAGA | | | | | | |
| | TGACG | | | | | | |
| X_ | GCTGTGTCTGGTGGC | 1398 | | | X | 154533589 | − |
| 154533589 | CATGGAGAAGCCCGC | | | | | | |
| | CTCCACCAACTCAGA | | | | | | |
| | TGACG | | | | | | |
| X_ | GCTGTGTCTGGTGGC | 1399 | | | X | 154533589 | − |
| 154533589 | CATGGAGAAGCCCGC | | | | | | |
| | CTCCACCAACTCAGA | | | | | | |
| | TGACG | | | | | | |
| X_ | ATGCTGTGTCTGGTG | 1400 | | | X | 154533591 | − |
| 154533591 | GCCATGGAGAAGCCC | | | | | | |
| | GCCTCCACCAACTCA | | | | | | |
| | GATGA | | | | | | |
| X_ | ATGCTGTGTCTGGTG | 1401 | | | X | 154533591 | − |
| 154533591 | GCCATGGAGAAGCCC | | | | | | |
| | GCCTCCACCAACTCA | | | | | | |
| | GATGA | | | | | | |
| X_ | ATGCTGTGTCTGGTG | 1402 | | | X | 154533591 | − |
| 154533591 | GCCATGGAGAAGCCC | | | | | | |
| | GCCTCCACCAACTCA | | | | | | |
| | GATGA | | | | | | |
| X_ | GATGCTGTGTCTGGT | 1403 | | | X | 154533592 | − |
| 154533592 | GGCCATGGAGAAGCC | | | | | | |
| | CGCCTCCACCAACTC | | | | | | |
| | AGATG | | | | | | |
| X_ | GATGCTGTGTCTGGT | 1404 | | | X | 154533592 | − |
| 154533592 | GGCCATGGAGAAGCC | | | | | | |
| | CGCCTCCACCAACTC | | | | | | |
| | AGATG | | | | | | |
| X_ | GATGCTGTGTCTGGT | 1405 | | | X | 154533592 | − |
| 154533592 | GGCCATGGAGAAGCC | | | | | | |
| | CGCCTCCACCAACTC | | | | | | |
| | AGATG | | | | | | |
| rs13785 | GCAGATGCTGTGTCT | 1406 | GCAGATGCTGTGTCT | 2111 | X | 154533596 | − |
| 2318 | GGTGGCCATGGAGAA | | GGTGGCCATGGAGAA | | | | |
| | GCCCGCCYYCDCCAA | | GCCCGCCYYCDCCAA | | | | |
| | CTCAG | | CTCAC | | | | |
| rs13785 | GCAGATGCTGTGTCT | 1407 | GCAGATGCTGTGTCT | 2112 | X | 154533596 | − |
| 2318 | GGTGGCCATGGAGAA | | GGTGGCCATGGAGAA | | | | |
| | GCCCGCCYYCDCCAA | | GCCCGCCYYCDCCAA | | | | |
| | CTCAG | | CTCAC | | | | |
| rs13785 | GCAGATGCTGTGTCT | 1408 | GCAGATGCTGTGTCT | 2113 | X | 154533596 | − |
| 2318 | GGTGGCCATGGAGAA | | GGTGGCCATGGAGAA | | | | |
| | GCCCGCCYYCDCCAA | | GCCCGCCYYCDCCAA | | | | |
| | CTCAG | | CTCAC | | | | |
| X_ | CCACCTACTGCAGAT | 1409 | CCACCTACTGCAGAT | 2114 | X | 154533605 | − |
| 154533605 | GCTGTGTCTGGTGGC | | GCTGTGTCTGGTGGC | | | | |
| | CATGGAGAAGCCCGC | | CATGGAGAAGCCCGC | | | | |
| | CTCCA | | CTCCG | | | | |
| X_ | CCACCTACTGCAGAT | 1410 | CCACCTACTGCAGAT | 2115 | X | 154533605 | − |
| 154533605 | GCTGTGTCTGGTGGC | | GCTGTGTCTGGTGGC | | | | |
| | CATGGAGAAGCCCGC | | CATGGAGAAGCCCGC | | | | |
| | CTCCA | | CTCCG | | | | |
| X_ | CCACCTACTGCAGAT | 1411 | CCACCTACTGCAGAT | 2116 | X | 154533605 | − |
| 154533605 | GCTGTGTCTGGTGGC | | GCTGTGTCTGGTGGC | | | | |
| | CATGGAGAAGCCCGC | | CATGGAGAAGCCCGC | | | | |
| | CTCCA | | CTCCG | | | | |
| X_ | GAACCACCTACTGCA | 1412 | | | X | 154533607 | − |
| 154533607 | GATGCTGTGTCTGGT | | | | | | |
| | GGCCATGGAGAASYC | | | | | | |
| | CGCCY | | | | | | |
| X_ | GAACCACCTACTGCA | 1413 | | | X | 154533607 | − |
| 154533607 | GATGCTGTGTCTGGT | | | | | | |
| | GGCCATGGAGAASYC | | | | | | |
| | CGCCY | | | | | | |
| X_ | GAACCACCTACTGCA | 1414 | | | X | 154533607 | − |
| 154533607 | GATGCTGTGTCTGGT | | | | | | |
| | GGCCATGGAGAASYC | | | | | | |
| | CGCCY | | | | | | |
| X_ | AGAACCACCTACTGC | 1415 | | | X | 154533608 | − |
| 154533608 | AGATGCTGTGTCTGG | | | | | | |
| | TGGCCATGGWGAASY | | | | | | |
| | CCGCC | | | | | | |
| X_ | AGAACCACCTACTGC | 1416 | | | X | 154533608 | − |
| 154533608 | AGATGCTGTGTCTGG | | | | | | |
| | TGGCCATGGWGAASY | | | | | | |
| | CCGCC | | | | | | |
| X_ | AGAACCACCTACTGC | 1417 | | | X | 154533608 | − |
| 154533608 | AGATGCTGTGTCTGG | | | | | | |
| | TGGCCATGGWGAASY | | | | | | |
| | CCGCC | | | | | | |
| X_ | TGCACCCCCTACCTT | 1418 | | | X | 154533614 | + |
| 154533614 | CTCATCACGGACGTC | | | | | | |
| | ATCTGAGTTGGHGRR | | | | | | |
| | GGCGG | | | | | | |
| X_ | TGCACCCCCTACCTT | 1419 | | | X | 154533614 | + |
| 154533614 | CTCATCACGGACGTC | | | | | | |
| | ATCTGAGTTGGHGRR | | | | | | |
| | GGCGG | | | | | | |
| X_ | TGCACCCCCTACCTT | 1420 | | | X | 154533614 | + |
| 154533614 | CTCATCACGGACGTC | | | | | | |
| | ATCTGAGTTGGTGGA | | | | | | |
| | GGCGG | | | | | | |
| X_ | CACCCCCTACCTTCT | 1421 | CACCCCCTACCTTCT | 2117 | X | 154533615 | + |
| 154533615 | CATCACGGACGTCAT | | CATCACGGACGTCAT | | | | |
| | CTGAGTTGGTGGAGG | | CTGAGTTGGTGGAGG | | | | |
| | CGGGG | | CGGGC | | | | |
| X_ | CACCCCCTACCTTCT | 1422 | CACCCCCTACCTTCT | 2118 | X | 154533615 | + |
| 154533615 | CATCACGGACGTCAT | | CATCACGGACGTCAT | | | | |
| | CTGAGTTGGTGGAGG | | CTGAGTTGGTGGAGG | | | | |
| | CGGGG | | CGGGC | | | | |
| X_ | CACCCCCTACCTTCT | 1423 | CACCCCCTACCTTCT | 2119 | X | 154533615 | + |
| 154533615 | CATCACGGACGTCAT | | CATCACGGACGTCAT | | | | |
| | CTGAGTTGGTGGAGG | | CTGAGTTGGTGGAGG | | | | |
| | CGGGG | | CGGGC | | | | |
| X_ | CCCTACCTTCTCATC | 1424 | CCCTACCTTCTCATC | 2120 | X | 154533619 | + |
| 154533619 | ACGGACGTCATCTGA | | ACGGACGTCATCTGA | | | | |
| | GTTGGTGGAGGCGGG | | GTTGGTGGAGGCGGG | | | | |
| | CTTCT | | CTTCA | | | | |
| X_ | CCCTACCTTCTCATC | 1425 | CCCTACCTTCTCATC | 2121 | X | 154533619 | + |
| 154533619 | ACGGACGTCATCTGA | | ACGGACGTCATCTGA | | | | |
| | GTTGGTGGRGGCGGR | | GTTGGTGGRGGCGGR | | | | |
| | STTCT | | STTCA | | | | |
| X_ | CCCTACCTTCTCATC | 1426 | CCCTACCTTCTCATC | 2122 | X | 154533619 | + |
| 154533619 | ACGGACGTCATCTGA | | ACGGACGTCATCTGA | | | | |
| | GTTGGTGGAGGCGGG | | GTTGGTGGAGGCGGG | | | | |
| | CTTCT | | CTTCA | | | | |
| X_ | CCCTACCTTCTCATC | 1427 | | | X | 154533620 | + |
| 154533620 | ACGGACGTCATCTGA | | | | | | |
| | GTTGGTGGAGGCGGG | | | | | | |
| | CTTCT | | | | | | |
| X_ | CCCTACCTTCTCATC | 1428 | | | X | 154533620 | + |
| 154533620 | ACGGACGTCATCTGA | | | | | | |
| | GTTGGTGGAGGCGGG | | | | | | |
| | CTTCT | | | | | | |
| X_ | CCCTACCTTCTCATC | 1429 | | | X | 154533620 | + |
| 154533620 | ACGGACGTCATCTGA | | | | | | |
| | GTTGGTGGAGGCGGG | | | | | | |
| | CTTCT | | | | | | |
| X_ | TCATCACGGACGTCA | 1430 | TCATCACGGACGTCA | 2123 | X | 154533629 | + |
| 154533629 | TCTGAGTTGGTGGAG | | TCTGAGTTGGTGGAG | | | | |
| | GCGGGCTTCTCCATG | | GCGGGCTTCTCCATG | | | | |
| | GCCAC | | GCCAG | | | | |
| X_ | TCATCACGGACGTCA | 1431 | TCATCACGGACGTCA | 2124 | X | 154533629 | + |
| 154533629 | TCTGAGTTGGTGGAG | | TCTGAGTTGGTGGAG | | | | |
| | GCGGGCTTCTCCATG | | GCGGGCTTCTCCATG | | | | |
| | GCCAC | | GCCAG | | | | |
| X_ | TCATCACGGACGTCA | 1432 | TCATCACGGACGTCA | 2125 | X | 154533629 | + |
| 154533629 | TCTGAGTTGGTGGAG | | TCTGAGTTGGTGGAG | | | | |
| | GCGGGCTTCTCCATG | | GCGGGCTTCTCCATG | | | | |
| | GCCAC | | GCCAG | | | | |
| rs13785 | CACGGACGTCATCTG | 1433 | | | X | 154533634 | + |
| 2346 | AGTTGGTGGAGGCGG | | | | | | |
| | GCTTCTCCATGGCCA | | | | | | |
| | CCAGA | | | | | | |
| rs13785 | CACGGACGTCATCTG | 1434 | | | X | 154533634 | + |
| 2346 | AGTTGGTGGAGGCGG | | | | | | |
| | GCTTCTCCATGGCCA | | | | | | |
| | CCAGA | | | | | | |
| rs13785 | CACGGACGTCATCTG | 1435 | | | X | 154533634 | + |
| 2346 | AGTTGGTGGAGGCGG | | | | | | |
| | GCTTCTCCATGGCCA | | | | | | |
| | CCAGA | | | | | | |
| X_ | CTTTGGCACTGAGGG | 1436 | CTTTGGCACTGAGGG | 2126 | X | 154534036 | − |
| 154534036 | TCGCGGGGGCTATTT | | TCGCGGGGGCTATTT | | | | |
| | CGATGAATTTGGGAT | | CGATGAATTTGGGAT | | | | |
| | CATCG | | CATCC | | | | |
| X_ | CTTTGGCACTGAGGG | 1437 | CTTTGGCACTGAGGG | 2127 | X | 154534036 | − |
| 154534036 | TCGCGGGGGCTATTT | | TCGCGGGGGCTATTT | | | | |
| | CGATGAATTTGGGAT | | CGATGAATTTGGGAT | | | | |
| | CATCG | | CATCC | | | | |
| X_ | CTTTGGCACTGAGGG | 1438 | CTTTGGCACTGAGGG | 2128 | X | 154534036 | − |
| 154534036 | TCGCGGGGGCTATTT | | TCGCGGGGGCTATTT | | | | |
| | CGATGAATTTGGGAT | | CGATGAATTTGGGAT | | | | |
| | CATCG | | CATCC | | | | |
| X_ | GCTCTCACCGGATGA | 1439 | | | X | 154534075 | + |
| 154534075 | TCCCAAATTCATCGA | | | | | | |
| | AATAGCCCCCGCGAC | | | | | | |
| | CCTCA | | | | | | |
| X_ | GCTCTCACCGGATGA | 1440 | | | X | 154534075 | + |
| 154534075 | TCCCAAATTCATCGA | | | | | | |
| | AATAGCCCCCGCGAC | | | | | | |
| | CCTCA | | | | | | |
| X_ | GCTCTCACCGGATGA | 1441 | | | X | 154534075 | + |
| 154534075 | TCCCAAATTCATCGA | | | | | | |
| | AATAGCCCCCGCGAC | | | | | | |
| | CCTCA | | | | | | |
| X_ | TCCCAAATTCATCGA | 1442 | | | X | 154534092 | + |
| 154534092 | AATAGCCCCCGCGAC | | | | | | |
| | CCTCAGTGCCAAAGG | | | | | | |
| | GCTCC | | | | | | |
| X_ | TCCCAAATTCATCGA | 1443 | | | X | 154534092 | + |
| 154534092 | AATAGCCCCCGCGAC | | | | | | |
| | CCTCAGTGCCAAAGG | | | | | | |
| | GCTCC | | | | | | |
| X_ | TCCCAAATTCATCGA | 1444 | | | X | 154534092 | + |
| 154534092 | AATAGCCCCCGCGAC | | | | | | |
| | CCTCAGTGCCAAAGG | | | | | | |
| | GCTCC | | | | | | |
| rs78275 | ATCGAAATAGCCCCC | 1445 | | | X | 154534102 | + |
| 7170 | GCGACCCTCAGTGCC | | | | | | |
| | AAAGGGCTCCTTGAA | | | | | | |
| | GGTGA | | | | | | |
| rs78275 | ATCGAAATAGCCCCC | 1446 | | | X | 154534102 | + |
| 7170 | GCGACCCTCAGTGCC | | | | | | |
| | AAAGGGCTCCYTGAA | | | | | | |
| | GGTGA | | | | | | |
| rs78275 | ATCGAAATAGCCCCC | 1447 | | | X | 154534102 | + |
| 7170 | GCGACCCTCAGTGCC | | | | | | |
| | AAAGGGCTCCYTGAA | | | | | | |
| | GGTGA | | | | | | |
| X_ | AGCCCCCGCGACCCT | 1448 | | | X | 154534110 | + |
| 154534110 | CAGTGCCAAAGGGCT | | | | | | |
| | CCTTGAAGGTGAGGA | | | | | | |
| | TAACG | | | | | | |
| X_ | AGCCCCCGCGACCCT | 1449 | | | X | 154534110 | + |
| 154534110 | CAGTGCCAAAGGGCT | | | | | | |
| | CCTTGAAGGTGAGGA | | | | | | |
| | TAACG | | | | | | |
| X_ | AGCCCCCGCGACCCT | 1450 | | | X | 154534110 | + |
| 154534110 | CAGTGCCAAAGGGCT | | | | | | |
| | CCTTGAAGGTGAGGA | | | | | | |
| | TAACG | | | | | | |
| X_ | ACCCTCAGTGCCAAA | 1451 | | | X | 154534119 | + |
| 154534119 | GGGCTCCTTGAAGGT | | | | | | |
| | GARGATAACGYAGGC | | | | | | |
| | GATGT | | | | | | |
| X_ | ACCCTCAGTGCCAAA | 1452 | | | X | 154534119 | + |
| 154534119 | GGGCTCCTTGAAGGT | | | | | | |
| | GAGGATAACGCAGGC | | | | | | |
| | GATGT | | | | | | |
| X_ | ACCCTCAGTGCCAAA | 1453 | | | X | 154534119 | + |
| 154534119 | GGGCTCCTTGAAGGT | | | | | | |
| | GAGGATAACGCAGGC | | | | | | |
| | GATGT | | | | | | |
| rs13785 | AGTGCCAAAGGGCTC | 1454 | AGTGCCAAAGGGCTC | 2129 | X | 154534125 | + |
| 2328 | CTTGAAGGTGAGGAT | | CTTGAAGGTGAGGAT | | | | |
| | AACGCAGGCGATGTT | | AACGCAGGCGATGTT | | | | |
| | GTCCA | | GTCCC | | | | |
| rs13785 | AGTGCCAAAGGGCTC | 1455 | AGTGCCAAAGGGCTC | 2130 | X | 154534125 | + |
| 2328 | CTTGAAGGTGAGGAT | | CTTGAAGGTGAGGAT | | | | |
| | AACGCAGGCGATGTT | | AACGCAGGCGATGTT | | | | |
| | GTCCA | | GTCCC | | | | |
| rs13785 | AGTGCCAAAGGGCTC | 1456 | AGTGCCAAAGGGCTC | 2131 | X | 154534125 | + |
| 2328 | CTTGAAGGTGAGGAT | | CTTGAAGGTGAGGAT | | | | |
| | AACGCAGGCGATGTT | | AACGCAGGCGATGTT | | | | |
| | GTCCA | | GTCCC | | | | |
| X_ | TTGCCCCTCCCTGCA | 1457 | | | X | 154534126 | − |
| 154534126 | GATTTGCCAACAGGA | | | | | | |
| | TCTTCGGCCCCATCT | | | | | | |
| | GGAAC | | | | | | |
| X_ | AGTGCCAAAGGGCTC | 1458 | | | X | 154534126 | + |
| 154534126 | CTTGAAGGTGAGGAT | | | | | | |
| | AACGCAGGCGATGTT | | | | | | |
| | GTCCC | | | | | | |
| X_ | AGTGCCAAAGGGCTC | 1459 | | | X | 154534126 | + |
| 154534126 | CTTGAAGGTGAGGAT | | | | | | |
| | AACGCAGGCGATGTT | | | | | | |
| | GTCCC | | | | | | |
| rs13785 | CGCAGGCGATGTTGT | 1460 | CGCAGGCGATGTTGT | 2132 | X | 154534157 | + |
| 2319 | CCCGGTTCCAGATGG | | CCCGGTTCCAGATGG | | | | |
| | GGCCGAAGATCCTGT | | GGCCGAAGATCCTGT | | | | |
| | TGGCA | | TGGCC | | | | |
| rs13785 | CGCAGGCGATGTTGT | 1461 | CGCAGGCGATGTTGT | 2133 | X | 154534157 | + |
| 2319 | CCCGGTTCCAGATGG | | CCCGGTTCCAGATGG | | | | |
| | GGCCGAAGATCCTGT | | GGCCGAAGATCCTGT | | | | |
| | TGGCA | | TGGCC | | | | |
| rs13785 | CGCAGGCGATGTTGT | 1462 | CGCAGGCGATGTTGT | 2134 | X | 154534157 | + |
| 2319 | CCCGGTTCCAGATGG | | CCCGGTTCCAGATGG | | | | |
| | GGCCGAAGATCCTGT | | GGCCGAAGATCCTGT | | | | |
| | TGGCA | | TGGCC | | | | |
| rs13785 | CTACCGCATCGACCA | 1463 | CTACCGCATCGACCA | 2135 | X | 154534345 | − |
| 2326 | CTACCTGGGCAAGGA | | CTACCTGGGCAAGGA | | | | |
| | GATGGTGCAGAACCT | | GATGGTGCAGAACCT | | | | |
| | CRTGT | | CRTGG | | | | |
| rs13785 | CTACCGCATCGACCA | 1464 | CTACCGCATCGACCA | 2136 | X | 154534345 | − |
| 2326 | CTACCTGGGCAAGGA | | CTACCTGGGCAAGGA | | | | |
| | GATGGTGCAGAACCT | | GATGGTGCAGAACCT | | | | |
| | CATGT | | CATGG | | | | |
| rs13785 | CTACCGCATCGACCA | 1465 | CTACCGCATCGACCA | 2137 | X | 154534345 | − |
| 2326 | CTACCTGGGCAAGGA | | CTACCTGGGCAAGGA | | | | |
| | GATGGTGCAGAACCT | | GATGGTGCAGAACCT | | | | |
| | CATGT | | CATGG | | | | |
| rs78275 | AGATCTACCGCATCG | 1466 | | | X | 154534348 | − |
| 4619 | ACCACTACCTGGGCA | | | | | | |
| | AGGAGATGGTGCAGA | | | | | | |
| | ACCTC | | | | | | |
| rs78275 | AGATCTACCGCATCG | 1467 | | | X | 154534348 | − |
| 4619 | ACCACTACCTGGGCA | | | | | | |
| | AGGAGATGGTGCAGA | | | | | | |
| | ACCTC | | | | | | |
| rs78275 | AGATCTACCGCATCG | 1468 | | | X | 154534348 | − |
| 4619 | ACCACTACCTGGGCA | | | | | | |
| | AGGAGATGGTGCAGA | | | | | | |
| | ACCTC | | | | | | |
| rs78186 | CCTCAGCACCATGAG | 1469 | | | X | 154534387 | + |
| 5768 | GTTCTGCACCATCTC | | | | | | |
| | CTTGCCCAGGTAGTG | | | | | | |
| | GTCGA | | | | | | |
| rs78186 | CCTCAGCACCATGAG | 1470 | | | X | 154534387 | + |
| 5768 | GTTCTGCACCATCTC | | | | | | |
| | CTTGCCCAGGTAGTG | | | | | | |
| | GTCGA | | | | | | |
| rs78186 | GGCTGTCCAACCACA | 1471 | | | X | 154534387 | − |
| 5768 | TCTCCTCCCTGTTCC | | | | | | |
| | GTGAGGACCAGATCT | | | | | | |
| | ACCGC | | | | | | |
| rs13785 | CAGCACCATGAGGTT | 1472 | CAGCACCATGAGGTT | 2138 | X | 154534389 | + |
| 2332 | CTGCACCATCTCCTT | | CTGCACCATCTCCTT | | | | |
| | GCCCAGGTAGTGGTC | | GCCCAGGTAGTGGTC | | | | |
| | GAYGG | | GAYGC | | | | |
| rs13785 | CAGCACCATGAGGTT | 1473 | CAGCACCATGAGGTT | 2139 | X | 154534389 | + |
| 2332 | CTGCACCATCTCCTT | | CTGCACCATCTCCTT | | | | |
| | GCCCAGGTAGTGGTC | | GCCCAGGTAGTGGTC | | | | |
| | GAYGG | | GAYGC | | | | |
| rs13785 | CAGCACCATGAGGTT | 1474 | CAGCACCATGAGGTT | 2140 | X | 154534389 | + |
| 2332 | CTGCACCATCTCCTT | | CTGCACCATCTCCTT | | | | |
| | GCCCAGGTAGTGGTC | | GCCCAGGTAGTGGTC | | | | |
| | GAYGG | | GAYGC | | | | |
| rs13785 | AGCACCATGAGGTTC | 1475 | AGCACCATGAGGTTC | 2141 | X | 154534390 | + |
| 2330 | TGCACCATCTCCTTG | | TGCACCATCTCCTTG | | | | |
| | CCCAGGTAGTGGTCG | | CCCAGGTAGTGGTCG | | | | |
| | AYGCA | | AYGCG | | | | |
| rs13785 | CAGCACCATGAGGTT | 1476 | | | X | 154534390 | + |
| 2330 | CTGCACCATCTCCTT | | | | | | |
| | GCCCAGGTAGTGGTC | | | | | | |
| | GATGC | | | | | | |
| rs13785 | CAGCACCATGAGGTT | 1477 | | | X | 154534390 | + |
| 2330 | CTGCACCATCTCCTT | | | | | | |
| | GCCCAGGTAGTGGTC | | | | | | |
| | GATGC | | | | | | |
| X_ | CCATCTCCTTGCCCA | 1478 | CCATCTCCTTGCCCA | 2142 | X | 154534409 | + |
| 154534409 | GGTAGTGGTCGAYGB | | GGTAGTGGTCGAYGB | | | | |
| | RGTAGATCTGGTCCT | | RGTAGATCTGGTCCT | | | | |
| | CACGC | | CACGG | | | | |
| X_ | CCATCTCCTTGCCCA | 1479 | CCATCTCCTTGCCCA | 2143 | X | 154534409 | + |
| 154534409 | GGTAGTGGTCGAYGB | | GGTAGTGGTCGAYGB | | | | |
| | RGTAGATCTGGTCCT | | RGTAGATCTGGTCCT | | | | |
| | CACGC | | CACGG | | | | |
| X_ | CCATCTCCTTGCCCA | 1480 | CCATCTCCTTGCCCA | 2144 | X | 154534409 | + |
| 154534409 | GGTAGTGGTCGATGC | | GGTAGTGGTCGATGC | | | | |
| | GGTAGATCTGGTCCT | | GGTAGATCTGGTCCT | | | | |
| | CACGC | | CACGG | | | | |
| X_ | CCCAGGTAGTGGTCG | 1481 | CCCAGGTAGTGGTCG | 2145 | X | 154534418 | + |
| 154534418 | ATGCGGTAGATCTGG | | ATGCGGTAGATCTGG | | | | |
| | TCCTCACGSAACAGG | | TCCTCACGSAACAGG | | | | |
| | GAGGA | | GAGAT | | | | |
| X_ | CCCAGGTAGTGGTCG | 1482 | CCCAGGTAGTGGTCG | 2146 | X | 154534418 | + |
| 154534418 | ATGCGGTAGATCTGG | | ATGCGGTAGATCTGG | | | | |
| | TCCTCACGGAACAGG | | TCCTCACGGAACAGG | | | | |
| | GAGGA | | GAGAT | | | | |
| X_ | CCCAGGTAGTGGTCG | 1483 | CCCAGGTAGTGGTCG | 2147 | X | 154534418 | + |
| 154534418 | ATGCGGTAGATCTGG | | ATGCGGTAGATCTGG | | | | |
| | TCCTCACGGAACAGG | | TCCTCACGGAACAGG | | | | |
| | GAGGA | | GAGAT | | | | |
| rs50308 | TGCCCAGGTAGTGGT | 1484 | | | X | 154534419 | + |
| 68 | CGATGCGGTAGATCT | | | | | | |
| | GGTCCTCACGGAACA | | | | | | |
| | GGGAG | | | | | | |
| rs26760 | GCGGTAGATCTGGTC | 1485 | | | X | 154534438 | + |
| 6836 | CTCACGGAACAGGGA | | | | | | |
| | GRAGATGTGGTTGGA | | | | | | |
| | CAGCC | | | | | | |
| rs26760 | GCGGTAGATCTGGTC | 1486 | | | X | 154534438 | + |
| 6836 | CTCACGGAACAGGGA | | | | | | |
| | GRAGATGTGGTTGGA | | | | | | |
| | CAGCC | | | | | | |
| rs26760 | GCGGTAGATCTGGTC | 1487 | | | X | 154534438 | + |
| 6836 | CTCACGGAACAGGGA | | | | | | |
| | GGAGATGTGGTTGGA | | | | | | |
| | CAGCC | | | | | | |
| rs50308 | GTAGATCTGGTCCTC | 1488 | GTAGATCTGGTCCTC | 2148 | X | 154534440 | + |
| 72 | ACGGAACAGGGAGGA | | ACGGAACAGGGAGGA | | | | |
| | GATGTGGTTGGACAG | | GATGTGGTTGGACAG | | | | |
| | CCGGT | | CCGGA | | | | |
| rs50308 | GTAGATCTGGTCCTC | 1489 | GTAGATCTGGTCCTC | 2149 | X | 154534440 | + |
| 72 | ACGGAACAGGGAGRA | | ACGGAACAGGGAGRA | | | | |
| | GATGTGGTTGGACAG | | GATGTGGTTGGACAG | | | | |
| | CCRGA | | CCRGT | | | | |
| rs50308 | GTAGATCTGGTCCTC | 1490 | GTAGATCTGGTCCTC | 2150 | X | 154534440 | + |
| 72 | ACGGAACAGGGAGGA | | ACGGAACAGGGAGGA | | | | |
| | GATGTGGTTGGACAG | | GATGTGGTTGGACAG | | | | |
| | CCGGA | | CCGGT | | | | |
| X_ | TGGTCCTCACGGAAC | 1491 | TGGTCCTCACGGAAC | 2151 | X | 154534447 | + |
| 154534447 | AGGGAGGAGATGTGG | | AGGGAGGAGATGTGG | | | | |
| | TTGGACAGCCRGWCA | | TTGGACAGCCRGWCA | | | | |
| | GAGCT | | GAGCA | | | | |
| X_ | TGGTCCTCACGGAAC | 1492 | TGGTCCTCACGGAAC | 2152 | X | 154534447 | + |
| 154534447 | AGGGAGGAGATGTGG | | AGGGAGGAGATGTGG | | | | |
| | TTGGACAGCCRGWCA | | TTGGACAGCCRGWCA | | | | |
| | GAGCT | | GAGCA | | | | |
| X_ | TGGTCCTCACGGAAC | 1493 | TGGTCCTCACGGAAC | 2153 | X | 154534447 | + |
| 154534447 | AGGGAGGAGATGTGG | | AGGGAGGAGATGTGG | | | | |
| | TTGGACAGCCRGWCA | | TTGGACAGCCRGWCA | | | | |
| | GAGCT | | GAGCA | | | | |
| X_ | TGGTCCTCACGGAAC | 1494 | TGGTCCTCACGGAAC | 2154 | X | 154534447 | + |
| 154534447 | AGGGAGGAGATGTGG | | AGGGAGGAGATGTGG | | | | |
| | TTGGACAGCCRGWCA | | TTGGACAGCCRGWCA | | | | |
| | GAGCT | | GAGCA | | | | |
| X_ | CACGGAACAGGGAGG | 1495 | | | X | 154534455 | + |
| 154534455 | AGATGTGGTTGGACA | | | | | | |
| | GCCGGTCAGAGCTCT | | | | | | |
| | GCAGG | | | | | | |
| X_ | CACGGAACAGGGAGG | 1496 | | | X | 154534455 | + |
| 154534455 | AGATGTGGTTGGACA | | | | | | |
| | GCCGGTCAGAGCTCT | | | | | | |
| | GCAGG | | | | | | |
| X_ | CACGGAACAGGGAGG | 1497 | | | X | 154534455 | + |
| 154534455 | AGATGTGGTTGGACA | | | | | | |
| | GCCGGTCAGAGCTCT | | | | | | |
| | GCAGG | | | | | | |
| X_ | GGGAGGAGATGTGGT | 1498 | GGGAGGAGATGTGGT | 2155 | X | 154534463 | + |
| 154534463 | TGGACAGCCGGTCAG | | TGGACAGCCGGTCAG | | | | |
| | AGCTCTGCAGGTCCC | | AGCTCTGCAGGTCCC | | | | |
| | TCCCC | | TCCCG | | | | |
| X_ | GGGAGGAGATGTGGT | 1499 | GGGAGGAGATGTGGT | 2156 | X | 154534463 | + |
| 154534463 | TGGACAGCCGGTCAG | | TGGACAGCCGGTCAG | | | | |
| | AGCTCTGCAGGTCCC | | AGCTCTGCAGGTCCC | | | | |
| | TCCCC | | TCCCG | | | | |
| X_ | GGGAGGAGATGTGGT | 1500 | GGGAGGAGATGTGGT | 2157 | X | 154534463 | + |
| 154534463 | TGGACAGCCGGTCAG | | TGGACAGCCGGTCAG | | | | |
| | AGCTCTGCAGGTCCC | | AGCTCTGCAGGTCCC | | | | |
| | TCCCC | | TCCCG | | | | |
| X_ | CTCCGGGCTCCCAGC | 1501 | | | X | 154534463 | − |
| 154534463 | AGAGGCTGGAACCGC | | | | | | |
| | ATCATCGTGGAGAAG | | | | | | |
| | CCCTT | | | | | | |
| rs13785 | GGAGGAGATGTGGTT | 1502 | | | X | 154534465 | + |
| 2343 | GGACAGCCGGTCAGA | | | | | | |
| | GCTCTGCAGGTCCCT | | | | | | |
| | CCCGA | | | | | | |
| rs13785 | GGAGGAGATGTGGTT | 1503 | | | X | 154534465 | + |
| 2343 | GGACAGCCGGTCAGA | | | | | | |
| | GCTCTGCAGGTCCCT | | | | | | |
| | CCCGA | | | | | | |
| rs13785 | GGAGGAGATGTGGTT | 1504 | | | X | 154534465 | + |
| 2343 | GGACAGCCGGTCAGA | | | | | | |
| | GCTCTGCAGGTCCCT | | | | | | |
| | CCCGA | | | | | | |
| X_ | GGAGATGTGGTTGGA | 1505 | | | X | 154534468 | + |
| 154534468 | CAGCCGGTCAGAGCT | | | | | | |
| | CTGCAGGYCCCTCCC | | | | | | |
| | SARGG | | | | | | |
| X_ | GGAGATGTGGTTGGA | 1506 | | | X | 154534468 | + |
| 154534468 | CAGCCGGTCAGAGCT | | | | | | |
| | CTGCAGGTCCCTCCC | | | | | | |
| | GAAGG | | | | | | |
| X_ | GGAGATGTGGTTGGA | 1507 | | | X | 154534468 | + |
| 154534468 | CAGCCGGTCAGAGCT | | | | | | |
| | CTGCAGGTCCCTCCC | | | | | | |
| | GAAGG | | | | | | |
| X_ | GCCGGTCAGAGCTCT | 1508 | | | X | 154534485 | + |
| 154534485 | GCAGGTCCCTCCCSA | | | | | | |
| | RGGRCTTCTCCACGA | | | | | | |
| | TGATG | | | | | | |
| X_ | GCCGGTCAGAGCTCT | 1509 | | | X | 154534485 | + |
| 154534485 | GCAGGTCCCTCCCSA | | | | | | |
| | RGGRCTTCTCCACGA | | | | | | |
| | TGATG | | | | | | |
| X_ | GCCGGTCAGAGCTCT | 1510 | | | X | 154534485 | + |
| 154534485 | GCAGGTCCCTCCCSA | | | | | | |
| | RGGRCTTCTCCACGA | | | | | | |
| | TGATG | | | | | | |
| X_ | CCGGTCAGAGCTCTG | 1511 | | | X | 154534486 | + |
| 154534486 | CAGGTCCCTCCCSAR | | | | | | |
| | GGRCTTCTCCACGAT | | | | | | |
| | GATGC | | | | | | |
| X_ | CCGGTCAGAGCTCTG | 1512 | | | X | 154534486 | + |
| 154534486 | CAGGTCCCTCCCSAR | | | | | | |
| | GGRCTTCTCCACGAT | | | | | | |
| | GATGC | | | | | | |
| X_ | CCGGTCAGAGCTCTG | 1513 | | | X | 154534486 | + |
| 154534486 | CAGGTCCCTCCCGAA | | | | | | |
| | GGGCTTCTCCACGAT | | | | | | |
| | GATGC | | | | | | |
| X_ | CCGGTCAGAGCTCTG | 1514 | | | X | 154534486 | + |
| 154534486 | CAGGTCCCTCCCGAA | | | | | | |
| | GGGCTTCTCCACGAT | | | | | | |
| | GATGC | | | | | | |
| rs13785 | GTCAGAGCTCTGCAG | 1515 | | | X | 154534489 | + |
| 2331 | GTCCCTCCCGAAGGG | | | | | | |
| | CTTCTCCACGATGAT | | | | | | |
| | GCGGT | | | | | | |
| rs13785 | GTCAGAGCTCTGCAG | 1516 | | | X | 154534489 | + |
| 2331 | GTCCCTCCCGAAGGG | | | | | | |
| | CTTCTCCACGATGAT | | | | | | |
| | GCGGT | | | | | | |
| rs13785 | GTCAGAGCTCTGCAG | 1517 | | | X | 154534489 | + |
| 2331 | GTCCCTCCCGAAGGG | | | | | | |
| | CTTCTCCACGATGAT | | | | | | |
| | GCGGT | | | | | | |
| X_ | AGCTCTGCAGGTCCC | 1518 | | | X | 154534494 | + |
| 154534494 | TCCCGAAGGGCTTCT | | | | | | |
| | CCACGATGATGCGGT | | | | | | |
| | TCCAG | | | | | | |
| X_ | CCCCGAAGAGGGGTT | 1519 | | | X | 154534494 | − |
| 154534494 | CAAGGGGGTAACGCA | | | | | | |
| | GCTCCGGGCTCCCAG | | | | | | |
| | CAGAR | | | | | | |
| X_ | CCCCGAAGAGGGGTT | 1520 | | | X | 154534494 | − |
| 154534494 | CAAGGGGGTAACGCA | | | | | | |
| | GCTCCGGGCTCCCAG | | | | | | |
| | CAGAR | | | | | | |
| rs13785 | GCTCTGCAGGTCCCT | 1521 | | | X | 154534495 | + |
| 2314 | CCCGAAGGGCTTCTC | | | | | | |
| | CACGATGATGCGGTT | | | | | | |
| | CCAGC | | | | | | |
| rs13785 | GCTCTGCAGGTCCCT | 1522 | | | X | 154534495 | + |
| 2314 | CCCGAAGGGCTTCTC | | | | | | |
| | CACGATGATGCGGTT | | | | | | |
| | CCAGC | | | | | | |
| rs13785 | GCTCTGCAGGTCCCT | 1523 | | | X | 154534495 | + |
| 2314 | CCCGAAGGGCTTCTC | | | | | | |
| | CACGATGATGCGGTT | | | | | | |
| | CCAGC | | | | | | |
| rs13785 | GCTCTGCAGGTCCCT | 1524 | | | X | 154534495 | + |
| 2314 | CCCGAAGGGCTTCTC | | | | | | |
| | CACGATGATGCGGTT | | | | | | |
| | CCAGC | | | | | | |
| rs37091 | CCCCGACCGTCTACG | 1525 | CCCCGACCGTCTACG | 2158 | X | 154535176 | − |
| 8918 | AGGCCGTCACCAAGA | | AGGCCGTCACCAAGA | | | | |
| | ACATTCACGAGTCCT | | ACATTCACGAGTCCT | | | | |
| | GCATC | | GCATG | | | | |
| rs37091 | CCCCGACCGTCTACG | 1526 | CCCCGACCGTCTACG | 2159 | X | 154535176 | − |
| 8918 | AGGCCGTCACCAAGA | | AGGCCGTCACCAAGA | | | | |
| | ACATTCACGAGTCCT | | ACATTCACGAGTCCT | | | | |
| | GCATC | | GCATG | | | | |
| rs37091 | CCCCGACCGTCTACG | 1527 | CCCCGACCGTCTACG | 2160 | X | 154535176 | − |
| 8918 | AGGCCGTCACCAAGA | | AGGCCGTCACCAAGA | | | | |
| | ACATTCACGAGTCCT | | ACATTCACGAGTCCT | | | | |
| | GCATC | | GCATG | | | | |
| rs78248 | CTTGCCCCCGACCGT | 1528 | | | X | 154535180 | − |
| 7723 | CTACGAGGCCGTCAC | | | | | | |
| | CAAGAACATTCACGA | | | | | | |
| | GTCCT | | | | | | |
| rs78248 | CCTGGCAGGCGGGAA | 1529 | | | X | 154535180 | + |
| 7723 | GGGAGGGCAACGGCA | | | | | | |
| | AGCCTTACATCTGGC | | | | | | |
| | TCATG | | | | | | |
| rs78248 | CCTGGCAGGCGGGAA | 1530 | | | X | 154535180 | + |
| 7723 | GGGAGGGCAACGGCA | | | | | | |
| | AGCCTTACATCTGGC | | | | | | |
| | TCATG | | | | | | |
| rs13785 | ACCTGGCCTTGCCCC | 1531 | | | X | 154535187 | − |
| 2313 | CGACCGTCTACRAGG | | | | | | |
| | CCGTCACCAAGAACA | | | | | | |
| | TTSAC | | | | | | |
| rs13785 | ACCTGGCCTTGCCCC | 1532 | | | X | 154535187 | − |
| 2313 | CGACCGTCTACGAGG | | | | | | |
| | CCGTCACCAAGAACA | | | | | | |
| | TTCAC | | | | | | |
| rs13785 | GGCGGGAAGGGAGGG | 1533 | | | X | 154535187 | + |
| 2313 | CAACGGCAAGCCTTA | | | | | | |
| | CATCTGGCTCATGCA | | | | | | |
| | GGACT | | | | | | |
| X_ | CTACCTGGCCTTGCC | 1534 | CTACCTGGCCTTGCC | 2161 | X | 154535190 | − |
| 154535190 | CCCGACCGTCTACRA | | CCCGACCGTCTACRA | | | | |
| | GGCCGTCACCAAGAA | | GGCCGTCACCAAGAA | | | | |
| | CATTG | | CATTC | | | | |
| X_ | CTACCTGGCCTTGCC | 1535 | CTACCTGGCCTTGCC | 2162 | X | 154535190 | − |
| 154535190 | CCCGACCGTCTACRA | | CCCGACCGTCTACRA | | | | |
| | GGCCGTCACCAAGAA | | GGCCGTCACCAAGAA | | | | |
| | CATTG | | CATTC | | | | |
| X_ | CTACCTGGCCTTGCC | 1536 | CTACCTGGCCTTGCC | 2163 | X | 154535190 | − |
| 154535190 | CCCGACCGTCTACRA | | CCCGACCGTCTACRA | | | | |
| | GGCCGTCACCAAGAA | | GGCCGTCACCAAGAA | | | | |
| | CATTG | | CATTC | | | | |
| X_ | GCCTTACATCTGGCT | 1537 | | | X | 154535211 | + |
| 154535211 | CATGCAGGACTCGTG | | | | | | |
| | AATGTTCTTGGTGAC | | | | | | |
| | GGCCT | | | | | | |
| X_ | GCCTTACATCTGGCT | 1538 | | | X | 154535211 | + |
| 154535211 | CATGCAGGACTCGTG | | | | | | |
| | AATGTTCTTGGTGAC | | | | | | |
| | GGCCT | | | | | | |
| X_ | GCCTTACATCTGGCT | 1539 | | | X | 154535211 | + |
| 154535211 | CATGCAGGACTCGTG | | | | | | |
| | AATGTTCTTGGTGAC | | | | | | |
| | GGCCT | | | | | | |
| X_ | GTTCTTGGTGACGGC | 1540 | | | X | 154535244 | + |
| 154535244 | CTCGTAGACGGTCGG | | | | | | |
| | GGGCAAGGCCAGGTA | | | | | | |
| | GAAGA | | | | | | |
| X_ | GTTCTTGGTGACGGC | 1541 | | | X | 154535244 | + |
| 154535244 | CTCGTAGACGGTCGG | | | | | | |
| | GGGCAAGGCCAGGTA | | | | | | |
| | GAAGA | | | | | | |
| X_ | GCCTCAACAGCCACA | 1542 | | | X | 154535244 | − |
| 154535244 | TGAATGCCCTCCACC | | | | | | |
| | TGGGGTCACAGGCCA | | | | | | |
| | ACCGC | | | | | | |
| X_ | AGCGCCTCAACAGCC | 1543 | | | X | 154535247 | − |
| 154535247 | ACATGAATGCCCTCC | | | | | | |
| | ACCTGGGGTCACAGG | | | | | | |
| | CCAAC | | | | | | |
| X_ | AGCGCCTCAACAGCC | 1544 | | | X | 154535247 | − |
| 154535247 | ACATGAATGCCCTCC | | | | | | |
| | ACCTGGGGTCACAGG | | | | | | |
| | CCAAC | | | | | | |
| X_ | AGCGCCTCAACAGCC | 1545 | | | X | 154535247 | − |
| 154535247 | ACATGAATGCCCTCC | | | | | | |
| | ACCTGGGGTCACAGG | | | | | | |
| | CCAAC | | | | | | |
| rs78232 | CCAGCGCCTCAACAG | 1546 | | | X | 154535249 | − |
| 2505 | CCACATGAATGCCCT | | | | | | |
| | CCACCTGGGGTCACA | | | | | | |
| | GGCCA | | | | | | |
| rs78232 | CCAGCGCCTCAACAG | 1547 | | | X | 154535249 | − |
| 2505 | CCACATGAATGCCCT | | | | | | |
| | CCACCTGGGGTCACA | | | | | | |
| | GGCCA | | | | | | |
| rs78232 | CCAGCGCCTCAACAG | 1548 | | | X | 154535249 | − |
| 2505 | CCACATGAATGCCCT | | | | | | |
| | CCACCTGGGGTCACA | | | | | | |
| | GGCCA | | | | | | |
| rs13785 | GTAGACGGTCGGGGG | 1549 | GTAGACGGTCGGGGG | 2164 | X | 154535261 | + |
| 2341 | CAAGGCCAGGTAGAA | | CAAGGCCAGGTAGAA | | | | |
| | GARGCRGKTGGCCTG | | GARGCRGKTGGCCTG | | | | |
| | TGACA | | TGACC | | | | |
| rs13785 | GTAGACGGTCGGGGG | 1550 | GTAGACGGTCGGGGG | 2165 | X | 154535261 | + |
| 2341 | CAAGGCCAGGTAGAA | | CAAGGCCAGGTAGAA | | | | |
| | GARGCRGKTGGCCTG | | GARGCRGKTGGCCTG | | | | |
| | TGACA | | TGACC | | | | |
| rs13785 | GTAGACGGTCGGGGG | 1551 | GTAGACGGTCGGGGG | 2166 | X | 154535261 | + |
| 2341 | CAAGGCCAGGTAGAA | | CAAGGCCAGGTAGAA | | | | |
| | GARGCRGKTGGCCTG | | GARGCRGKTGGCCTG | | | | |
| | TGACA | | TGACC | | | | |
| X_ | GTCGGGGGCAAGGCC | 1552 | | | X | 154535269 | + |
| 154535269 | AGGTAGAAGAGGCGG | | | | | | |
| | TTGGCCTGTGACCCC | | | | | | |
| | AGGTG | | | | | | |
| X_ | GTCGGGGGCAAGGCC | 1553 | | | X | 154535269 | + |
| 154535269 | AGGTAGAAGAGGCGG | | | | | | |
| | TTGGCCTGTGACCCC | | | | | | |
| | AGGTG | | | | | | |
| X_ | GTCGGGGGCAAGGCC | 1554 | | | X | 154535269 | + |
| 154535269 | AGGTAGAAGAGGCGG | | | | | | |
| | TTGGCCTGTGACCCC | | | | | | |
| | AGGTG | | | | | | |
| rs78365 | CGGGGGCAAGGCCAG | 1555 | CGGGGGCAAGGCCAG | 2167 | X | 154535270 | + |
| 220 | GTAGAAGAGGCGGTT | | GTAGAAGAGGCGGTT | | | | |
| | GGCCTGTGACCCCAG | | GGCCTGTGACCCCAG | | | | |
| | GTGGA | | GTGGC | | | | |
| rs78365 | GTACGATGATGCAGC | 1556 | | | X | 154535270 | − |
| 220 | CTCCTACCAGCGCCT | | | | | | |
| | CAACAGCCACATGAA | | | | | | |
| | TGCCC | | | | | | |
| rs78365 | GTACGATGATGCAGC | 1557 | | | X | 154535270 | − |
| 220 | CTCCTACCAGCGCCT | | | | | | |
| | CAACAGCCACATGAA | | | | | | |
| | TGCCC | | | | | | |
| X_ | GGCAAGGCCAGGTAG | 1558 | GGCAAGGCCAGGTAG | 2168 | X | 154535274 | + |
| 154535274 | AAGAGGCGGTTGGCC | | AAGAGGCGGTTGGCC | | | | |
| | TGTGACCCCAGGTGG | | TGTGACCCCAGGTGG | | | | |
| | AGGGA | | AGGGC | | | | |
| X_ | GCCAGTACGATGATG | 1559 | | | X | 154535274 | − |
| 154535274 | CAGCCTCCTACCAGC | | | | | | |
| | GCCTCAACAGCCACA | | | | | | |
| | TKRAT | | | | | | |
| X_ | CCAGTACGATGATGC | 1560 | CCAGTACGATGATGC | 2169 | X | 154535274 | − |
| 154535274 | AGCCTCCTACCAGCG | | AGCCTCCTACCAGCG | | | | |
| | CCTCAACAGCCACAT | | CCTCAACAGCCACAT | | | | |
| | KRATT | | KRATG | | | | |
| rs10508 | TGGCCAGTACGATGA | 1561 | TGGCCAGTACGATGA | 2170 | X | 154535277 | − |
| 29 | TGCAGCCTCCYACCA | | TGCAGCCTCCYACCA | | | | |
| | GCGCCTCAACAGCCA | | GCGCCTCAACAGCCA | | | | |
| | CATKA | | CATKG | | | | |
| rs10508 | CAAGGCCAGGTAGAA | 1562 | | | X | 154535277 | + |
| 29 | GAGGCGGTTGGCCTG | | | | | | |
| | TGACCCCAGGTGGAG | | | | | | |
| | GGCAT | | | | | | |
| rs10508 | AAGGCCAGGTAGAAG | 1563 | AAGGCCAGGTAGAAG | 2171 | X | 154535277 | + |
| 29 | AGGCGGTTGGCCTGT | | AGGCGGTTGGCCTGT | | | | |
| | GACCCCAGGTGGAGG | | GACCCCAGGTGGAGG | | | | |
| | GCATT | | GCATC | | | | |
| X_ | GCTGGCCAGTACGAT | 1564 | | | X | 154535278 | − |
| 154535278 | GATGCAGCCTCCYAC | | | | | | |
| | CAGCGCCTCAACAGC | | | | | | |
| | CACAT | | | | | | |
| X_ | GCTGGCCAGTACGAT | 1565 | | | X | 154535278 | − |
| 154535278 | GATGCAGCCTCCYAC | | | | | | |
| | CAGCGCCTCAACAGC | | | | | | |
| | CACAT | | | | | | |
| X_ | GCTGGCCAGTACGAT | 1566 | | | X | 154535278 | − |
| 154535278 | GATGCAGCCTCCYAC | | | | | | |
| | CAGCGCCTCAACAGC | | | | | | |
| | CACAT | | | | | | |
| X_ | GGCCTGTGACCCCAG | 1567 | | | X | 154535301 | + |
| 154535301 | GTGGAGGGCATTCAT | | | | | | |
| | GTGGCTGTTGAGGCG | | | | | | |
| | CTGGT | | | | | | |
| X_ | GGCCTGTGACCCCAG | 1568 | | | X | 154535301 | + |
| 154535301 | GTGGAGGGCATTCAT | | | | | | |
| | GTGGCTGTTGAGGCG | | | | | | |
| | CTGGT | | | | | | |
| X_ | GGCCTGTGACCCCAG | 1569 | | | X | 154535301 | + |
| 154535301 | GTGGAGGGCATTCAT | | | | | | |
| | GTGGCTGTTGAGGCG | | | | | | |
| | CTGGT | | | | | | |
| rs50308 | TCAAGCTGGAGGACT | 1570 | | | X | 154535316 | − |
| 70 | TCTTTGCCCGCAACT | | | | | | |
| | CCTATGTGGCTGGCC | | | | | | |
| | AGTAC | | | | | | |
| rs50308 | TCAAGCTGGAGGACT | 1571 | | | X | 154535316 | − |
| 70 | TCTTTGCCCGCAACT | | | | | | |
| | CCTATGTGGCTGGCC | | | | | | |
| | AGTAC | | | | | | |
| rs50308 | TCAAGCTGGAGGACT | 1572 | | | X | 154535316 | − |
| 70 | TCTTTGCCCGCAACT | | | | | | |
| | CCTATGTGGCTGGCC | | | | | | |
| | AGTAC | | | | | | |
| X_ | GTGGCTGTTGAGGCG | 1573 | GTGGCTGTTGAGGCG | 2172 | X | 154535330 | + |
| 154535330 | CTGGTAGGAGGCTGC | | CTGGTAGGAGGCTGC | | | | |
| | ATCATCGTACTGGCC | | ATCATCGTACTGGCC | | | | |
| | AGCCA | | AGCCT | | | | |
| X_ | GTGGCTGTTGAGGCG | 1574 | GTGGCTGTTGAGGCG | 2173 | X | 154535330 | + |
| 154535330 | CTGGTAGGAGGCTGC | | CTGGTAGGAGGCTGC | | | | |
| | ATCATYGTACTGGCC | | ATCATYGTACTGGCC | | | | |
| | AGCCA | | AGCCT | | | | |
| X_ | GTGGCTGTTGAGGCG | 1575 | GTGGCTGTTGAGGCG | 2174 | X | 154535330 | + |
| 154535330 | CTGGTAGGAGGCTGC | | CTGGTAGGAGGCTGC | | | | |
| | ATCATYGTACTGGCC | | ATCATYGTACTGGCC | | | | |
| | AGCCA | | AGCCT | | | | |
| X_ | GTGGCTGTTGAGGCG | 1576 | GTGGCTGTTGAGGCG | 2175 | X | 154535330 | + |
| 154535330 | CTGGTAGGAGGCTGC | | CTGGTAGGAGGCTGC | | | | |
| | ATCATCGTACTGGCC | | ATCATCGTACTGGCC | | | | |
| | AGCCA | | AGCCT | | | | |
| rs26760 | GCCACCCCAGAGGAG | 1577 | GCCACCCCAGAGGAG | 2176 | X | 154535336 | − |
| 6835 | AAGCTCAAGCTGGAG | | AAGCTCAAGCTGGAG | | | | |
| | GACTTCTTTGCCCRC | | GACTTCTTTGCCCRC | | | | |
| | AACTG | | AACTC | | | | |
| rs26760 | GTTGAGGCGCTGGTA | 1578 | GTTGAGGCGCTGGTA | 2177 | X | 154535336 | + |
| 6835 | GGAGGCTGCATCATC | | GGAGGCTGCATCATC | | | | |
| | GTACTGGCCAGCCAC | | GTACTGGCCAGCCAC | | | | |
| | ATAGC | | ATAGG | | | | |
| rs26760 | GCCACCCCAGAGGAG | 1579 | GCCACCCCAGAGGAG | 2178 | X | 154535336 | − |
| 6835 | AAGCTCAAGCTGGAG | | AAGCTCAAGCTGGAG | | | | |
| | GACTTCTTTGCCCGC | | GACTTCTTTGCCCGC | | | | |
| | AACTC | | AACTG | | | | |
| rs18127 | GGCGCTGGTAGGAGG | 1580 | | | X | 154535342 | + |
| 7621 | CTGCATCATCGTACT | | | | | | |
| | GGCCAGCCACATAGG | | | | | | |
| | AGTTG | | | | | | |
| rs18127 | CTCCCAGGCCACCCC | 1581 | | | X | 154535342 | − |
| 7621 | AGAGGAGAAGCTCAA | | | | | | |
| | GCTGGAGGACTTCTT | | | | | | |
| | TGCCC | | | | | | |
| rs18127 | CTCCCAGGCCACCCC | 1582 | | | X | 154535342 | − |
| 7621 | AGAGGAGAAGCTCAA | | | | | | |
| | GCTGGAGGACTTCTT | | | | | | |
| | TGCCC | | | | | | |
| X_ | CTGGCCAGCCACATA | 1583 | | | X | 154535369 | + |
| 154535369 | GGAGTTGCGGGCAAA | | | | | | |
| | GAAGTCCTCCAGCTT | | | | | | |
| | GAGCT | | | | | | |
| X_ | CTGGCCAGCCACATA | 1584 | | | X | 154535369 | + |
| 154535369 | GGAGTTGCGGGCAAA | | | | | | |
| | GAAGTCCTCCAGCTT | | | | | | |
| | GAGCT | | | | | | |
| X_ | CTGGCCAGCCACATA | 1585 | | | X | 154535369 | + |
| 154535369 | GGAGTTGCGGGCAAA | | | | | | |
| | GAAGTCCTCCAGCTT | | | | | | |
| | GAGCT | | | | | | |
| X_ | CACATAGGAGTTGCG | 1586 | | | X | 154535379 | + |
| 154535379 | GGCAAAGAAGTCCTC | | | | | | |
| | CAGCTTGAGCTTCTC | | | | | | |
| | CTCTG | | | | | | |
| X_ | CACATAGGAGTTGCG | 1587 | | | X | 154535379 | + |
| 154535379 | GGCAAAGAAGTCCTC | | | | | | |
| | CAGCTTGAGCTTCTC | | | | | | |
| | CTCTG | | | | | | |
| X_ | CACATAGGAGTTGCG | 1588 | | | X | 154535379 | + |
| 154535379 | GGCAAAGAAGTCCTC | | | | | | |
| | CAGCTTGAGCTTCTC | | | | | | |
| | CTCTG | | | | | | |
| rs78230 | TGGGGGAGGCCCTGA | 1589 | | | X | 154535962 | + |
| 8266 | CACCACCCACCTTGA | | | | | | |
| | AGAAGGGCTCACTCT | | | | | | |
| | GTTTG | | | | | | |
| rs78230 | TGGGGGAGGCCCTGA | 1590 | | | X | 154535962 | + |
| 8266 | CACCACCCACCTTGA | | | | | | |
| | AGAAGGGCTCACTCT | | | | | | |
| | GTTTG | | | | | | |
| rs78230 | TGGGGGAGGCCCTGA | 1591 | | | X | 154535962 | + |
| 8266 | CACCACCCACCTTGA | | | | | | |
| | AGAAGGGCTCACTCT | | | | | | |
| | GTTTG | | | | | | |
| rs13868 | ACACCTTCATCGTGG | 1592 | | | X | 154535963 | − |
| 7036 | GCTATGCCCGTTCCC | | | | | | |
| | GCCTCACAGTGGCTG | | | | | | |
| | ACATC | | | | | | |
| rs13868 | GGGGGAGGCCCTGAC | 1593 | | | X | 154535963 | + |
| 7036 | ACCACCCACCTTGAA | | | | | | |
| | GAAGGGCTCACTCTG | | | | | | |
| | TTTGC | | | | | | |
| rs13868 | ACACCTTCATCGTGG | 1594 | | | X | 154535963 | − |
| 7036 | GCTATGCCCGTTCCC | | | | | | |
| | GCCTCACAGTGGCTG | | | | | | |
| | ACATC | | | | | | |
| X_ | CACCCACCTTGAAGA | 1595 | | | X | 154535980 | + |
| 154535980 | AGGGCTCACTCTGTT | | | | | | |
| | TGCGGATGTCAGCCA | | | | | | |
| | CTGTG | | | | | | |
| X_ | CACCCACCTTGAAGA | 1596 | | | X | 154535980 | + |
| 154535980 | AGGGCTCACTCTGTT | | | | | | |
| | TGCGGATGTCAGCCA | | | | | | |
| | CTGTG | | | | | | |
| X_ | CACCCACCTTGAAGA | 1597 | | | X | 154535980 | + |
| 154535980 | AGGGCTCACTCTGTT | | | | | | |
| | TGCGGATGTCAGCCA | | | | | | |
| | CTGTG | | | | | | |
| rs78209 | GTGGCTGTTCCGGGA | 1598 | | | X | 154535995 | − |
| 0947 | TGGCCTTCTGCCCGA | | | | | | |
| | AAACACCTTCATCGT | | | | | | |
| | GGGCT | | | | | | |
| rs78209 | GTGGCTGTTCCGGGA | 1599 | | | X | 154535995 | − |
| 0947 | TGGCCTTCTGCCCGA | | | | | | |
| | AAACACCTTCATCGT | | | | | | |
| | GGGCT | | | | | | |
| rs78209 | AGGGCTCACTCTGTT | 1600 | | | X | 154535995 | + |
| 0947 | TGCGGATGTCAGCCA | | | | | | |
| | CTGTGAGGCGGGAAC | | | | | | |
| | GGGCA | | | | | | |
| rs13785 | GGGCTCACTCTGTTT | 1601 | | | X | 154535996 | + |
| 2349 | GCGGATGTCAGCCAC | | | | | | |
| | TGTGAGGCGGGAACG | | | | | | |
| | GGCAT | | | | | | |
| rs13785 | GGTGGCTGTTCCGGG | 1602 | | | X | 154535996 | − |
| 2349 | ATGGCCTTCTGCCCG | | | | | | |
| | AAAACACCTTCATCG | | | | | | |
| | TGGGC | | | | | | |
| rs13785 | GGGCTCACTCTGTTT | 1603 | | | X | 154535996 | + |
| 2349 | GCGGATGTCAGCCAC | | | | | | |
| | TGTGAGGCGGGAACG | | | | | | |
| | GGCAT | | | | | | |
| rs10508 | ACTCTGTTTGCGGAT | 1604 | | | X | 154536002 | + |
| 28 | GTCAGCCACTGTGAG | | | | | | |
| | GCGGGAACGGGCATA | | | | | | |
| | GCCCA | | | | | | |
| rs10508 | ACTCTGTTTGCGGAT | 1605 | | | X | 154536002 | + |
| 28 | GTCAGCCACTGTGAG | | | | | | |
| | GCGGGAACGGGCATA | | | | | | |
| | GCCCA | | | | | | |
| rs10508 | CCCTCAGGTGGCTGT | 1606 | | | X | 154536002 | − |
| 28 | TCCGGGATGGCCTTC | | | | | | |
| | TGCCCGAAAACACCT | | | | | | |
| | TCATC | | | | | | |
| X_ | TTGCGGATGTCAGCC | 1607 | TTGCGGATGTCAGCC | 2179 | X | 154536008 | + |
| 154536008 | ACTGTGAGGCGGGAA | | ACTGTGAGGCGGGAA | | | | |
| | CGGGCAYRGCCCAYG | | CGGGCAYRGCCCAYG | | | | |
| | ATGAA | | ATGAT | | | | |
| X_ | TTGCGGATGTCAGCC | 1608 | TTGCGGATGTCAGCC | 2180 | X | 154536008 | + |
| 154536008 | ACTGTGAGGCGGGAA | | ACTGTGAGGCGGGAA | | | | |
| | CGGGCATAGCCCACG | | CGGGCATAGCCCACG | | | | |
| | ATGAA | | ATGAT | | | | |
| X_ | TTGCGGATGTCAGCC | 1609 | TTGCGGATGTCAGCC | 2181 | X | 154536008 | + |
| 154536008 | ACTGTGAGGCGGGAA | | ACTGTGAGGCGGGAA | | | | |
| | CGGGCATAGCCCACG | | CGGGCATAGCCCACG | | | | |
| | ATGAA | | ATGAT | | | | |
| X_ | AGCCACTGTGAGGCG | 1610 | AGCCACTGTGAGGCG | 2182 | X | 154536019 | + |
| 154536019 | GGAACGGGCATAGCC | | GGAACGGGCATAGCC | | | | |
| | CACGATGAAGGTGTT | | CACGATGAAGGTGTT | | | | |
| | TTCGA | | TTCGG | | | | |
| X_ | AGCCACTGTGAGGCG | 1611 | AGCCACTGTGAGGCG | 2183 | X | 154536019 | + |
| 154536019 | GGAACGGGCATAGCC | | GGAACGGGCATAGCC | | | | |
| | CACGATGAAGGTGTT | | CACGATGAAGGTGTT | | | | |
| | TTCGA | | TTCGG | | | | |
| X_ | AGCCACTGTGAGGCG | 1612 | AGCCACTGTGAGGCG | 2184 | X | 154536019 | + |
| 154536019 | GGAACGGGCATAGCC | | GGAACGGGCATAGCC | | | | |
| | CACGATGAAGGTGTT | | CACGATGAAGGTGTT | | | | |
| | TTCGA | | TTCGG | | | | |
| X_ | AGCCACTGTGAGGCG | 1613 | AGCCACTGTGAGGCG | 2185 | X | 154536019 | + |
| 154536019 | GGAACGGGCATAGCC | | GGAACGGGCATAGCC | | | | |
| | CACGATGAAGGTGTT | | CACGATGAAGGTGTT | | | | |
| | TTCGA | | TTCGG | | | | |
| X_ | ACTGTGAGGCGGGAA | 1614 | | | X | 154536021 | + |
| 154536021 | CGGGCATAGCCCACG | | | | | | |
| | ATGAAGGTGTTTTCG | | | | | | |
| | GGCAG | | | | | | |
| X_ | ACTGTGAGGCGGGAA | 1615 | | | X | 154536021 | + |
| 154536021 | CGGGCATAGCCCACG | | | | | | |
| | ATGAAGGTGTTTTCG | | | | | | |
| | GGCAG | | | | | | |
| X_ | ACTGTGAGGCGGGAA | 1616 | | | X | 154536021 | + |
| 154536021 | CGGGCATAGCCCACG | | | | | | |
| | ATGAAGGTGTTTTCG | | | | | | |
| | GGCAG | | | | | | |
| X_ | CTGTGAGGCGGGAAC | 1617 | | | X | 154536025 | + |
| 154536025 | GGGCATAGCCCACGA | | | | | | |
| | TGAAGGTGTTTTCGG | | | | | | |
| | GCAGA | | | | | | |
| X_ | CTGTGAGGCGGGAAC | 1618 | | | X | 154536025 | + |
| 154536025 | GGGCATAGCCCACGA | | | | | | |
| | TGAAGGTGTTTTCGG | | | | | | |
| | GCAGA | | | | | | |
| X_ | GCACTGGTTACAGCT | 1619 | | | X | 154536025 | − |
| 154536025 | GTGCCCTGCCCTCAG | | | | | | |
| | GTGGCTGTTCYRGRA | | | | | | |
| | TGGCC | | | | | | |
| rs13785 | GCGGGAACGGGCATA | 1620 | | | X | 154536032 | + |
| 2315 | GCCCACGATGAAGGT | | | | | | |
| | GTTTTCGGGCAGAAG | | | | | | |
| | GCCAT | | | | | | |
| rs13785 | GCGGGAACGGGCATA | 1621 | | | X | 154536032 | + |
| 2315 | GCCCACGATGAWGGT | | | | | | |
| | GTTTTCGDGCAGARG | | | | | | |
| | GCCAT | | | | | | |
| rs13785 | GCGGGAACGGGCATA | 1622 | | | X | 154536032 | + |
| 2315 | GCCCACGATGAAGGT | | | | | | |
| | GTTTTCGGGCAGAAG | | | | | | |
| | GCCAT | | | | | | |
| rs13785 | GCGGGAACGGGCATA | 1623 | | | X | 154536032 | + |
| 2315 | GCCCACGATGAAGGT | | | | | | |
| | GTTTTCGGGCAGAAG | | | | | | |
| | GCCAT | | | | | | |
| X_ | GGGAACGGGCATAGC | 1624 | | | X | 154536034 | + |
| 154536034 | CCACGATGAAGGTGT | | | | | | |
| | TTTCGGGCAGAAGGC | | | | | | |
| | CATCC | | | | | | |
| X_ | GGGAACGGGCATAGC | 1625 | | | X | 154536034 | + |
| 154536034 | CCACGATGAAGGTGT | | | | | | |
| | TTTCGDGCAGARGGC | | | | | | |
| | CATYC | | | | | | |
| X_ | GGGAACGGGCATAGC | 1626 | | | X | 154536034 | + |
| 154536034 | CCACGATGAAGGTGT | | | | | | |
| | TTTCGGGCAGAAGGC | | | | | | |
| | CATCC | | | | | | |
| X_ | GGAACGGGCATAGCC | 1627 | | | X | 154536035 | + |
| 154536035 | CACGATGAAGGTGTT | | | | | | |
| | TTCGGGCAGAAGGCC | | | | | | |
| | ATCCC | | | | | | |
| X_ | GGAACGGGCATAGCC | 1628 | | | X | 154536035 | + |
| 154536035 | CACGATGAAGGTGTT | | | | | | |
| | TTCGGGCAGAAGGCC | | | | | | |
| | ATCCC | | | | | | |
| X_ | GGAACGGGCATAGCC | 1629 | | | X | 154536035 | + |
| 154536035 | CACGATGAAGGTGTT | | | | | | |
| | TTCGGGCAGAAGGCC | | | | | | |
| | ATCCC | | | | | | |
| X_ | AGCCCACGATGAAGG | 1630 | AGCCCACGATGAAGG | 2186 | X | 154536045 | + |
| 154536045 | TGTTTTCGGGCAGAA | | TGTTTTCGGGCAGAA | | | | |
| | GGCCATYCYRGAACA | | GGCCATYCYRGAACA | | | | |
| | GCCAC | | GCCAG | | | | |
| X_ | AGCCCACGATGAAGG | 1631 | AGCCCACGATGAAGG | 2187 | X | 154536045 | + |
| 154536045 | TGTTTTCGGGCAGAA | | TGTTTTCGGGCAGAA | | | | |
| | GGCCATYCYRGAACA | | GGCCATYCYRGAACA | | | | |
| | GCCAC | | GCCAG | | | | |
| X_ | AGCCCACGATGAAGG | 1632 | AGCCCACGATGAAGG | 2188 | X | 154536045 | + |
| 154536045 | TGTTTTCGGGCAGAA | | TGTTTTCGGGCAGAA | | | | |
| | GGCCATCCCGGAACA | | GGCCATCCCGGAACA | | | | |
| | GCCAC | | GCCAG | | | | |
| X_ | CACACCTGTTCCCTC | 1633 | | | X | 154536151 | − |
| 154536151 | TGCCACAGGGTGACC | | | | | | |
| | TGGCCAAGAAGAAGA | | | | | | |
| | TCTAC | | | | | | |
| X_ | CACACCTGTTCCCTC | 1634 | | | X | 154536151 | − |
| 154536151 | TGCCACAGGGTGACC | | | | | | |
| | TGRSCAAGAAGAAGA | | | | | | |
| | YCTAC | | | | | | |
| X_ | ACACCTGTTCCCTCT | 1635 | ACACCTGTTCCCTCT | 2189 | X | 154536151 | − |
| 154536151 | GCCACAGGGTGACCT | | GCCACAGGGTGACCT | | | | |
| | GGCCAAGAAGAAGAT | | GGCCAAGAAGAAGAT | | | | |
| | CTACT | | CTACC | | | | |
| rs76645 | GGGAGGTCACAGGGG | 1636 | | | X | 154536156 | + |
| 461 | CAGTGGTGGGACACA | | | | | | |
| | CTTACCAGATGGTGG | | | | | | |
| | GGTAG | | | | | | |
| rs76645 | GGGAGGTCACAGGGG | 1637 | | | X | 154536156 | + |
| 461 | CAGTGGTGGGACACA | | | | | | |
| | CTTACCAGATGGTGG | | | | | | |
| | RGTAG | | | | | | |
| rs76645 | GGGAGGTCACAGGGG | 1638 | | | X | 154536156 | + |
| 461 | CAGTGGTGGGACACA | | | | | | |
| | CTTACCAGATGGTGG | | | | | | |
| | RGTAG | | | | | | |
| rs78478 | CAGCCACTTCTAACC | 1639 | CAGCCACTTCTAACC | 2190 | X | 154536168 | − |
| 128 | ACACACCTGTTCCCT | | ACACACCTGTTCCCT | | | | |
| | CTGCCACAGGGTGAC | | CTGCCACAGGGTGAC | | | | |
| | CTGGG | | CTGGC | | | | |
| X_ | GCAGTGGTGGGACAC | 1640 | GCAGTGGTGGGACAC | 2191 | X | 154536169 | + |
| 154536169 | ACTTACCAGATGGTG | | ACTTACCAGATGGTG | | | | |
| | GGGTAGATCTTCTTC | | GGGTAGATCTTCTTC | | | | |
| | TTGGT | | TTGGC | | | | |
| X_ | GCAGTGGTGGGACAC | 1641 | GCAGTGGTGGGACAC | 2192 | X | 154536169 | + |
| 154536169 | ACTTACCAGATGGTG | | ACTTACCAGATGGTG | | | | |
| | GRGTAGRTCTTCTTC | | GRGTAGRTCTTCTTC | | | | |
| | TTGGT | | TTGGC | | | | |
| X_ | CCAGCCACTTCTAAC | 1642 | CCAGCCACTTCTAAC | 2193 | X | 154536169 | − |
| 154536169 | CACACACCTGTTCCC | | CACACACCTGTTCCC | | | | |
| | TCTGCCACAGGGTGA | | TCTGCCACAGGGTGA | | | | |
| | CCTGA | | CCTGG | | | | |
| X_ | ACTTCCTGGCTTTTA | 1643 | | | X | 154546046 | + |
| 154546046 | AGATTGGGGCCTGGG | | | | | | |
| | AGATACTCACCGATG | | | | | | |
| | CACCC | | | | | | |
| X_ | ACTTCCTGGCTTTTA | 1644 | | | X | 154546046 | + |
| 154546046 | AGATTGGGGCCTGGG | | | | | | |
| | AGATACTCACCGATG | | | | | | |
| | CACCC | | | | | | |
| X_ | ACTTCCTGGCTTTTA | 1645 | | | X | 154546046 | + |
| 154546046 | AGATTGGGGCCTGGG | | | | | | |
| | AGATACTCACCGATG | | | | | | |
| | CACCC | | | | | | |
| rs13785 | CTTTTAAGATTGGGG | 1646 | CTTTTAAGATTGGGG | 2194 | X | 154546050 | + |
| 2338 | CCTGGGAGATACTCA | | CCTGGGAGATACTCA | | | | |
| | CCGATGCACCCRTGA | | CCGATGCACCCRTGA | | | | |
| | TGATG | | TGAAT | | | | |
| rs13785 | CTTTTAAGATTGGGG | 1647 | CTTTTAAGATTGGGG | 2195 | X | 154546050 | + |
| 2338 | CCTGGGAGATACTCA | | CCTGGGAGATACTCA | | | | |
| | CCGATGCACCCRTGA | | CCGATGCACCCRTGA | | | | |
| | TGATG | | TGAAT | | | | |
| rs13785 | CTTTTAAGATTGGGG | 1648 | CTTTTAAGATTGGGG | 2196 | X | 154546050 | + |
| 2338 | CCTGGGAGATACTCA | | CCTGGGAGATACTCA | | | | |
| | CCGATGCACCCATGA | | CCGATGCACCCATGA | | | | |
| | TGATG | | TGAAT | | | | |
| X_ | CGGGAAGAGCTTTTC | 1649 | | | X | 154546057 | − |
| 154546057 | CAGGGCGATGCCTTC | | | | | | |
| | CATCAGTCGGATACA | | | | | | |
| | CACAT | | | | | | |
| X_ | CGGGAAGAGCTTTTC | 1650 | | | X | 154546057 | − |
| 154546057 | CAGGGCGATGCCTTC | | | | | | |
| | CATCAGTCGGATACA | | | | | | |
| | CACAT | | | | | | |
| X_ | CGGGAAGAGCTTTTC | 1651 | | | X | 154546057 | − |
| 154546057 | CAGGGCGATGCCTTC | | | | | | |
| | CATCAGTCGGATACA | | | | | | |
| | CACAT | | | | | | |
| rs13785 | CCTGCGGGAAGAGCT | 1652 | | | X | 154546061 | − |
| 2340 | TTTCCAGGGCGATGC | | | | | | |
| | CTTCCATCAGTCGGA | | | | | | |
| | TACAC | | | | | | |
| rs13785 | CCTGCGGGAAGAGCT | 1653 | | | X | 154546061 | − |
| 2340 | TTTCCAGGGCGATGC | | | | | | |
| | CTTCCATCAGTCGGA | | | | | | |
| | TACAC | | | | | | |
| rs13785 | CCTGCGGGAAGAGCT | 1654 | | | X | 154546061 | − |
| 2340 | TTTCCAGGGCGATGC | | | | | | |
| | CTTCCATCAGTCGGA | | | | | | |
| | TACAC | | | | | | |
| X_ | ATCCGACTGATGGAA | 1655 | | | X | 154546116 | + |
| 154546116 | GGCATCGCCCTGGAA | | | | | | |
| | AAGCTCTTCCCGCAG | | | | | | |
| | GATCC | | | | | | |
| X_ | ATCCGACTGATGGAA | 1656 | | | X | 154546116 | + |
| 154546116 | GGCATCGCCCTGGAA | | | | | | |
| | AAGCTCTTCCCGCAG | | | | | | |
| | GATCC | | | | | | |
| X_ | ATCCGACTGATGGAA | 1657 | | | X | 154546116 | + |
| 154546116 | GGCATCGCCCTGGAA | | | | | | |
| | AAGCTCTTCCCGCAG | | | | | | |
| | GATCC | | | | | | |
| X_ | TTCCACCAGACAGCG | 1658 | | | X | 154546122 | − |
| 154546122 | TCATGGCAGAGCAGG | | | | | | |
| | TGGCCCTGAGCCGGA | | | | | | |
| | CCCAG | | | | | | |
| X_ | TTCCACCAGACAGCG | 1659 | | | X | 154546122 | − |
| 154546122 | TCATGGCAGAGCAGG | | | | | | |
| | TGGCCCTGAGCYGGA | | | | | | |
| | CCCAG | | | | | | |
| X_ | TTCCACCAGACAGCG | 1660 | | | X | 154546122 | − |
| 154546122 | TCATGGCAGAGCAGG | | | | | | |
| | TGGCCCTGAGCCGGA | | | | | | |
| | CCCAG | | | | | | |
| X_ | GGCATCGCCCTGGAA | 1661 | | | X | 154546131 | + |
| 154546131 | AAGCTCTTCCCGCAG | | | | | | |
| | GATCCCGCACACCTG | | | | | | |
| | GGTCC | | | | | | |
| X_ | GGCATCGCCCTGGAA | 1662 | | | X | 154546131 | + |
| 154546131 | AAGCTCTTCCCGCAG | | | | | | |
| | GATCCYGCACAMCTG | | | | | | |
| | GGTCC | | | | | | |
| X_ | GCATCGCCCTGGAAA | 1663 | GCATCGCCCTGGAAA | 2197 | X | 154546131 | + |
| 154546131 | AGCTCTTCCCGCAGG | | AGCTCTTCCCGCAGG | | | | |
| | ATCCYGCACAMCTGG | | ATCCYGCACAMCTGG | | | | |
| | GTCCA | | GTCCG | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of genotyping one or more pharmacogenomic markers, the method comprising:
obtaining a nucleic acid sample;
amplifying a first portion of the genomic DNA sample by whole genome amplification (WGA), thereby producing a WGA sample portion;
amplifying a second portion of the genomic DNA sample by a targeted gene amplification (TGA) method that selectively amplifies one or more pharmacogenomic genes or fragments thereof, thereby producing a TGA sample portion;
optionally combining the whole genome amplified sample portion and the target amplified sample portion to produce a combined WGA/TGA sample portion;
fragmenting the WGA or WGA/TGA sample;
hybridizing the WGA and TGA samples or the WGA/TGA combined sample to a plurality of probes complementary to one or more of the pharmacogenomic genes or fragments thereof; and
detecting hybridization, thereby genotyping a pharmacogenomic marker.

2. The method of claim 1, wherein the nucleic acid sample is a genomic DNA sample is from a human subject.

3. The method of claim 1, wherein the one or more pharmacogenomic genes are selected from the group consisting of ABCB1, ABCC4, ABCG2, ACE, ALDH1A1, ALK, BCKDK, BCR, BRAF, C8orf34, CACNA1S, CES1, CFTR, CHRNA3, COL22A1, COQ2, CRHR1, CYP19A1, CYP21A2, CYP2A6, CYP2A7P1, CYP2B6, CYP2C19, CYP2C8, CYP2C9, CYP2D6, CYP2E1, CYP3A, CYP3A5, CYP4F2, DPYD, DYNC2H1, EGFR, EPHX1, ERBB2, F5, FCGR3A, FKBP5, G6PD, GGCX, GSTM1, GSTT1, HCP5, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PRSS53, PSORS1C1, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT1A5, UGT2B17, UGT2B28, VDR, VKORC1, YEATS4, ZNRD1-AS1, and combinations thereof.

4. The method of claim 3,
wherein the one or more pharmacogenomic genes are selected from the group consisting of BCKDK, CACNA1S, CFTR, CYP2A7P1, CYP2B6, CYP2B6, CYP2C19, CYP2C9, CYP2D6, CYP3A, CYP3A5, CYP4F2, DPYD, F5, G6PD, HCP5, HLA-A, IFNL3, NUDT15, PRSS53, PSORS1C1, RYR1, SLCO1B1, TPMT, UGT1A1, VKORC1, ZNRD1-AS1, and combinations thereof; or
wherein the one or more pharmacogenomic genes are selected from the group consisting of ABCB1, ABCC4, ABCG2, ACE, ALDH1A1, ALK, BCR, BRAF, C8orf34, CACNA1S, CES1, CFTR, CHRNA3, COL22A1, COQ2, CRHR1, CYP19A1, CYP21A2, CYP2A6, CYP2B6, CYP2C19, CYP2C8, CYP2C9, CYP2D6, CYP2E1, CYP3A5, CYP4F2, DPYD, DYNC2H1, EGFR, EPHX1, ERBB2, F5, FCGR3A, FKBP5, G6PD, GGCX, GSTM1, GSTT1, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT2B17, UGT2B28, VDR, VKORC1, YEATS4, and combinations thereof.

5. (canceled)

6. The method of claim 1, wherein the one or more pharmacogenomic markers are selected from those in Table 9.

7. The method of claim 6,
wherein the one or more pharmacogenomic markers are copy number variants in ABCB1, ABCC4, ABCG2, ACE, ALDH1A1, ALK, BCR, BRAF, C8orf34, CACNA1S, CES1, CFTR, CHRNA3, COL22A1, COQ2, CRHR1, CYP19A1, CYP21A2, CYP2A6, CYP2B6, CYP2C19, CYP2C8, CYP2C9, CYP2D6, CYP2E1, CYP3A5, CYP4F2, DPYD, DYNC2H1, EGFR, EPHX1, ERBB2, F5, FCGR3A, FKBP5, G6PD, GGCX, GSTM1, GSTT1, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT2B17, UGT2B28, VDR, VKORC1, YEATS4, and combinations thereof, or
wherein the one or more pharmacogenomic markers are selected from the group consisting of those in Table 9, copy number variants in ABCB1, ABCC4, ABCG2, ACE, ALDH1A1, ALK, BCR, BRAF, C8orf34, CACNA1S, CES1, CFTR, CHRNA3, COL22A1, COQ2, CRHR1, CYP19A1, CYP21A2, CYP2A6, CYP2B6, CYP2C19, CYP2C8, CYP2C9, CYP2D6, CYP2E1, CYP3A5, CYP4F2, DPYD, DYNC2H1, EGFR, EPHX1, ERBB2, F5, FCGR3A, FKBP5, G6PD, GGCX, GSTM1, GSTT1, HLA-A, HLA-B, HLA-C, HLA-DPB1, HLA-DQA1, HLA-DRB1, HMGCR, IFNL3, IFNL4, KCNIP4, KIF6, KIT, LPA, MTRR, NEDD4L, NQO1, NUDT15, OPRM1, POLG, POR, PRKCA, PTGFR, RYR1, SCN1A, SCN5A, SEMA3C, SLC19A1, SLC28A3, SLC6A4, SLCO1B1, SLCO1B3, SOD2, SULT1A1, TANC1, TBXAS1, TMEM43, TPMT, TXNRD2, TYMS, UGT1A1, UGT1A4, UGT2B17, UGT2B28, VDR, VKORC1, YEATS4, and combinations thereof.

8. (canceled)

9. The method of claim 1, wherein amplifying a second portion of the genomic DNA sample comprises amplifying one or more regions of one or more pharmacogenomic genes or fragments thereof that differ in their nucleotide sequence from one or more pseudogenes of the one or more pharmacogenomic genes, optionally wherein the one or more regions of the one or more pharmacogenomic genes or fragments thereof share at least 99%, 98%, 97%, 96%, 95%, 94%, 93%, 92%, 91%, or 90% identity in its nucleic acid sequence with the corresponding one or more pseudogenes or fragments thereof.

10. (canceled)

11. The method of claim 1, wherein:
detecting hybridization comprises single-base extension (SBE), allele-specific primer extension (ASPE), or both SBE and ASPE, and/or
wherein amplifying comprises a PCR reaction with a dNTP mixture comprising dATP, dTTP, dGTP, dCTP, and dUTP and a dUTP incorporating polymerase; and/or
wherein fragmenting comprises incubating with uracil DNA glycosylase (UDG).

12. (canceled)

13. (canceled)

14. A method of selecting a drug treatment for a patient in need thereof, the method comprising:
identifying a patient in need of a drug treatment;
determining, or having determined, the genotype of pharmacogenomic marker(s) according to the method of claim 1; and
based on said genotyping, selecting a drug treatment for the patient.

15. The method of claim 14,
wherein selecting a drug treatment for the patient comprises determining that a drug is suitable for administration to the patient by identifying one or more drug interactions with the genotype of one or more the pharmacogenomic markers, and, optionally, administering a drug with a positive treatment outcome associated with one or more of the genotypes of the one or more pharmacogenomic markers and/or not administering a drug with a negative treatment outcome associated with one or more of the genotypes of the one or more pharmacogenomic markers, or
wherein selecting a drug treatment includes determining that a drug is not suitable for administration to the patient and, optionally, not administering the drug.

16. (canceled)

17. An array composition comprising:
a solid surface; and
one or more nucleic acids selected from the group consisting of nucleic acids comprising or consisting of the sequences of SEQ ID NOS: 22-2217, wherein the one or more nucleic acids are bound to the solid surface.

18. The array composition of claim 17, comprising:
(i) at least 100 nucleic acids selected from the group consisting of nucleic acids comprising or consisting of the sequences of SEQ ID NOS: 22-2217; or
(ii) at least 500 nucleic acids selected from the group consisting of nucleic acids comprising or consisting of the sequences of SEQ ID NOS: 22-2217;
(iii) at least 1000 nucleic acids selected from the group consisting of nucleic acids comprising or consisting of the sequences of SEQ ID NOS: 22-2217; or
(iv) at least 1500 nucleic acids selected from the group consisting of nucleic acids comprising or consisting of the sequences of SEQ ID NOS: 22-2217.

19. A composition comprising one or more nucleic acids selected from the group of nucleic acids comprising or consisting of SEQ ID NOS: 4-19 and, optionally, a polymerase and/or a target nucleic acid.

20. The composition of claim 19, wherein the composition comprises nucleic acids comprising or consisting of:
(i) SEQ ID NO:4 and SEQ ID NO:5;
(ii) SEQ ID NO:6 and SEQ ID NO:7;
(iii) SEQ ID NO:8 and SEQ ID NO:9;
(iv) SEQ ID NO:10 and SEQ ID NO:11;
(v) SEQ ID NO:12 and SEQ ID NO:13;
(vi) SEQ ID NO:14 and SEQ ID NO:15;
(vii) SEQ ID NO:16 and SEQ ID NO:17; and/or
(viii) SEQ ID NO:18 and SEQ ID NO:19.

21. The composition of claim 19, wherein the composition comprises oligonucleotides comprising or consisting of the sequences of SEQ ID NOS: 4, 5, 6, 7, 8, 9, 10, 11, 12, 13, 14, 15, 16, 17, 18, and 19.

22. A kit comprising the composition of claim 19.

23. The kit of claim 22, wherein the composition comprises or consists of:
(i) SEQ ID NO:4 and SEQ ID NO:5;
(ii) SEQ ID NO:6 and SEQ ID NO:7;
(iii) SEQ ID NO:8 and SEQ ID NO:9;
(iv) SEQ ID NO:10 and SEQ ID NO:11;
(v) SEQ ID NO:12 and SEQ ID NO:13;
(vi) SEQ ID NO:14 and SEQ ID NO:15;
(vii) SEQ ID NO:16 and SEQ ID NO:17; and/or
(viii) SEQ ID NO:18 and SEQ ID NO:19.

24. The kit of claim 22, further comprising:
a dNTP mixture; and
a polymerase,
optionally wherein the dNTP mixture comprises dUTP and the polymerase is a dUTP incorporating polymerase.

25. (canceled)

26. The kit of claim 24, further comprising:
uracil DNA nucleotide glycosylase and/or
the array composition of claim 20 or claim 21; and/or
random oligonucleotides for WGA.

27. (canceled)

28. (canceled)

29. A method of amplifying a target nucleic acid by polymerase chain reaction, the method comprising contacting the target nucleic acid with the composition of claim 19, wherein the composition comprises a polymerase.
